(19) United States
(12) Patent Application Publication
(10) Pub. No.: US 2026/0274920 A1
(43) Pub. Date: Sep. 17, 2026

(54) ENGINEERED ARTIFICIAL ANTIGEN PRESENTING CELLS FOR TUMOR INFILTRATING LYMPHOCYTE EXPANSION

(71) Applicant: Iovance Biotherapeutics, Inc., San Carlos, CA (US)

(72) Inventors: Anand Veerapathran, Tampa, FL (US); Aishwary Gokuldass, Chennai (IN); Brian Rabinovich, Winchester, MA (US); Michael T. Lotze, Pittsburgh, PA (US)

(21) Appl. No.: 19/699,401

(22) Filed: Jun. 5, 2026

Related U.S. Application Data
(63) Continuation of application No. 17/229,127, filed on Apr. 13, 2021, now Pat. No. 12,673,982.
Application No. 17/229,127 is a continuation of application No. 16/526,353, filed on Jul. 30, 2019, now Pat. No. 11,667,890.
Application No. 16/526,353 is a continuation of application No. 15/800,967, filed on Nov. 1, 2017, now Pat. No. 10,415,015.
Application No. 15/800,967 is a continuation of application No. PCT/US17/59271, filed on Oct. 31, 2017.
(60) Provisional application No. 62/415,274, filed on Oct. 31, 2016.
(60) Provisional application No. 62/438,600, filed on Dec. 23, 2016.
(60) Provisional application No. 62/475,053, filed on Mar. 22, 2017.
(60) Provisional application No. 62/481,831, filed on Apr. 5, 2017.

(51) Int. Cl. C07K 14/705; A61K 39/00; A61K 40/11; A61K 40/42; A61P 35/00; C07K 16/42; C12N 5/078; C12N 5/0781; C12N 5/0784

(52) U.S. Cl. CPC C07K 14/70575; A61K 40/11; A61K 40/42; A61P 35/00; C07K 14/70532; C07K 16/4283; C12N 5/0635; C12N 5/0639; C12N 5/065; A61K 2039/515; C07K 2317/622; C12N 2501/06; C12N 2501/2302; C12N 2501/25; C12N 2501/51; C12N 2501/515; C12N 2501/998; C12N 2502/11; C12N 2502/99

(57) ABSTRACT

In some embodiments, compositions and methods relating to isolated artificial antigen presenting cells (aAPCs) are disclosed, including aAPCs comprising a myeloid cell transduced with one or more viral vectors, such as a MOLM-14 or a EM-3 myeloid cell, wherein the myeloid cell endogenously expresses HLA-A/B/C, ICOS-L, and CD58, and wherein the one or more viral vectors comprise a nucleic acid encoding CD86 and a nucleic acid encoding 4-1BBL and/or OX40L and transduce the myeloid cell to express CD86 and 4-1BBL and/or OX40L proteins. In some embodiments, methods of expanding tumor infiltrating lymphocytes (TILs) with aAPCs and methods of treating cancers using TILs after expansion with aAPCs are also disclosed.

### CROSS-REFERENCE TO RELATED APPLICATIONS

This application is a continuation of U.S. patent application Ser. No. 17/229,127, filed Apr. 13, 2021, which is a continuation of U.S. patent application Ser. No. 16/526,353, filed on Jul. 30, 2019, now U.S. Pat. No. 11,667,890 issued on Jun. 6, 2023, which is a continuation of U.S. patent application Ser. No. 15/800,967, filed Nov. 1, 2017, now U.S. Pat. No. 10,415,015 issued on Sep. 17, 2019, which is a continuation of International Application No. PCT/US17/59271, filed Oct. 31, 2017, which claims the benefit of priority to U.S. Provisional Application No. 62/481,831, filed Apr. 5, 2017, U.S. Provisional Application No. 62/475,053, filed Mar. 22, 2017, U.S. Provisional Application No. 62/438,600, filed Dec. 23, 2016, and U.S. Provisional Application No. 62/415,274, filed Oct. 31, 2016, the entireties of which are incorporated herein by reference.

### SEQUENCE LISTING

The instant application contains a Sequence Listing which has been submitted electronically in XML file format and is hereby incorporated by reference in its entirety. Said XML file, created on Jun. 1, 2026, is named 5002-US04_Sequence_Listing. XML and is 180,224 bytes in size.

### FIELD OF THE INVENTION

Engineered artificial antigen presenting cells (aAPCs) for expansion of tumor infiltrating lymphocytes are disclosed.

### BACKGROUND OF THE INVENTION

Treatment of bulky, refractory cancers using adoptive autologous transfer of tumor infiltrating lymphocytes (TILs) represents a powerful approach to therapy for patients with poor prognoses. Gattinoni, et al., Nat. Rev. Immunol. 2006, 6, 383-393. A large number of TILs are required for successful immunotherapy, and a robust and reliable process is needed for commercialization. This has been a challenge to achieve because of technical, logistical, and regulatory issues with cell expansion. IL-2-based TIL expansion followed by a “rapid expansion process” (REP) has become a preferred method for TIL expansion because of its speed and efficiency. Dudley, et al., Science 2002, 298, 850-54; Dudley, et al., J. Clin. Oncol. 2005, 23, 2346-57; Dudley, et al., J. Clin. Oncol. 2008, 26, 5233-39; Riddell, et al., Science 1992, 257, 238-41; Dudley, et al., J. Immunother. 2003, 26, 332-42. However, although REP can result in a 1,000-fold expansion of TILs over a 14-day period, it requires a large excess (e.g., 200-fold) of irradiated allogeneic peripheral blood mononuclear cells (PBMCs), often from multiple donors, as feeder cells, as well as anti-CD3 antibody (OKT-3) and high doses of IL-2. Dudley, et al., J. Immunother. 2003, 26, 332-42. Despite their high performance, PBMCs have multiple drawbacks, including the large numbers of allogeneic PBMCs required, the need to obtain PBMCs by leukapheresis from multiple healthy donors, the resulting interdonor variability in PBMC viability after cryopreservation and variable TIL expansion results, the risk of undetected viral pathogens causing downstream patient infections, and the extensive and costly laboratory testing of each individual donor cell product to confirm sterility and quality (including viral contaminant testing) and to test expansion properties.

Unfortunately, aAPCs developed for use in the expansion of TILs have suffered from poor performance when compared to PBMCs, including alterations of the phenotypic properties of the input TILs, as well as poor expansion performance and/or high variability in expansion results. Because of the large number of potential cells that might be adapted for use as aAPCs and the unpredictability of identifying suitable candidates, the focus of aAPC development for polyclonal TILs to date has been solely on the well-established K562 cell line. Butler and Hirano, Immunol. Rev. 2014, 257, 191-209. For example, K562 cells modified to express 4-1BBL (CD137L) were tested in pre-REP culture (but not in REP culture) to determine enhancement of TIL expansion from tumor digest, but PBMCs were still required to be used in conjunction with K562 cells to obtain TIL expansion. Friedman, et al., J. Immunother. 2011, 34, 651-661. Other engineered K562 cells modified to express CD64, CD86, and 4-1BBL were tested and achieved TIL expansion that was at best comparable to PBMCs, and most likely less than PBMCs, and also suffered from skewing of the polyclonal TIL phenotype to a less favorable CD8⁺/CD4⁺ T cell ratio. Ye, et al., J. Translat. Med. 2011, 9, 131. Recently, K562 cells modified to express CD86, 4-1BBL (CD137L), high affinity Fc receptor (CD64) and membrane-bound IL-15 have also been shown to propagate TIL (post-REP) at equivalent numbers compared to PBMC feeders, but with the additional complexity of membrane-bound IL-15. Forget, et al., J. Immunother. 2014, 37, 448-60. Other systems developed have lacked critical costimulatory molecules, have led to unfavorable T cell phenotypic skewing, or have required additional interleukins (such as IL-21). Butler and Hirano, Immunol. Rev. 2014, 257, 191-209. Overall, K562 modified aAPCs have not been shown to provide for consistent expansion of TILs with acceptable variability while also performing better than PBMCs in other measures including overall expansion cell counts. Alternative aAPCs besides K562 cells have been successful in other cell expansion methods, but have not achieved the same performance as PBMCs with the unique polyclonal subset of cells that make up TILs. Maus, et al., Nat. Biotechnol. 2002, 20, 143-148; Suhoski, et al., Mol. Ther. 2007, 15, 981-988.

The MOLM-14 human leukemia cell line was established from the peripheral blood of a patient with relapsed acute monocytic leukemia, and initial phenotypic characterization indicated the presence of at least the following markers: CD4, CD9, CD11a, CD13, CD14, CD15, CD32, CD33, CD64, CD65, CD87, CD92, CD93, CD116, CD118, and CD155. Matsuo, et al., Leukemia 1997, 11, 1469-77. Additional phenotypic characterization of MOLM-14 found higher levels of HLA-A/B/C, CD64, CD80, ICOS-L, CD58, and lower levels of CD86. MOLM-14 cells and the closely-related MOLM-13 cells have not been previously reported as useful aAPCs for the expansion of cells for tumor immunotherapy applications.

The EM-3 human cell line was established from the bone marrow of a patient with Philadelphia chromosome-positive CML. Konopka, et al., Proc. Nat'l Acad. Sci. USA 1985, 82, 1810-4. EM-3 cells and the closely-related EM-2 cell line have not been previously reported as useful aAPCs for the expansion of cells for tumor immunotherapy applications. Phenotypic characterization for EM-3 cells indicates the presence of at least the following markers: CD13, CD15, and CD33.

The present invention provides the unexpected finding that engineered myeloid lineage cells, including MOLM-13, MOLM-14, EM-3, and EM-2 cells, transduced with additional costimulatory molecules, including CD86 (B7-2), 4-1BBL (CD137L), and OX40L (CD134L), provide for superior and highly efficient expansions of TILs in large numbers with minimal variability, reduced cost, and no reliance on human blood samples as a source of PBMCs, with the benefit of using an aAPC which can be produced efficiently from a master cell bank. CD86 and 4-1BBL are costimulatory molecules that provide costimulatory signals for T cell activation. The MOLM-14, MOLM-13, EM-3, and/or EM-2 cells transduced with additional costimulatory molecules are useful, for example, in the expansion of TILs for use in cancer immunotherapy and other therapies.

### SUMMARY OF THE INVENTION

In an embodiment, the invention provides an artificial antigen presenting cell (aAPC) comprising a myeloid cell transduced with one or more vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein.

In an embodiment, each of the CD86 protein and the 4-1BBL protein are human proteins.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the aAPC can stimulate and expand a tumor infiltrating lymphocyte (TIL) contacted with the aAPC.

It will be apparent that in certain embodiments of the invention, the nucleic acid molecule encoding CD86 may be comprised in a different viral vector to the nucleic acid molecule encoding 4-1BBL or the same viral vector.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the aAPC expands a population of TILs by at least 50-fold over a period of 7 days in a cell culture medium comprising IL-2 at a concentration of about 3000 IU/mL and OKT-3 antibody at a concentration of about 30 ng/mL.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the aAPC can stimulate and expand a T cell contacted with the aAPC.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the myeloid cell endogenously expresses HLA-A/B/C, ICOS-L, and CD58.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the myeloid cell is essentially devoid of membrane-bound IL-15.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the myeloid cell is a MOLM-14 cell.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the myeloid cell is a MOLM-13 cell.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the myeloid cell is a EM-3 cell.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the myeloid cell is a EM-2 cell.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the CD86 protein comprises an amino acid sequence as set forth in SEQ ID NO:8, or an amino acid sequence comprising one or more conservative amino acid substitutions thereof, and the 4-1BBL protein comprises SEQ ID NO:9, or an amino acid sequence comprising one or more conservative amino acid substitutions thereof.

In an embodiment, the invention provides an aAPC comprising a myeloid cell transduced with one or more viral vectors, wherein the one or more viral vectors comprise a nucleic acid molecule encoding CD86 and a nucleic acid molecule encoding 4-1BBL, and wherein the myeloid cell expresses a CD86 protein and a 4-1BBL protein, wherein the nucleic acid molecule encoding CD86 comprises a nucleic acid sequence as set forth in SEQ ID NO:16 and the nucleic acid molecule encoding 4-1BBL comprises a nucleic acid sequence as set forth in SEQ ID NO: 19.

In an embodiment, the invention provides a method of expanding tumor infiltrating lymphocytes (TILs), the method comprising the step of contacting a population of TILs with an aAPC comprising a myeloid cell transduced with one or more viral vectors, wherein the one or more viral vectors comprise a nucleic acid molecule encoding CD86 and a nucleic acid molecule encoding 4-1BBL, wherein the myeloid cell expresses a CD86 protein and a 4-1BBL protein, and wherein the population of TILs is expanded. In an embodiment, the method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and

(b) contacting the population of TILs with the population of aAPCs in a cell culture medium.

In an embodiment, the foregoing method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and
(b) contacting the population of TILs with the population of aAPCs in a cell culture medium, wherein the cell culture medium further comprises IL-2 at an initial concentration of about 3000 IU/mL and OKT-3 antibody at an initial concentration of about 30 ng/ml.

In an embodiment, the foregoing method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and
(b) contacting the population of TILs with the population of aAPCs in a cell culture medium, wherein the population of APCs expands the population of TILs by at least 50-fold over a period of 7 days in a cell culture medium.

In an embodiment, the foregoing method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and
(b) contacting the population of TILs with the population of aAPCs in a cell culture medium, wherein the myeloid cell endogenously expresses HLA-A/B/C, ICOS-L, and CD58.

In an embodiment, the foregoing method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and
(b) contacting the population of TILs with the population of aAPCs in a cell culture medium, wherein the myeloid cell is a MOLM-14 cell.

In an embodiment, the foregoing method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and
(b) contacting the population of TILs with the population of aAPCs in a cell culture medium, wherein the myeloid cell is a MOLM-13 cell.

In an embodiment, the foregoing method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and
(b) contacting the population of TILs with the population of aAPCs in a cell culture medium, wherein the myeloid cell is a EM-3 cell.

In an embodiment, the foregoing method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and
(b) contacting the population of TILs with the population of aAPCs in a cell culture medium, wherein the myeloid cell is a EM-2 cell.

In an embodiment, the foregoing method is an in vitro or an ex vivo method.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid molecule encoding CD86 and a nucleic acid molecule encoding 4-1BBL, and wherein the myeloid cell expresses a CD86 protein and a 4-1BBL protein, and
(b) contacting the population of TILs with the population of aAPCs in a cell culture medium, wherein the CD86 protein comprises an amino acid sequence as set forth in SEQ ID NO: 8, or comprises an amino acid sequence comprising one or more conservative amino acid substitutions thereof, and the 4-1BBL protein comprises an amino acid sequence as set forth in SEQ ID NO:9, or comprises an amino acid sequence comprising one or conservative amino acid substitutions thereof.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium, wherein the nucleic acid encoding CD86 comprises a nucleic acid sequence as set forth in SEQ ID NO:16 and the nucleic acid encoding 4-1BBL comprises a nucleic acid sequence as set forth in SEQ ID NO:19.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium, wherein the expansion is performed using a gas permeable container.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium, wherein the ratio of the population of TILs to the population of aAPCs is between 1 to 200 and 1 to 400.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium, wherein the ratio of the population of TILs to the population of aAPCs is about 1 to 300.

In an embodiment, the invention provides a method of expanding tumor infiltrating lymphocytes (TILs), the method comprising contacting a population of TILs comprising a population of TILs with a myeloid artificial antigen presenting cell (aAPC), wherein the myeloid aAPC comprises at least two co-stimulatory ligands that specifically bind with at least two co-stimulatory molecules on the TILs, wherein binding of the co-stimulatory molecules with the co-stimulatory ligand induces proliferation of the TILs, thereby specifically expanding TILs, and wherein the at least two co-stimulatory ligands comprise CD86 and 4-1BBL. In an embodiment, the foregoing method is an in vitro or ex vivo method.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, and wherein the myeloid aAPCs are transduced to express a CD86 protein and a 4-1BBL protein.

In an embodiment, the invention provides a population of tumor infiltrating lymphocytes (TILs) for use in treating cancer, wherein the TILs are a second population of TILs and are obtainable from a method comprising the steps of:
(a) performing a rapid expansion of a first population of TILs using a population of myeloid artificial antigen presenting cells (myeloid aAPCs) in a cell culture medium to obtain the second population of TILs, wherein the TILs are/have been obtained from a tumor resected from a patient, and wherein the second population of TILs is at least 50-fold greater in number than the first population of TILs after 7 days from the start of the rapid expansion; and
wherein the myeloid aAPCs endogenously expresses HLA-A/B/C, ICOS-L, and CD58, and wherein the myeloid aAPCs are transduced to express a CD86 protein and a 4-1BBL protein.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, wherein the myeloid aAPCs are transduced to express a CD86 protein and a 4-1BBL protein, wherein the myeloid aAPCs comprise MOLM-14 cells transduced with one or more viral vectors, and wherein the one or more viral vectors comprise a nucleic acid encoding CD86 and a nucleic acid encoding 4-1BBL, and wherein the MOLM-14 cells express a CD86 protein and a 4-1BBL protein.

In an embodiment, the invention provides a population of tumor infiltrating cells (TILs) for use in treating a cancer, wherein the population of TILs is a second population of TILs and is obtainable by a process comprising:
(a) performing a rapid expansion of a first population of TILs using a population of myeloid artificial antigen presenting cells (myeloid aAPCs) in a cell culture medium to obtain the second population of TILs, wherein the first population of TILs are/have been obtained from a tumor resected from a patient, wherein the second population of TILs is at least 50-fold greater in number than the first population of TILs after 7 days from the start of the rapid expansion;
wherein the myeloid aAPCs endogenously expresses HLA-A/B/C, ICOS-L, and CD58, wherein the myeloid aAPCs are transduced to express a CD86 protein and a 4-1BBL protein, wherein the myeloid aAPCs comprise MOLM-14 cells transduced with one or more viral vectors, and wherein the one or more viral vectors comprise a nucleic acid encoding CD86 and a nucleic acid encoding 4-1BBL, and wherein the MOLM-14 cells express a CD86 protein and a 4-1BBL protein.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, wherein the myeloid aAPCs are transduced to express a CD86 protein and a 4-1BBL protein, wherein the myeloid aAPCs comprise EM-3 cells transduced with one or more viral vectors, and wherein the one or more viral vectors comprise a nucleic acid encoding CD86 and a nucleic acid encoding 4-1BBL, and wherein the EM-3 cells express a CD86 protein and a 4-1BBL protein.

In an embodiment, the invention provides a population of tumor infiltrating lymphocytes (TILs) for use in treating a cancer, the population of TILs being a second population of TILs and obtainable by a process comprising:
(a) performing a rapid expansion of a first population of TILs using a population of myeloid artificial antigen presenting cells (myeloid aAPCs) in a cell culture medium to obtain the second population of TILs, wherein the first population of TILs are/have been obtained from a tumor resected from a patient, and wherein the second population of TILs is at least 50-fold greater in number than the first population of TILs after 7 days from the start of the rapid expansion; and
wherein the myeloid aAPCs endogenously expresses HLA-A/B/C, ICOS-L, and CD58, wherein the myeloid aAPCs are transduced to express a CD86 protein and a 4-1BBL protein, wherein the myeloid aAPCs comprise EM-3 cells transduced with one or more viral vectors, and wherein the one or more viral vectors comprise a nucleic acid encoding CD86 and a nucleic acid encoding 4-1BBL, and wherein the EM-3 cells express a CD86 protein and a 4-1BBL protein.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, wherein the myeloid aAPCs are transduced to express a CD86 protein and a 4-1BBL protein, and wherein the rapid expansion is performed over a period not greater than 14 days.

In an embodiment, the invention provides a population of tumor infiltrating lymphocytes (TILs) for use in treating a cancer, wherein the population of TILs is a second population and is obtainable by a method comprising the steps of:
(a) performing a rapid expansion of the first population of TILs using a population of myeloid artificial antigen presenting cells (myeloid aAPCs) in a cell culture medium to obtain the second population of TILs, wherein the second population of TILs is at least 50-fold greater in number than the first population of TILs after 7 days from the start of the rapid expansion, wherein the myeloid aAPCs endogenously express HLA-A/B/C, ICOS-L and CD58, wherein the myeloid aAPCs are transduced to express a CD86 protein and a 4-1BBL protein, and wherein the rapid expansion is performed over a period not greater than 14 days.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, wherein the myeloid aAPCs are transduced to express a CD86 protein and a 4-1BBL protein, and wherein the cell culture medium further comprises IL-2 at an initial concentration of about 3000 IU/mL and OKT-3 antibody at an initial concentration of about 30 ng/ml.

In an embodiment, the invention provides a population of tumor infiltrating lymphocytes (TILs) for use in treating a cancer, the population of TILs being a second population of TILs and obtainable by a process comprising:
(a) performing a rapid expansion of a first population of TILs using a population of myeloid artificial antigen presenting cells (myeloid aAPCs) in a cell culture medium to obtain the second population of TILs, wherein the first population of TILs are/have been obtained from a tumor resected from a patient, and wherein the second population of TILs is at least 50-fold greater in number than the first population of TILs after 7 days from the start of the rapid expansion; and wherein the myeloid aAPCs endogenously express HLA-A/B/C, ICOS-L, and CD58, wherein the myeloid aAPCs are transduced to express a CD86 protein and a 4-1BBL protein, and wherein the cell culture medium further comprises IL-2 at an initial concentration of about 3000 IU/mL and OKT-3 antibody at an initial concentration of about 30 ng/ml.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, wherein the myeloid aAPCs are transduced to express a CD86 protein and a 4-1BBL protein, and wherein the expansion is performed using a gas permeable container.

In an embodiment, the invention provides a population of tumor infiltrating lymphocytes (TILs) for use in treating a cancer, the population of TILs being a second population of TILs and obtainable by a process comprising:
(a) performing a rapid expansion of a first population of TILs using a population of myeloid artificial antigen presenting cells (myeloid aAPCs) in a cell culture medium to obtain the second population of TILs, wherein the first population of TILs are/have been obtained from a tumor resected from a patient, and wherein the second population of TILs is at least 50-fold greater in number than the first population of TILs after 7 days from the start of the rapid expansion; and wherein the myeloid aAPCs endogenously express HLA-A/B/C, ICOS-L, and CD58, wherein the myeloid aAPCs are transduced to express a CD86 protein and a 4-1BBL protein, and wherein the expansion is performed using a gas permeable container.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, wherein the myeloid aAPCs are transduced to express a CD86 protein and a 4-1BBL protein, and wherein the ratio of the second population of TILs to the population of aAPCs is between 1 to 200 and 1 to 400.

In an embodiment, the invention provides a population of tumor infiltrating cells (TILs) for use in treating a cancer, the population of TILs being a second population of TILs and obtainable by a process comprising the steps of:
(a) performing a rapid expansion of a first population of TILs using a population of myeloid artificial antigen presenting cells (myeloid aAPCs) in a cell culture medium to obtain the second population of TILs, wherein the first population of TILs is/has been obtained from a tumor resected from a patient, and wherein the second population of TILs is at least 50-fold greater in number than the first population of TILs after 7 days from the start of the rapid expansion; and
wherein the myeloid aAPCs endogenously expresses HLA-A/B/C, ICOS-L, and CD58, wherein the myeloid aAPCs are transduced to express a CD86 protein and a 4-1BBL protein, and wherein the ratio of the second population of TILs to the population of aAPCs is between 1 to 200 and 1 to 400. In certain embodiments, the ratio of the second population of TILs to the population of aAPCs is about 1 to 300.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, wherein the myeloid aAPCs are transduced to express a CD86 protein and a 4-1BBL protein, and wherein the ratio of the second population of TILs to the population of aAPCs is about 1 to 300.

In an embodiment, the invention provides a method of treating a cancer with a population of tumor infiltrating lymphocytes (TILs) comprising the steps of:
(a) obtaining a first population of TILs from a tumor resected from a patient;
(b) performing a rapid expansion of the first population of TILs using a population of myeloid artificial antigen presenting cells (myeloid aAPCs) in a cell culture medium to obtain a second population of TILs, wherein the second population of TILs is at least 50-fold greater in number than the first population of TILs after 7 days from the start of the rapid expansion; and
(c) administering a therapeutically effective portion of the second population of TILs to a patient with the cancer;
wherein the myeloid aAPCs endogenously expresses HLA-A/B/C, ICOS-L, and CD58, wherein the myeloid aAPCs are transduced to express a CD86 protein and a 4-1BBL protein, wherein the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer, renal cancer, and renal cell carcinoma.

In an embodiment, the invention provides a population of tumor infiltrating lymphocytes (TILs) for use in treating a cancer, the population of TILs being a second population of TILs and obtainable by a method comprising the steps of:
(a) performing a rapid expansion of a first population of TILs using a population of myeloid artificial antigen presenting cells (myeloid aAPCs) in a cell culture medium to obtain the second population of TILs, wherein the first population of TILs is/has been obtained from a tumor resected from a patient, and wherein the second population of TILs is at least 50-fold greater in number than the first population of TILs after 7 days from the start of the rapid expansion; and
wherein the myeloid aAPCs endogenously expresses HLA-A/B/C, ICOS-L, and CD58, wherein the myeloid aAPCs are transduced to express a CD86 protein and a 4-1BBL protein, wherein the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer, renal cancer, and renal cell carcinoma.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3;
(d) administering a therapeutically effective portion of the third population of TILs to a patient with the cancer.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3;
(d) administering a therapeutically effective portion of the third population of TILs to a patient with the cancer,
wherein the myeloid aAPCs comprise MOLM-14 cells transduced with one or more viral vectors, wherein the one or more viral vectors comprise a nucleic acid encoding CD86 and a nucleic acid encoding 4-1BBL, and wherein the MOLM-14 cells express a CD86 protein and a 4-1BBL protein.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3;
(d) administering a therapeutically effective portion of the third population of TILs to a patient with the cancer,
wherein the myeloid aAPCs comprise EM-3 cells transduced with one or more viral vectors, wherein the one or more viral vectors comprise a nucleic acid encoding CD86 and a nucleic acid encoding 4-1BBL, and wherein the EM-3 cells express a CD86 protein and a 4-1BBL protein.

In an embodiment, the invention provides a population of tumor infiltrating lymphocytes (TILs) for use in treating a cancer, wherein the population of TILs is a third population of TILs and obtainable by a method comprising the steps of:
(a) performing an initial expansion of a first population of TILs in a first cell culture medium to obtain a second population of TILs, wherein the first population of TILs is/has been obtained from a tumor resected from a patient, and wherein the second population of TILs is at least 5-fold greater in number than the first population of TILs, and wherein the first cell culture medium comprises IL-2;
(b) performing a rapid expansion of the second population of TILs using a population of myeloid artificial antigen presenting cells (aAPCs) in a second cell culture medium to obtain the third population of TILs, wherein the third population of TILs is at least 50-fold greater in number than the second population of TILs after 7 days from the start of the rapid expansion; and wherein the second cell culture medium comprises IL-2 and OKT-3.

In an embodiment, the myeloid aAPCs comprise MOLM-14 cells transduced with one or more viral vectors, wherein the one or more viral vectors comprise a nucleic acid encoding CD86 and a nucleic acid encoding 4-1BBL, and wherein the MOLM-14 cells express a CD86 protein and a 4-1BBL protein. In an embodiment, the myeloid cells comprise MOLM-13 cells transduced with one or more viral vectors, wherein the one or more viral vectors comprise a nucleic acid encoding CD86 and a nucleic acid encoding 4-1BBL, and wherein the MOLM-13 cells express a CD86 protein and a 4-1BBL protein. In certain embodiments, the myeloid cells comprise EM-3 cells transduced with one or more viral vectors, wherein the one or more viral vectors comprise a nucleic acid encoding CD86 and a nucleic acid encoding 4-1BBL, and wherein the EM-3 cells express a CD86 protein and a 4-1BBL protein. In certain embodiments, the myeloid cells comprise EM-2 cells transduced with one or more viral vectors, wherein the one or more viral vectors comprise a nucleic acid encoding CD86 and a nucleic acid encoding 4-1BBL, and wherein the EM-2 cells express a CD86 protein and a 4-1BBL protein.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3;
(d) treating the patient with a non-myeloablative lymphodepletion regimen, wherein the non-myeloablative lymphodepletion regimen comprises the steps of administration of cyclophosphamide at a dose of 60 mg/m²/day for two days followed by administration of fludarabine at a dose of 25 mg/m²/day for five days;
(e) administering a therapeutically effective portion of the third population of TILs to a patient with the cancer; and
(f) treating the patient with a high-dose IL-2 regimen, wherein the high-dose IL-2 regimen comprises 600,000 or 720,000 IU/kg of aldesleukin administered as a 15-minute bolus intravenous infusion every eight hours until tolerance;
wherein the myeloid aAPCs comprise MOLM-14 cells transduced with one or more viral vectors, wherein the one or more viral vectors comprise a nucleic acid encoding CD86 and a nucleic acid encoding 4-1BBL, and wherein the MOLM-14 cells express a CD86 protein and a 4-1BBL protein.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3;
(d) treating the patient with a non-myeloablative lymphodepletion regimen, wherein the non-myeloablative lymphodepletion regimen comprises the steps of administration of cyclophosphamide at a dose of 60 mg/m²/day for two days followed by administration of fludarabine at a dose of 25 mg/m²/day for five days;
(e) administering a therapeutically effective portion of the third population of TILs to a patient with the cancer; and
(f) treating the patient with a high-dose IL-2 regimen, wherein the high-dose IL-2 regimen comprises 600,000 or 720,000 IU/kg of aldesleukin administered as a 15-minute bolus intravenous infusion every eight hours until tolerance;
wherein the myeloid aAPCs comprise EM-3 cells transduced with one or more viral vectors, wherein the one or more viral vectors comprise a nucleic acid encoding CD86 and a nucleic acid encoding 4-1BBL, and wherein the EM-3 cells express a CD86 protein and a 4-1BBL protein.

In an embodiment, the invention provides a population of tumor infiltrating lymphocytes (TILs) for use in treating a cancer, wherein the population of TILs are a third population of TILs and obtainable by a method comprising the steps of:
(a) an initial expansion of a first population of TILs in a first cell culture medium to obtain a second population of TILs, wherein the first population of TILs is/has been obtained from a tumor resected from a patient, and wherein the second population of TILs is at least 5-fold greater in number than the first population of TILs, and wherein the first cell culture medium comprises IL-2; and
(b) performing a rapid expansion of the second population of TILs using a population of myeloid artificial antigen presenting cells (aAPCs) in a second cell culture medium to obtain the third population of TILs, wherein the third population of TILs is at least 50-fold greater in number than the second population of TILs after 7 days from the start of the rapid expansion; and wherein the second cell culture medium comprises IL-2 and OKT-3;
   and further wherein the population of TILs is for administration to a patient in combination with a non-myeloablative lymphodepletion regimen, wherein the non-myeloablative lymphodepletion regimen comprises cyclophosphamide which is for administration at a dose of 60 mg/m²/day for two days followed by fludarabine which is for administration at a dose of 25 mg/m²/day for five days and further wherein the population of TILs is for administration in combination with a high-dose IL-2 regimen, wherein the high-dose IL-2 regimen comprises 600,000 or 720,000 IU/kg of aldesleukin for administration as a 15-minute bolus intravenous infusion every eight hours until tolerance. In certain embodiments, the population of TILs is for administration prior to the high-dose IL-2 regimen and subsequent to the non-myeloablative lymphodepletion regimen.

In certain embodiments, the myeloid aAPCs comprise MOLM-14 cells transduced with one or more viral vectors, wherein the one or more viral vectors comprise a nucleic acid encoding CD86 and a nucleic acid encoding 4-1BBL, and wherein the MOLM-14 cells express a CD86 protein and a 4-1BBL protein, the myeloid aAPCs comprise MOLM-13 cells transduced with one or more viral vectors, wherein the one or more viral vectors comprise a nucleic acid encoding CD86 and a nucleic acid encoding 4-1BBL, and wherein the MOLM-13 cells express a CD86 protein and a 4-1BBL protein. In certain embodiments, the myeloid aAPCs comprise EM-3 cells transduced with one or more viral vectors, wherein the one or more viral vectors comprise a nucleic acid encoding CD86 and a nucleic acid encoding 4-1BBL, and wherein the EM-3 cells express a CD86 protein and a 4-1BBL protein.

In an embodiment, the population of TILs is for use in the treating of a cancer selected from the group consisting of melanoma, ovarian cancer, cervical cancer, non-small-cell lung cancer (NSCLC), lung cancer, bladder cancer, breast cancer, cancer caused by human papilloma virus, head and neck cancer, renal cancer, and renal cell carcinoma.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3; and
(d) administering a therapeutically effective portion of the third population of TILs to a patient with the cancer,
wherein IL-2 is present at an initial concentration of about 3000 IU/mL and OKT-3 antibody is present at an initial concentration of about 30 ng/mL in the second cell culture medium.

In an embodiment, the invention provides a population of tumor infiltrating lymphocytes (TILs) for use in treating a cancer, wherein the population of TILs is a third population of TILs and is obtainable by a method comprising the steps:
(a) performing an initial expansion of a first population of TILs in a first cell culture medium to obtain a second population of TILs, wherein the first population of TILs is/has been obtained from a tumor resected from a patient, and wherein the second population of TILs is at least 5-fold greater in number than the first population of TILs, and wherein the first cell culture medium comprises IL-2; and
(b) performing a rapid expansion of the second population of TILs using a population of myeloid artificial antigen presenting cells (aAPCs) in a second cell culture medium to obtain the third population of TILs, wherein the third population of TILs is at least 50-fold greater in number than the second population of TILs after 7 days from the start of the rapid expansion; and wherein the second cell culture medium comprises IL-2 and OKT-3; wherein IL-2 is present at an initial concentration of about 3000 IU/mL and OKT-3 antibody is present at an initial concentration of about 30 ng/ml in the second cell culture medium.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3; and
(d) administering a therapeutically effective portion of the third population of TILs to a patient with the cancer, wherein the rapid expansion is performed over a period not greater than 14 days.

In an embodiment, the invention provides a population of tumor infiltrating lymphocytes (TILs) for use in treating a cancer, wherein the population of TILs is a third population of TILs and is obtainable by a method comprising the steps:
(a) performing an initial expansion of a first population of TILs in a first cell culture medium to obtain a second population of TILs, wherein the first population of TILs is/has been obtained from a tumor resected from a patient, and wherein the second population of TILs is at least 5-fold greater in number than the first population of TILs, and wherein the first cell culture medium comprises IL-2; and
(b) performing a rapid expansion of the second population of TILs using a population of myeloid artificial antigen presenting cells (aAPCs) in a second cell culture medium to obtain the third population of TILs, wherein the third population of TILs is at least 50-fold greater in number than the second population of TILs after 7 days from the start of the rapid expansion; and wherein the second cell culture medium comprises IL-2 and OKT-3; wherein the rapid expansion is performed over a period not greater than 14 days.

In embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3; and
(d) administering a therapeutically effective portion of the third population of TILs to a patient with the cancer,
wherein the initial expansion is performed using a gas permeable container.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3; and
(d) administering a therapeutically effective portion of the third population of TILs to a patient with the cancer,
wherein the rapid expansion is performed using a gas permeable container.

In an embodiment, the invention provides a population of tumor infiltrating lymphocytes (TILs) for use in treating a cancer, wherein the population of TILs is a third population of TILs and is obtainable by a method comprising the steps:
(a) performing an initial expansion of a first population of TILs in a first cell culture medium to obtain a second population of TILs, wherein the first population of TILs is/has been obtained from a tumor resected from a patient, and wherein the second population of TILs is at least 5-fold greater in number than the first population of TILs, and wherein the first cell culture medium comprises IL-2;
(b) performing a rapid expansion of the second population of TILs using a population of myeloid artificial antigen presenting cells (aAPCs) in a second cell culture medium to obtain the third population of TILs, wherein the third population of TILs is at least 50-fold greater in number than the second population of TILs after 7 days from the start of the rapid expansion; and wherein the second cell culture medium comprises IL-2 and OKT-3; wherein the initial expansion and/or the rapid expansion is performed using a gas-permeable container.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3;
(d) administering a therapeutically effective portion of the third population of TILs to a patient with the cancer,
wherein the ratio of the second population of TILs to the population of aAPCs in the rapid expansion is between 1 to 80 and 1 to 400.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3;
(d) administering a therapeutically effective portion of the third population of TILs to a patient with the cancer,
wherein the ratio of the second population of TILs to the population of aAPCs in the rapid expansion is about 1 to 300.

In an embodiment, the invention provides a population of tumor infiltrating lymphocytes (TILs) for use in treating a cancer, wherein the population of TILs is a third population of TILs and is obtainable by a method comprising the steps:
(a) performing an initial expansion of a first population of TILs in a first cell culture medium to obtain a second population of TILs, wherein the first population of TILs is/has been obtained from a tumor resected from a patient, and wherein the second population of TILs is at least 5-fold greater in number than the first population of TILs, and wherein the first cell culture medium comprises IL-2;
(b) performing a rapid expansion of the second population of TILs using a population of myeloid artificial antigen presenting cells (aAPCs) in a second cell culture medium to obtain the third population of TILs, wherein the third population of TILs is at least 50-fold greater in number than the second population of TILs after 7 days from the start of the rapid expansion; and wherein the second cell culture medium comprises IL-2 and OKT-3, and wherein the ratio of the second population of TILs to the population of aAPCs in the rapid expansion is between 1 to 80 and 1 to 400.

In an embodiment, the ratio of the second population of TILs to the population of aAPCs in the rapid expansion is about 1 to 300.

In an embodiment, the invention provides a method of treating a cancer with a population of tumor infiltrating lymphocytes (TILs) comprising the steps of:
(a) obtaining a first population of TILs from a tumor resected from a patient;
(b) performing an initial expansion of the first population of TILs in a first cell culture medium to obtain a second population of TILs, wherein the second population of TILs is at least 5-fold greater in number than the first population of TILs, and wherein the first cell culture medium comprises IL-2;
(c) performing a rapid expansion of the second population of TILs using a population of myeloid artificial antigen presenting cells (aAPCs) in a second cell culture medium to obtain a third population of TILs, wherein the third population of TILs is at least 50-fold greater in number than the second population of TILs after 7 days from the start of the rapid expansion; and wherein the second cell culture medium comprises IL-2 and OKT-3;
(d) administering a therapeutically effective portion of the third population of TILs to a patient with the cancer,
wherein the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer, renal cancer, and renal cell carcinoma.

In an embodiment, the invention provides a kit for specifically inducing proliferation of a tumor infiltrating lymphocyte expressing a known co-stimulatory molecule, the kit comprising an effective amount of an aAPC, wherein said aAPC comprises a MOLM-14 cell or a EM-3 cell transduced using a lentiviral vector (LV), wherein the LV comprises a nucleic acid encoding at least one co-stimulatory ligand that specifically binds said known co-stimulatory molecule, wherein binding of the known co-stimulatory molecule with said co-stimulatory ligand stimulates and expands said T cell, the kit further comprising an applicator and an instructional material for the use of said kit.

In an embodiment, the invention provides a method for assessing the potency of tumor infiltrating lymphocytes (TILs) comprising the steps of:
(a) providing a plurality of mouse mastocytoma P815 cells expressing the endogenous CD16 Fc receptor, wherein the P815 cells are transduced with a lentiviral vector based on enhanced green fluorescent protein (GFP) and Firefly Luciferase;
(b) co-culturing the plurality of P815 cells TILs with and without OKT-3 to assess T cell receptor (TCR) activation (specific killing) or lymphokine activated killing (LAK, non-specific killing), respectively;
(c) incubating for four hours;
(d) adding Luciferin and incubating for 5 minutes;
(e) reading bioluminescence intensity using a luminometer; and
(f) and calculating percent cytotoxicity and survival.

### BRIEF DESCRIPTION OF THE DRAWINGS

The foregoing summary, as well as the following detailed description of the invention, will be better understood when read in conjunction with the appended drawings.

FIG. 1 illustrates the results of rapid expansion of TILs using irradiated allogeneic PBMC feeder cells. Each TIL line (M1015T and M1016T) (1.3×10⁵ cells) was co-cultured with 46 different irradiated feeders (1.3×10⁷ cells), IL-2 (3000 IU/mL) and OKT-3 (30 ng/ml) in a T25 flask for 7 days. The fold expansion value for TILs was calculated on Day 7. The figure shows the number of fold expansions for two TIL lines in separate stimulation experiments, with 46 different feeder lots tested, and highlights the variability of expansion results using PBMC feeder cells.

FIG. 2 illustrates a vector diagram of the pLV430G human 4-1BBL vector.

FIG. 3 illustrates a diagram of the 4-1BBL PCRP (polymerase chain reaction product) portion of the pLV430G human 4-1BBL vector.

FIG. 4 illustrates a vector diagram of the pLV430G human CD86 vector.

FIG. 5 illustrates a diagram of the CD86 PCRP portion of the pLV430G human CD86 vector.

FIG. 6 illustrates a vector diagram of the pDONR221 human CD86 donor vector.

FIG. 7 illustrates a vector diagram of the pDONR221 human 4-1BBL donor vector.

FIG. 8 illustrates a vector diagram of the pLV430G empty vector.

FIG. 9 illustrates a vector diagram of the pDONR221 empty vector.

FIG. 10 illustrates a vector diagram of the psPAX2 helper plasmid for lentivirus production.

FIG. 11 illustrates a vector diagram of the pCIGO-VSV.G helper plasmid for lentivirus production.

FIG. 12 illustrates the results of flow cytometry experiments on MOLM-14 cells before lentiviral transfection (“Untransfected”) and after transfection (“Transfected”), confirming the expression of CD137 and CD86 on engineered MOLM-14 cells.

FIG. 13 illustrates the results of rapid expansion of TILs using irradiated parental unmodified MOLM-14 cells (“Parent MOLM14”), engineered MOLM-14 cells (CD86/4-1BBL, “Engineered MOLM14”), or PBMC feeders (“Feeders”) for TIL lot M1032-T2. TIL were co-cultured with PBMC feeders or parental or engineered MOLM14 cells at 1:100 ratios with OKT-3 (30 ng/ml) and IL-2 (3000 IU/mL). Cells were counted and split on Day 6 and 11. Each dot represents cell numbers determined on Day 0, 6, 11 and 14 respectively. A logarithmic scale is used.

FIG. 14 illustrates results as shown in FIG. 13, depicted using a linear scale.

FIG. 15 illustrates results for TIL lot M1033-T6 with other parameters as given in FIG. 13, using a logarithmic scale.

FIG. 16 illustrates results as shown in FIG. 14, depicted using a linear scale.

FIG. 17 illustrates the results of rapid expansions of TILs using engineered MOLM-14 cells expressing CD86 and 4-1BBL (“TIL+Engineered MOLM14 (CD86/41BB)+OKT3”) or irradiated PBMC feeders (“TIL+Feeders+OKT3”). TIL were co-cultured with PBMC feeders or engineered MOLM-14 cells (aMOLM14) at 1:100 ratios plus OKT-3 (30 ng/ml) and IL-2 (3000 IU/mL). Cells were counted and split on Day 6 and 11. Each point represents cell numbers determined on Day 14.

FIG. 18 illustrates the results of experiments in which TILs (2×10⁴) were cultured with different ratios (1:10, 1:30, and 1:100, denoted “10”, “30”, and “100”, respectively) of parental MOLM-14 (“MOLM14”) cells, MOLM-14 cells transduced to express CD86 and 4-1BBL (“aMOLM14”), or PBMC feeders (“PBMC+”), each with OKT-3 (30 ng/ml) and IL-2 (3000 IU/mL) in wells of a 24-well G-Rex plate. A control was performed using only OKT-3 (30 ng/mL) and IL-2 (3000 IU/mL) (“PBMC-”). Each condition was cultured in triplicate. Cultures were fed with fresh media and IL-2 on Day 4 and 7. Viable cells were counted on Day 7. The bar graph represented here shows the mean plus standard deviation (SD) of viable cell numbers counted on Day 11. The p-value was calculated by the student ‘t’ test.

FIG. 19 illustrates the results of TILs cultured with different ratios (1:30, 1:100, and 1:300, denoted “30”, “100”, and “300”, respectively) of PBMC feeders (“PBMC”), parental MOLM-14 cells (“MOLM14”), or MOLM-14 cells transduced to express CD86 and 4-1BBL (“aMOLM14”), each with OKT-3 (30 ng/ml) and IL-2 (3000 IU/mL) in the single 24 well G-Rex culture plates. Viable cells were counted on day 11 and plotted. Other conditions are as in FIG. 18.

FIG. 20 illustrates the results of TILs cultured with different ratios (1:50, 1:100, and 1:200, denoted “50”, “100”, and “200”, respectively) of PBMC feeders (“PBMC”), parental MOLM-14 cells (“MOLM14”), or MOLM-14 cells transduced to express CD86 and 4-1BBL (“aMOLM14”), each with OKT-3 (30 ng/mL) and IL-2 (3000 IU/mL) in the single 24 well G-Rex culture plates. Cells were counted on day 14. Other conditions are as in FIG. 18.

FIG. 21 illustrates the results of TILs cultured with different ratios (1:100, 1:200, 1:400, and 1:800, denoted “100”, “200”, “400”, and “800”, respectively) of PBMC feeders (“PBMC”), parental MOLM-14 cells (“MOLM14”), or MOLM-14 cells transduced to express CD86 and 4-1BBL (“aMOLM14”), each with OKT-3 (30 ng/ml) and IL-2 (3000 IU/mL) in the single 24 well G-Rex culture plates. Cells were counted on day 14. Other conditions are as in FIG. 18.

FIG. 22 illustrates a sunburst visualization showing fine distribution of Live, T cell receptor (TCR) a/B, CD4, CD8, CD27, CD28, and CD57 TILs expanded with PBMC feeders.

FIG. 23 illustrates a sunburst visualization showing fine distribution of Live, TCR α/β, CD4, CD8, CD27, CD28, and CD57 TILs expanded with aMOLM14 aAPCs.

FIG. 24 depicts a flow cytometry contour plot showing memory subset (CD45RA+/−, CCR7+/−) gated on Live, TCR α/β+, CD4⁺, or CD8⁺ TILs.

FIG. 25 illustrates phenotypic characterization of the T cell subset, CD4⁺ and CD8⁺ post-REP TILs (expanded with aMOLM14 aAPCs) gated on CD3⁺ cells using a SPADE tree. The color gradient is proportional to the mean fluorescence intensity (MFI) of LAG3, TIM3, PD1, and CD137.

FIG. 26 illustrates phenotypic characterization of the T cell subset, CD4⁺ and CD8⁺ post-REP TILs (expanded with aMOLM14 aAPCs) gated on CD3⁺ cells using a SPADE tree. The color gradient is proportional to the MFI CD69, CD154, KLRG1, and TIGIT

FIG. 27 illustrates oxygen consumption rate (OCR) of TIL after expansion with Feeders or aMOLM14 measured during a mitochondrial stress test. Each data point represents mean±standard error of the mean (SEM) measured in triplicate.

FIG. 28 illustrates extracellular acidification rate (ECAR) of TIL after expansion with Feeders or aMOLM14 measured during a mitochondrial stress test. Each data point represents mean±SEM measured in triplicate.

FIG. 29 illustrates a vector diagram of the destination vector pLV4301G.

FIG. 30 illustrates a vector diagram of donor vector 1, pMK 7c12 anti mFC scFv CoOp ECORV SacII L1R5.

FIG. 31 illustrates a vector diagram of donor vector 2, pMK hCD8a scaffold TN L5 L2.

FIG. 32 illustrates a vector diagram of final vector used for lentiviral production, pLV4301G 7C12 scFv mIgG hCD8 flag.

FIG. 33 illustrates a vector diagram of the destination vector pLV4301G.

FIG. 34 illustrates a vector diagram of donor vector 1, pMK 8B3 anti mFC scFv CoOp ECORV SacII L1R5.

FIG. 35 illustrates a vector diagram of donor vector 2, pMK hCD8a scaffold TN L5 L2.

FIG. 36 illustrates a vector diagram of final vector used for lentiviral production, pLV4301G 8B3 scFv mIgG hCD8 flag.

FIG. 37 illustrates the results of flow cytometry experiments on EM-3 cells before lentiviral transfection (“Untransfected”) and after transfection (“Transfected”), confirming the expression of CD137 and CD86 on engineered EM-3 cells.

FIG. 38 illustrates the results of experiments wherein TILs were co-cultured with aEM3 (7C12 or 8B3) at a ratio of 1:100 plus OKT-3 (30 ng/ml) and IL-2 (3000 IU/mL). Cells were counted on Day 11 and 14.

FIG. 39 illustrates the results of experiments wherein TILs were co-cultured with aEM3 (7C12 or 8B3) at a ratio of 1:100 plus OKT-3 (30 ng/mL) and IL-2 (3000 IU/mL). Cells were counted on Day 11 and 14.

FIG. 40 illustrates the results of experiments wherein TILs were co-cultured with aEM3 or PBMC feeders at a 1:100 ratio with IL-2 (3000 IU/mL), with or without OKT-3 (30 ng/ml). The bar graph shows cell numbers determined on Day 11.

FIG. 41 illustrates the results of TIL expansions with EM-3 aAPCs at different TIL:aAPC ratios.

FIG. 42 illustrates the results of TIL expansions with EM-3 aAPCs. TILs (2×10⁴) were co-cultured with five different PBMC feeder lots or aEM3 (in triplicate) at a 1:100 ratio with IL-2 (3000 IU/mL) in a G-Rex 24 well plate. Viable cells were counted on Day 14. The graph shows viable cell numbers (mean) with 95% confidence interval counted on Day 14.

FIG. 43 illustrates the results of TIL expansions with EM-3 aAPCs and MOLM-14 aAPCs. TILs (2×10⁴) were co-cultured with five different PBMC feeder lots or aMOLM14 (in triplicate) or aEM3 (also in triplicate) at 1:100 ratio with IL-2 (3000 IU/mL) in a G-Rex 24 well plate. The graph shows viable cell numbers (mean) with 95% confidence interval counted on Day 14.

FIG. 44 illustrates a sunburst visualization to show fine distribution of Live, TCR α/β, CD4⁺, and CD8⁺ TILs expanded with aEM3 aAPCs or PBMC feeders (TIL batch M1054).

FIG. 45 illustrates the sunburst visualization to show fine distribution of Live, TCR α/β, CD4⁺, and CD8⁺ TILs expanded with aEM3 aAPCs or PBMC feeders (TIL batch M1055).

FIG. 46 illustrates the CD4⁺ and CD8⁺ SPADE tree of TILs expanded with aEM3 aAPCs or PBMC feeders using CD3⁺ cells. The color gradient is proportional to the MFI of LAG-3, TIM-3, PD-1, and CD137.

FIG. 47 illustrates the CD4⁺ and CD8⁺ SPADE tree of TILs expanded with aEM3 aAPCs or PBMC feeders using CD3⁺ cells. The color gradient is proportional to the MFI of CD69, CD154, KLRG1, and TIGIT.

FIG. 48 illustrates a summary of spare respiratory capacity measured by the Seahorse XF Mito stress test.

FIG. 49 illustrates a summary of glycolytic reserve measured by the Seahorse XF Mito stress test.

FIG. 50 illustrates a mitochondrial stain of live TILs expanded against PBMC or aEM3 using MitoTracker dye, which stains mitochondria in live cells and for which accumulation is dependent upon membrane potential. TILs expanded against PBMC or aEM3 were stained L/D Aqua followed by MitoTracker red dye. Data shown are MitoTracker positive (MFI) cells gated on live population.

FIG. 51 illustrates results of a P815 BRLA for cytotoxic potency and functional activity, comparing TILs expanded with PBMC feeders to TILs expanded using aMOLM14 aAPCs.

FIG. 52 illustrates results of a P815 BRLA for cytotoxic potency and functional activity, comparing TILs expanded with PBMC feeders to TILs expanded using aEM3 aAPCs.

FIG. 53 illustrates IFN-γ release for two batches of TILs following overnight stimulation (“S”) with microbeads coated with anti-CD3/CD28/4-1BB in comparison to unstimulated (“US”) TILs, comparing TILs expanded with PBMC feeders to TILs expanded using aMOLM14 aAPCs. * p<0.05, ** p<0.005, *** p<0.001, ns=not significant.

FIG. 54 illustrates IFN-γ release for three batches of TILs following overnight stimulation (“S”) with microbeads coated with anti-CD3/CD28/4-1BB in comparison to unstimulated (“US”) TILs, comparing TILs expanded with PBMC feeders to TILs expanded using aEM3 aAPCs. * p<0.05, ** p<0.005, *** p<0.001, ns=not significant.

FIG. 55 illustrates Granzyme B release for two batches of TILs following overnight stimulation (“S”) with microbeads coated with anti-CD3/CD28/4-1BB in comparison to unstimulated (“US”) TILs, comparing TILs expanded with PBMC feeders to TILs expanded using aMOLM14 aAPCs. * p<0.05, ** p<0.005, *** p<0.001, ns=not significant.

FIG. 56 illustrates Granzyme B release for three batches of TILs following overnight stimulation (“S”) with microbeads coated with anti-CD3/CD28/4-1BB in comparison to unstimulated (“US”) TILs, comparing TILs expanded with PBMC feeders to TILs expanded using aEM3 aAPCs. * p<0.05, ** p<0.005, *** p<0.001, ns=not significant.

FIG. 57 illustrates a TIL expansion and treatment process. aAPCs of the present invention may be used in both the pre-REP stage (top half of figure) or REP stage (bottom half of figure) and may be added when IL-2 is added to each cell culture. Step 1 refers to the addition of 4 tumor fragments into 10 G-Rex 10 flasks. At step 2, approximately 40×10⁶ TILs or greater are obtained. At step 3, a split occurs into 36 G-Rex 100 flasks for REP. TILs are harvested by centrifugation at step 4. Fresh TIL product is obtained at step 5 after a total process time of approximate 43 days, at which point TILs may be infused into a patient.

FIG. 58 illustrates a treatment protocol for use with TILs expanded with aAPCs. Surgery (and tumor resection) occurs at the start, and lymphodepletion chemo refers to non-myeloablative lymphodepletion with chemotherapy as described elsewhere herein.

FIG. 59 illustrates Bioluminescent Redirected Lysis Assay (BRLA) results, showing percentage cytotoxicity of TIL batch M1033T-1 when co-cultured with P815 Clone G6 (with and without anti-CD3) at individual effector:target ratios.

FIG. 60 illustrates enzyme-linked immunosorbent assay (ELISA) data showing amount of IFN-γ released against different ratios of effector to target cells.

FIG. 61 illustrates LAMP1(%) expressed by TIL batch M1033T-1 when co-cultured with P815 Clone G6 in the presence of anti-CD3 at a ratio of 1:1 effector to target cells for 4 hr and 24 hr co-culture.

FIG. 62 illustrates BRLA results for TIL batch M1030. Cytotoxicity (measured as LU₅₀/1×10⁶ TIL) by BRLA is 26±16.

FIG. 63 illustrates standard chromium release assay for TIL batch M1030. Cytotoxicity (measured as LU₅₀/1×10⁶ TIL) by the chromium release assay is 22.

FIG. 64 illustrates BRLA results for TIL batch M1053, showing the lytic units of the TILs by BRLA as 70±17.

FIG. 65 illustrates standard chromium release assay results for TIL batch M1053, also showing lytic unit of the TILs by chromium assay as 14±5. Comparison of this result with FIG. 64 shows the comparable performance of the BRLA and chromium release assay.

FIG. 66 illustrates the linear relationship between IFN-γ release and cytotoxic potential of TILs.

FIG. 67 illustrates ELISpot results for IFN-γ.

FIG. 68 illustrates enzymatic IFN-γ release for TIL batch M1053.

FIG. 69 illustrates enzymatic IFN-γ release for TIL batch M1030.

FIG. 70 illustrates ELISpot data showing Granzyme B release by M1053T and M1030T. This data confirms the potency of the TILs shown by the BRLA.

FIG. 71 illustrates enzymatic Granzyme B release for TIL batch M1053.

FIG. 72 illustrates enzymatic Granzyme B release for TIL batch M1030.

FIG. 73 illustrates ELISpot data showing TNF-α release by M1053T and M1030T. This data confirms the potency of the TILs shown by the BRLA.

FIG. 74 illustrates enzymatic TNF-α release for TIL batch M1053.

FIG. 75 illustrates enzymatic TNF-α release for TIL batch M1030.

FIG. 76 illustrates changes in cell populations of aEM3 cells (C712 (A) and 8B5 (B)) when weaning such cell populations off of FBS to hAB serum media.

FIG. 77 illustrates changes in cell populations of during freeze-thaw-recovery cycles with aEM3 cell populations suspended in various freezing media.

FIG. 78 illustrates the growth of aEM3 cells in gas permeable cell culture flasks over an eight-day time course.

FIG. 79 illustrates a flow panel analysis to determine the purity of aEM3 cells.

FIG. 80 illustrates the results of a flow panel analysis used to determine the purity of aEM3 cells.

FIG. 81 illustrates the differences in cytokine expression between aEM3 feeder cells and PBMC feeders stimulated by OKT3.

FIG. 82 illustrates that TIL may advantageously expanded (pre-REP) with serum free media (i.e., CTS Optmizer) to provide increased cell numbers as compared to CM1.

FIG. 83 and FIG. 84 illustrate that TIL may advantageously expanded with serum free media (i.e., CTS Optmizer) to provide increased cell numbers as compared to CM1 at Day 11 (PreREP) (FIG. 83) and Day 22 (Pre- and Post-REP) (FIG. 84).

FIG. 85 illustrates that aAPC cells (i.e., aEM3 cells) can be grown and using serum free media. Specifically, CTS OpTimizer and Prime-TCDM were found to be effective in growing aEM3 as compared to cDMEM (10% hSerum). Data shown were mean+SD of five separate experiments. The p value was calculated by the student t-test. * P<0.05.

FIG. 86 and FIG. 87 illustrate the results of two experiments that demonstrate the rapid recovery of aEM3 cells from the TIL-R3 cell line on day 3 following cryopreservation. FIG. 86 illustrates the total cell counts for experiment one and FIG. 87 illustrates the total cell counts for experiment two.

FIG. 88 illustrates the growth of aEM3 cells from the TIL-R3 cell line following cryopreservation where the cells were plated and grown for 9 days. Cell counts were measured every three days post thaw.

FIG. 89 illustrates the growth of aEM3 cells from the TIL-R3 cell line following cryopreservation where the cells were plated in GREX 10 flasks and grown for 8 days. Cell counts were measured every four days post thaw.

FIG. 90 illustrates a vector diagram of the pLenti-C-Myc-DDK human OX40L vector.

FIG. 91 illustrates the results of flow cytometry analysis of TILs expanded in a REP with the aEM3 cell line and PBMC feeders, showing that TILs cultured with aEM3 promotes CD8⁺ TIL skewness.

FIG. 92 illustrates the numbers of viable cells obtained from experiments wherein TILs were expanded in a REP with the aEM3 cell line and PBMC feeders.

FIG. 93 illustrates the numbers of CD3⁺ cells obtained from experiments wherein TILs were expanded in a REP with the aEM3 cell line and PBMC feeders.

FIG. 94 illustrates the numbers of CD3⁻ cells obtained from experiments wherein TILs were expanded in a REP with the aEM3 cell line and PBMC feeders.

FIG. 95 illustrates the results of telomere length analysis using a qPCR method.

FIG. 96 illustrates a schematic diagram of an embodiment of an aAPC of the present invention.

FIG. 97 illustrates a schematic diagram of an embodiment of an aAPC of the present invention.

FIG. 98 illustrates a schematic diagram of an embodiment of an aAPC of the present invention.

### BRIEF DESCRIPTION OF THE SEQUENCE LISTING

SEQ ID NO: 1 is an amino acid sequence for the heavy chain of muromonab.

SEQ ID NO:2 is an amino acid sequence for the light chain of muromonab.

SEQ ID NO:3 is an amino acid sequence for recombinant human IL-2.

SEQ ID NO:4 is an amino acid sequence for aldesleukin.

SEQ ID NO:5 is an amino acid sequence for recombinant human IL-7.

SEQ ID NO:6 is an amino acid sequence for recombinant human IL-15.

SEQ ID NO:7 is an amino acid sequence for recombinant IL-21.

SEQ ID NO:8 is the amino acid sequence of human CD86.

SEQ ID NO:9 is the amino acid sequence of human 4-1BBL (CD137L).

SEQ ID NO: 10 is the amino acid sequence of human OX40L (CD134L).

SEQ ID NO:11 is the amino acid sequence of human CD28.

SEQ ID NO:12 is the amino acid sequence of human CTLA-4.

SEQ ID NO:13 is the amino acid sequence of human 4-1BB (CD137).

SEQ ID NO: 14 is the amino acid sequence of human OX40 (CD134).

SEQ ID NO:15 is a nucleotide sequence for the pLV430G 4-1BBL empty vector.

SEQ ID NO: 16 is a nucleotide sequence for the 4-1BBL COOP portion of the pLV430G human 4-1BBL vector.

SEQ ID NO: 17 is a nucleotide sequence for the 4-1BBL PCRP.

SEQ ID NO: 18 is a nucleotide sequence for the pLV430G hCD86 empty vector.

SEQ ID NO: 19 is a nucleotide sequence for the hCD86 COOP portion of the pLV430G human hCD86 vector.

SEQ ID NO:20 is a nucleotide sequence for the hCD86 COOP B1 B2 PCRP portion of the pLV430G human hCD86 vector.

SEQ ID NO:21 is a nucleotide sequence for the pDONR221 hCD86 vector.

SEQ ID NO:22 is a nucleotide sequence for the pDONR221 4-1BBL vector.

SEQ ID NO:23 is a nucleotide sequence for the pLV430G vector.

SEQ ID NO:24 is a nucleotide sequence for the pDONR221 vector.

SEQ ID NO:25 is a nucleotide sequence for the psPAX2 helper plasmid for lentiviral production.

SEQ ID NO:26 is a nucleotide sequence for the pCIGO-VSV.G helper plasmid for lentiviral production.

SEQ ID NO:27 is the amino acid sequence of the mFc-7C12 scFv clone.

SEQ ID NO:28 is the amino acid sequence of the mFc-8B3 scFv clone.

SEQ ID NO:29 is a nucleotide sequence for the mFC-7C12 scFv.

SEQ ID NO:30 is a nucleotide sequence for the mFC-8B3 scFv.

SEQ ID NO:31 is a nucleotide sequence for the destination vector pLV4301G.

SEQ ID NO:32 is a nucleotide sequence for the donor vector 1, pMK 7c12 anti mFC scFv CoOp ECORV SacII L1R5.

SEQ ID NO:33 is a nucleotide sequence for the donor vector 2, pMK hCD8a scaffold TN L5 L2.

SEQ ID NO:34 is a nucleotide sequence for the final vector used for lentiviral production, pLV4301G 7C12 scFv mIgG hCD8 flag.

SEQ ID NO:35 is a nucleotide sequence for the destination vector, pLV4301G.

SEQ ID NO:36 is a nucleotide sequence for the donor vector 1, pMK 8B3 anti mFC scFv CoOp ECORV SacII L1R5.

SEQ ID NO:37 is a nucleotide sequence for the donor vector 2, pMK hCD8a scaffold TN L5 L2.

SEQ ID NO:38 is a nucleotide sequence for the final vector used for lentiviral production, pLV4301G 8B3 scFv mIgG hCD8 flag.

SEQ ID NO:39 is a nucleotide sequence for pLenti-C-Myc-DDK OX40L vector for lentiviral production.

SEQ ID NO:40 is a nucleotide sequence for Tel-1b primer used for quantitative polymerase chain reaction measurements of telomere length.

SEQ ID NO:41 is a nucleotide sequence for Tel-2b primer used for quantitative polymerase chain reaction measurements of telomere length.

SEQ ID NO:42 is a nucleotide sequence for Tel-1b primer used for quantitative polymerase chain reaction measurements of telomere length.

SEQ ID NO:43 is a nucleotide sequence for Tel-1b primer used for quantitative polymerase chain reaction measurements of telomere length.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

The terms “co-administration,” “co-administering,” “administered in combination with,” “administering in combination with,” “simultaneous,” and “concurrent,” as used herein, encompass administration of two or more active pharmaceutical ingredients to a human subject so that both active pharmaceutical ingredients and/or their metabolites are present in the human subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present is also encompassed in the methods of the invention.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “ex vivo” refers to an event which involves treating or performing a procedure on a cell, tissue and/or organ which has been removed from a subject's body. Aptly, the cell, tissue and/or organ may be returned to the subject's body in a method of surgery or treatment.

The term “antigen” refers to a substance that induces an immune response. In some embodiments, an antigen is a molecule capable of being bound by an antibody or a T cell receptor (TCR) if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by other antigens.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the human subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit in a human subject. A prophylactic effect includes delaying or eliminating the appearance of a disease or condition, delaying or eliminating the onset of symptoms of a disease or condition, slowing, halting, or reversing the progression of a disease or condition, or any combination thereof.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are described herein.

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and MI macrophages. TILs include both primary and secondary TILs. “Primary TILs” are those that are obtained from patient tissue samples as outlined herein (sometimes referred to herein as “freshly harvested” or “a first population of TILs”), and “secondary TILs” are any TIL cell populations that have been expanded or proliferated as discussed herein, including, but not limited to bulk TILs and expanded TILs (“REP TILs” or “post-REP TILs”, or “second population of TILs” or “third population of TILs” where appropriate).

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally, and alternatively, TILs can be functionally defined by their ability to infiltrate solid tumors upon reintroduction into a patient.

By “cryopreserved TILs” herein is meant that TILs are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” herein is meant a population of TILs that was previously cryopreserved and then treated to return to room temperature or higher, including but not limited to cell culture temperatures or temperatures wherein TILs may be administered to a patient.

By “population of cells” (including TILs) herein is meant a number of cells that share common traits.

The term “central memory T cell” refers to a subset of T cells that in the human are CD45R0+ and constitutively express CCR7 (CCR7^{hi}) and CD62L (CD62^{hi}). The surface phenotype of central memory T cells also includes TCR, CD3, CD127 (IL-7R), and IL-15R. Transcription factors for central memory T cells include BCL-6, BCL-6B, MBD2, and BMI1. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7 (CCR7^{lo}) and are heterogeneous or low for CD62L expression (CD62L^{lo}). The surface phenotype of central memory T cells also includes TCR, CD3, CD127 (IL-7R), and IL-15R. Transcription factors for central memory T cells include BLIMP1. Effector memory T cells rapidly secret high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perforin.

The terms “sequence identity,” “percent identity,” and “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

The term “conservative amino acid substitutions” means amino acid sequence modifications which do not abrogate the binding of an antibody to an antigen or a protein to its ligand. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in a 4-1BBL or CD86 protein is preferably replaced with another amino acid residue from the same class. Methods of identifying amino acid conservative substitutions which do not eliminate antigen or ligand binding are well-known in the art (see, e.g., Brummell, et al., Biochemistry 1993, 32, 1180-1187; Kobayashi, et al., Protein Eng. 1999, 12, 879-884 (1999); and Burks, et al., Proc. Natl. Acad. Sci. USA 1997, 94, 412-417).

The term “retrovirus” refers to RNA viruses that utilize reverse transcriptase during their replication cycle, wherein retroviral genomic RNA is converted into double-stranded DNA by reverse transcriptase. The double-stranded DNA form is integrated into the chromosome of the infected cell (a “provirus”). The provirus serves as a template for RNA polymerase II and directs the expression of RNA molecules which encode the structural proteins and enzymes needed to produce new viral particles. At each end of the provirus are structures called “long terminal repeats” or “LTRs.” The LTR contains numerous regulatory signals including transcriptional control elements, polyadenylation signals and sequences needed for replication and integration of the viral genome. Several genera included within the family Retroviridae, including Cisternavirus A, Oncovirus A, Oncovirus B, Oncovirus C, Oncovirus D, Lentivirus, Gammaretrovirus, and Spumavirus. Some of the retroviruses are oncogenic (i.e., tumorigenic), while others are not. The oncoviruses induce sarcomas, leukemias, lymphomas, and mammary carcinomas in susceptible species. Retroviruses infect a wide variety of species, and may be transmitted both horizontally and vertically. Because they are integrated into the host DNA, they are capable of transmitting sequences of host DNA from cell to cell. Example gammaretroviral vectors include those derived from the amphotropic Moloney murine leukemia virus (MLV-A), which use cell surface phosphate transporter receptors for entry and then permanently integrate into proliferating cell chromosomes. The amphotropic MLV vector system has been well established and is a popular tool for gene delivery (See, e.g., Gordon and Anderson, Curr. Op. Biotechnol., 1994, 5, 611-616 and Miller, et al., Meth. Enzymol., 1993, 217, 581-599, the disclosures of which are incorporated herein by reference.

The term “lentivirus” refers to a genus that includes HIV (human immunodeficiency virus; including HIV type 1, and HIV type 2), visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T cells).

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1 clone, also known as T3 and CD38. Other anti-CD3 antibodies include, for example, otelixizumab, teplizumab, and visilizumab.

The term “OKT-3” (also referred to herein as “OKT3”) refers to a monoclonal antibody or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotec GmbH, Bergisch Gladbach, Germany) and muromonab or variants, conservative amino acid substitutions, glycoforms, or biosimilars thereof. The amino acid sequences of the heavy and light chains of muromonab are given in Table 1 (SEQ ID NO: 1 and SEQ ID NO:2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab.

| Identifier | | |
|---|---|---|
| (Description) | Sequence (One-Letter Amino Acid Symbols) | |
| SEQ ID NO: 1 | QVQLQQSGAE LARPGASVKM SCKASGYTFT RYTMHWVKQR PGQGLEWIGY INPSRGYTNY | 60 |
| (Muromonab heavy | NQKFKDKATL TTDKSSSTAY MQLSSLTSED SAVYYCARYY DDHYCLDYWG QGTTLTVSSA | 120 |
| chain) | KTTAPSVYPL APVCGGTTGS SVTLGCLVKG YFPEPVTLTW NSGSLSSGVH TFPAVLQSDL | 180 |
| | YTLSSSVTVT SSTWPSQSIT CNVAHPASST KVDKKIEPRP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 2 | QIVLTQSPAI MSASPGEKVT MTCSASSSVS YMNWYQQKSG TSPKRWIYDT SKLASGVPAH | 60 |
| (Muromonab light | FRGSGSGTSY SLTISGMEAE DAATYYCQQW SSNPFTFGSG TKLEINRADT APTVSIFPPS | 120 |
| chain) | SEQLTSGGAS VVCFLNNFYP KDINVKWKID GSERQNGVLN SWTDQDSKDS TYSMSSTLTL | 180 |
| | TKDEYERHNS YTCEATHKTS TSPIVKSFNR NEC | 213 |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO: 3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL2 prodrug NKTR-214, available from Nektar Therapeutics, South San Francisco, CA, USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, NJ, USA (Cat. No. CYT-254) and ThermoFisher Scientific, Inc., Waltham, MA, USA (human IL-7 recombinant protein, Cat. No. Gibco PHC0071). The amino acid sequence of recombinant human IL-7 suitable for use in the invention is given in Table 2 (SEQ ID NO:5).

The term “IL-15” (also referred to herein as “IL15”) refers to the T cell growth factor known as interleukin-15, and includes all forms of IL-2 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, NJ, USA (Cat. No. CYT-230-b) and ThermoFisher Scientific, Inc., Waltham, MA, USA (human IL-15 recombinant protein, Cat. No. 34-8159-82). The amino acid sequence of recombinant human IL-15 suitable for use in the invention is given in Table 2 (SEQ ID NO: 6).

The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4⁺ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, NJ, USA (Cat. No. CYT-408-b) and ThermoFisher Scientific, Inc., Waltham, MA, USA (human IL-21 recombinant protein, Cat. No. 14-8219-80). The amino acid sequence of recombinant human IL-21 suitable for use in the invention is given in Table 2 (SEQ ID NO:7).

TABLE 2
Amino acid sequences of interleukins.

| Identifier | | |
|---|---|---|
| (Description) | Sequence (One-Letter Amino Acid Symbols) | |
| SEQ ID NO: 3 | MAPTSSSTKK TQLQLEHLLL DLQMILNGIN NYKNPELTRM LTFKFYMPKK ATELKHLQCL | 60 |
| (recombinant | EEELKPLEEV LNLAQSKNFH LRPRDLISNI NVIVLELKGS ETTFMCEYAD ETATIVEFLN | 120 |
| human IL-2 | RWITFCQSII STLT | 134 |
| (rhIL-2)) | | |
| SEQ ID NO: 4 | PTSSSTKKTQ LQLEHLLLDL QMILNGINNY KNPELTRMLT FKFYMPKKAT ELKHLQCLEE | 60 |
| (aldesleukin) | ELKPLEEVLN LAQSKNFHLR PRDLISNINV IVLELEGSET TFMCEYADET ATIVEFLNRW | 120 |
| | ITFSQSIIST LT | 132 |
| SEQ ID NO: 5 | MDCDIEGEDG KQYESVLMVS IDQLLDSMKE IGSNCLNNEF NFFKRHICDA NKEGMFLFRA | 60 |
| (recombinant | ARKLRQFLKM NSTGDFDLHL LEVSEGTTIL LNCTGQVKGR KPAALGEAQP TKSLEENKSL | 120 |
| human IL-7 | KEQKKLNDLC FLKRLLQEIK TCWNKILMGT KEH | 153 |
| (rhIL-7)) | | |
| SEQ ID NO: 6 | MNWVNVISDL KKIEDLIQSM HIDATLYTES DVHPSCKVTA MKCFLLELQV ISLESGDASI | 60 |
| (recombinant | HDTVENLIIL ANNSLSSNGN VTESGCKECE ELEEKNIKEF LQSFVHIVQM FINTS | 115 |
| human IL-15 | | |
| (rhIL-15)) | | |
| SEQ ID NO: 7 | MQDRHMIRMR QLIDIVDQLK NYVNDLVPEF LPAPEDVETN CEWSAFSCFQ KAQLKSANTG | 60 |
| (recombinant | NNERIINVSI KKLKRKPPST NAGRRQKHRL TCPSCDSYEK KPPKEFLERF KSLLQHMIHQ | 120 |
| human IL-21 | HLSSRTHGSE DS | 132 |
| (rhIL-21)) | | |

The term “myeloid cell” as used herein refers to cells of the myeloid lineage or derived therefrom. The myeloid lineage includes a number of morphologically, phenotypically, and functionally distinct cell types including different subsets of granulocytes (neutrophils, eosinophils, and basophils), monocytes, macrophages, erythrocytes, megakaryocytes, and mast cells. In certain embodiments, the myeloid cell is a cell derived from a cell line of myeloid lineage.

“MOLM-14” refers to a human leukemia cell line which was established from the peripheral blood of a patient with relapsed acute monocytic leukemia, and initial phenotypic characterization indicated the presence of at least the following markers: CD4, CD9, CD11a, CD13, CD14, CD15, CD32, CD33, CD64, CD65, CD87, CD92, CD93, CD116, CD118, and CD155. Matsuo, et al., Leukemia 1997, 11, 1469-77. Additional phenotypic characterization of MOLM-14 found higher levels of HLA-A/B/C, CD64, CD80, ICOS-L, CD58, and lower levels of CD86. The MOLM-14 cell line is deposited at DSMZ under Accession No. ACC777. The closely related MOLM-13 cell line is deposited at DSMZ under Accession No. ACC554. As used herein the term “MOLM-14 cell” refers to a MOLM-14 cell and/or a cell derived from the deposited MOLM-14 parental cell line. As used herein the term “MOLM-13 cell” refers to a MOLM-13 cell and/or a cell derived from the deposited MOLM-13 parental cell line.

“EM-3” refers to a human cell line was established from the bone marrow of a patient with Philadelphia chromosome-positive CML. Konopka, et al., Proc. Nat'l Acad. Sci. USA 1985, 82, 1810-4. Phenotypic characterization for EM-3 cells indicates the presence of at least the following markers: CD13, CD15, and CD33. The EM-3 cell line is deposited at DSMZ under Accession No. ACC134 whilst the closely related EM-2 cell line is deposited at DSMZ under Accession No. ACC135. As used herein the term “EM-3 cell” refers to a EM-3 cell and/or a cell derived from the deposited EM-3 parental cell line.

As used herein, the term “a CD86 protein” may refer to a protein comprising an amino acid sequence as set forth in SEQ ID NO:8 or a protein comprising an amino acid sequence having at least 90% sequence identity to the amino acid sequence depicted in SEQ ID NO:8, e.g., 91%, 92%, 93%, 94%, 95%, 96%, 97%, 98% or 99%.

As used herein, the term “4-1BBL” or “CD137L” may refer to a protein comprising an amino acid sequence as set forth in SEQ ID NO:9 or a protein comprising an amino acid sequence having at least 90% sequence identity to the amino acid sequence depicted in SEQ ID NO: 9, e.g., 91%, 92%, 93%, 94%, 95%, 96%, 97%, 98% or 99%.

As used herein, the term “OX40L” or “CD137L” may refer to a protein comprising an amino acid sequence as set forth in SEQ ID NO: 10 or a protein comprising an amino acid sequence having at least 90% sequence identity to the amino acid sequence depicted in SEQ ID NO: 10, e.g., 91%, 92%, 93%, 94%, 95%, 96%, 97%, 98% or 99%.

The term “biosimilar” means a biological product, including a monoclonal antibody or fusion protein, that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference IL-2 protein is aldesleukin (PROLEUKIN), a protein approved by drug regulatory authorities with reference to aldesleukin is a “biosimilar to” aldesleukin or is a “biosimilar thereof” of aldesleukin. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10 (4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

As used herein, the term “variant” encompasses but is not limited to proteins, antibodies or fusion proteins which comprise an amino acid sequence which differs from the amino acid sequence of a reference protein or antibody by way of one or more substitutions, deletions and/or additions at certain positions within or adjacent to the amino acid sequence of the reference protein or antibody. The variant may comprise one or more conservative substitutions in its amino acid sequence as compared to the amino acid sequence of a reference protein or antibody. Conservative substitutions may involve, e.g., the substitution of similarly charged or uncharged amino acids. The variant retains the ability to specifically bind to the antigen of the reference protein or antibody. The term “variant” also includes pegylated antibodies or proteins.

“Pegylation” refers to a modified antibody, or a fragment thereof, or protein that typically is reacted with polyethylene glycol (PEG), such as a reactive ester or aldehyde derivative of PEG, under conditions in which one or more PEG groups become attached to the antibody, antibody fragment, or protein. Pegylation may, for example, increase the biological (e.g., serum) half life of the antibody or protein. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. The antibody or protein to be pegylated may be an aglycosylated antibody. Methods for pegylation are known in the art and can be applied to the antibodies and proteins described herein, as described for example in European Patent Nos. EP 0154316 and EP 0401384.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

### Artificial Antigen Presenting Cells

In an embodiment, the invention includes an isolated artificial antigen presenting cell (aAPC) comprising a cell that expresses HLA-A/B/C, CD64, CD80, ICOS-L, and CD58, and is modified to express one or more costimulatory molecules. In an embodiment, the invention includes an aAPC comprising a MOLM-14 cell that is modified to express one or more costimulatory molecules. In an embodiment, the invention includes an aAPC comprising a MOLM-13 cell that is modified to express one or more costimulatory molecules.

In an embodiment, the invention includes an aAPC comprising a MOLM-14 cell that endogenously expresses HLA-A/B/C, CD64, CD80, ICOS-L, and CD58, wherein the cell is modified to express a CD86 protein comprising an amino acid sequence as set forth in SEQ ID NO: 8, and conservative amino acid substitutions thereof, and a 4-1BBL protein comprising an amino acid sequence as set forth in SEQ ID NO:9, and conservative amino acid substitutions thereof, and wherein the CD86 protein and the 4-1BBL protein are expressed on the surface of the MOLM-14 cell.

In an embodiment, the invention includes an aAPC comprising a MOLM-14 cell transduced with one or more viral vectors, wherein the one or more viral vectors comprise a nucleic acid encoding CD86 and a nucleic acid encoding 4-1BBL, and wherein the MOLM-14 cell expresses CD86 and 4-1BBL. In an embodiment, the invention includes an aAPC comprising a MOLM-13 cell transduced with one or more viral vectors, wherein the one or more viral vectors comprise a nucleic acid encoding CD86 and a nucleic acid encoding 4-1BBL, and wherein the MOLM-13 cell expresses CD86 and 4-1BBL. In an embodiment, the invention includes a method of preparing any of the foregoing embodiments of aAPCs.

In an embodiment, the invention includes an aAPC comprising a MOLM-14 cell modified to express a CD86 protein comprising an amino acid sequence as set forth in SEQ ID NO: 8 and a 4-1BBL protein comprising an amino acid sequence as set forth in SEQ ID NO:9, wherein the CD86 protein and the 4-1BBL protein are expressed on the surface of the MOLM-14 cell. In an embodiment, the invention includes an aAPC comprising a MOLM-13 cell modified to express a CD86 protein comprising an amino acid sequence as set forth in SEQ ID NO:8, and conservative amino acid substitutions thereof, and a 4-1BBL protein comprising an amino acid sequence as set forth in SEQ ID NO:9, and conservative amino acid substitutions thereof, wherein the CD86 protein and the 4-1BBL protein are expressed on the surface of the MOLM-13 cell. In an embodiment, the invention includes a method of preparing any of the foregoing embodiments of aAPCs.

In an embodiment, the invention includes an aAPC comprising a MOLM-14 cell modified to express a CD86 protein comprising a sequence with greater than 99% identity to an amino acid sequence as set forth in SEQ ID NO:8 and a 4-1BBL protein comprising a sequence with greater than 99% identity to an amino acid sequence as set forth in SEQ ID NO:9, wherein the CD86 protein and the 4-1BBL protein are expressed on the surface of the MOLM-14 cell. In an embodiment, the invention includes an aAPC comprising a MOLM-14 cell modified to express a CD86 protein comprising a sequence with greater than 98% identity to an amino acid sequence as set forth in SEQ ID NO:8 and a 4-1BBL protein comprising a sequence with greater than 98% identity to an amino acid sequence as set forth in SEQ ID NO:9, wherein the CD86 protein and the 4-1BBL protein are expressed on the surface of the MOLM-14 cell. In an embodiment, the invention includes an aAPC comprising a MOLM-14 cell modified to express a CD86 protein comprising a sequence with greater than 97% identity to an amino acid sequence as set forth in SEQ ID NO:8 and a 4-1BBL protein comprising a sequence with greater than 97% identity to an amino acid sequence as set forth in SEQ ID NO:9, wherein the CD86 protein and the 4-1BBL protein are expressed on the surface of the MOLM-14 cell. In an embodiment, the invention includes an aAPC comprising a MOLM-14 cell modified to express a CD86 protein comprising a sequence with greater than 96% identity to an amino acid sequence as set forth in SEQ ID NO:8 and a 4-1BBL protein comprising a sequence with greater than 96% identity to an amino acid sequence as set forth in SEQ ID NO:9, wherein the CD86 protein and the 4-1BBL protein are expressed on the surface of the MOLM-14 cell. In an embodiment, the invention includes an aAPC comprising a MOLM-14 cell modified to express a CD86 protein comprising a sequence with greater than 95% identity to an amino acid sequence as set forth in SEQ ID NO:8 and a 4-1BBL protein comprising a sequence with greater than 95% identity to an amino acid sequence as set forth in SEQ ID NO:9, wherein the CD86 protein and the 4-1BBL protein are expressed on the surface of the MOLM-14 cell. In an embodiment, the invention includes an aAPC comprising a MOLM-14 cell modified to express a CD86 protein comprising a sequence with greater than 90% identity to an amino acid sequence as set forth in SEQ ID NO:8 and a 4-1BBL protein comprising a sequence with greater than 90% identity to an amino acid sequence as set forth in SEQ ID NO:9, wherein the CD86 protein and the 4-1BBL protein are expressed on the surface of the MOLM-14 cell. In an embodiment, the invention includes a method of preparing any of the foregoing embodiments of aAPCs.

In an embodiment, the invention includes an aAPC comprising a MOLM-13 cell modified to express a CD86 protein comprising an amino acid sequence as set forth in SEQ ID NO: 8 and a 4-1BBL protein comprising an amino acid sequence as set forth in SEQ ID NO:9, wherein the CD86 protein and the 4-1BBL protein are expressed on the surface of the MOLM-13 cell. In an embodiment, the invention includes an aAPC comprising a MOLM-13 cell modified to express a CD86 protein comprising an amino acid sequence as set forth in SEQ ID NO:8, and conservative amino acid substitutions thereof, and a 4-1BBL protein comprising an amino acid sequence as set forth in SEQ ID NO:9, and conservative amino acid substitutions thereof, wherein the CD86 protein and the 4-1BBL protein are expressed on the surface of the MOLM-13 cell. In an embodiment, the invention includes a method of preparing any of the foregoing embodiments of aAPCs.

In an embodiment, the invention includes an aAPC comprising a MOLM-13 cell modified to express a CD86 protein comprising a sequence with greater than 99% identity to an amino acid sequence as set forth in SEQ ID NO:8 and a 4-1BBL protein comprising a sequence with greater than 99% identity to an amino acid sequence as set forth in SEQ ID NO:9, wherein the CD86 protein and the 4-1BBL protein are expressed on the surface of the MOLM-13 cell. In an embodiment, the invention includes an aAPC comprising a MOLM-13 cell modified to express a CD86 protein comprising a sequence with greater than 98% identity to an amino acid sequence as set forth in SEQ ID NO:8 and a 4-1BBL protein comprising a sequence with greater than 98% identity to an amino acid sequence as set forth in SEQ ID NO:9, wherein the CD86 protein and the 4-1BBL protein are expressed on the surface of the MOLM-13 cell. In an embodiment, the invention includes an aAPC comprising a MOLM-13 cell modified to express a CD86 protein comprising a sequence with greater than 97% identity to an amino acid sequence as set forth in SEQ ID NO:8 and a 4-1BBL protein comprising a sequence with greater than 97% identity to an amino acid sequence as set forth in SEQ ID NO:9, wherein the CD86 protein and the 4-1BBL protein are expressed on the surface of the MOLM-13 cell. In an embodiment, the invention includes an aAPC comprising a MOLM-13 cell modified to express a CD86 protein comprising a sequence with greater than 96% identity to an amino acid sequence as set forth in SEQ ID NO:8 and a 4-1BBL protein comprising a sequence with greater than 96% identity to an amino acid sequence as set forth in SEQ ID NO:9, wherein the CD86 protein and the 4-1BBL protein are expressed on the surface of the MOLM-13 cell. In an embodiment, the invention includes an aAPC comprising a MOLM-13 cell modified to express a CD86 protein comprising a sequence with greater than 95% identity to an amino acid sequence as set forth in SEQ ID NO:8 and a 4-1BBL protein comprising a sequence with greater than 95% identity to an amino acid sequence as set forth in SEQ ID NO:9, wherein the CD86 protein and the 4-1BBL protein are expressed on the surface of the MOLM-13 cell. In an embodiment, the invention includes an aAPC comprising a MOLM-13 cell modified to express a CD86 protein comprising a sequence with greater than 90% identity to an amino acid sequence as set forth in SEQ ID NO:8 and a 4-1BBL protein comprising a sequence with greater than 90% identity to an amino acid sequence as set forth in SEQ ID NO:9, wherein the CD86 protein and the 4-1BBL protein are expressed on the surface of the MOLM-13 cell. In an embodiment, the invention includes a method of preparing any of the foregoing embodiments of aAPCs.

In an embodiment, the invention includes an aAPC comprising a MOLM-14 cell transduced with one or more viral vectors, wherein the one or more viral vectors comprise a nucleic acid encoding CD86 and a nucleic acid encoding OX40L, and wherein the MOLM-14 cell expresses CD86 and OX40L. In an embodiment, the invention includes an aAPC comprising a MOLM-13 cell transduced with one or more viral vectors, wherein the one or more viral vectors comprise a nucleic acid encoding CD86 and a nucleic acid encoding OX40L, and wherein the MOLM-13 cell expresses CD86 and OX40L. In an embodiment, the invention includes a method of preparing any of the foregoing embodiments of aAPCs.

In an embodiment, the invention includes an aAPC comprising a MOLM-14 cell modified to express a CD86 protein comprising an amino acid sequence as set forth in SEQ ID NO: 8 and a OX40L protein comprising an amino acid sequence as set forth in SEQ ID NO:10, wherein the CD86 protein and the OX40L protein are expressed on the surface of the MOLM-14 cell. In an embodiment, the invention includes an aAPC comprising a MOLM-13 cell modified to express a CD86 protein comprising an amino acid sequence as set forth in SEQ ID NO:8, and conservative amino acid substitutions thereof, and a OX40L protein comprising an amino acid sequence as set forth in SEQ ID NO:10, and conservative amino acid substitutions thereof, wherein the CD86 protein and the OX40L protein are expressed on the surface of the MOLM-13 cell. In an embodiment, the invention includes a method of preparing any of the foregoing embodiments of aAPCs.

In an embodiment, the invention includes an aAPC comprising a MOLM-14 cell modified to express a CD86 protein comprising a sequence with greater than 99% identity to an amino acid sequence as set forth in SEQ ID NO:8 and a OX40L protein comprising a sequence with greater than 99% identity to an amino acid sequence as set forth in SEQ ID NO:10, wherein the CD86 protein and the OX40L protein are expressed on the surface of the MOLM-14 cell. In an embodiment, the invention includes an aAPC comprising a MOLM-14 cell modified to express a CD86 protein comprising a sequence with greater than 98% identity to an amino acid sequence as set forth in SEQ ID NO:8 and a OX40L protein comprising a sequence with greater than 98% identity to an amino acid sequence as set forth in SEQ ID NO:10, wherein the CD86 protein and the OX40L protein are expressed on the surface of the MOLM-14 cell. In an embodiment, the invention includes an aAPC comprising a MOLM-14 cell modified to express a CD86 protein comprising a sequence with greater than 97% identity to an amino acid sequence as set forth in SEQ ID NO:8 and a OX40L protein comprising a sequence with greater than 97% identity to an amino acid sequence as set forth in SEQ ID NO:10, wherein the CD86 protein and the OX40L protein are expressed on the surface of the MOLM-14 cell. In an embodiment, the invention includes an aAPC comprising a MOLM-14 cell modified to express a CD86 protein comprising a sequence with greater than 96% identity to an amino acid sequence as set forth in SEQ ID NO:8 and a OX40L protein comprising a sequence with greater than 96% identity to an amino acid sequence as set forth in SEQ ID NO: 10, wherein the CD86 protein and the OX40L protein are expressed on the surface of the MOLM-14 cell. In an embodiment, the invention includes an aAPC comprising a MOLM-14 cell modified to express a CD86 protein comprising a sequence with greater than 95% identity to an amino acid sequence as set forth in SEQ ID NO:8 and a OX40L protein comprising a sequence with greater than 95% identity to an amino acid sequence as set forth in SEQ ID NO:10, wherein the CD86 protein and the OX40L protein are expressed on the surface of the MOLM-14 cell. In an embodiment, the invention includes an aAPC comprising a MOLM-14 cell modified to express a CD86 protein comprising a sequence with greater than 90% identity to an amino acid sequence as set forth in SEQ ID NO:8 and a OX40L protein comprising a sequence with greater than 90% identity to an amino acid sequence as set forth in SEQ ID NO:10, wherein the CD86 protein and the OX40L protein are expressed on the surface of the MOLM-14 cell. In an embodiment, the invention includes a method of preparing any of the foregoing embodiments of aAPCs.

In any of the foregoing embodiments, it will be understood that an aAPC comprising a MOLM-14 or MOLM-13 cell may be modified to express both OX40L and 4-1BBL.

The sequences for human CD86, human 4-1BBL (CD137L), and human OX40L (CD134L) are given in Table 3.

TABLE 3
Amino acid sequences for human CD86, human 4-1BBL, and human OX40L.

| Identifier | | |
|---|---|---|
| (Description) | Sequence (One-Letter Amino Acid Symbols) | |
| SEQ ID NO: 8 | MGLSNILFVM AFLLSGAAPL KIQAYFNETA DLPCQFANSQ NQSLSELVVF WQDQENLVLN | 60 |
| (human CD86) | EVYLGKEKFD SVHSKYMGRT SFDSDSWTLR LHNLQIKDKG LYQCIIHHKK PTGMIRIHQM | 120 |
| | NSELSVLANF SQPEIVPISN ITENVYINLT CSSIHGYPEP KKMSVLLRTK NSTIEYDGIM | 180 |
| | QKSQDNVTEL YDVSISLSVS FPDVTSNMTI FCILETDKTR LLSSPFSIEL EDPQPPPDHI | 240 |
| | PWITAVLPTV IICVMVFCLI LWKWKKKKRP RNSYKCGTNT MEREESEQTK KREKIHIPER | 300 |
| | SDEAQRVFKS SKTSSCDKSD TCF | 323 |
| SEQ ID NO: 9 | MEYASDASLD PEAPWPPAPA ARACRVLPWA LVAGLLLLLL LAAACAVFLA CPWAVSGARA | 60 |
| (human 4-1BBL, | SPGSAASPRL REGPELSPDD PAGLLDLRQG MFAQLVAQNV LLIDGPLSWY SDPGLAGVSL | 120 |
| CD137) | TGGLSYKEDT KELVVAKAGV YYVFFQLELR RVVAGEGSGS VSLALHLQPL RSAAGAAALA | 180 |
| | LTVDLPPASS EARNSAFGFQ GRLLHLSAGQ RLGVHLHTEA RARHAWQLTQ GATVLGLFRV | 240 |
| | TPEIPAGLPS PRSE | 254 |
| SEQ ID NO: 10 | MERVQPLEEN VGNAARPRFE RNKLLLVASV IQGLGLLLCF TYICLHFSAL QVSHRYPRIQ | 60 |
| (human OX40L, | SIKVQFTEYK KEKGFILTSQ KEDEIMKVQN NSVIINCDGF YLISLKGYFS QEVNISLHYQ | 120 |
| CD134L) | KDEEPLFQLK KVRSWSLMV ASLTYKDKVY LNVTTDNTSL DDFHWGGEL ILIHQNPGEF | 180 |
| | CVL | 183 |

In an embodiment, the invention includes an aAPC comprising a MOLM-14 cell modified to express a first protein that binds to a second protein comprising an amino acid sequence as set forth in SEQ ID NO:13, and conservative amino acid substitutions thereof, and a third protein that binds to a fourth protein comprising an amino acid sequence as set forth in SEQ ID NO: 11 or SEQ ID NO:12, and conservative amino acid substitutions thereof. In an embodiment, the invention includes an aAPC comprising a MOLM-13 cell modified to express a first protein that binds to a second protein comprising an amino acid sequence as set forth in SEQ ID NO:13, and conservative amino acid substitutions thereof, and a third protein that binds to a fourth protein comprising an amino acid sequence as set forth in SEQ ID NO: 11 or SEQ ID NO: 12, and conservative amino acid substitutions thereof. In an embodiment, the invention includes a method of preparing any of the foregoing embodiments of aAPCs.

In an embodiment, the invention includes an aAPC comprising a MOLM-14 cell modified to express a first protein that binds to a second protein comprising a sequence with greater than 99% identity to an amino acid sequence as set forth in SEQ ID NO:13 and a third protein that binds to a fourth protein comprising a sequence with greater than 99% identity to an amino acid sequence as set forth in SEQ ID NO:11 or SEQ ID NO:12. In an embodiment, the invention includes an aAPC comprising a MOLM-14 cell modified to express a first protein that binds to a second protein comprising a sequence with greater than 98% identity to an amino acid sequence as set forth in SEQ ID NO:13 and a third protein that binds to a fourth protein comprising a sequence with greater than 98% identity to an amino acid sequence as set forth in SEQ ID NO:11 or SEQ ID NO:12. In an embodiment, the invention includes an aAPC comprising a MOLM-14 cell modified to express a first protein that binds to a second protein comprising a sequence with greater than 97% identity to an amino acid sequence as set forth in SEQ ID NO:13 and a third protein that binds to a fourth protein comprising a sequence with greater than 97% identity to an amino acid sequence as set forth in SEQ ID NO:11 or SEQ ID NO: 12. In an embodiment, the invention includes an aAPC comprising a MOLM-14 cell modified to express a first protein that binds to a second protein comprising a sequence with greater than 96% identity to an amino acid sequence as set forth in SEQ ID NO:13 and a third protein that binds to a fourth protein comprising a sequence with greater than 96% identity to an amino acid sequence as set forth in SEQ ID NO:11 or SEQ ID NO:12. In an embodiment, the invention includes an aAPC comprising a MOLM-14 cell modified to express a first protein that binds to a second protein comprising a sequence with greater than 95% identity to an amino acid sequence as set forth in SEQ ID NO:13 and a third protein that binds to a fourth protein comprising a sequence with greater than 95% identity to an amino acid sequence as set forth in SEQ ID NO:11 or SEQ ID NO:12. In an embodiment, the invention includes an aAPC comprising a MOLM-14 cell modified to express a first protein that binds to a second protein comprising a sequence with greater than 90% identity to an amino acid sequence as set forth in SEQ ID NO:13 and a third protein that binds to a fourth protein comprising a sequence with greater than 90% identity to an amino acid sequence as set forth in SEQ ID NO:11 or SEQ ID NO: 12. In an embodiment, the invention includes a method of preparing any of the foregoing embodiments of aAPCs.

In an embodiment, the invention includes an aAPC comprising a MOLM-13 cell modified to express a first protein that binds to a second protein comprising a sequence with greater than 99% identity to an amino acid sequence as set forth in SEQ ID NO:13 and a third protein that binds to a fourth protein comprising a sequence with greater than 99% identity to an amino acid sequence as set forth in SEQ ID NO:11 or SEQ ID NO:12. In an embodiment, the invention includes an aAPC comprising a MOLM-13 cell modified to express a first protein that binds to a second protein comprising a sequence with greater than 98% identity to an amino acid sequence as set forth in SEQ ID NO:13 and a third protein that binds to a fourth protein comprising a sequence with greater than 98% identity to an amino acid sequence as set forth in SEQ ID NO:11 or SEQ ID NO:12. In an embodiment, the invention includes an aAPC comprising a MOLM-13 cell modified to express a first protein that binds to a second protein comprising a sequence with greater than 97% identity to an amino acid sequence as set forth in SEQ ID NO:13 and a third protein that binds to a fourth protein comprising a sequence with greater than 97% identity to an amino acid sequence as set forth in SEQ ID NO:11 or SEQ ID NO: 12. In an embodiment, the invention includes an aAPC comprising a MOLM-13 cell modified to express a first protein that binds to a second protein comprising a sequence with greater than 96% identity to an amino acid sequence as set forth in SEQ ID NO:13 and a third protein that binds to a fourth protein comprising a sequence with greater than 96% identity to an amino acid sequence as set forth in SEQ ID NO:11 or SEQ ID NO:12. In an embodiment, the invention includes an aAPC comprising a MOLM-13 cell modified to express a first protein that binds to a second protein comprising a sequence with greater than 95% identity to an amino acid sequence as set forth in SEQ ID NO:13 and a third protein that binds to a fourth protein comprising a sequence with greater than 95% identity to an amino acid sequence as set forth in SEQ ID NO:11 or SEQ ID NO:12. In an embodiment, the invention includes an aAPC comprising a MOLM-13 cell modified to express a first protein that binds to a second protein comprising a sequence with greater than 90% identity to an amino acid sequence as set forth in SEQ ID NO:13 and a third protein that binds to a fourth protein comprising a sequence with greater than 90% identity to an amino acid sequence as set forth in SEQ ID NO:11 or SEQ ID NO: 12. In an embodiment, the invention includes a method of preparing any of the foregoing embodiments of aAPCs.

In an embodiment, the invention includes an aAPC comprising a MOLM-14 cell modified to express a first protein that binds to a second protein comprising an amino acid sequence as set forth in SEQ ID NO:14, and conservative amino acid substitutions thereof, and a third protein that binds to a fourth protein comprising an amino acid sequence as set forth in SEQ ID NO: 11 or SEQ ID NO:12, and conservative amino acid substitutions thereof. In an embodiment, the invention includes an aAPC comprising a MOLM-13 cell modified to express a first protein that binds to a second protein comprising an amino acid sequence as set forth in SEQ ID NO:14, and conservative amino acid substitutions thereof, and a third protein that binds to a fourth protein comprising an amino acid sequence as set forth in SEQ ID NO:11 or SEQ ID NO: 12, and conservative amino acid substitutions thereof. In an embodiment, the invention includes a method of preparing any of the foregoing embodiments of aAPCs.

In an embodiment, the invention includes an aAPC comprising a MOLM-14 cell modified to express a first protein that binds to a second protein comprising a sequence with greater than 99% identity to an amino acid sequence as set forth in SEQ ID NO:14 and a third protein that binds to a fourth protein comprising a sequence with greater than 99% identity to an amino acid sequence as set forth in SEQ ID NO:11 or SEQ ID NO:12. In an embodiment, the invention includes an aAPC comprising a MOLM-14 cell modified to express a first protein that binds to a second protein comprising a sequence with greater than 98% identity to an amino acid sequence as set forth in SEQ ID NO:14 and a third protein that binds to a fourth protein comprising a sequence with greater than 98% identity to an amino acid sequence as set forth in SEQ ID NO:11 or SEQ ID NO:12. In an embodiment, the invention includes an aAPC comprising a MOLM-14 cell modified to express a first protein that binds to a second protein comprising a sequence with greater than 97% identity to an amino acid sequence as set forth in SEQ ID NO:14 and a third protein that binds to a fourth protein comprising a sequence with greater than 97% identity to an amino acid sequence as set forth in SEQ ID NO:11 or SEQ ID NO: 12. In an embodiment, the invention includes an aAPC comprising a MOLM-14 cell modified to express a first protein that binds to a second protein comprising a sequence with greater than 96% identity to an amino acid sequence as set forth in SEQ ID NO:14 and a third protein that binds to a fourth protein comprising a sequence with greater than 96% identity to an amino acid sequence as set forth in SEQ ID NO:11 or SEQ ID NO:12. In an embodiment, the invention includes an aAPC comprising a MOLM-14 cell modified to express a first protein that binds to a second protein comprising a sequence with greater than 95% identity to an amino acid sequence as set forth in SEQ ID NO:14 and a third protein that binds to a fourth protein comprising a sequence with greater than 95% identity to an amino acid sequence as set forth in SEQ ID NO:11 or SEQ ID NO:12. In an embodiment, the invention includes an aAPC comprising a MOLM-14 cell modified to express a first protein that binds to a second protein comprising a sequence with greater than 90% identity to an amino acid sequence as set forth in SEQ ID NO:14 and a third protein that binds to a fourth protein comprising a sequence with greater than 90% identity to an amino acid sequence as set forth in SEQ ID NO:11 or SEQ ID NO: 12. In an embodiment, the invention includes a method of preparing any of the foregoing embodiments of aAPCs.

In an embodiment, the invention includes an aAPC comprising a MOLM-13 cell modified to express a first protein that binds to a second protein comprising a sequence with greater than 99% identity to an amino acid sequence as set forth in SEQ ID NO:14 and a third protein that binds to a fourth protein comprising a sequence with greater than 99% identity to an amino acid sequence as set forth in SEQ ID NO:11 or SEQ ID NO:12. In an embodiment, the invention includes an aAPC comprising a MOLM-13 cell modified to express a first protein that binds to a second protein comprising a sequence with greater than 98% identity to an amino acid sequence as set forth in SEQ ID NO:14 and a third protein that binds to a fourth protein comprising a sequence with greater than 98% identity to an amino acid sequence as set forth in SEQ ID NO:11 or SEQ ID NO:12. In an embodiment, the invention includes an aAPC comprising a MOLM-13 cell modified to express a first protein that binds to a second protein comprising a sequence with greater than 97% identity to an amino acid sequence as set forth in SEQ ID NO:14 and a third protein that binds to a fourth protein comprising a sequence with greater than 97% identity to an amino acid sequence as set forth in SEQ ID NO:11 or SEQ ID NO: 12. In an embodiment, the invention includes an aAPC comprising a MOLM-13 cell modified to express a first protein that binds to a second protein comprising a sequence with greater than 96% identity to an amino acid sequence as set forth in SEQ ID NO:14 and a third protein that binds to a fourth protein comprising a sequence with greater than 96% identity to an amino acid sequence as set forth in SEQ ID NO:11 or SEQ ID NO:12. In an embodiment, the invention includes an aAPC comprising a MOLM-13 cell modified to express a first protein that binds to a second protein comprising a sequence with greater than 95% identity to an amino acid sequence as set forth in SEQ ID NO: 14 and a third protein that binds to a fourth protein comprising a sequence with greater than 95% identity to an amino acid sequence as set forth in SEQ ID NO:11 or SEQ ID NO:12. In an embodiment, the invention includes an aAPC comprising a MOLM-13 cell modified to express a first protein that binds to a second protein comprising a sequence with greater than 90% identity to an amino acid sequence as set forth in SEQ ID NO:14 and a third protein that binds to a fourth protein comprising a sequence with greater than 90% identity to an amino acid sequence as set forth in SEQ ID NO:11 or SEQ ID NO: 12. In an embodiment, the invention includes a method of preparing any of the foregoing embodiments of aAPCs.

The sequences for the ligands to which human CD86 binds (CD28 and CTLA-4), the ligand to which human 4-1BBL binds (4-1BB), and the ligand to which human OX40L binds (OX40) are given in Table 4.

TABLE 4
Amino acid sequences for human CD28, human CTLA-4, human 4-IBB, and human
OX40.

| Identifier | | |
|---|---|---|
| (Description) | Sequence (One-Letter Amino Acid Symbols) | |
| SEQ ID NO: 11 | MLRLLLALNL FPSIQVTGNK ILVKQSPMLV AYDNAVNLSC KYSYNLFSRE FRASLHKGLD | 60 |
| (human CD28) | SAVEVCVVYG NYSQQLQVYS KTGENCDGKL GNESVTFYLQ NLYVNQTDIY FCKIEVMYPP | 120 |
| | PYLDNEKSNG TIIHVKGKHL CPSPLFPGPS KPFWVLVVVG GVLACYSLLV TVAFIIFWVR | 180 |
| | SKRSRLLHSD YMNMTPRRPG PTRKHYQPYA PPRDFAAYRS | 220 |
| SEQ ID NO: 12 | MACLGFQRHK AQLNLATRTW PCTLLFFLLF IPVECKAMHV AQPAVVLASS RGIASFVCEY | 60 |
| (human CTLA-4) | ASPGKATEVR VTVLRQADSQ VTEVCAATYM MGNELTFLDD SICTGTSSGN QVNLTIQGLR | 120 |
| | AMDTGLYICK VELMYPPPYY LGIGNGTQIY VIDPEPCPDS DFLLWILAAV SSGLFFYSFL | 180 |
| | LTAVSLSKML KKRSPLTTGV YVKMPPTEPE CEKQFQPYFI PIN | 223 |
| SEQ ID NO: 13 | MGNSCYNIVA TLLLVLNFER TRSLQDPCSN CPAGTFCDNN RNQICSPCPP NSFSSAGGQR | 60 |
| (human 4-1BB) | TCDICRQCKG VERTRKECSS TSNAECDCTP GFHCLGAGCS MCEQDCKQGQ ELTKKGCKDC | 120 |
| | CFGTENDQKR GICRPWTNCS LDGKSVLVNG THERDVVCGP SPADLSPGAS SVTPPAPARE | 180 |
| | PGHSPQIISF FLALTSTALL FLLFFLTLRF SVVKRGRKKL LYIFKQPFMR PVQTTQEEDG | 240 |
| | CSCRFPEEEE GGCEL | 255 |
| SEQ ID NO: 14 | MCVGARRLGR GPCAALLLLG LGLSTVTGLH CVGDTYPSND RCCHECRPGN GMVSRCSRSQ | 60 |
| (human OX40) | NTVCRPCGPG FYNDVVSSKP CKPCTWCNLR SGSERKQLCT ATQDTVCRCR AGTQPLDSYK | 120 |
| | PGVDCAPCPP GHFSPGDNQA CKPWTNCTLA GKHTLQPASN SSDAICEDRD PPATQPQETQ | 180 |
| | GPPARPITVQ PTEAWPRTSQ GPSTRPVEVP GGRAVAAILG LGLVLGLLGP LAILLALYLL | 240 |
| | RRDQRLPPDA HKPPGGGSFR TPIQEEQADA HSTLAKI | 277 |

In an embodiment, the invention includes an isolated artificial antigen presenting cell (aAPC) comprising a cell that expresses HLA-A/B/C, ICOS-L, and CD58, and is modified to express one or more costimulatory molecules, wherein the aAPC is derived from an EM-3 parental cell line. In an embodiment, the invention includes an aAPC comprising an EM-3 cell that is modified to express one or more costimulatory molecules. In an embodiment, the invention includes an aAPC comprising an EM-2 cell that is modified to express one or more costimulatory molecules.

In an embodiment, the invention includes an aAPC comprising an EM-3 cell that expresses HLA-A/B/C, ICOS-L, and CD58, wherein the cell is modified to express a CD86 protein comprising an amino acid sequence as set forth in SEQ ID NO:8, and conservative amino acid substitutions thereof, and a 4-1BBL protein comprising an amino acid sequence as set forth in SEQ ID NO:9, and conservative amino acid substitutions thereof, and wherein the CD86 protein and the 4-1BBL protein are expressed on the surface of the EM-3 cell.

In an embodiment, the invention includes an aAPC comprising an EM-3 cell transduced with one or more viral vectors, wherein the one or more viral vectors comprise a nucleic acid encoding CD86 and a nucleic acid encoding 4-1BBL, and wherein the EM-3 cell expresses CD86 and 4-1BBL. In an embodiment, the invention includes a method of preparing any of the foregoing embodiments of aAPCs.

In an embodiment, the invention includes an aAPC comprising an EM-3 cell modified to express a CD86 protein comprising an amino acid sequence as set forth in SEQ ID NO:8 and a 4-1BBL protein comprising an amino acid sequence as set forth in SEQ ID NO:9, wherein the CD86 protein and the 4-1BBL protein are expressed on the surface of the EM-3 cell. In an embodiment, the invention includes a method of preparing any of the foregoing embodiments of aAPCs.

In an embodiment, the invention includes an aAPC comprising an EM-3 cell modified to express a CD86 protein comprising a sequence with greater than 99% identity to an amino acid sequence as set forth in SEQ ID NO:8 and a 4-1BBL protein comprising a sequence with greater than 99% identity to an amino acid sequence as set forth in SEQ ID NO:9, wherein the CD86 protein and the 4-1BBL protein are expressed on the surface of the EM-3 cell. In an embodiment, the invention includes an aAPC comprising a EM-3 cell modified to express a CD86 protein comprising a sequence with greater than 98% identity to an amino acid sequence as set forth in SEQ ID NO:8 and a 4-1BBL protein comprising a sequence with greater than 98% identity to an amino acid sequence as set forth in SEQ ID NO:9, wherein the CD86 protein and the 4-1BBL protein are expressed on the surface of the EM-3 cell. In an embodiment, the invention includes an aAPC comprising a EM-3 cell modified to express a CD86 protein comprising a sequence with greater than 97% identity to an amino acid sequence as set forth in SEQ ID NO:8 and a 4-1BBL protein comprising a sequence with greater than 97% identity to an amino acid sequence as set forth in SEQ ID NO:9, wherein the CD86 protein and the 4-1BBL protein are expressed on the surface of the EM-3 cell. In an embodiment, the invention includes an aAPC comprising a EM-3 cell modified to express a CD86 protein comprising a sequence with greater than 96% identity to an amino acid sequence as set forth in SEQ ID NO:8 and a 4-1BBL protein comprising a sequence with greater than 96% identity to an amino acid sequence as set forth in SEQ ID NO:9, wherein the CD86 protein and the 4-1BBL protein are expressed on the surface of the EM-3 cell. In an embodiment, the invention includes an aAPC comprising a EM-3 cell modified to express a CD86 protein comprising a sequence with greater than 95% identity to an amino acid sequence as set forth in SEQ ID NO:8 and a 4-1BBL protein comprising a sequence with greater than 95% identity to an amino acid sequence as set forth in SEQ ID NO:9, wherein the CD86 protein and the 4-1BBL protein are expressed on the surface of the EM-3 cell. In an embodiment, the invention includes an aAPC comprising a EM-3 cell modified to express a CD86 protein comprising a sequence with greater than 90% identity to an amino acid sequence as set forth in SEQ ID NO:8 and a 4-1BBL protein comprising a sequence with greater than 90% identity to an amino acid sequence as set forth in SEQ ID NO:9, wherein the CD86 protein and the 4-1BBL protein are expressed on the surface of the EM-3 cell. In an embodiment, the invention includes a method of preparing any of the foregoing embodiments of aAPCs.

In an embodiment, the invention includes an aAPC comprising an EM-3 cell modified to express a first protein that binds to a second protein comprising an amino acid sequence as set forth in SEQ ID NO:13, and conservative amino acid substitutions thereof, and a third protein that binds to a fourth protein comprising an amino acid sequence as set forth in SEQ ID NO:11 or SEQ ID NO:12, and conservative amino acid substitutions thereof. In an embodiment, the invention includes a method of preparing any of the foregoing embodiments of aAPCs.

In an embodiment, the invention includes an aAPC comprising an EM-3 cell modified to express a first protein that binds to a second protein comprising a sequence with greater than 99% identity to an amino acid sequence as set forth in SEQ ID NO:13 and a third protein that binds to a fourth protein comprising a sequence with greater than 99% identity to an amino acid sequence as set forth in SEQ ID NO:11 or SEQ ID NO:12. In an embodiment, the invention includes an aAPC comprising an EM-3 cell modified to express a first protein that binds to a second protein comprising a sequence with greater than 98% identity to an amino acid sequence as set forth in SEQ ID NO:13 and a third protein that binds to a fourth protein comprising a sequence with greater than 98% identity to an amino acid sequence as set forth in SEQ ID NO:11 or SEQ ID NO:12. In an embodiment, the invention includes an aAPC comprising an EM-3 modified to express a first protein that binds to a second protein comprising a sequence with greater than 97% identity to an amino acid sequence as set forth in SEQ ID NO: 13 and a third protein that binds to a fourth protein comprising a sequence with greater than 97% identity to an amino acid sequence as set forth in SEQ ID NO:11 or SEQ ID NO:12. In an embodiment, the invention includes an aAPC comprising an EM-3 cell modified to express a first protein that binds to a second protein comprising a sequence with greater than 96% identity to an amino acid sequence as set forth in SEQ ID NO:13 and a third protein that binds to a fourth protein comprising a sequence with greater than 96% identity to an amino acid sequence as set forth in SEQ ID NO:11 or SEQ ID NO:12. In an embodiment, the invention includes an aAPC comprising an EM-3 cell modified to express a first protein that binds to a second protein comprising a sequence with greater than 95% identity to an amino acid sequence as set forth in SEQ ID NO:13 and a third protein that binds to a fourth protein comprising a sequence with greater than 95% identity to an amino acid sequence as set forth in SEQ ID NO:11 or SEQ ID NO: 12. In an embodiment, the invention includes an aAPC comprising an EM-3 cell modified to express a first protein that binds to a second protein comprising a sequence with greater than 90% identity to an amino acid sequence as set forth in SEQ ID NO: 13 and a third protein that binds to a fourth protein comprising a sequence with greater than 90% identity to an amino acid sequence as set forth in SEQ ID NO:11 or SEQ ID NO:12. In an embodiment, the invention includes a method of preparing any of the foregoing embodiments of aAPCs.

In an embodiment, the invention includes an aAPC comprising an EM-3 cell modified to express a single chain fragment variable (scFv) binding domain, such as clones 7C12 and 8B3 described herein, to bind the Fc domain of a monoclonal antibody, such as OKT-3, providing an additional proliferative signal.

In an embodiment, the invention includes an aAPC comprising an EM-2 cell modified to express a CD86 protein comprising an amino acid sequence as set forth in SEQ ID NO:8 and a 4-1BBL protein comprising an amino acid sequence as set forth in SEQ ID NO:9, wherein the CD86 protein and the 4-1BBL protein are expressed on the surface of the EM-2 cell. In an embodiment, the invention includes a method of preparing any of the foregoing embodiments of aAPCs.

In an embodiment, the invention includes an aAPC comprising an EM-2 cell modified to express a CD86 protein comprising a sequence with greater than 99% identity to an amino acid sequence as set forth in SEQ ID NO:8 and a 4-1BBL protein comprising a sequence with greater than 99% identity to an amino acid sequence as set forth in SEQ ID NO:9, wherein the CD86 protein and the 4-1BBL protein are expressed on the surface of the EM-2 cell. In an embodiment, the invention includes an aAPC comprising a EM-2 cell modified to express a CD86 protein comprising a sequence with greater than 98% identity to an amino acid sequence as set forth in SEQ ID NO:8 and a 4-1BBL protein comprising a sequence with greater than 98% identity to an amino acid sequence as set forth in SEQ ID NO:9, wherein the CD86 protein and the 4-1BBL protein are expressed on the surface of the EM-2 cell. In an embodiment, the invention includes an aAPC comprising a EM-2 cell modified to express a CD86 protein comprising a sequence with greater than 97% identity to an amino acid sequence as set forth in SEQ ID NO:8 and a 4-1BBL protein comprising a sequence with greater than 97% identity to an amino acid sequence as set forth in SEQ ID NO:9, wherein the CD86 protein and the 4-1BBL protein are expressed on the surface of the EM-2 cell. In an embodiment, the invention includes an aAPC comprising a EM-2 cell modified to express a CD86 protein comprising a sequence with greater than 96% identity to an amino acid sequence as set forth in SEQ ID NO:8 and a 4-1BBL protein comprising a sequence with greater than 96% identity to an amino acid sequence as set forth in SEQ ID NO:9, wherein the CD86 protein and the 4-1BBL protein are expressed on the surface of the EM-2 cell. In an embodiment, the invention includes an aAPC comprising a EM-2 cell modified to express a CD86 protein comprising a sequence with greater than 95% identity to an amino acid sequence as set forth in SEQ ID NO:8 and a 4-1BBL protein comprising a sequence with greater than 95% identity to an amino acid sequence as set forth in SEQ ID NO:9, wherein the CD86 protein and the 4-1BBL protein are expressed on the surface of the EM-2 cell. In an embodiment, the invention includes an aAPC comprising a EM-2 cell modified to express a CD86 protein comprising a sequence with greater than 90% identity to an amino acid sequence as set forth in SEQ ID NO:8 and a 4-1BBL protein comprising a sequence with greater than 90% identity to an amino acid sequence as set forth in SEQ ID NO:9, wherein the CD86 protein and the 4-1BBL protein are expressed on the surface of the EM-2 cell. In an embodiment, the invention includes a method of preparing any of the foregoing embodiments of aAPCs.

In an embodiment, the invention includes an aAPC comprising an EM-2 cell modified to express a first protein that binds to a second protein comprising an amino acid sequence as set forth in SEQ ID NO: 13, and conservative amino acid substitutions thereof, and a third protein that binds to a fourth protein comprising an amino acid sequence as set forth in SEQ ID NO:11 or SEQ ID NO:12, and conservative amino acid substitutions thereof. In an embodiment, the invention includes a method of preparing any of the foregoing embodiments of aAPCs.

In an embodiment, the invention includes an aAPC comprising an EM-2 cell modified to express a first protein that binds to a second protein comprising a sequence with greater than 99% identity to an amino acid sequence as set forth in SEQ ID NO:13 and a third protein that binds to a fourth protein comprising a sequence with greater than 99% identity to an amino acid sequence as set forth in SEQ ID NO:11 or SEQ ID NO:12. In an embodiment, the invention includes an aAPC comprising an EM-2 cell modified to express a first protein that binds to a second protein comprising a sequence with greater than 98% identity to an amino acid sequence as set forth in SEQ ID NO:13 and a third protein that binds to a fourth protein comprising a sequence with greater than 98% identity to an amino acid sequence as set forth in SEQ ID NO:11 or SEQ ID NO:12. In an embodiment, the invention includes an aAPC comprising an EM-2 modified to express a first protein that binds to a second protein comprising a sequence with greater than 97% identity to an amino acid sequence as set forth in SEQ ID NO:13 and a third protein that binds to a fourth protein comprising a sequence with greater than 97% identity to an amino acid sequence as set forth in SEQ ID NO:11 or SEQ ID NO:12. In an embodiment, the invention includes an aAPC comprising an EM-2 cell modified to express a first protein that binds to a second protein comprising a sequence with greater than 96% identity to an amino acid sequence as set forth in SEQ ID NO:13 and a third protein that binds to a fourth protein comprising a sequence with greater than 96% identity to an amino acid sequence as set forth in SEQ ID NO:11 or SEQ ID NO:12. In an embodiment, the invention includes an aAPC comprising an EM-2 cell modified to express a first protein that binds to a second protein comprising a sequence with greater than 95% identity to an amino acid sequence as set forth in SEQ ID NO:13 and a third protein that binds to a fourth protein comprising a sequence with greater than 95% identity to an amino acid sequence as set forth in SEQ ID NO:11 or SEQ ID NO: 12. In an embodiment, the invention includes an aAPC comprising an EM-2 cell modified to express a first protein that binds to a second protein comprising a sequence with greater than 90% identity to an amino acid sequence as set forth in SEQ ID NO:13 and a third protein that binds to a fourth protein comprising a sequence with greater than 90% identity to an amino acid sequence as set forth in SEQ ID NO:11 or SEQ ID NO:12. In an embodiment, the invention includes a method of preparing any of the foregoing embodiments of aAPCs.

In an embodiment, the invention includes an aAPC comprising an EM-2 cell modified to express a single chain fragment variable (scFv) binding domain, such as clones 7C12 and 8B3 described herein, to bind the Fc domain of a monoclonal antibody, such as OKT-3, providing an additional proliferative signal.

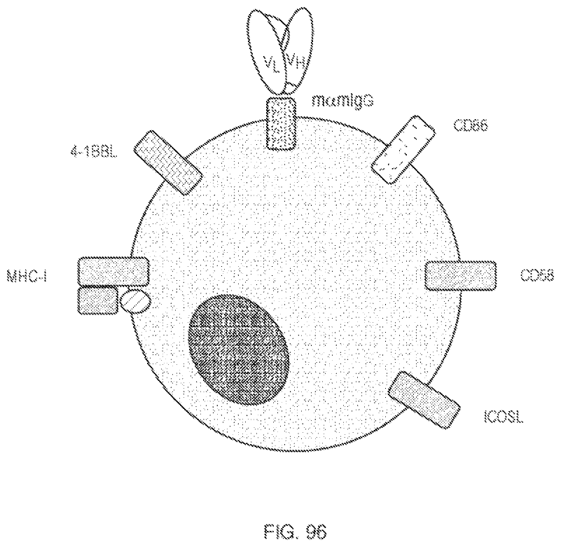

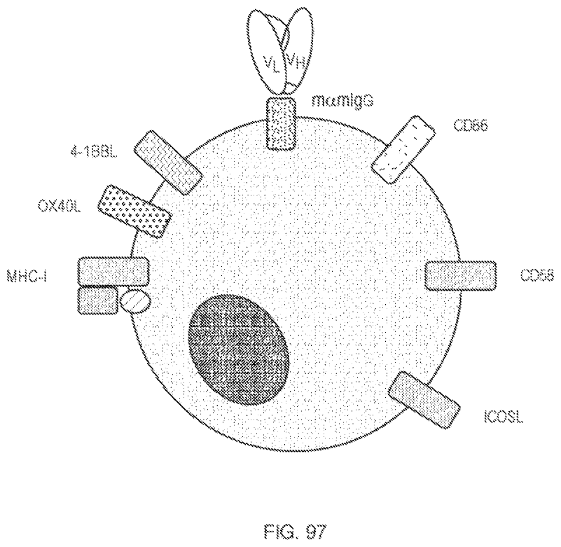

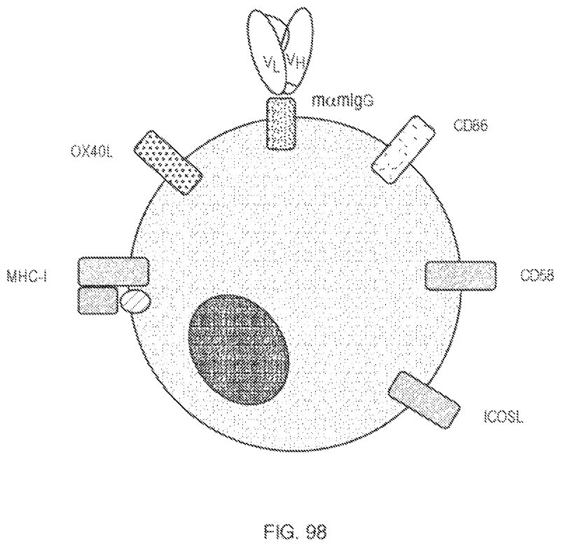

In an embodiment, the invention includes an aAPC comprising an EM-3 or an EM-2 cell modified as depicted in FIG. 96. In an embodiment, the invention includes an aAPC comprising an EM-3 or an EM-2 cell modified as depicted in FIG. 97. In an embodiment, the invention includes an aAPC comprising an EM-3 or an EM-2 cell modified as depicted in FIG. 98.

In an embodiment, the invention includes an aAPC comprising an EM-3 cell that expresses HLA-A/B/C, ICOS-L, and CD58, wherein the cell is modified to express a CD86 protein comprising an amino acid sequence as set forth in SEQ ID NO:8, and conservative amino acid substitutions thereof, and a OX40L protein comprising an amino acid sequence as set forth in SEQ ID NO:10, and conservative amino acid substitutions thereof, and wherein the CD86 protein and the OX40L protein are expressed on the surface of the EM-3 cell.

In an embodiment, the invention includes an aAPC comprising an EM-3 cell transduced with one or more viral vectors, wherein the one or more viral vectors comprise a nucleic acid encoding CD86 and a nucleic acid encoding OX40L, and wherein the EM-3 cell expresses CD86 and OX40L. In an embodiment, the invention includes a method of preparing any of the foregoing embodiments of aAPCs.

In an embodiment, the invention includes an aAPC comprising an EM-3 cell modified to express a CD86 protein comprising an amino acid sequence as set forth in SEQ ID NO:8 and a OX40L protein comprising an amino acid sequence as set forth in SEQ ID NO:10, wherein the CD86 protein and the OX40L protein are expressed on the surface of the EM-3 cell. In an embodiment, the invention includes a method of preparing any of the foregoing embodiments of aAPCs.

In an embodiment, the invention includes an aAPC comprising an EM-3 cell modified to express a CD86 protein comprising a sequence with greater than 99% identity to an amino acid sequence as set forth in SEQ ID NO:8 and a OX40L protein comprising a sequence with greater than 99% identity to an amino acid sequence as set forth in SEQ ID NO:10, wherein the CD86 protein and the OX40L protein are expressed on the surface of the EM-3 cell. In an embodiment, the invention includes an aAPC comprising a EM-3 cell modified to express a CD86 protein comprising a sequence with greater than 98% identity to an amino acid sequence as set forth in SEQ ID NO:8 and a OX40L protein comprising a sequence with greater than 98% identity to an amino acid sequence as set forth in SEQ ID NO:10, wherein the CD86 protein and the OX40L protein are expressed on the surface of the EM-3 cell. In an embodiment, the invention includes an aAPC comprising a EM-3 cell modified to express a CD86 protein comprising a sequence with greater than 97% identity to an amino acid sequence as set forth in SEQ ID NO:8 and a OX40L protein comprising a sequence with greater than 97% identity to an amino acid sequence as set forth in SEQ ID NO:10, wherein the CD86 protein and the OX40L protein are expressed on the surface of the EM-3 cell. In an embodiment, the invention includes an aAPC comprising a EM-3 cell modified to express a CD86 protein comprising a sequence with greater than 96% identity to an amino acid sequence as set forth in SEQ ID NO:8 and a OX40L protein comprising a sequence with greater than 96% identity to an amino acid sequence as set forth in SEQ ID NO:10, wherein the CD86 protein and the OX40L protein are expressed on the surface of the EM-3 cell. In an embodiment, the invention includes an aAPC comprising a EM-3 cell modified to express a CD86 protein comprising a sequence with greater than 95% identity to an amino acid sequence as set forth in SEQ ID NO:8 and a OX40L protein comprising a sequence with greater than 95% identity to an amino acid sequence as set forth in SEQ ID NO: 10, wherein the CD86 protein and the OX40L protein are expressed on the surface of the EM-3 cell. In an embodiment, the invention includes an aAPC comprising a EM-3 cell modified to express a CD86 protein comprising a sequence with greater than 90% identity to an amino acid sequence as set forth in SEQ ID NO:8 and a OX40L protein comprising a sequence with greater than 90% identity to an amino acid sequence as set forth in SEQ ID NO:10, wherein the CD86 protein and the OX40L protein are expressed on the surface of the EM-3 cell. In an embodiment, the invention includes a method of preparing any of the foregoing embodiments of aAPCs.

In an embodiment, the invention includes an aAPC comprising an EM-3 cell modified to express a first protein that binds to a second protein comprising an amino acid sequence as set forth in SEQ ID NO:14, and conservative amino acid substitutions thereof, and a third protein that binds to a fourth protein comprising an amino acid sequence as set forth in SEQ ID NO:11 or SEQ ID NO:12, and conservative amino acid substitutions thereof. In an embodiment, the invention includes a method of preparing any of the foregoing embodiments of aAPCs.

In an embodiment, the invention includes an aAPC comprising an EM-3 cell modified to express a first protein that binds to a second protein comprising a sequence with greater than 99% identity to an amino acid sequence as set forth in SEQ ID NO:14 and a third protein that binds to a fourth protein comprising a sequence with greater than 99% identity to an amino acid sequence as set forth in SEQ ID NO:11 or SEQ ID NO:12. In an embodiment, the invention includes an aAPC comprising an EM-3 cell modified to express a first protein that binds to a second protein comprising a sequence with greater than 98% identity to an amino acid sequence as set forth in SEQ ID NO:14 and a third protein that binds to a fourth protein comprising a sequence with greater than 98% identity to an amino acid sequence as set forth in SEQ ID NO:11 or SEQ ID NO:12. In an embodiment, the invention includes an aAPC comprising an EM-3 modified to express a first protein that binds to a second protein comprising a sequence with greater than 97% identity to an amino acid sequence as set forth in SEQ ID NO: 14 and a third protein that binds to a fourth protein comprising a sequence with greater than 97% identity to an amino acid sequence as set forth in SEQ ID NO:11 or SEQ ID NO:12. In an embodiment, the invention includes an aAPC comprising an EM-3 cell modified to express a first protein that binds to a second protein comprising a sequence with greater than 96% identity to an amino acid sequence as set forth in SEQ ID NO:14 and a third protein that binds to a fourth protein comprising a sequence with greater than 96% identity to an amino acid sequence as set forth in SEQ ID NO:11 or SEQ ID NO:12. In an embodiment, the invention includes an aAPC comprising an EM-3 cell modified to express a first protein that binds to a second protein comprising a sequence with greater than 95% identity to an amino acid sequence as set forth in SEQ ID NO:14 and a third protein that binds to a fourth protein comprising a sequence with greater than 95% identity to an amino acid sequence as set forth in SEQ ID NO:11 or SEQ ID NO: 12. In an embodiment, the invention includes an aAPC comprising an EM-3 cell modified to express a first protein that binds to a second protein comprising a sequence with greater than 90% identity to an amino acid sequence as set forth in SEQ ID NO: 14 and a third protein that binds to a fourth protein comprising a sequence with greater than 90% identity to an amino acid sequence as set forth in SEQ ID NO:11 or SEQ ID NO:12. In an embodiment, the invention includes a method of preparing any of the foregoing embodiments of aAPCs.

In an embodiment, the invention includes an aAPC comprising an EM-3 cell modified to express a single chain fragment variable (scFv) binding domain, such as clones 7C12 and 8B3 described herein, to bind the Fc domain of a monoclonal antibody, such as OKT-3, providing an additional proliferative signal.

In an embodiment, the invention includes an aAPC comprising an EM-2 cell modified to express a CD86 protein comprising an amino acid sequence as set forth in SEQ ID NO:8 and a OX40L protein comprising an amino acid sequence as set forth in SEQ ID NO:10, wherein the CD86 protein and the OX40L protein are expressed on the surface of the EM-2 cell. In an embodiment, the invention includes a method of preparing any of the foregoing embodiments of aAPCs.

In an embodiment, the invention includes an aAPC comprising an EM-2 cell modified to express a CD86 protein comprising a sequence with greater than 99% identity to an amino acid sequence as set forth in SEQ ID NO:8 and a OX40L protein comprising a sequence with greater than 99% identity to an amino acid sequence as set forth in SEQ ID NO:10, wherein the CD86 protein and the OX40L protein are expressed on the surface of the EM-2 cell. In an embodiment, the invention includes an aAPC comprising a EM-2 cell modified to express a CD86 protein comprising a sequence with greater than 98% identity to an amino acid sequence as set forth in SEQ ID NO:8 and a OX40L protein comprising a sequence with greater than 98% identity to an amino acid sequence as set forth in SEQ ID NO:10, wherein the CD86 protein and the OX40L protein are expressed on the surface of the EM-2 cell. In an embodiment, the invention includes an aAPC comprising a EM-2 cell modified to express a CD86 protein comprising a sequence with greater than 97% identity to an amino acid sequence as set forth in SEQ ID NO:8 and a OX40L protein comprising a sequence with greater than 97% identity to an amino acid sequence as set forth in SEQ ID NO:10, wherein the CD86 protein and the OX40L protein are expressed on the surface of the EM-2 cell. In an embodiment, the invention includes an aAPC comprising a EM-2 cell modified to express a CD86 protein comprising a sequence with greater than 96% identity to an amino acid sequence as set forth in SEQ ID NO:8 and a OX40L protein comprising a sequence with greater than 96% identity to an amino acid sequence as set forth in SEQ ID NO:10, wherein the CD86 protein and the OX40L protein are expressed on the surface of the EM-2 cell. In an embodiment, the invention includes an aAPC comprising a EM-2 cell modified to express a CD86 protein comprising a sequence with greater than 95% identity to an amino acid sequence as set forth in SEQ ID NO:8 and a OX40L protein comprising a sequence with greater than 95% identity to an amino acid sequence as set forth in SEQ ID NO: 10, wherein the CD86 protein and the OX40L protein are expressed on the surface of the EM-2 cell. In an embodiment, the invention includes an aAPC comprising a EM-2 cell modified to express a CD86 protein comprising a sequence with greater than 90% identity to an amino acid sequence as set forth in SEQ ID NO:8 and a OX40L protein comprising a sequence with greater than 90% identity to an amino acid sequence as set forth in SEQ ID NO:10, wherein the CD86 protein and the OX40L protein are expressed on the surface of the EM-2 cell. In an embodiment, the invention includes a method of preparing any of the foregoing embodiments of aAPCs.

In an embodiment, the invention includes an aAPC comprising an EM-2 cell modified to express a first protein that binds to a second protein comprising an amino acid sequence as set forth in SEQ ID NO:14, and conservative amino acid substitutions thereof, and a third protein that binds to a fourth protein comprising an amino acid sequence as set forth in SEQ ID NO:11 or SEQ ID NO:12, and conservative amino acid substitutions thereof. In an embodiment, the invention includes a method of preparing any of the foregoing embodiments of aAPCs.

In an embodiment, the invention includes an aAPC comprising an EM-2 cell modified to express a first protein that binds to a second protein comprising a sequence with greater than 99% identity to an amino acid sequence as set forth in SEQ ID NO:14 and a third protein that binds to a fourth protein comprising a sequence with greater than 99% identity to an amino acid sequence as set forth in SEQ ID NO:11 or SEQ ID NO:12. In an embodiment, the invention includes an aAPC comprising an EM-2 cell modified to express a first protein that binds to a second protein comprising a sequence with greater than 98% identity to an amino acid sequence as set forth in SEQ ID NO:14 and a third protein that binds to a fourth protein comprising a sequence with greater than 98% identity to an amino acid sequence as set forth in SEQ ID NO:11 or SEQ ID NO:12. In an embodiment, the invention includes an aAPC comprising an EM-2 modified to express a first protein that binds to a second protein comprising a sequence with greater than 97% identity to an amino acid sequence as set forth in SEQ ID NO:14 and a third protein that binds to a fourth protein comprising a sequence with greater than 97% identity to an amino acid sequence as set forth in SEQ ID NO:11 or SEQ ID NO:12. In an embodiment, the invention includes an aAPC comprising an EM-2 cell modified to express a first protein that binds to a second protein comprising a sequence with greater than 96% identity to an amino acid sequence as set forth in SEQ ID NO:14 and a third protein that binds to a fourth protein comprising a sequence with greater than 96% identity to an amino acid sequence as set forth in SEQ ID NO:11 or SEQ ID NO:12. In an embodiment, the invention includes an aAPC comprising an EM-2 cell modified to express a first protein that binds to a second protein comprising a sequence with greater than 95% identity to an amino acid sequence as set forth in SEQ ID NO:14 and a third protein that binds to a fourth protein comprising a sequence with greater than 95% identity to an amino acid sequence as set forth in SEQ ID NO:11 or SEQ ID NO: 12. In an embodiment, the invention includes an aAPC comprising an EM-2 cell modified to express a first protein that binds to a second protein comprising a sequence with greater than 90% identity to an amino acid sequence as set forth in SEQ ID NO: 14 and a third protein that binds to a fourth protein comprising a sequence with greater than 90% identity to an amino acid sequence as set forth in SEQ ID NO:11 or SEQ ID NO:12. In an embodiment, the invention includes a method of preparing any of the foregoing embodiments of aAPCs.

In an embodiment, the invention includes an aAPC comprising an EM-2 cell modified to express a single chain fragment variable (scFv) binding domain, such as clones 7C12 and 8B3 described herein, to bind the Fc domain of a monoclonal antibody, such as OKT-3, providing an additional proliferative signal.

In an embodiment, the invention includes an aAPC comprising an EM-3 or an EM-2 cell modified as depicted in FIG. 96. In an embodiment, the invention includes an aAPC comprising an EM-3 or an EM-2 cell modified as depicted in FIG. 97. In an embodiment, the invention includes an aAPC comprising an EM-3 or an EM-2 cell modified as depicted in FIG. 98.

In any of the foregoing embodiments, it is understood that an aAPC comprising an EM-3 or EM-2 cell may be modified to express both OX40L and 4-1BBL.

In an embodiment, the invention includes an isolated artificial antigen presenting cell (aAPC) comprising a cell that expresses CD58, and is modified to express one or more costimulatory molecules, wherein the aAPC is derived from a K562-lineage parental cell line. In an embodiment, the invention includes an aAPC comprising a K562-lineage cell that is modified to express one or more costimulatory molecules. In an embodiment, the K562 lineage parental cell line is deposited under accession no. ATCC CCL-243 and also at European Collection of Authenticated Cell Cultures (ECACCECACC 89121407).

In an embodiment, the invention includes an aAPC comprising a K562-lineage cell that expresses CD58, wherein the cell is modified to express a CD86 protein comprising an amino acid sequence as set forth in SEQ ID NO:8, and conservative amino acid substitutions thereof, and a 4-1BBL protein comprising an amino acid sequence as set forth in SEQ ID NO:9, and conservative amino acid substitutions thereof, and wherein the CD86 protein and the 4-1BBL protein are expressed on the surface of the K562-lineage cell.

In an embodiment, the invention includes an aAPC comprising a K562-lineage cell transduced with one or more viral vectors, wherein the one or more viral vectors comprise a nucleic acid encoding CD86 and a nucleic acid encoding 4-1BBL, and wherein the K562-lineage cell expresses CD86 and 4-1BBL. In an embodiment, the invention includes a method of preparing any of the foregoing embodiments of aAPCs.

In an embodiment, the invention includes an aAPC comprising a K562-lineage cell modified to express a CD86 protein comprising an amino acid sequence as set forth in SEQ ID NO: 8 and a 4-1BBL protein comprising an amino acid sequence as set forth in SEQ ID NO:9, wherein the CD86 protein and the 4-1BBL protein are expressed on the surface of the K562-lineage cell. In an embodiment, the invention includes a method of preparing any of the foregoing embodiments of aAPCs.

In an embodiment, the invention includes an aAPC comprising a K562-lineage cell modified to express a CD86 protein comprising a sequence with greater than 99% identity to an amino acid sequence as set forth in SEQ ID NO:8 and a 4-1BBL protein comprising a sequence with greater than 99% identity to an amino acid sequence as set forth in SEQ ID NO:9, wherein the CD86 protein and the 4-1BBL protein are expressed on the surface of the K562-lineage cell. In an embodiment, the invention includes an aAPC comprising a K562-lineage cell modified to express a CD86 protein comprising a sequence with greater than 98% identity to an amino acid sequence as set forth in SEQ ID NO:8 and a 4-1BBL protein comprising a sequence with greater than 98% identity to an amino acid sequence as set forth in SEQ ID NO:9, wherein the CD86 protein and the 4-1BBL protein are expressed on the surface of the K562-lineage cell. In an embodiment, the invention includes an aAPC comprising a K562-lineage cell modified to express a CD86 protein comprising a sequence with greater than 97% identity to an amino acid sequence as set forth in SEQ ID NO:8 and a 4-1BBL protein comprising a sequence with greater than 97% identity to an amino acid sequence as set forth in SEQ ID NO:9, wherein the CD86 protein and the 4-1BBL protein are expressed on the surface of the K562-lineage cell. In an embodiment, the invention includes an aAPC comprising a K562-lineage cell modified to express a CD86 protein comprising a sequence with greater than 96% identity to an amino acid sequence as set forth in SEQ ID NO:8 and a 4-1BBL protein comprising a sequence with greater than 96% identity to an amino acid sequence as set forth in SEQ ID NO:9, wherein the CD86 protein and the 4-1BBL protein are expressed on the surface of the K562-lineage cell. In an embodiment, the invention includes an aAPC comprising a K562-lineage cell modified to express a CD86 protein comprising a sequence with greater than 95% identity to an amino acid sequence as set forth in SEQ ID NO:8 and a 4-1BBL protein comprising a sequence with greater than 95% identity to an amino acid sequence as set forth in SEQ ID NO:9, wherein the CD86 protein and the 4-1BBL protein are expressed on the surface of the K562-lineage cell. In an embodiment, the invention includes an aAPC comprising a K562-lineage cell modified to express a CD86 protein comprising a sequence with greater than 90% identity to an amino acid sequence as set forth in SEQ ID NO:8 and a 4-1BBL protein comprising a sequence with greater than 90% identity to an amino acid sequence as set forth in SEQ ID NO:9, wherein the CD86 protein and the 4-1BBL protein are expressed on the surface of the K562-lineage cell. In an embodiment, the invention includes a method of preparing any of the foregoing embodiments of aAPCs.

In an embodiment, the invention includes an aAPC comprising a K562-lineage cell modified to express a first protein that binds to a second protein comprising an amino acid sequence as set forth in SEQ ID NO:11, and conservative amino acid substitutions thereof, and a third protein that binds to a fourth protein comprising an amino acid sequence as set forth in SEQ ID NO: 12 or SEQ ID NO:13, and conservative amino acid substitutions thereof. In an embodiment, the invention includes a method of preparing any of the foregoing embodiments of aAPCs.

In an embodiment, the invention includes an aAPC comprising a K562-lineage cell modified to express a first protein that binds to a second protein comprising a sequence with greater than 99% identity to an amino acid sequence as set forth in SEQ ID NO:11 and a third protein that binds to a fourth protein comprising a sequence with greater than 99% identity to an amino acid sequence as set forth in SEQ ID NO:12 or SEQ ID NO:13. In an embodiment, the invention includes an aAPC comprising a K562-lineage cell modified to express a first protein that binds to a second protein comprising a sequence with greater than 98% identity to an amino acid sequence as set forth in SEQ ID NO:11 and a third protein that binds to a fourth protein comprising a sequence with greater than 98% identity to an amino acid sequence as set forth in SEQ ID NO:12 or SEQ ID NO:13. In an embodiment, the invention includes an aAPC comprising a K562-lineage modified to express a first protein that binds to a second protein comprising a sequence with greater than 97% identity to an amino acid sequence as set forth in SEQ ID NO:11 and a third protein that binds to a fourth protein comprising a sequence with greater than 97% identity to an amino acid sequence as set forth in SEQ ID NO: 12 or SEQ ID NO: 13. In an embodiment, the invention includes an aAPC comprising a K562-lineage cell modified to express a first protein that binds to a second protein comprising a sequence with greater than 96% identity to an amino acid sequence as set forth in SEQ ID NO:11 and a third protein that binds to a fourth protein comprising a sequence with greater than 96% identity to an amino acid sequence as set forth in SEQ ID NO:12 or SEQ ID NO:13. In an embodiment, the invention includes an aAPC comprising a K562-lineage cell modified to express a first protein that binds to a second protein comprising a sequence with greater than 95% identity to an amino acid sequence as set forth in SEQ ID NO:11 and a third protein that binds to a fourth protein comprising a sequence with greater than 95% identity to an amino acid sequence as set forth in SEQ ID NO:12 or SEQ ID NO:13. In an embodiment, the invention includes an aAPC comprising a K562-lineage cell modified to express a first protein that binds to a second protein comprising a sequence with greater than 90% identity to an amino acid sequence as set forth in SEQ ID NO:11 and a third protein that binds to a fourth protein comprising a sequence with greater than 90% identity to an amino acid sequence as set forth in SEQ ID NO: 12 or SEQ ID NO: 13. In an embodiment, the invention includes a method of preparing any of the foregoing embodiments of aAPCs.

In an embodiment, the invention includes an aAPC comprising an K562-lineage cell modified to express a single chain fragment variable (scFv) binding domain, such as clones 7C12 and 8B3 described herein, to bind the Fc domain of a monoclonal antibody, such as OKT-3, providing an additional proliferative signal.

### Methods of Preparing Artificial Antigen Presenting Cells

In an embodiment, a method of preparing an aAPC includes the step of stable incorporation of genes for production of CD86 and 4-1BBL. In an embodiment, a method of preparing an aAPC includes the step of retroviral transduction. In an embodiment, a method of preparing an aAPC includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In an embodiment, a method of preparing an aAPC includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In an embodiment, a method of preparing an aAPC includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In an embodiment, a method of preparing an aAPC includes the step of stable incorporation of genes for transient production of CD86 and 4-1BBL. In an embodiment, a method of preparing an aAPC includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. In an embodiment, a method of preparing an aAPC includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In an embodiment, a method of preparing an aAPC includes the step of liposomal transfection. Liposomal transfection methods, such as methods that employ a 1:1 (w/w) liposome formulation of the cationic lipid N-[1-(2,3-dioleyloxy) propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of preparing an aAPC includes the step of transfection using methods described in U.S. Pat. Nos. 5,766,902; 6,025,337; 6,410,517; 6,475,994; and 7,189,705; the disclosures of each of which are incorporated by reference herein.

In an embodiment, the aAPC is transduced by first using the Gateway cloning method (commercially available from ThermoFisher, Inc.) to prepare vector for lentiviral transduction, followed by lentiviral transduction using the vector and one or more associated helper plasmids, as is also described elsewhere herein. In the Gateway cloning method, a gene is selected (such as CD86) and is then provided with primers and amplified using PCR technology with the help of an attB tagged primer pair. The PCR fragment is then combined with a donor vector (pDONR, such as pDONR221) that includes attP sites to provide an entry clone, using the BP reaction. An integration reaction between the attB and the attP sites combines the PCR fragment with the donor vector. The resulting entry clone contains the gene of interest flanked by attL sites. The LR reaction is then used to combine the entry clone with a destination vector to produce an expression vector. In the LR reaction, a recombination reaction is used to link the entry clone with the destination vector (such as pLV430G) using the attL and attR sites and a clonase enzyme. The attL sites are already found in the entry clone, while the destination vector includes the attR sites. The LR reaction is carried out to transfer the sequence of interest into one or more destination vectors in simultaneous reactions.

In some embodiments, the aAPCs described herein may be grown and maintained under serum-based media and/or serum free media. According to an exemplary method, aAPCs may be cultured in 24 well plates at a cell density of about 1×10⁶ cells per well for 3 to 5 days. The cells may then be isolated and/or washed by centrifugation and resuspended in media or cryopreserved in an appropriate cryopreservation media (e.g., CryoStor 10 (BioLife Solutions)) and stored in a −80° C. freezer.

In some embodiments, the aAPCs described herein may be grown in the presence of serum-based media. In some embodiments, the aAPCs described herein by may be grown in the presence of serum-based media that includes human serum (hSerum) containing media (e.g., cDMEM with 10% hSerum). In some embodiments, the aAPCs grown in the presence of serum-based media may be selected from the group consisting of aMOLM-13 cells, aMOLM-14 cells, and aEM3 cells.

In some embodiments, the aAPCs described herein may be grown in the presence of serum free media. In some embodiments, the serum free media may be selected from the group consisting of CTS Optmizer (ThermoFisher), Xvivo-20 (Lonza), Prime T Cell CDM (Irvine), XFSM (MesenCult), and the like. In some embodiments, the aAPCs grown in the presence of serum free media may be selected from the group consisting of aMOLM-13 cells, aMOLM-14 cells, and aEM3 cells.

### Methods of Expanding Tumor Infiltrating Lymphocytes and T Cells

In an embodiment, the invention includes a method of expanding tumor infiltrating lymphocytes (TILs), the method comprising contacting a population of TILs comprising at least one TIL with an aAPC described herein, wherein said aAPC comprises at least one co-stimulatory ligand that specifically binds with a co-stimulatory molecule expressed on the cellular surface of the TILs, wherein binding of said co-stimulatory molecule with said co-stimulatory ligand induces proliferation of the TILs, thereby specifically expanding TILs.

In an embodiment, the invention provides a method of expanding a population of tumor infiltrating lymphocytes (TILs) using any of the aAPCs of the present disclosure, the method comprising the steps as described in Jin, et al., J. Immunotherapy 2012, 35, 283-292, the disclosure of which is incorporated by reference herein. For example, the tumor may be placed in enzyme media and mechanically dissociated for approximately 1 minute. The mixture may then be incubated for 30 minutes at 37° C. in 5% CO₂ and then mechanically disrupted again for approximately 1 minute. After incubation for 30 minutes at 37° C. in 5% CO₂, the tumor may be mechanically disrupted a third time for approximately 1 minute. If after the third mechanical disruption, large pieces of tissue are present, 1 or 2 additional mechanical dissociations may be applied to the sample, with or without 30 additional minutes of incubation at 37° C. in 5% CO₂. At the end of the final incubation, if the cell suspension contains a large number of red blood cells or dead cells, a density gradient separation using Ficoll may be performed to remove these cells. TIL cultures were initiated in 24-well plates (Costar 24-well cell culture cluster, flat bottom; Corning Incorporated, Corning, NY), each well may be seeded with 1×10⁶ tumor digest cells or one tumor fragment approximately 1 to 8 mm³ in size in 2 mL of complete medium (CM) with IL-2 (6000 IU/mL; Chiron Corp., Emeryville, CA). CM consists of RPMI 1640 with GlutaMAX, supplemented with 10% human AB serum, 25 mM Hepes, and 10 mg/mL gentamicin. Cultures may be initiated in gas-permeable flasks with a 40 mL capacity and a 10 cm² gas-permeable silicon bottom (G-Rex 10; Wilson Wolf Manufacturing, New Brighton, each flask may be loaded with 10-40×10⁶ viable tumor digest cells or 5-30 tumor fragments in 10-40 mL of CM with IL-2. G-Rex 10 and 24-well plates may be incubated in a humidified incubator at 37° C. in 5% CO₂ and 5 days after culture initiation, half the media may be removed and replaced with fresh CM and IL-2 and after day 5, half the media may be changed every 2-3 days. Rapid expansion protocol (REP) of TILs may be performed using T-175 flasks and gas-permeable bags or gas-permeable G-Rex flasks, as described elsewhere herein, using the aAPCs of the present disclosure. For REP in T-175 flasks, 1×10⁶ TILs may be suspended in 150 mL of media in each flask. The TIL may be cultured with aAPCs of the present disclosure at a ratio described herein, in a 1 to 1 mixture of CM and AIM-V medium (50/50 medium), supplemented with 3000 IU/mL of IL-2 and 30 ng/mL of anti-CD3 antibody (OKT-3). The T-175 flasks may be incubated at 37° C. in 5% CO₂. Half the media may be changed on day 5 using 50/50 medium with 3000 IU/mL of IL-2. On day 7, cells from 2 T-175 flasks may be combined in a 3 L bag and 300 mL of AIM-V with 5% human AB serum and 3000 IU/mL of IL-2 may be added to the 300 mL of TIL suspension. The number of cells in each bag may be counted every day or two days, and fresh media may be added to keep the cell count between 0.5 and 2.0×10⁶ cells/mL. For REP in 500 mL capacity flasks with 100 cm² gas-permeable silicon bottoms (e.g., G-Rex 100, Wilson Wolf Manufacturing, as described elsewhere herein), 5×10⁶ or 10×10⁶ TILs may be cultured with aAPCs at a ratio described herein (e.g., 1 to 100) in 400 mL of 50/50 medium, supplemented with 3000 IU/mL of IL-2 and 30 ng/ml of anti-CD3 antibody (OKT-3). The G-Rex100 flasks may be incubated at 37° C. in 5% CO₂. On day five, 250 mL of supernatant may be removed and placed into centrifuge bottles and centrifuged at 1500 rpm (491 g) for 10 minutes. The obtained TIL pellets may be resuspended with 150 mL of fresh 50/50 medium with 3000 IU/mL of IL-2 and added back to the G-Rex 100 flasks. When TIL are expanded serially in G-Rex 100 flasks, on day seven the TIL in each G-Rex100 are suspended in the 300 mL of media present in each flask and the cell suspension may be divided into three 100 mL aliquots that may be used to seed 3 G-Rex100 flasks. About 150 mL of AIM-V with 5% human AB serum and 3000 IU/mL of IL-2 may then be added to each flask. G-Rex100 flasks may then be incubated at 37° C. in 5% CO₂, and after four days, 150 mL of AIM-V with 3000 IU/mL of IL-2 may be added to each G-Rex100 flask. After this, the REP may be completed by harvesting cells on day 14 of culture.

As described herein, TILs may be expanded advantageously in the presence of serum free media. In some embodiments, the TIL expansion methods described herein may include the use of serum free media rather than serum-based media (e.g., complete media or CM1). In some embodiments, the TIL expansion methods described herein may use serum free media rather than serum-based media. In some embodiments, the serum free media may be selected from the group consisting of CTS Optmizer (ThermoFisher), Xvivo-20 (Lonza), Prime T Cell CDM (Irvine), and the like.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the cell culture medium further comprises IL-2 at an initial concentration of about 3000 IU/mL and OKT-3 antibody at an initial concentration of about 30 ng/mL.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the population of APCs expands the population of TILs by at least 50-fold over a period of 7 days in a cell culture medium.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the myeloid cell endogenously expresses HLA-A/B/C, ICOS-L, and CD58.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the myeloid cell is a MOLM-14 cell.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the myeloid cell is a MOLM-13 cell.

In an embodiment, the invention provides a method of expanding a population of tumor infiltrating lymphocytes (TILs), the method comprising the steps of:
(c) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(d) contacting the population of TILs with the population of aAPCs in a cell culture medium,
wherein the myeloid cell is a EM-3 cell.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the CD86 protein comprises an amino acid sequence as set forth in SEQ ID NO:8, or conservative amino acid substitutions thereof, and the 4-1BBL protein comprises an amino acid sequence as set forth in SEQ ID NO:9, or conservative amino acid substitutions thereof.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the nucleic acid encoding CD86 comprises a nucleic acid sequence as set forth in SEQ ID NO:19 and the nucleic acid encoding 4-1BBL comprises a nucleic acid sequence as set forth in SEQ ID NO:16.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the expansion is performed using a gas permeable container.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the ratio of the population of TILs to the population of aAPCs is between 1 to 200 and 1 to 400.

In an embodiment, the invention provides a method of expanding a population of tumor infiltrating lymphocytes (TILs), the method comprising the steps of:
(a) transducing a myeloid cell with one or more viral vectors to obtain a population of artificial antigen presenting cells (aAPCs), wherein the one or more viral vectors comprise a nucleic acid encoding CD86 and a nucleic acid encoding 4-1BBL, and wherein the myeloid cell expresses a CD86 protein and a 4-1BBL protein, and
(b) contacting the population of TILs with the population of aAPCs in a cell culture medium,
wherein the ratio of the population of TILs to the population of aAPCs is about 1 to 300.

In an embodiment, the invention provides a method of expanding tumor infiltrating lymphocytes (TILs), the method comprising contacting a population of TILs comprising a population of TILs with a myeloid artificial antigen presenting cell (aAPC), wherein the myeloid aAPC comprises at least two co-stimulatory ligands that specifically bind with at least two co-stimulatory molecule on the TILs, wherein binding of the co-stimulatory molecules with the co-stimulatory ligand induces proliferation of the TILs, thereby specifically expanding TILs, and wherein the at least two co-stimulatory ligands comprise CD86 and 4-1BBL.

In any of the foregoing embodiments, the aAPC may further comprise OX40L in addition to 4-1BBL, or may comprise OX40L instead of 4-1BBL.

In an embodiment, a method of expanding or treating a cancer includes a step wherein TILs are obtained from a patient tumor sample. A patient tumor sample may be obtained using methods known in the art. For example, TILs may be cultured from enzymatic tumor digests and tumor fragments (about 1 to about 8 mm³ in size) from sharp dissection. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/ml of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

In an embodiment, REP can be performed in a gas permeable container using the aAPCs of the present disclosure by any suitable method. For example, TILs can be rapidly expanded using non-specific T cell receptor stimulation in the presence of interleukin-2 (IL-2) or interleukin-15 (IL-15). The non-specific T cell receptor stimulus can include, for example, about 30 ng/mL of an anti-CD3 antibody, e.g. OKT-3, a monoclonal anti-CD3 antibody (commercially available from Ortho-McNeil, Raritan, NJ, USA or Miltenyi Biotech, Auburn, CA, USA) or UHCT-1 (commercially available from BioLegend, San Diego, CA, USA). TILs can be rapidly expanded by further stimulation of the TILs in vitro with one or more antigens, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2.

In an embodiment, a method for expanding TILs may include using about 5000 mL to about 25000 mL of cell culture medium, about 5000 mL to about 10000 mL of cell culture medium, or about 5800 mL to about 8700 mL of cell culture medium. In an embodiment, a method for expanding TILs may include using about 1000 mL to about 2000 mL of cell medium, about 2000 mL to about 3000 mL of cell culture medium, about 3000 mL to about 4000 mL of cell culture medium, about 4000 mL to about 5000 mL of cell culture medium, about 5000 mL to about 6000 mL of cell culture medium, about 6000 mL to about 7000 mL of cell culture medium, about 7000 mL to about 8000 mL of cell culture medium, about 8000 mL to about 9000 mL of cell culture medium, about 9000 mL to about 10000 mL of cell culture medium, about 10000 mL to about 15000 mL of cell culture medium, about 15000 mL to about 20000 mL of cell culture medium, or about 20000 mL to about 25000 mL of cell culture medium. In an embodiment, expanding the number of TILs uses no more than one type of cell culture medium. Any suitable cell culture medium may be used, e.g., AIM-V cell medium (L-glutamine, 50 μM streptomycin sulfate, and 10 μM gentamicin sulfate) cell culture medium (Invitrogen, Carlsbad, CA, USA). In this regard, the inventive methods advantageously reduce the amount of medium and the number of types of medium required to expand the number of TIL. In an embodiment, expanding the number of TIL may comprise feeding the cells no more frequently than every third or fourth day. Expanding the number of cells in a gas permeable container simplifies the procedures necessary to expand the number of cells by reducing the feeding frequency necessary to expand the cells.

In an embodiment, the rapid expansion is performed using a gas permeable container. Such embodiments allow for cell populations to expand from about 5×10⁵ cells/cm² to between 10×10⁶ and 30×10⁶ cells/cm². In an embodiment, this expansion occurs without feeding. In an embodiment, this expansion occurs without feeding so long as medium resides at a height of about 10 cm in a gas-permeable flask. In an embodiment this is without feeding but with the addition of one or more cytokines. In an embodiment, the cytokine can be added as a bolus without any need to mix the cytokine with the medium. Such containers, devices, and methods are known in the art and have been used to expand TILs, and include those described in U.S. Patent Application Publication No. US 2014/0377739 A1, International Patent Application Publication No. WO 2014/210036 A1, U.S. Patent Application Publication No. US 2013/0115617 A1, International Publication No. WO 2013/188427 A1, U.S. Patent Application Publication No. US 2011/0136228 A1, U.S. Pat. No. 8,809,050, International Patent Application Publication No. WO 2011/072088 A2, U.S. Patent Application Publication No. US 2016/0208216 A1, U.S. Patent Application Publication No. US 2012/0244133 A1, International Patent Application Publication No. WO 2012/129201 A1, U.S. Patent Application Publication No. US 2013/0102075 A1, U.S. Pat. No. 8,956,860, International Patent Application Publication No. WO 2013/173835 A1, and U.S. Patent Application Publication No. US 2015/0175966 A1, the disclosures of which are incorporated herein by reference. Such processes are also described in Jin, et al., J. Immunotherapy 2012, 35, 283-292, the disclosure of which is incorporated by reference herein.

In an embodiment, the gas permeable container is a G-Rex 10 flask (Wilson Wolf Manufacturing Corporation, New Brighton, MN, USA). In an embodiment, the gas permeable container includes a 10 cm² gas permeable culture surface. In an embodiment, the gas permeable container includes a 40 mL cell culture medium capacity. In an embodiment, the gas permeable container provides 100 to 300 million TILs after 2 medium exchanges.

In an embodiment, the gas permeable container is a G-Rex 100 flask (Wilson Wolf Manufacturing Corporation, New Brighton, MN, USA). In an embodiment, the gas permeable container includes a 100 cm² gas permeable culture surface. In an embodiment, the gas permeable container includes a 450 mL cell culture medium capacity. In an embodiment, the gas permeable container provides 1 to 3 billion TILs after 2 medium exchanges.

In an embodiment, the gas permeable container is a G-Rex 100M flask (Wilson Wolf Manufacturing Corporation, New Brighton, MN, USA). In an embodiment, the gas permeable container includes a 100 cm² gas permeable culture surface. In an embodiment, the gas permeable container includes a 1000 mL cell culture medium capacity. In an embodiment, the gas permeable container provides 1 to 3 billion TILs without medium exchange.

In an embodiment, the gas permeable container is a G-Rex 100 L flask (Wilson Wolf Manufacturing Corporation, New Brighton, MN, USA). In an embodiment, the gas permeable container includes a 100 cm² gas permeable culture surface. In an embodiment, the gas permeable container includes a 2000 mL cell culture medium capacity. In an embodiment, the gas permeable container provides 1 to 3 billion TILs without medium exchange.

In an embodiment, the gas permeable container is a G-Rex 24 well plate (Wilson Wolf Manufacturing Corporation, New Brighton, MN, USA). In an embodiment, the gas permeable container includes a plate with wells, wherein each well includes a 2 cm² gas permeable culture surface. In an embodiment, the gas permeable container includes a plate with wells, wherein each well includes a 8 mL cell culture medium capacity. In an embodiment, the gas permeable container provides 20 to 60 million cells per well after 2 medium exchanges.

In an embodiment, the gas permeable container is a G-Rex 6 well plate (Wilson Wolf Manufacturing Corporation, New Brighton, MN, USA). In an embodiment, the gas permeable container includes a plate with wells, wherein each well includes a 10 cm² gas permeable culture surface. In an embodiment, the gas permeable container includes a plate with wells, wherein each well includes a 40 mL cell culture medium capacity. In an embodiment, the gas permeable container provides 100 to 300 million cells per well after 2 medium exchanges.

In an embodiment, the cell medium in the first and/or second gas permeable container is unfiltered. The use of unfiltered cell medium may simplify the procedures necessary to expand the number of cells. In an embodiment, the cell medium in the first and/or second gas permeable container lacks beta-mercaptoethanol (BME).

In an embodiment, the duration of the method comprising obtaining a tumor tissue sample from the mammal; culturing the tumor tissue sample in a first gas permeable container containing cell medium therein; obtaining TILs from the tumor tissue sample; expanding the number of TILs in a second gas permeable container containing cell medium therein using aAPCs for a duration of about 14 to about 42 days, e.g., about 28 days.

In an embodiment, the rapid expansion uses about 1×10⁹ to about 1×10¹¹ aAPCs. In an embodiment, the rapid expansion uses about 1×10⁹ aAPCs. In an embodiment, the rapid expansion uses about 1×10¹⁰ aAPCs. In an embodiment, the rapid expansion uses about 1×10¹¹ aAPCs.

In an embodiment, the ratio of TILs to aAPCs (TIL:aAPC) is selected from the group consisting of 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:225, 1:250, 1:275, 1:300, 1:350, 1:400, 1:450, and 1:500. In a preferred embodiment, the ratio of TILs to aAPCs (TIL:aAPC) is about 1:90. In a preferred embodiment, the ratio of TILs to aAPCs (TIL:aAPC) is about 1:95. In a preferred embodiment, the ratio of TILs to aAPCs (TIL:aAPC) is about 1:100. In a preferred embodiment, the ratio of TILs to aAPCs (TIL:aAPC) is about 1:105. In a preferred embodiment, the ratio of TILs to aAPCs (TIL:aAPC) is about 1:110.

In an embodiment, the ratio of TILs to aAPCs in the rapid expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to aAPCs in the rapid expansion is between 1 to 50 and 1 to 300. In an embodiment, the ratio of TILs to aAPCs in the rapid expansion is between 1 to 100 and 1 to 200.

In an embodiment, the cell culture medium further comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In an embodiment, the cell culture medium comprises an OKT-3 antibody. In a preferred embodiment, the cell culture medium comprises about 30 ng/ml of OKT-3 antibody. In an embodiment, the cell culture medium comprises about 0.1 ng/mL, about 0.5 ng/ml, about 1 ng/ml, about 2.5 ng/ml, about 5 ng/mL, about 7.5 ng/ml, about 10 ng/ml, about 15 ng/ml, about 20 ng/mL, about 25 ng/ml, about 30 ng/mL, about 35 ng/ml, about 40 ng/ml, about 50 ng/mL, about 60 ng/mL, about 70 ng/ml, about 80 ng/mL, about 90 ng/mL, about 100 ng/ml, about 200 ng/mL, about 500 ng/mL, and about 1 μg/mL of OKT-3 antibody. In an embodiment, the cell culture medium comprises between 0.1 ng/mL and 1 ng/ml, between 1 ng/ml and 5 ng/mL, between 5 ng/ml and 10 ng/mL, between 10 ng/mL and 20 ng/mL, between 20 ng/ml and 30 ng/mL, between 30 ng/mL and 40 ng/mL, between 40 ng/ml and 50 ng/mL, and between 50 ng/ml and 100 ng/ml of OKT-3 antibody.

In an embodiment, a rapid expansion process for TILs may be performed using T-175 flasks and gas permeable bags as previously described (Tran, et al., J. Immunother. 2008, 31, 742-51; Dudley, et al., J. Immunother. 2003, 26, 332-42) or gas permeable cultureware (G-Rex flasks, commercially available from Wilson Wolf Manufacturing Corporation, New Brighton, MN, USA). For TIL rapid expansion in T-175 flasks, 1×10⁶ TILs suspended in 150 mL of media may be added to each T-175 flask. The TILs may be cultured with aAPCs at a ratio of 1 TIL to 100 aAPCs and the cells were cultured in a 1 to 1 mixture of CM and AIM-V medium, supplemented with 3000 IU (international units) per mL of IL-2 and 30 ng per ml of anti-CD3 antibody (e.g., OKT-3). The T-175 flasks may be incubated at 37° C. in 5% CO₂. Half the media may be exchanged on day 5 using 50/50 medium with 3000 IU per mL of IL-2. On day 7 cells from two T-175 flasks may be combined in a 3 liter bag and 300 mL of AIM V with 5% human AB serum and 3000 IU per mL of IL-2 was added to the 300 ml of TIL suspension. The number of cells in each bag was counted every day or two and fresh media was added to keep the cell count between 0.5 and 2.0×10⁶ cells/mL.

In an embodiment, for TIL rapid expansions in 500 mL capacity gas permeable flasks with 100 cm gas-permeable silicon bottoms (G-Rex 100, commercially available from Wilson Wolf Manufacturing Corporation, New Brighton, MN, USA), 5×10⁶ or 10×10⁶ TIL may be cultured with aAPCs at a ratio of 1 to 100 in 400 mL of 50/50 medium, supplemented with 5% human AB serum, 3000 IU per mL of IL-2 and 30 ng per mL of anti-CD3 (OKT-3). The G-Rex 100 flasks may be incubated at 37° C. in 5% CO₂. On day 5, 250 mL of supernatant may be removed and placed into centrifuge bottles and centrifuged at 1500 rpm (revolutions per minute; 491×g) for 10 minutes. The TIL pellets may be re-suspended with 150 mL of fresh medium with 5% human AB serum, 3000 IU per mL of IL-2, and added back to the original G-Rex 100 flasks. When TIL are expanded serially in G-Rex 100 flasks, on day 7 the TIL in each G-Rex 100 may be suspended in the 300 mL of media present in each flask and the cell suspension may be divided into 3 100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-Rex 100 flask. The cells may be harvested on day 14 of culture.

In an embodiment, TILs may be prepared as follows. 2 mm³ tumor fragments are cultured in complete media (CM) comprised of AIM-V medium (Invitrogen Life Technologies, Carlsbad, CA) supplemented with 2 mM glutamine (Mediatech, Inc. Manassas, VA), 100 U/mL penicillin (Invitrogen Life Technologies), 100 μg/mL streptomycin (Invitrogen Life Technologies), 5% heat-inactivated human AB serum (Valley Biomedical, Inc. Winchester, VA) and 600 IU/mL rhIL-2 (Chiron, Emeryville, CA). For enzymatic digestion of solid tumors, tumor specimens were diced into RPMI-1640, washed and centrifuged at 800 rpm for 5 minutes at 15-22° C., and resuspended in enzymatic digestion buffer (0.2 mg/mL Collagenase and 30 units/ml of DNase in RPMI-1640) followed by overnight rotation at room temperature. TILs established from fragments may be grown for 3-4 weeks in CM and expanded fresh or cryopreserved in heat-inactivated HAB serum with 10% dimethylsulfoxide (DMSO) and stored at −180° C. until the time of study. Tumor associated lymphocytes (TAL) obtained from ascites collections were seeded at 3×10⁶ cells/well of a 24 well plate in CM. TIL growth was inspected about every other day using a low-power inverted microscope.

In an embodiment, TILs are expanded in gas-permeable containers. Gas-permeable containers have been used to expand TILs using PBMCs using methods, compositions, and devices known in the art, including those described in U.S. Patent Application Publication No. U.S. Patent Application Publication No. 2005/0106717 A1, the disclosures of which are incorporated herein by reference. In an embodiment, TILs are expanded in gas-permeable bags. In an embodiment, TILs are expanded using a cell expansion system that expands TILs in gas permeable bags, such as the Xuri Cell Expansion System W25 (GE Healthcare). In an embodiment, TILs are expanded using a cell expansion system that expands TILs in gas permeable bags, such as the WAVE Bioreactor System, also known as the Xuri Cell Expansion System W5 (GE Healthcare). In an embodiment, the cell expansion system includes a gas permeable cell bag with a volume selected from the group consisting of about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, about 10 L, about 11 L, about 12 L, about 13 L, about 14 L, about 15 L, about 16 L, about 17 L, about 18 L, about 19 L, about 20 L, about 25 L, and about 30 L. In an embodiment, the cell expansion system includes a gas permeable cell bag with a volume range selected from the group consisting of between 50 and 150 mL, between 150 and 250 mL, between 250 and 350 mL, between 350 and 450 mL, between 450 and 550 mL, between 550 and 650 mL, between 650 and 750 mL, between 750 and 850 mL, between 850 and 950 mL, and between 950 and 1050 mL. In an embodiment, the cell expansion system includes a gas permeable cell bag with a volume range selected from the group consisting of between 1 L and 2 L, between 2 L and 3 L, between 3 L and 4 L, between 4 L and 5 L, between 5 L and 6 L, between 6 L and 7 L, between 7 L and 8 L, between 8 L and 9 L, between 9 L and 10 L, between 10 L and 11 L, between 11 L and 12 L, between 12 L and 13 L, between 13 L and 14 L, between 14 L and 15 L, between 15 L and 16 L, between 16 L and 17 L, between 17 L and 18 L, between 18 L and 19 L, and between 19 L and 20 L. In an embodiment, the cell expansion system includes a gas permeable cell bag with a volume range selected from the group consisting of between 0.5 L and 5 L, between 5 L and 10 L, between 10 L and 15 L, between 15 L and 20 L, between 20 L and 25 L, and between 25 L and 30 L. In an embodiment, the cell expansion system utilizes a rocking time of about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, and about 28 days. In an embodiment, the cell expansion system utilizes a rocking time of between 30 minutes and 1 hour, between 1 hour and 12 hours, between 12 hours and 1 day, between 1 day and 7 days, between 7 days and 14 days, between 14 days and 21 days, and between 21 days and 28 days. In an embodiment, the cell expansion system utilizes a rocking rate of about 2 rocks/minute, about 5 rocks/minute, about 10 rocks/minute, about 20 rocks/minute, about 30 rocks/minute, and about 40 rocks/minute. In an embodiment, the cell expansion system utilizes a rocking rate of between 2 rocks/minute and 5 rocks/minute, 5 rocks/minute and 10 rocks/minute, 10 rocks/minute and 20 rocks/minute, 20 rocks/minute and 30 rocks/minute, and 30 rocks/minute and 40 rocks/minute. In an embodiment, the cell expansion system utilizes a rocking angle of about 2°, about 3°, about 4°, about 5°, about 6°, about 7°, about 8°, about 9°, about 10°, about 11°, and about 12°. In an embodiment, the cell expansion system utilizes a rocking angle of between 2° and 3°, between 3° and 4°, between 4° and 5°, between 5° and 6°, between 6° and 7°, between 7° and 8°, between 8° and 9°, between 9° and 10°, between 10° and 11°, and between 11° and 12°.

In an embodiment, a method of expanding TILs using aAPCs further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

In an embodiment, the aAPCs of the present invention may be used to expand T cells. Any of the foregoing embodiments of the present invention described for the expansion of TILs may also be applied to the expansion of T cells. In an embodiment, the aAPCs of the present invention may be used to expand CD8⁺ T cells. In an embodiment, the aAPCs of the present invention may be used to expand CD4⁺ T cells. In an embodiment, the aAPCs of the present invention may be used to expand T cells transduced with a chimeric antigen receptor (CAR-T). In an embodiment, the aAPCs of the present invention may be used to expand T cells comprising a modified T cell receptor (TCR). The CAR-T cells may be targeted against any suitable antigen, including CD19, as described in the art, e.g., in U.S. Pat. Nos. 7,070,995; 7,446,190; 8,399,645; 8,916,381; and 9,328,156; the disclosures of which are incorporated by reference herein. The modified TCR cells may be targeted against any suitable antigen, including NY-ESO-1, TRP-1, TRP-2, tyrosinase cancer antigen, MAGE-A3, SSX-2, and VEGFR2, or antigenic portions thereof, as described in the art, e.g., in U.S. Pat. Nos. 8,367,804 and 7,569,664, the disclosures of which are incorporated by reference herein.

### Methods of Treating Cancers and Other Diseases

The compositions and methods described herein can be used in a method for treating diseases. In an embodiment, they are for use in treating hyperproliferative disorders. They may also be used in treating other disorders as described herein and in the following paragraphs. The TILs, populations and compositions thereof described herein may be for use in the treatment of a disease. In an embodiment, the TILs, populations and compositions described herein are for use in the treatment of a hyperproliferative disorder.

In some embodiments, the hyperproliferative disorder is cancer. In some embodiments, the hyperproliferative disorder is a solid tumor cancer. In some embodiments, the solid tumor cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer, renal cancer, and renal cell carcinoma, pancreatic cancer, and glioblastoma. In some embodiments, the hyperproliferative disorder is a hematological malignancy. In some embodiments, the hematological malignancy is selected from the group consisting of chronic lymphocytic leukemia, acute lymphoblastic leukemia, diffuse large B cell lymphoma, non-Hodgkin's lymphoma, Hodgkin's lymphoma, follicular lymphoma, and mantle cell lymphoma.

In an embodiment, the invention includes a method of treating a cancer with a population of tumor infiltrating lymphocytes (TILs) comprising the steps of: (a) obtaining a first population of TILs from a tumor resected from a patient; (b) performing a rapid expansion of the first population of TILs using a population of artificial antigen presenting cells (aAPCs) in a cell culture medium to obtain a second population of TILs, wherein the second population of TILs is at least 50-fold greater in number than the first population of TILs; and (c) administering a therapeutically effective portion of the second population of TILs to a patient with the cancer. In an embodiment, the aAPCs comprise MOLM-14 cells transduced with one or more viral vectors, wherein the one or more viral vectors comprise a nucleic acid encoding CD86 and a nucleic acid encoding 4-1BBL, and wherein the MOLM-14 cells express a CD86 protein and a 4-1BBL protein. In an embodiment, the rapid expansion is performed over a period not greater than 14 days.

In an embodiment, the invention includes a method of treating a cancer with a population of tumor infiltrating lymphocytes (TILs) comprising the steps of: (a) obtaining a first population of TILs from a tumor resected from a patient; (b) performing an initial expansion of the first population of TILs using a first population of artificial antigen presenting cells (aAPCs) in a first cell culture medium to obtain a second population of TILs, wherein the second population of TILs is at least 10-fold greater in number than the first population of TILs, and wherein the first cell culture medium comprises IL-2; (c) performing a rapid expansion of the second population of TILs using a second population of aAPCs in a second cell culture medium to obtain a third population of TILs, wherein the third population of TILs is at least 50-fold greater in number than the first population of TILs; and wherein the second cell culture medium comprises IL-2 and OKT-3; (d) administering a therapeutically effective portion of the third population of TILs to a patient with the cancer. In an embodiment, the aAPCs comprise MOLM-14 cells transduced with one or more viral vectors, wherein the one or more viral vectors comprise a nucleic acid encoding CD86 and a nucleic acid encoding 4-1BBL, and wherein the MOLM-14 cells express a CD86 protein and a 4-1BBL protein. In an embodiment, the rapid expansion is performed over a period not greater than 14 days. In an embodiment, the initial expansion is performed using a gas permeable container.

In an embodiment, the invention includes a method of treating a cancer with a population of tumor infiltrating lymphocytes (TILs) comprising the steps of: (a) obtaining a first population of TILs from a tumor resected from a patient; (b) performing an initial expansion of the first population of TILs in a first cell culture medium to obtain a second population of TILs, wherein the second population of TILs is at least 10-fold greater in number than the first population of TILs, and wherein the first cell culture medium comprises IL-2; (c) performing a rapid expansion of the second population of TILs using a population of artificial antigen presenting cells (aAPCs) in a second cell culture medium to obtain a third population of TILs, wherein the third population of TILs is at least 50-fold greater in number than the first population of TILs; and wherein the second cell culture medium comprises IL-2 and OKT-3; (d) administering a therapeutically effective portion of the third population of TILs to a patient with the cancer. In an embodiment, the aAPCs comprise MOLM-14 cells transduced with one or more viral vectors, wherein the one or more viral vectors comprise a nucleic acid encoding CD86 and a nucleic acid encoding 4-1BBL, and wherein the MOLM-14 cells express a CD86 protein and a 4-1BBL protein. In an embodiment, the rapid expansion is performed over a period not greater than 14 days.

In an embodiment, the invention includes a method of treating a cancer with a population of TILs, wherein a patient is pre-treated with non-myeloablative chemotherapy prior to an infusion of TILs according to the present disclosure. In an embodiment, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In an embodiment, after non-myeloablative chemotherapy and TIL infusion (at day 0) according to the present disclosure, the patient receives an intravenous infusion of IL-2 intravenously at 720,000 IU/kg every 8 hours to physiologic tolerance.

Efficacy of the compounds and combinations of compounds described herein in treating, preventing and/or managing the indicated diseases or disorders can be tested using various models known in the art, which provide guidance for treatment of human disease. For example, models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32.

Non-Myeloablative Lymphodepletion with Chemotherapy

In an embodiment, the invention includes a method of treating a cancer with a population of TILs, wherein a patient is pre-treated with non-myeloablative chemotherapy prior to an infusion of TILs according to the present disclosure. In an embodiment, the invention provides a population of TILs obtainable by a method described herein for use in treating a cancer, wherein the population of TILs is for treating a patient which is pre-treated with non-myeloablative chemotherapy. In an embodiment, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In an embodiment, after non-myeloablative chemotherapy and TIL infusion (at day 0) according to the present disclosure, the patient receives an intravenous infusion of IL-2 (aldesleukin, commercially available as PROLEUKIN) intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the aAPC-expanded TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol. 2008, 26, 5233-5239, and Dudley, et al., J. Clin. Oncol. 2005, 23, 2346-2357, all of which are incorporated by reference herein in their entireties.

In some embodiments, the fludarabine is administered at a concentration of 0.5 μg/mL-10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 g/ml-10 μg/ml by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day, 200 mg/m²/day, 225 mg/m²/day, 250 mg/m²/day, 275 mg/m²/day, or 300 mg/m²/day. In some embodiments, the cyclophosphamide is administered intravenously (i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide are together to a patient. In some embodiments, fludarabine is administered at 25 mg/m²/day i.v. and cyclophosphamide is administered at 250 mg/m²/day i.v. over 4 days.

In an embodiment, the lymphodepletion is performed by administration of cyclophosphamide at a dose of 60 mg/m²/day for two days followed by administration of fludarabine at a dose of 25 mg/m²/day for five days.

### Pharmaceutical Compositions, Dosages, and Dosing Regimens

In an embodiment, TILs expanded using aAPCs of the present disclosure are administered to a patient as a pharmaceutical composition. In an embodiment, the pharmaceutical composition is a suspension of TILs in a sterile buffer. TILs expanded using aAPCs of the present disclosure may be administered by any suitable route as known in the art. Preferably, the TILs are administered as a single infusion, such as an intra-arterial or intravenous infusion, which preferably lasts approximately 30 to 60 minutes. Other suitable routes of administration include intraperitoneal, intrathecal, and intralymphatic administration.

Any suitable dose of TILs can be administered. Preferably, from about 2.3×10¹⁰ to about 13.7×10¹⁰ TILs are administered, with an average of around 7.8×10¹⁰ TILs, particularly if the cancer is melanoma. In an embodiment, about 1.2×10¹⁰ to about 4.3×10¹⁰ of TILs are administered.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In an embodiment, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of embodiments of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1—Variability in Expansion of Tumor Infiltrating Lymphocytes Using PBMC Feeder Cells

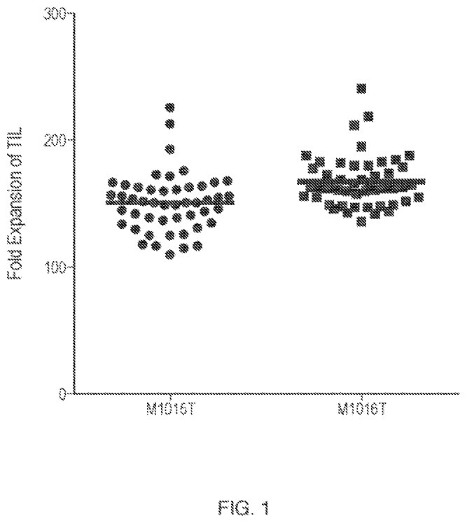

The variability in TIL expansion obtained by use of PBMC feeder cells may be demonstrated by comparing the results of multiple TIL expansions on the same line of TILs obtained from a patient. FIG. 1 illustrates typical results of rapid expansion of TILs using irradiated allogeneic PBMC feeder cells (PBMC feeders). Two TIL lines labeled M1015T and M1016T (1.3×10⁵ cells) were co-cultured with 46 different irradiated feeder cell lots (1.3×10⁷), IL-2 (3000 IU/mL, recombinant human IL-2 (e.g., aldesleukin or equivalent), CellGenix, Inc., Portsmouth, NH, USA) and OKT-3 (30 ng/mL, MACS GMP CD3 pure, Miltenyi Biotec GmbH, Bergisch Gladbach, Germany) in a T25 flask for 7 days. The fold expansion value for TILs was calculated on Day 7. The figure shows the number of fold expansions for the two TIL lines in separate stimulation experiments. For each TIL line, 46 different PBMC feeder lots were tested. The results range over more than 100-fold for each TIL line, and highlight the variability of expansion results using PBMC feeder cells. The aAPCs of the present invention offer reduced variability in expansion performance compared to PBMC feeders, as well as other advantages, as shown in the following examples.

### Example 2—Selection of Myeloid Cells for aAPC Development

Phenotypic characterization was performed on various myeloid-lineage cell lines to identify potential candidates for further modification into aAPCs for TIL expansion. The results are summarized in Table 5. The MOLM-14 cell line exhibited endogenous expression of CD64, and was selected for further development. The EM-3 cell line was selected based on the observation of endogenous expression of ICOS-L (which was not observed for the EM-2 cell line, despite being taken from the same patient).

TABLE 5
Summary of costimulatory molecules expressed endogenously on candidate cell lines
for aAPCs. CML refers to chronic myeloid leukemia, and AML refers to acute myeloid
leukemia. “Pop” refers to the population of cells observed to express the marker
(1/2 pop = 50%).

| | | | | | K562 | |
|---|---|---|---|---|---|---|
| | EM-2 | EM-3 | | | myeloid | MOLM-14 |
| Cell line | Myeloid blast | Myeloid blast | KG1-246 | KG1-8031 | erythro-leukemia, | AML |
| Origin | crisis, CML | crisis, CML | AML | AML | CML | |
| HLA-A/B/C | + | + | + | + | − | + |
| CD64 | − | − | − | − | − | + |
| CD80 | − | − | − | − | − | + |
| ICOS-L | − | + | − | − | − | + |
| 4-1BBL | − | − | − | − | − | − |
| PD-Ll | − | − | − | − | − | − |
| CD58 | + | + | + | + | + | + |
| CD86 | − | − | − | − | − | + (l/2 pop) |

### Example 3—Preparation of MOLM-14 Artificial Antigen Presenting Cells (aMOLM14 aAPCs)

MOLM-14 cells were obtained from Leibniz-Institut DSMZ-Deutsche Sammlung von Mikroorganismen und Zellkulturen GmbH. To develop MOLM-14 based aAPCs, MOLM-14 cells were engineered with the costimulatory molecules CD86 and 4-1BBL (CD137L). Human CD86 (hCD86) and human 4-1BBL (h4-1BBL) genes were cloned into commercially-available PLV430G and co-transfected with PDONR221 vectors (Invitrogen/Thermo Fisher Scientific, Carlsbad, CA, USA) using a lentiviral transduction method. The gateway cloning method was used as described in Katzen, Expert Opin. Drug Disc. 2007, 4, 571-589, to clone hCD86 and hCD137L genes onto the PLV430G and PDONR221 vectors. The 293T cell line (human embryonic kidney cells transformed with large T antigen) was used for lentiviral production, transduced to MOLM-14 cells. The transfected cells were sorted (S3e Cell Sorter, Bio-Rad, Hercules, CA, USA) using APC-conjugated CD86 and PE-conjugated CD137L to isolate and enrich the cells. The enriched cells were checked for purity by flow cytometry.

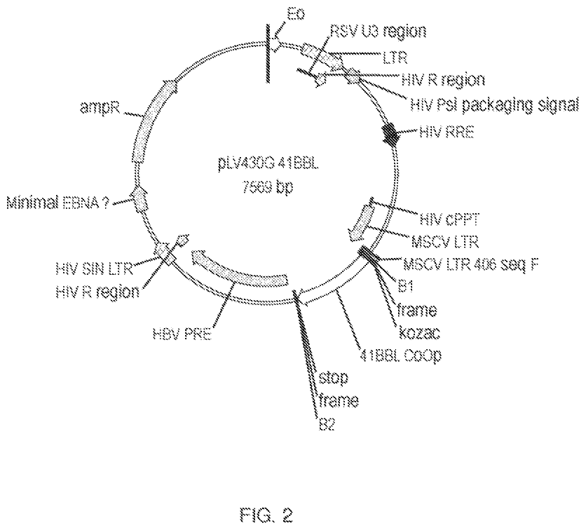

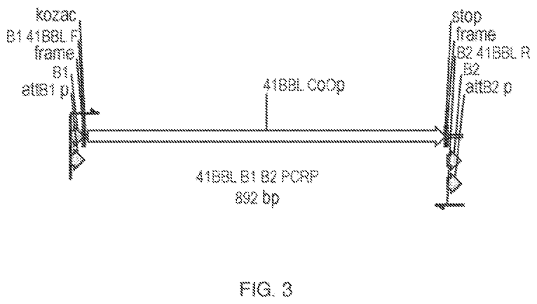

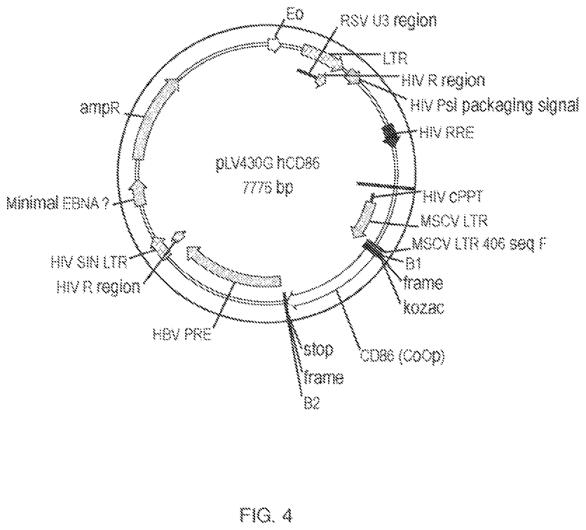

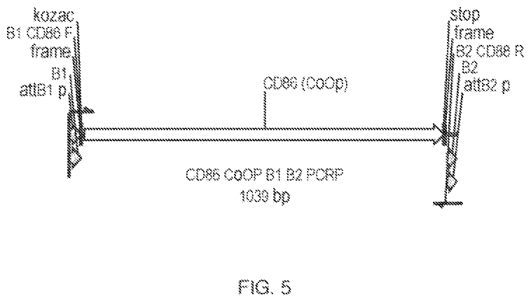

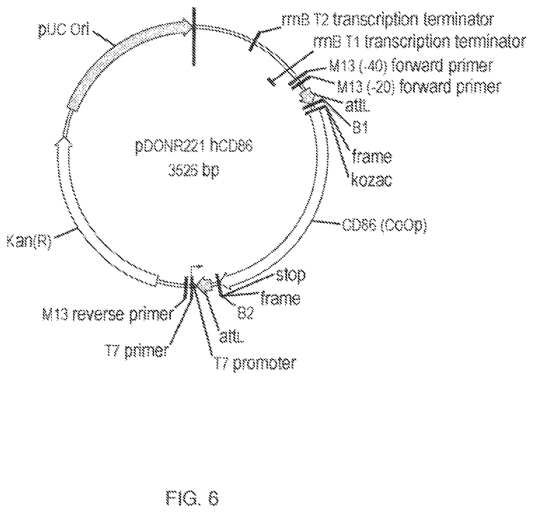

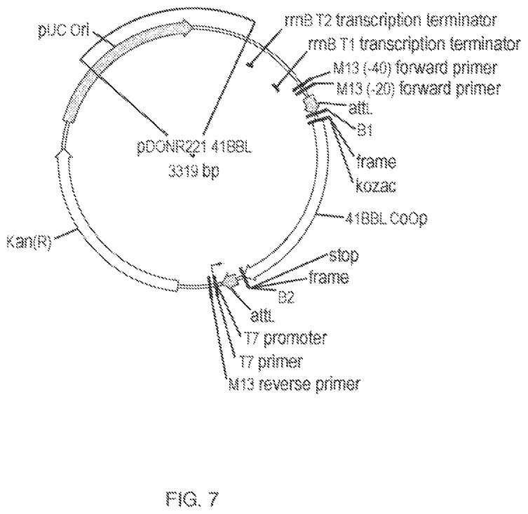

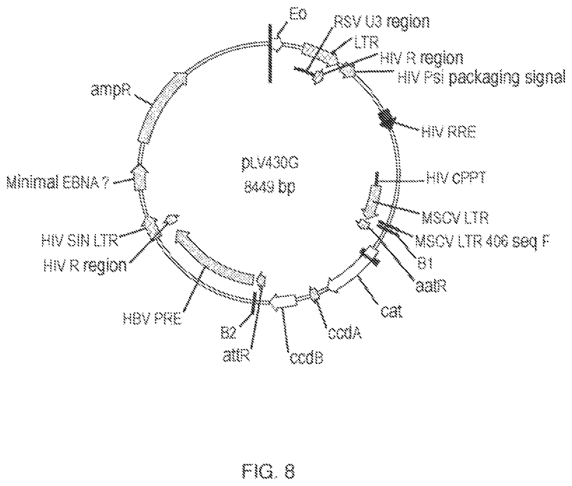

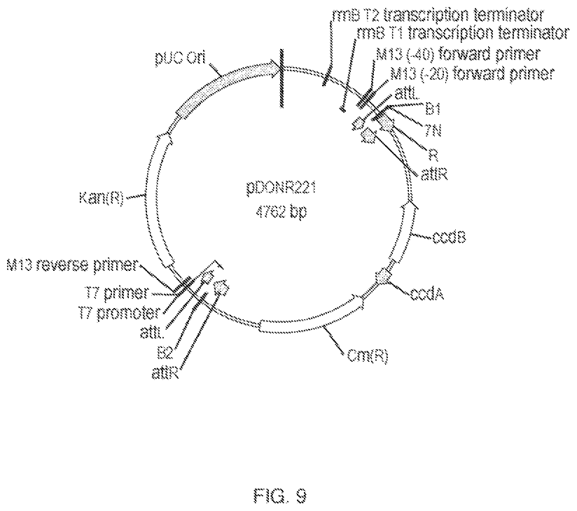

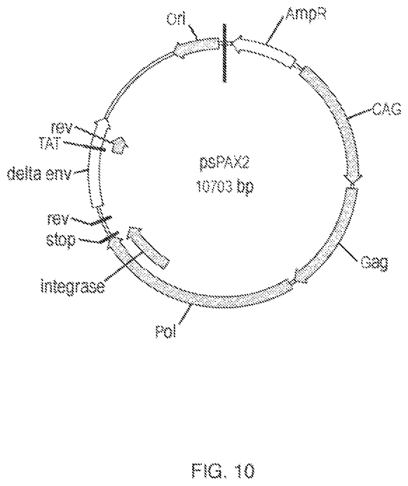

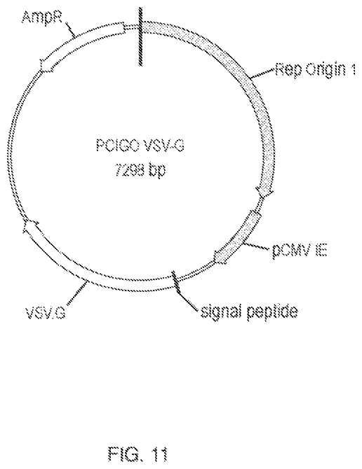

The vectors and portions thereof used for cloning are depicted in FIG. 2 to FIG. 11, and the nucleotide sequences for each vector are given in Table 6. The pLV430G human 4-1BBL vector is illustrated in FIG. 2, with the polymerase chain reaction product (PCRP) portion shown in FIG. 3. The pLV430G human CD86 vector is illustrated in FIG. 4, with the PCRP portion shown in FIG. 5. The pDONR221 human CD86 donor and human 4-1BBL donor vectors are shown in FIG. 6 and FIG. 7, respectively. Diagrams of the empty pLV430G destination vector and empty pDONR221 donor vector for the Gateway cloning method are shown in FIG. 8 and FIG. 9, respectively. FIG. 10 and FIG. 11 illustrate vector diagrams of the psPAX2 and pCIGO-VSV.G helper plasmids used for lentivirus production.

TABLE 6
Nucleotide sequences for preparation of lentivirus for transduction of aAPCs.

| Identifier | | |
|---|---|---|
| (Description) | Sequence | |
| SEQ ID NO: 15 | cgataaccct aattcgatag catatgcttc ccgttgggta acatatgcta ttgaattagg | 60 |
| (pLV430G human | gttagtctgg atagtatata ctactacccg ggaagcatat gctacccgtt tagggttcac | 120 |
| 4-1BBL vector) | cggtgatgcc ggccacgatg cgtccggcgt agaggatcta atgtgagtta gctcactcat | 180 |
| | taggcacccc aggctttaca ctttatgctt ccggctcgta tgttgtgtgg aattgtgagc | 240 |
| | ggataacaat ttcacacagg aaacagctat gaccatgatt acgccaagcg cgcaattaac | 300 |
| | cctcactaaa gggaacaaaa gctggagctg caagcttaat gtagtcttat gcaatactct | 360 |
| | tgtagtcttg caacatggta acgatgagtt agcaacatgc cttacaagga gagaaaaagc | 420 |
| | accgtgcatg ccgattggtg gaagtaaggt ggtacgatcg tgccttatta ggaaggcaac | 480 |
| | agacgggtct gacatggatt ggacgaacca ctgaattgcc gcattgcaga gatattgtat | 540 |
| | ttaagtgcct agctcgatac ataaacgggt ctctctggtt agaccagatc tgagcctggg | 600 |
| | agctctctgg ctaactaggg aacccactgc ttaagcctca ataaagcttg ccttgagtgc | 660 |
| | ttcaagtagt gtgtgcccgt ctgttgtgtg actctggtaa ctagagatcc ctcagaccct | 720 |
| | tttagtcagt gtggaaaatc tctagcagtg gcgcccgaac agggacttga aagcgaaagg | 780 |
| | gaaaccagag gagctctctc gacgcaggac tcggcttgct gaagcgcgca cggcaagagg | 840 |
| | cgaggggcgg cgactggtga gtacgccaaa aattttgact agcggaggct agaaggagag | 900 |
| | agatgggtgc gagagcgtca gtattaagcg ggggagaatt agatcgcgat gggaaaaaat | 960 |
| | tcggttaagg ccagggggaa agaaaaaata taaattaaaa catatagtat gggcaagcag | 1020 |
| | ggagctagaa cgattcgcag ttaatcctgg cctgttagaa acatcagaag gctgtagaca | 1080 |
| | aatactggga cagctacaac catcccttca gacaggatca gaagaactta gatcattata | 1140 |
| | taatacagta gcaaccctct attgtgtgca tcaaaggata gagataaaag acaccaagga | 1200 |
| | agctttagac aagatagagg aagagcaaaa caaaagtaag accaccgcac agcaagcggc | 1260 |
| | cgctgatctt cagacctgga ggaggagata tgagggacaa ttggagaagt gaattatata | 1320 |
| | aatataaagt agtaaaaatt gaaccattag gagtagcacc caccaaggca aagagaagag | 1380 |
| | tggtgcagag agaaaaaaga gcagtgggaa taggagcttt gttccttggg ttcttgggag | 1440 |
| | cagcaggaag cactatgggc gcagcgtcaa tgacgctgac ggtacaggcc agacaattat | 1500 |
| | tgtctggtat agtgcagcag cagaacaatt tgctgagggc tattgaggcg caacagcatc | 1560 |
| | tgttgcaact cacagtctgg ggcatcaagc agctccaggc aagaatcctg gctgtggaaa | 1620 |
| | gatacctaaa ggatcaacag ctcctgggga tttggggttg ctctggaaaa ctcatttgca | 1680 |
| | ccactgctgt gccttggaat gctagttgga gtaataaatc tctggaacag atttggaatc | 1740 |
| | acacgacctg gatggagtgg gacagagaaa ttaacaatta cacaagctta atacactcct | 1800 |
| | taattgaaga atcgcaaaac cagcaagaaa agaatgaaca agaattattg gaattagata | 1860 |
| | aatgggcaag tttgtggaat tggtttaaca taacaaattg gctgtggtat ataaaattat | 1920 |
| | tcataatgat agtaggaggc ttggtaggtt taagaatagt ttttgctgta ctttctatag | 1980 |
| | tgaatagagt taggcaggga tattcaccat tatcgtttca gacccacctc ccaaccccga | 2040 |
| | ggggacccga caggcccgaa ggaatagaag aagaaggtgg agagagagac agagacagat | 2100 |
| | ccattcgatt agtgaacgga tctcgacggt atcggtttta aaagaaaagg ggggattggg | 2160 |
| | gggtacagtg caggggaaag aatagtagac ataatagcaa cagacataca aactaaagaa | 2220 |
| | ttacaaaaac aaattacaaa aattcaaaat tttatcgatt ttatttagtc tccagaaaaa | 2280 |
| | ggggggaatg aaagacccca cctgtaggtt tggcaagcta gcttaagtaa cgccattttg | 2340 |
| | caaggcatgg aaaatacata actgagaata gagaagttca gatcaaggtt aggaacagag | 2400 |
| | agacagcaga atatgggcca aacaggatat ctgtggtaag cagttcctgc cccggctcag | 2460 |
| | ggccaagaac agatggtccc cagatgcggt cccgccctca gcagtttcta gagaaccatc | 2520 |
| | agatgtttcc agggtgcccc aaggacctga aatgaccctg tgccttattt gaactaacca | 2580 |
| | atcagttcgc ttctcgcttc tgttcgcgcg cttctgctcc ccgagctcaa taaaagagcc | 2640 |
| | cacaacccct cactcggcgc gccagtcctc cgatagactg cgtcgcccgg gtaccgatat | 2700 |
| | caacaagttt gtacaaaaaa gcaggcttcg ccaccatgga atacgcctct gatgccagcc | 2760 |
| | tggaccccga agctccttgg cctcctgccc ctagagccag agcctgtaga gtgctgcctt | 2820 |
| | gggctctggt ggctggcctt ctccttctgc tgctgctggc cgctgcctgc gctgtgtttc | 2880 |
| | tggcttgtcc ttgggccgtg tcaggcgcca gagcttctcc tggatctgcc gccagcccca | 2940 |
| | gactgagaga gggacctgag ctgagccccg atgatcctgc cggactgctg gatctgagac | 3000 |
| | agggcatgtt cgcccagctg gtggcccaga acgtgctgct gatcgatggc cccctgagct | 3060 |
| | ggtacagcga tcctggactg gctggcgtgt cactgacagg cggcctgagc tacaaagagg | 3120 |
| | acaccaaaga actggtggtg gccaaggccg gcgtgtacta cgtgttcttt cagctggaac | 3180 |
| | tgcggagagt ggtggccggc gaaggatccg gctctgtgtc tctggcactg catctgcagc | 3240 |
| | ccctgagatc tgctgcaggc gctgctgcac tggccctgac agtggacctg cctccagcct | 3300 |
| | ctagcgaggc cagaaactcc gcattcgggt ttcaaggcag actgctgcac ctgtctgccg | 3360 |
| | gccagagact gggagtgcat ctgcacacag aggccagagc cagacacgcc tggcagctga | 3420 |
| | cacagggcgc tacagtgctg ggcctgttca gagtgacccc cgaaattcca gccggcctgc | 3480 |
| | ccagccctag aagcgagtag gacccagctt tcttgtacaa agtggtgatt cgagttaatt | 3540 |
| | aagctagcct agtgccattt gttcagtggt tcgtagggct ttcccccact gtttggcttt | 3600 |
| | cagttatatg gatgatgtgg tattgggggc caagtctgta cagcatcttg agtccctttt | 3660 |
| | taccgctgtt accaattttc ttttgtcttt gggtatacat ttaaacccta acaaaacaaa | 3720 |
| | gagatggggt tactctctaa attttatggg ttatgtcatt ggatgttatg ggtccttgcc | 3780 |
| | acaagaacac atcatacaaa aaatcaaaga atgttttaga aaacttccta ttaacaggcc | 3840 |
| | tattgattgg aaagtatgtc aacgaattgt gggtcttttg ggttttgctg ccccttttac | 3900 |
| | acaatgtggt tatcctgcgt tgatgccttt gtatgcatgt attcaatcta agcaggcttt | 3960 |
| | cactttctcg ccaacttaca aggcctttct gtgtaaacaa tacctgaacc tttaccccgt | 4020 |
| | tgcccggcaa cggccaggtc tgtgccaagt gtttgctgac gcaaccccca ctggctgggg | 4080 |
| | cttggtcatg ggccatcagc gcatgcgtgg aaccttttcg gctcctctgc cgatccatac | 4140 |
| | tgcggaactc ctagccgctt gttttgctcg cagcaggtct ggagcaaaca ttatcgggac | 4200 |
| | tgataactct gttgtcctat cccgcaaata tacatcgttt ccatggctgc taggctgtgc | 4260 |
| | tgccaactgg atcctgcgcg ggacgtcctt tgtttacgtc ccgtcggcgc tgaatcctgc | 4320 |
| | ggacgaccct tctcggggtc gcttgggact ctctcgtccc cttctccgtc tgccgttccg | 4380 |
| | accgaccacg gggcgcacct ctctttacgc ggactccccg tctgtgcctt ctcatctgcc | 4440 |
| | ggaccgtgtg cacttcgctt cacctctgca cgtcgcatgg agaccaccgt gaacgcccac | 4500 |
| | caaatattgc ccaaggtctt acataagagg actcttggac tctcagcaat gtcaacgacc | 4560 |
| | gaccttgagg catacttcaa agactgtttg tttaaagact gggaggagtt gggggaggag | 4620 |
| | attaggttaa aggtctttgt actaggaggc tgtaggcata aattggtctg cgcaccagca | 4680 |
| | ccatggcgca atcactagag cggggtacct ttaagaccaa tgacttacaa ggcagctgta | 4740 |
| | gatcttagcc actttttaaa agaaaagggg ggactggaag ggctaattca ctcccaacga | 4800 |
| | agacaagatc tgctttttgc ttgtactggg tctctctggt tagaccagat ctgagcctgg | 4860 |
| | gagctctctg gctaactagg gaacccactg cttaagcctc aataaagctt gccttgagtg | 4920 |
| | cttcaagtag tgtgtgcccg tctgttgtgt gactctggta actagagatc cctcagaccc | 4980 |
| | ttttagtcag tgtggaaaat ctctagcagt agtagttcat gtcatcttat tattcagtat | 5040 |
| | ttataacttg caaagaaatg aatatcagag agtgagagga acttgtttat tgcagcttat | 5100 |
| | aatggttaca aataaagcaa tagcatcaca aatttcacaa ataaagcatt tttttcactg | 5160 |
| | cattctagtt gtggtttgtc caaactcatc aatgtatctt atcatgtctg gctctagcta | 5220 |
| | tcccgcccct aactccgccc atcccgcccc taactccgcc cagttccgcc cattctccgc | 5280 |
| | cccatggctg actaattttt tttatttatg cagaggccga ggccggatcc cttgagtggc | 5340 |
| | tttcatcctg gagcagactt tgcagtctgt ggactgcaac acaacattgc ctttatgtgt | 5400 |
| | aactcttggc tgaagctctt acaccaatgc tgggggacat gtacctccca ggggcccagg | 5460 |
| | aagactacgg gaggctacac caacgtcaat cagaggggcc tgtgtagcta ccgataagcg | 5520 |
| | gaccctcaag agggcattag caatagtgtt tataaggccc ccttgttaat tcttgaagac | 5580 |
| | gaaagggcct cgtgatacgc ctatttttat aggttaatgt catgataata atggtttctt | 5640 |
| | agacgtcagg tggcactttt cggggaaatg tgcgcggaac ccctatttgt ttatttttct | 5700 |
| | aaatacattc aaatatgtat ccgctcatga gacaataacc ctgataaatg cttcaataat | 5760 |
| | attgaaaaag gaagagtatg agtattcaac atttccgtgt cgcccttatt cccttttttg | 5820 |
| | cggcattttg ccttcctgtt tttgctcacc cagaaacgct ggtgaaagta aaagatgctg | 5880 |
| | aagatcagtt gggtgcacga gtgggttaca tcgaactgga tctcaacagc ggtaagatcc | 5940 |
| | ttgagagttt tcgccccgaa gaacgttttc caatgatgag cacttttaaa gttctgctat | 6000 |
| | gtggcgcggt attatcccgt gttgacgccg ggcaagagca actcggtcgc cgcatacact | 6060 |
| | attctcagaa tgacttggtt gagtactcac cagtcacaga aaagcatctt acggatggca | 6120 |
| | tgacagtaag agaattatgc agtgctgcca taaccatgag tgataacact gcggccaact | 6180 |
| | tacttctgac aacgatcgga ggaccgaagg agctaaccgc ttttttgcac aacatggggg | 6240 |
| | atcatgtaac tcgccttgat cgttgggaac cggagctgaa tgaagccata ccaaacgacg | 6300 |
| | agcgtgacac cacgatgcct gcagcaatgg caacaacgtt gcgcaaacta ttaactggcg | 6360 |
| | aactacttac tctagcttcc cggcaacaat taatagactg gatggaggcg gataaagttg | 6420 |
| | caggaccact tctgcgctcg gcccttccgg ctggctggtt tattgctgat aaatctggag | 6480 |
| | ccggtgagcg tgggtctcgc ggtatcattg cagcactggg gccagatggt aagccctccc | 6540 |
| | gtatcgtagt tatctacacg acggggagtc aggcaactat ggatgaacga aatagacaga | 6600 |
| | tcgctgagat aggtgcctca ctgattaagc attggtaact gtcagaccaa gtttactcat | 6660 |
| | atatacttta gattgattta aaacttcatt tttaatttaa aaggatctag gtgaagatcc | 6720 |
| | tttttgataa tctcatgacc aaaatccctt aacgtgagtt ttcgttccac tgagcgtcag | 6780 |
| | accccgtaga aaagatcaaa ggatcttctt gagatccttt ttttctgcgc gtaatctgct | 6840 |
| | gcttgcaaac aaaaaaacca ccgctaccag cggtggtttg tttgccggat caagagctac | 6900 |
| | caactctttt tccgaaggta actggcttca gcagagcgca gataccaaat actgtccttc | 6960 |
| | tagtgtagcc gtagttaggc caccacttca agaactctgt agcaccgcct acatacctcg | 7020 |
| | ctctgctaat cctgttacca gtggctgctg ccagtggcga taagtcgtgt cttaccgggt | 7080 |
| | tggactcaag acgatagtta ccggataagg cgcagcggtc gggctgaacg gggggttcgt | 7140 |
| | gcacacagcc cagcttggag cgaacgacct acaccgaact gagataccta cagcgtgagc | 7200 |
| | attgagaaag cgccacgctt cccgaaggga gaaaggcgga caggtatccg gtaagcggca | 7260 |
| | gggtcggaac aggagagcgc acgagggagc ttccaggggg aaacgcctgg tatctttata | 7320 |
| | gtcctgtcgg gtttcgccac ctctgacttg agcgtcgatt tttgtgatgc tcgtcagggg | 7380 |
| | ggcggagcct atggaaaaac gccagcaacg cggccttttt acggttcctg gccttttgct | 7440 |
| | ggcctttttg aagctgtccc tgatggtcgt catctacctg cctggacagc atggcctgca | 7500 |
| | acgcgggcat cccgatgccg ccggaagcga gaagaatcat aatggggaag gccatccagc | 7560 |
| | ctcgcgtcg | 7569 |
| SEQ ID NO: 16 | atggaatacg cctctgatgc cagcctggac cccgaagctc cttggcctcc tgcccctaga | 60 |
| (4-1BBL CoOP) | gccagagcct gtagagtgct gccttgggct ctggtggctg gccttctcct tctgctgctg | 120 |
| | ctggccgctg cctgcgctgt gtttctggct tgtccttggg ccgtgtcagg cgccagagct | 180 |
| | tctcctggat ctgccgccag ccccagactg agagagggac ctgagctgag ccccgatgat | 240 |
| | cctgccggac tgctggatct gagacagggc atgttcgccc agctggtggc ccagaacgtg | 300 |
| | ctgctgatcg atggccccct gagctggtac agcgatcctg gactggctgg cgtgtcactg | 360 |
| | acaggcggcc tgagctacaa agaggacacc aaagaactgg tggtggccaa ggccggcgtg | 420 |
| | tactacgtgt tctttcagct ggaactgcgg agagtggtgg ccggcgaagg atccggctct | 480 |
| | gtgtctctgg cactgcatct gcagcccctg agatctgctg caggcgctgc tgcactggcc | 540 |
| | ctgacagtgg acctgcctcc agcctctagc gaggccagaa actccgcatt cgggtttcaa | 600 |
| | ggcagactgc tgcacctgtc tgccggccag agactgggag tgcatctgca cacagaggcc | 660 |
| | agagccagac acgcctggca gctgacacag ggcgctacag tgctgggcct gttcagagtg | 720 |
| | acccccgaaa ttccagccgg cctgcccagc cctagaagcg agtag | 765 |
| SEQ ID NO: 17 | ggggacaagt ttgtacaaaa aagcaggctt cgccaccatg gaatacgcct ctgatgccag | 60 |
| (4-1BBL FRCP) | cctggacccc gaagctcctt ggcctcctgc ccctagagcc agagcctgta gagtgctgcc | 120 |
| | ttgggctctg gtggctggcc ttctccttct gctgctgctg gccgctgcct gcgctgtgtt | 180 |
| | tctggcttgt ccttgggccg tgtcaggcgc cagagcttct cctggatctg ccgccagccc | 240 |
| | cagactgaga gagggacctg agctgagccc cgatgatcct gccggactgc tggatctgag | 300 |
| | acagggcatg ttcgcccagc tggtggccca gaacgtgctg ctgatcgatg gccccctgag | 360 |
| | ctggtacagc gatcctggac tggctggcgt gtcactgaca ggcggcctga gctacaaaga | 420 |
| | ggacaccaaa gaactggtgg tggccaaggc cggcgtgtac tacgtgttct ttcagctgga | 480 |
| | actgcggaga gtggtggccg gcgaaggatc cggctctgtg tctctggcac tgcatctgca | 540 |
| | gcccctgaga tctgctgcag gcgctgctgc actggccctg acagtggacc tgcctccagc | 600 |
| | ctctagcgag gccagaaact ccgcattcgg gtttcaaggc agactgctgc acctgtctgc | 660 |
| | cggccagaga ctgggagtgc atctgcacac agaggccaga gccagacacg cctggcagct | 720 |
| | gacacagggc gctacagtgc tgggcctgtt cagagtgacc cccgaaattc cagccggcct | 780 |
| | gcccagccct agaagcgagt aggacccagc tttcttgtac aaagtggtcc cc | 832 |
| SEQ ID NO: 18 | cgataaccct aattcgatag catatgcttc ccgttgggta acatatgcta ttgaattagg | 60 |
| (pLV430G human | gttagtctgg atagtatata ctactacccg ggaagcatat gctacccgtt tagggttcac | 120 |
| CD86 vector) | cggtgatgcc ggccacgatg cgtccggcgt agaggatcta atgtgagtta gctcactcat | 180 |
| | taggcacccc aggctttaca ctttatgctt ccggctcgta tgttgtgtgg aattgtgagc | 240 |
| | ggataacaat ttcacacagg aaacagctat gaccatgatt acgccaagcg cgcaattaac | 300 |
| | cctcactaaa gggaacaaaa gctggagctg caagcttaat gtagtcttat gcaatactct | 360 |
| | tgtagtcttg caacatggta acgatgagtt agcaacatgc cttacaagga gagaaaaagc | 420 |
| | accgtgcatg ccgattggtg gaagtaaggt ggtacgatcg tgccttatta ggaaggcaac | 480 |
| | agacgggtct gacatggatt ggacgaacca ctgaattgcc gcattgcaga gatattgtat | 540 |
| | ttaagtgcct agctcgatac ataaacgggt ctctctggtt agaccagatc tgagcctggg | 600 |
| | agctctctgg ctaactaggg aacccactgc ttaagcctca ataaagcttg ccttgagtgc | 660 |
| | ttcaagtagt gtgtgcccgt ctgttgtgtg actctggtaa ctagagatcc ctcagaccct | 720 |
| | tttagtcagt gtggaaaatc tctagcagtg gcgcccgaac agggacttga aagcgaaagg | 780 |
| | gaaaccagag gagctctctc gacgcaggac tcggcttgct gaagcgcgca cggcaagagg | 840 |
| | cgaggggcgg cgactggtga gtacgccaaa aattttgact agcggaggct agaaggagag | 900 |
| | agatgggtgc gagagcgtca gtattaagcg ggggagaatt agatcgcgat gggaaaaaat | 960 |
| | tcggttaagg ccagggggaa agaaaaaata taaattaaaa catatagtat gggcaagcag | 1020 |
| | ggagctagaa cgattcgcag ttaatcctgg cctgttagaa acatcagaag gctgtagaca | 1080 |
| | aatactggga cagctacaac catcccttca gacaggatca gaagaactta gatcattata | 1140 |
| | taatacagta gcaaccctct attgtgtgca tcaaaggata gagataaaag acaccaagga | 1200 |
| | agctttagac aagatagagg aagagcaaaa caaaagtaag accaccgcac agcaagcggc | 1260 |
| | cgctgatctt cagacctgga ggaggagata tgagggacaa ttggagaagt gaattatata | 1320 |
| | aatataaagt agtaaaaatt gaaccattag gagtagcacc caccaaggca aagagaagag | 1380 |
| | tggtgcagag agaaaaaaga gcagtgggaa taggagcttt gttccttggg ttcttgggag | 1440 |
| | cagcaggaag cactatgggc gcagcgtcaa tgacgctgac ggtacaggcc agacaattat | 1500 |
| | tgtctggtat agtgcagcag cagaacaatt tgctgagggc tattgaggcg caacagcatc | 1560 |
| | tgttgcaact cacagtctgg ggcatcaagc agctccaggc aagaatcctg gctgtggaaa | 1620 |
| | gatacctaaa ggatcaacag ctcctgggga tttggggttg ctctggaaaa ctcatttgca | 1680 |
| | ccactgctgt gccttggaat gctagttgga gtaataaatc tctggaacag atttggaatc | 1740 |
| | acacgacctg gatggagtgg gacagagaaa ttaacaatta cacaagctta atacactcct | 1800 |
| | taattgaaga atcgcaaaac cagcaagaaa agaatgaaca agaattattg gaattagata | 1860 |
| | aatgggcaag tttgtggaat tggtttaaca taacaaattg gctgtggtat ataaaattat | 1920 |
| | tcataatgat agtaggaggc ttggtaggtt taagaatagt ttttgctgta ctttctatag | 1980 |
| | tgaatagagt taggcaggga tattcaccat tatcgtttca gacccacctc ccaaccccga | 2040 |
| | ggggacccga caggcccgaa ggaatagaag aagaaggtgg agagagagac agagacagat | 2100 |
| | ccattcgatt agtgaacgga tctcgacggt atcggtttta aaagaaaagg ggggattggg | 2160 |
| | gggtacagtg caggggaaag aatagtagac ataatagcaa cagacataca aactaaagaa | 2220 |
| | ttacaaaaac aaattacaaa aattcaaaat tttatcgatt ttatttagtc tccagaaaaa | 2280 |
| | ggggggaatg aaagacccca cctgtaggtt tggcaagcta gcttaagtaa cgccattttg | 2340 |
| | caaggcatgg aaaatacata actgagaata gagaagttca gatcaaggtt aggaacagag | 2400 |
| | agacagcaga atatgggcca aacaggatat ctgtggtaag cagttcctgc cccggctcag | 2460 |
| | ggccaagaac agatggtccc cagatgcggt cccgccctca gcagtttcta gagaaccatc | 2520 |
| | agatgtttcc agggtgcccc aaggacctga aatgaccctg tgccttattt gaactaacca | 2580 |
| | atcagttcgc ttctcgcttc tgttcgcgcg cttctgctcc ccgagctcaa taaaagagcc | 2640 |
| | cacaacccct cactcggcgc gccagtcctc cgatagactg cgtcgcccgg gtaccgatat | 2700 |
| | caacaagttt gtacaaaaaa gcaggcttcg ccaccatggg cctgagcaac atcctgttcg | 2760 |
| | tgatggcctt cctgctgtcc ggagccgccc ctctgaagat ccaggcctac ttcaacgaga | 2820 |
| | ccgccgacct gccctgccag ttcgccaaca gccagaacca gagcctgagc gaactggtgg | 2880 |
| | tgttctggca ggaccaggaa aacctggtcc tgaacgaggt gtacctgggc aaagaaaagt | 2940 |
| | tcgacagcgt gcacagcaag tacatgggcc ggaccagctt cgacagcgac agctggaccc | 3000 |
| | tgcggctgca caacctgcag atcaaggaca agggcctgta ccagtgcatc atccaccaca | 3060 |
| | agaatccacc cggcatgatc agaatccacc agatgaacag cgagctgtcc gtgctggcca | 3120 |
| | acttcagcca gcccgagatc gtgcccatca gcaacatcac cgagaacgtg tacatcaacc | 3180 |
| | tgacctgcag cagcatccac ggctaccccg agcccaagaa aatgagcgtg ctgctgcgga | 3240 |
| | ccaagaacag caccatcgag tacgacggcg tgatgcagaa aagccaggac aacgtgaccg | 3300 |
| | agctgtacga cgtgagcatc agcctgagcg tgagcttccc cgacgtgacc agcaacatga | 3360 |
| | ccatcttttg catcctggaa accgacaaga cccggctgct gtccagcccc ttcagcatcg | 3420 |
| | agctggaaga tccccagccc cctcccgacc acatcccctg gatcaccgcc gtgctgccca | 3480 |
| | ccgtgatcat ctgcgtgatg gtgttctgcc tgatcctgtg gaagtggaag aagaagaagc | 3540 |
| | ggcctaggaa cagctacaag tgcggcacca acaccatgga acgggaggaa agcgagcaga | 3600 |
| | ccaagaagcg ggagaagatc cacatccccg agcggagcga cgaggcccag cgggtgttca | 3660 |
| | agagcagcaa gaccagcagc tgcgacaaga gcgacacctg cttctaggac ccagctttct | 3720 |
| | tgtacaaagt ggtgattcga gttaattaag ctagcctagt gccatttgtt cagtggttcg | 3780 |
| | tagggctttc ccccactgtt tggctttcag ttatatggat gatgtggtat tgggggccaa | 3840 |
| | gtctgtacag catcttgagt ccctttttac cgctgttacc aattttcttt tgtctttggg | 3900 |
| | tatacattta aaccctaaca aaacaaagag atggggttac tctctaaatt ttatgggtta | 3960 |
| | tgtcattgga tgttatgggt ccttgccaca agaacacatc atacaaaaaa tcaaagaatg | 4020 |
| | ttttagaaaa cttcctatta acaggcctat tgattggaaa gtatgtcaac gaattgtggg | 4080 |
| | tcttttgggt tttgctgccc cttttacaca atgtggttat cctgcgttga tgcctttgta | 4140 |
| | tgcatgtatt caatctaagc aggctttcac tttctcgcca acttacaagg cctttctgtg | 4200 |
| | taaacaatac ctgaaccttt accccgttgc ccggcaacgg ccaggtctgt gccaagtgtt | 4260 |
| | tgctgacgca acccccactg gctggggctt ggtcatgggc catcagcgca tgcgtggaac | 4320 |
| | cttttcggct cctctgccga tccatactgc ggaactccta gccgcttgtt ttgctcgcag | 4380 |
| | caggtctgga gcaaacatta tcgggactga taactctgtt gtcctatccc gcaaatatac | 4440 |
| | atcgtttcca tggctgctag gctgtgctgc caactggatc ctgcgcggga cgtcctttgt | 4500 |
| | ttacgtcccg tcggcgctga atcctgcgga cgacccttct cggggtcgct tgggactctc | 4560 |
| | tcgtcccctt ctccgtctgc cgttccgacc gaccacgggg cgcacctctc tttacgcgga | 4620 |
| | ctccccgtct gtgccttctc atctgccgga ccgtgtgcac ttcgcttcac ctctgcacgt | 4680 |
| | cgcatggaga ccaccgtgaa cgcccaccaa atattgccca aggtcttaca taagaggact | 4740 |
| | cttggactct cagcaatgtc aacgaccgac cttgaggcat acttcaaaga ctgtttgttt | 4800 |
| | aaagactggg aggagttggg ggaggagatt aggttaaagg tctttgtact aggaggctgt | 4860 |
| | aggcataaat tggtctgcgc accagcacca tggcgcaatc actagagcgg ggtaccttta | 4920 |
| | agaccaatga cttacaaggc agctgtagat cttagccact ttttaaaaga aaagggggga | 4980 |
| | ctggaagggc taattcactc ccaacgaaga caagatctgc tttttgcttg tactgggtct | 5040 |
| | ctctggttag accagatctg agcctgggag ctctctggct aactagggaa cccactgctt | 5100 |
| | aagcctcaat aaagcttgcc ttgagtgctt caagtagtgt gtgcccgtct gttgtgtgac | 5160 |
| | tctggtaact agagatccct cagacccttt tagtcagtgt ggaaaatctc tagcagtagt | 5220 |
| | agttcatgtc atcttattat tcagtattta taacttgcaa agaaatgaat atcagagagt | 5280 |
| | gagaggaact tgtttattgc agcttataat ggttacaaat aaagcaatag catcacaaat | 5340 |
| | ttcacaaata aagcattttt ttcactgcat tctagttgtg gtttgtccaa actcatcaat | 5400 |
| | gtatcttatc atgtctggct ctagctatcc cgcccctaac tccgcccatc ccgcccctaa | 5460 |
| | ctccgcccag ttccgcccat tctccgcccc atggctgact aatttttttt atttatgcag | 5520 |
| | aggccgaggc cggatccctt gagtggcttt catcctggag cagactttgc agtctgtgga | 5580 |
| | ctgcaacaca acattgcctt tatgtgtaac tcttggctga agctcttaca ccaatgctgg | 5640 |
| | gggacatgta cctcccaggg gcccaggaag actacgggag gctacaccaa cgtcaatcag | 5700 |
| | aggggcctgt gtagctaccg ataagcggac cctcaagagg gcattagcaa tagtgtttat | 5760 |
| | aaggccccct tgttaattct tgaagacgaa agggcctcgt gatacgccta tttttatagg | 5820 |
| | ttaatgtcat gataataatg gtttcttaga cgtcaggtgg cacttttcgg ggaaatgtgc | 5880 |
| | gcggaacccc tatttgttta tttttctaaa tacattcaaa tatgtatccg ctcatgagac | 5940 |
| | aataaccctg ataaatgctt caataatatt gaaaaaggaa gagtatgagt attcaacatt | 6000 |
| | tccgtgtcgc ccttattccc ttttttgcgg cattttgcct tcctgttttt gctcacccag | 6060 |
| | aaacgctggt gaaagtaaaa gatgctgaag atcagttggg tgcacgagtg ggttacatcg | 6120 |
| | aactggatct caacagcggt aagatccttg agagttttcg ccccgaagaa cgttttccaa | 6180 |
| | tgatgagcac ttttaaagtt ctgctatgtg gcgcggtatt atcccgtgtt gacgccgggc | 6240 |
| | aagagcaact cggtcgccgc atacactatt ctcagaatga cttggttgag tactcaccag | 6300 |
| | tcacagaaaa gcatcttacg gatggcatga cagtaagaga attatgcagt gctgccataa | 6360 |
| | ccatgagtga taacactgcg gccaacttac ttctgacaac gatcggagga ccgaaggagc | 6420 |
| | taaccgcttt tttgcacaac atgggggatc atgtaactcg ccttgatcgt tgggaaccgg | 6480 |
| | agctgaatga agccatacca aacgacgagc gtgacaccac gatgcctgca gcaatggcaa | 6540 |
| | caacgttgcg caaactatta actggcgaac tacttactct agcttcccgg caacaattaa | 6600 |
| | tagactggat ggaggcggat aaagttgcag gaccacttct gcgctcggcc cttccggctg | 6660 |
| | gctggtttat tgctgataaa tctggagccg gtgagcgtgg gtctcgcggt atcattgcag | 6720 |
| | cactggggcc agatggtaag ccctcccgta tcgtagttat ctacacgacg gggagtcagg | 6780 |
| | caactatgga tgaacgaaat agacagatcg ctgagatagg tgcctcactg attaagcatt | 6840 |
| | ggtaactgtc agaccaagtt tactcatata tactttagat tgatttaaaa cttcattttt | 6900 |
| | aatttaaaag gatctaggtg aagatccttt ttgataatct catgaccaaa atcccttaac | 6960 |
| | gtgagttttc gttccactga gcgtcagacc ccgtagaaaa gatcaaagga tcttcttgag | 7020 |
| | atcctttttt tctgcgcgta atctgctgct tgcaaacaaa aaaaccaccg ctaccagcgg | 7080 |
| | tggtttgttt gccggatcaa gagctaccaa ctctttttcc gaaggtaact ggcttcagca | 7140 |
| | gagcgcagat accaaatact gtccttctag tgtagccgta gttaggccac cacttcaaga | 7200 |
| | actctgtagc accgcctaca tacctcgctc tgctaatcct gttaccagtg gctgctgcca | 7260 |
| | gtggcgataa gtcgtgtctt accgggttgg actcaagacg atagttaccg gataaggcgc | 7320 |
| | agcggtcggg ctgaacgggg ggttcgtgca cacagcccag cttggagcga acgacctaca | 7380 |
| | ccgaactgag atacctacag cgtgagcatt gagaaagcgc cacgcttccc gaagggagaa | 7440 |
| | aggcggacag gtatccggta agcggcaggg tcggaacagg agagcgcacg agggagcttc | 7500 |
| | cagggggaaa cgcctggtat ctttatagtc ctgtcgggtt tcgccacctc tgacttgagc | 7560 |
| | gtcgattttt gtgatgctcg tcaggggggc ggagcctatg gaaaaacgcc agcaacgcgg | 7620 |
| | cctttttacg gttcctggcc ttttgctggc ctttttgaag ctgtccctga tggtcgtcat | 7680 |
| | ctacctgcct ggacagcatg gcctgcaacg cgggcatccc gatgccgccg gaagcgagaa | 7740 |
| | gaatcataat ggggaaggcc atccagcctc gcgtcg | 7776 |
| SEQ ID NO: 19 | atgggcctga gcaacatcct gttcgtgatg gccttcctgc tgtccggagc cgcccctctg | 60 |
| (CD86 CoOP) | aagatccagg cctacttcaa cgagaccgcc gacctgccct gccagttcgc caacagccag | 120 |
| | aaccagagcc tgagcgaact ggtggtgttc tggcaggacc aggaaaacct ggtcctgaac | 180 |
| | gaggtgtacc tgggcaaaga aaagttcgac agcgtgcaca gcaagtacat gggccggacc | 240 |
| | agcttcgaca gcgacagctg gaccctgcgg ctgcacaacc tgcagatcaa ggacaagggc | 300 |
| | ctgtaccagt gcatcatcca ccacaagaaa cccaccggca tgatcagaat ccaccagatg | 360 |
| | aacagcgagc tgtccgtgct ggccaacttc agccagcccg agatcgtgcc catcagcaac | 420 |
| | atcaccgaga acgtgtacat caacctgacc tgcagcagca tccacggcta ccccgagccc | 480 |
| | aagaaaatga gcgtgctgct gcggaccaag aacagcacca tcgagtacga cggcgtgatg | 540 |
| | cagaaaagcc aggacaacgt gaccgagctg tacgacgtga gcatcagcct gagcgtgagc | 600 |
| | ttccccgacg tgaccagcaa catgaccatc ttttgcatcc tggaaaccga caagacccgg | 660 |
| | ctgctgtcca gccccttcag catcgagctg gaagatcccc agccccctcc cgaccacatc | 720 |
| | ccctggatca ccgccgtgct gcccaccgtg atcatctgcg tgatggtgtt ctgcctgatc | 780 |
| | ctgtggaagt ggaagaagaa gaagcggcct aggaacagct acaagtgcgg caccaacacc | 840 |
| | atggaacggg aggaaagcga gcagaccaag aagcgggaga agatccacat ccccgagcgg | 900 |
| | agcgacgagg cccagcgggt gttcaagagc agcaagacca gcagctgcga caagagcgac | 960 |
| | acctgcttc | 969 |
| SEQ ID NO: 20 | ggggacaagt ttgtacaaaa aagcaggctt cgccaccatg ggcctgagca acatcctgtt | 60 |
| (CD86 PCRP) | cgtgatggcc ttcctgctgt ccggagccgc ccctctgaag atccaggcct acttcaacga | 120 |
| | gaccgccgac ctgccctgcc agttcgccaa cagccagaac cagagcctga gcgaactggt | 180 |
| | ggtgttctgg caggaccagg aaaacctggt cctgaacgag gtgtacctgg gcaaagaaaa | 240 |
| | gttcgacagc gtgcacagca agtacatggg ccggaccagc ttcgacagcg acagctggac | 300 |
| | cctgcggctg cacaacctgc agatcaagga caagggcctg taccagtgca tcatccacca | 360 |
| | caagaaaccc accggcatga tcagaatcca ccagatgaac agcgagctgt ccgtgctggc | 420 |
| | caacttcagc cagcccgaga tcgtgcccat cagcaacatc accgagaacg tgtacatcaa | 480 |
| | cctgacctgc agcagcatcc acggctaccc cgagcccaag aaaatgagcg tgctgctgcg | 540 |
| | gaccaagaac agcaccatcg agtacgacgg cgtgatgcag aaaagccagg acaacgtgac | 600 |
| | cgagctgtac gacgtgagca tcagcctgag cgtgagcttc cccgacgtga ccagcaacat | 660 |
| | gaccatcttt tgcatcctgg aaaccgacaa gacccggctg ctgtccagcc ccttcagcat | 720 |
| | cgagctggaa gatccccagc cccctcccga ccacatcccc tggatcaccg ccgtgctgcc | 780 |
| | caccgtgatc atctgcgtga tggtgttctg cctgatcctg tggaagtgga agaagaagaa | 840 |
| | gcggcctagg aacagctaca agtgcggcac caacaccatg gaacgggagg aaagcgagca | 900 |
| | gaccaagaag cgggagaaga tccacatccc cgagcggagc gacgaggccc agcgggtgtt | 960 |
| | caagagcagc aagaccagca gctgcgacaa gagcgacacc tgcttctagg acccagcttt | 1020 |
| | cttgtacaaa gtggtcccc | 1039 |
| SEQ ID NO: 21 | ctttcctgcg ttatcccctg attctgtgga taaccgtatt accgcctttg agtgagctga | 60 |
| (pDONR221 CD86 | taccgctcgc cgcagccgaa cgaccgagcg cagcgagtca gtgagcgagg aagcggaaga | 120 |
| vector) | gcgcccaata cgcaaaccgc ctctccccgc gcgttggccg attcattaat gcagctggca | 180 |
| | cgacaggttt cccgactgga aagcgggcag tgagcgcaac gcaattaata cgcgtaccgc | 240 |
| | tagccaggaa gagtttgtag aaacgcaaaa aggccatccg tcaggatggc cttctgctta | 300 |
| | gtttgatgcc tggcagttta tggcgggcgt cctgcccgcc accctccggg ccgttgcttc | 360 |
| | acaacgttca aatccgctcc cggcggattt gtcctactca ggagagcgtt caccgacaaa | 420 |
| | caacagataa aacgaaaggc ccagtcttcc gactgagcct ttcgttttat ttgatgcctg | 480 |
| | gcagttccct actctcgcgt taacgctagc atggatgttt tcccagtcac gacgttgtaa | 540 |
| | aacgacggcc agtcttaagc tcgggcccca aataatgatt ttattttgac tgatagtgac | 600 |
| | ctgttcgttg caacacattg atgagcaatg cttttttata atgcacaagt ttgtacaaaa | 660 |
| | aagcaggctt cgccaccatg ggcctgagca acatcctgtt cgtgatggcc ttcctgctgt | 720 |
| | ccggagccgc ccctctgaag atccaggcct acttcaacga gaccgccgac ctgccctgcc | 780 |
| | agttcgccaa cagccagaac cagagcctga gcgaactggt ggtgttctgg caggaccagg | 840 |
| | aaaacctggt cctgaacgag gtgtacctgg gcaaagaaaa gttcgacagc gtgcacagca | 900 |
| | agtacatggg ccggaccagc ttcgacagcg acagctggac cctgcggctg cacaacctgc | 960 |
| | agatcaagga caagggcctg taccagtgca tcatccacca caagaaaccc accggcatga | 1020 |
| | tcagaatcca ccagatgaac agcgagctgt ccgtgctggc caacttcagc cagcccgaga | 1080 |
| | tcgtgcccat cagcaacatc accgagaacg tgtacatcaa cctgacctgc agcagcatcc | 1140 |
| | acggctaccc cgagcccaag aaaatgagcg tgctgctgcg gaccaagaac agcaccatcg | 1200 |
| | agtacgacgg cgtgatgcag aaaagccagg acaacgtgac cgagctgtac gacgtgagca | 1260 |
| | tcagcctgag cgtgagcttc cccgacgtga ccagcaacat gaccatcttt tgcatcctgg | 1320 |
| | aaaccgacaa gacccggctg ctgtccagcc ccttcagcat cgagctggaa gatccccagc | 1380 |
| | cccctcccga ccacatcccc tggatcaccg ccgtgctgcc caccgtgatc atctgcgtga | 1440 |
| | tggtgttctg cctgatcctg tggaagtgga agaagaagaa gcggcctagg aacagctaca | 1500 |
| | agtgcggcac caacaccatg gaacgggagg aaagcgagca gaccaagaag cgggagaaga | 1560 |
| | tccacatccc cgagcggagc gacgaggccc agcgggtgtt caagagcagc aagaccagca | 1620 |
| | gctgcgacaa gagcgacacc tgcttctagg acccagcttt cttgtacaaa gtggtcatta | 1680 |
| | taagaaagca ttgcttatca atttgttgca acgaacaggt cactatcagt caaaataaaa | 1740 |
| | tcattatttg ccatccagct gatatcccct atagtgagtc gtattacatg gtcatagctg | 1800 |
| | tttcctggca gctctggccc gtgtctcaaa atctctgatg ttacattgca caagataaaa | 1860 |
| | taatatcatc atgaacaata aaactgtctg cttacataaa cagtaataca aggggtgtta | 1920 |
| | tgagccatat tcaacgggaa acgtcgaggc cgcgattaaa ttccaacatg gatgctgatt | 1980 |
| | tatatgggta taaatgggct cgcgataatg tcgggcaatc aggtgcgaca atctatcgct | 2040 |
| | tgtatgggaa gcccgatgcg ccagagttgt ttctgaaaca tggcaaaggt agcgttgcca | 2100 |
| | atgatgttac agatgagatg gtcagactaa actggctgac ggaatttatg cctcttccga | 2160 |
| | ccatcaagca ttttatccgt actcctgatg atgcatggtt actcaccact gcgatccccg | 2220 |
| | gaaaaacagc attccaggta ttagaagaat atcctgattc aggtgaaaat attgttgatg | 2280 |
| | cgctggcagt gttcctgcgc cggttgcatt cgattcctgt ttgtaattgt ccttttaaca | 2340 |
| | gcgatcgcgt atttcgtctc gctcaggcgc aatcacgaat gaataacggt ttggttgatg | 2400 |
| | cgagtgattt tgatgacgag cgtaatggct ggcctgttga acaagtctgg aaagaaatgc | 2460 |
| | ataaactttt gccattctca ccggattcag tcgtcactca tggtgatttc tcacttgata | 2520 |
| | accttatttt tgacgagggg aaattaatag gttgtattga tgttggacga gtcggaatcg | 2580 |
| | cagaccgata ccaggatctt gccatcctat ggaactgcct cggtgagttt tctccttcat | 2640 |
| | tacagaaacg gctttttcaa aaatatggta ttgataatcc tgatatgaat aaattgcagt | 2700 |
| | ttcatttgat gctcgatgag tttttctaat cagaattggt taattggttg taacactggc | 2760 |
| | agagcattac gctgacttga cgggacggcg caagctcatg accaaaatcc cttaacgtga | 2820 |
| | gttacgcgtc gttccactga gcgtcagacc ccgtagaaaa gatcaaagga tcttcttgag | 2880 |
| | atcctttttt tctgcgcgta atctgctgct tgcaaacaaa aaaaccaccg ctaccagcgg | 2940 |
| | tggtttgttt gccggatcaa gagctaccaa ctctttttcc gaaggtaact ggcttcagca | 3000 |
| | gagcgcagat accaaatact gttcttctag tgtagccgta gttaggccac cacttcaaga | 3060 |
| | actctgtagc accgcctaca tacctcgctc tgctaatcct gttaccagtg gctgctgcca | 3120 |
| | gtggcgataa gtcgtgtctt accgggttgg actcaagacg atagttaccg gataaggcgc | 3180 |
| | agcggtcggg ctgaacgggg ggttcgtgca cacagcccag cttggagcga acgacctaca | 3240 |
| | ccgaactgag atacctacag cgtgagctat gagaaagcgc cacgcttccc gaagggagaa | 3300 |
| | aggcggacag gtatccggta agcggcaggg tcggaacagg agagcgcacg agggagcttc | 3360 |
| | cagggggaaa cgcctggtat ctttatagtc ctgtcgggtt tcgccacctc tgacttgagc | 3420 |
| | gtcgattttt gtgatgctcg tcaggggggc ggagcctatg gaaaaacgcc agcaacgcgg | 3480 |
| | cctttttacg gttcctggcc ttttgctggc cttttgctca catgtt | 3526 |
| SEQ ID NO: 22 | ctttcctgcg ttatcccctg attctgtgga taaccgtatt accgcctttg agtgagctga | 60 |
| (pDONR221 4- | taccgctcgc cgcagccgaa cgaccgagcg cagcgagtca gtgagcgagg aagcggaaga | 120 |
| 1BBL vector) | gcgcccaata cgcaaaccgc ctctccccgc gcgttggccg attcattaat gcagctggca | 180 |
| | cgacaggttt cccgactgga aagcgggcag tgagcgcaac gcaattaata cgcgtaccgc | 240 |
| | tagccaggaa gagtttgtag aaacgcaaaa aggccatccg tcaggatggc cttctgctta | 300 |
| | gtttgatgcc tggcagttta tggcgggcgt cctgcccgcc accctccggg ccgttgcttc | 360 |
| | acaacgttca aatccgctcc cggcggattt gtcctactca ggagagcgtt caccgacaaa | 420 |
| | caacagataa aacgaaaggc ccagtcttcc gactgagcct ttcgttttat ttgatgcctg | 480 |
| | gcagttccct actctcgcgt taacgctagc atggatgttt tcccagtcac gacgttgtaa | 540 |
| | aacgacggcc agtcttaagc tcgggcccca aataatgatt ttattttgac tgatagtgac | 600 |
| | ctgttcgttg caacacattg atgagcaatg cttttttata atgcacaagt ttgtacaaaa | 660 |
| | aagcaggctt cgccaccatg gaatacgcct ctgatgccag cctggacccc gaagctcctt | 720 |
| | ggcctcctgc ccctagagcc agagcctgta gagtgctgcc ttgggctctg gtggctggcc | 780 |
| | ttctccttct gctgctgctg gccgctgcct gcgctgtgtt tctggcttgt ccttgggccg | 840 |
| | tgtcaggcgc cagagcttct cctggatctg ccgccagccc cagactgaga gagggacctg | 900 |
| | agctgagccc cgatgatcct gccggactgc tggatctgag acagggcatg ttcgcccagc | 960 |
| | tggtggccca gaacgtgctg ctgatcgatg gccccctgag ctggtacagc gatcctggac | 1020 |
| | tggctggcgt gtcactgaca ggcggcctga gctacaaaga ggacaccaaa gaactggtgg | 1080 |
| | tggccaaggc cggcgtgtac tacgtgttct ttcagctgga actgcggaga gtggtggccg | 1140 |
| | gcgaaggatc cggctctgtg tctctggcac tgcatctgca gcccctgaga tctgctgcag | 1200 |
| | gcgctgctgc actggccctg acagtggacc tgcctccagc ctctagcgag gccagaaact | 1260 |
| | ccgcattcgg gtttcaaggc agactgctgc acctgtctgc cggccagaga ctgggagtgc | 1320 |
| | atctgcacac agaggccaga gccagacacg cctggcagct gacacagggc gctacagtgc | 1380 |
| | tgggcctgtt cagagtgacc cccgaaattc cagccggcct gcccagccct agaagcgagt | 1440 |
| | aggacccagc tttcttgtac aaagtggtca ttataagaaa gcattgctta tcaatttgtt | 1500 |
| | gcaacgaaca ggtcactatc agtcaaaata aaatcattat ttgccatcca gctgatatcc | 1560 |
| | cctatagtga gtcgtattac atggtcatag ctgtttcctg gcagctctgg cccgtgtctc | 1620 |
| | aaaatctctg atgttacatt gcacaagata aaataatatc atcatgaaca ataaaactgt | 1680 |
| | ctgcttacat aaacagtaat acaaggggtg ttatgagcca tattcaacgg gaaacgtcga | 1740 |
| | ggccgcgatt aaattccaac atggatgctg atttatatgg gtataaatgg gctcgcgata | 1800 |
| | atgtcgggca atcaggtgcg acaatctatc gcttgtatgg gaagcccgat gcgccagagt | 1860 |
| | tgtttctgaa acatggcaaa ggtagcgttg ccaatgatgt tacagatgag atggtcagac | 1920 |
| | taaactggct gacggaattt atgcctcttc cgaccatcaa gcattttatc cgtactcctg | 1980 |
| | atgatgcatg gttactcacc actgcgatcc ccggaaaaac agcattccag gtattagaag | 2040 |
| | aatatcctga ttcaggtgaa aatattgttg atgcgctggc agtgttcctg cgccggttgc | 2100 |
| | attcgattcc tgtttgtaat tgtcctttta acagcgatcg cgtatttcgt ctcgctcagg | 2160 |
| | cgcaatcacg aatgaataac ggtttggttg atgcgagtga ttttgatgac gagcgtaatg | 2220 |
| | gctggcctgt tgaacaagtc tggaaagaaa tgcataaact tttgccattc tcaccggatt | 2280 |
| | cagtcgtcac tcatggtgat ttctcacttg ataaccttat ttttgacgag gggaaattaa | 2340 |
| | taggttgtat tgatgttgga cgagtcggaa tcgcagaccg ataccaggat cttgccatcc | 2400 |
| | tatggaactg cctcggtgag ttttctcctt cattacagaa acggcttttt caaaaatatg | 2460 |
| | gtattgataa tcctgatatg aataaattgc agtttcattt gatgctcgat gagtttttct | 2520 |
| | aatcagaatt ggttaattgg ttgtaacact ggcagagcat tacgctgact tgacgggacg | 2580 |
| | gcgcaagctc atgaccaaaa tcccttaacg tgagttacgc gtcgttccac tgagcgtcag | 2640 |
| | accccgtaga aaagatcaaa ggatcttctt gagatccttt ttttctgcgc gtaatctgct | 2700 |
| | gcttgcaaac aaaaaaacca ccgctaccag cggtggtttg tttgccggat caagagctac | 2760 |
| | caactctttt tccgaaggta actggcttca gcagagcgca gataccaaat actgttcttc | 2820 |
| | tagtgtagcc gtagttaggc caccacttca agaactctgt agcaccgcct acatacctcg | 2880 |
| | ctctgctaat cctgttacca gtggctgctg ccagtggcga taagtcgtgt cttaccgggt | 2940 |
| | tggactcaag acgatagtta ccggataagg cgcagcggtc gggctgaacg gggggttcgt | 3000 |
| | gcacacagcc cagcttggag cgaacgacct acaccgaact gagataccta cagcgtgagc | 3060 |
| | tatgagaaag cgccacgctt cccgaaggga gaaaggcgga caggtatccg gtaagcggca | 3120 |
| | gggtcggaac aggagagcgc acgagggagc ttccaggggg aaacgcctgg tatctttata | 3180 |
| | gtcctgtcgg gtttcgccac ctctgacttg agcgtcgatt tttgtgatgc tcgtcagggg | 3240 |
| | ggcggagcct atggaaaaac gccagcaacg cggccttttt acggttcctg gccttttgct | 3300 |
| | ggccttttgc tcacatgtt | 3319 |
| SEQ ID NO: 23 | cgataaccct aattcgatag catatgcttc ccgttgggta acatatgcta ttgaattagg | 60 |
| (pLV430G | gttagtctgg atagtatata ctactacccg ggaagcatat gctacccgtt tagggttcac | 120 |
| vector) | cggtgatgcc ggccacgatg cgtccggcgt agaggatcta atgtgagtta gctcactcat | 180 |
| | taggcacccc aggctttaca ctttatgctt ccggctcgta tgttgtgtgg aattgtgagc | 240 |
| | ggataacaat ttcacacagg aaacagctat gaccatgatt acgccaagcg cgcaattaac | 300 |
| | cctcactaaa gggaacaaaa gctggagctg caagcttaat gtagtcttat gcaatactct | 360 |
| | tgtagtcttg caacatggta acgatgagtt agcaacatgc cttacaagga gagaaaaagc | 420 |
| | accgtgcatg ccgattggtg gaagtaaggt ggtacgatcg tgccttatta ggaaggcaac | 480 |
| | agacgggtct gacatggatt ggacgaacca ctgaattgcc gcattgcaga gatattgtat | 540 |
| | ttaagtgcct agctcgatac ataaacgggt ctctctggtt agaccagatc tgagcctggg | 600 |
| | agctctctgg ctaactaggg aacccactgc ttaagcctca ataaagcttg ccttgagtgc | 660 |
| | ttcaagtagt gtgtgcccgt ctgttgtgtg actctggtaa ctagagatcc ctcagaccct | 720 |
| | tttagtcagt gtggaaaatc tctagcagtg gcgcccgaac agggacttga aagcgaaagg | 780 |
| | gaaaccagag gagctctctc gacgcaggac tcggcttgct gaagcgcgca cggcaagagg | 840 |
| | cgaggggcgg cgactggtga gtacgccaaa aattttgact agcggaggct agaaggagag | 900 |
| | agatgggtgc gagagcgtca gtattaagcg ggggagaatt agatcgcgat gggaaaaaat | 960 |
| | tcggttaagg ccagggggaa agaaaaaata taaattaaaa catatagtat gggcaagcag | 1020 |
| | ggagctagaa cgattcgcag ttaatcctgg cctgttagaa acatcagaag gctgtagaca | 1080 |
| | aatactggga cagctacaac catcccttca gacaggatca gaagaactta gatcattata | 1140 |
| | taatacagta gcaaccctct attgtgtgca tcaaaggata gagataaaag acaccaagga | 1200 |
| | agctttagac aagatagagg aagagcaaaa caaaagtaag accaccgcac agcaagcggc | 1260 |
| | cgctgatctt cagacctgga ggaggagata tgagggacaa ttggagaagt gaattatata | 1320 |
| | aatataaagt agtaaaaatt gaaccattag gagtagcacc caccaaggca aagagaagag | 1380 |
| | tggtgcagag agaaaaaaga gcagtgggaa taggagcttt gttccttggg ttcttgggag | 1440 |
| | cagcaggaag cactatgggc gcagcgtcaa tgacgctgac ggtacaggcc agacaattat | 1500 |
| | tgtctggtat agtgcagcag cagaacaatt tgctgagggc tattgaggcg caacagcatc | 1560 |
| | tgttgcaact cacagtctgg ggcatcaagc agctccaggc aagaatcctg gctgtggaaa | 1620 |
| | gatacctaaa ggatcaacag ctcctgggga tttggggttg ctctggaaaa ctcatttgca | 1680 |
| | ccactgctgt gccttggaat gctagttgga gtaataaatc tctggaacag atttggaatc | 1740 |
| | acacgacctg gatggagtgg gacagagaaa ttaacaatta cacaagctta atacactcct | 1800 |
| | taattgaaga atcgcaaaac cagcaagaaa agaatgaaca agaattattg gaattagata | 1860 |
| | aatgggcaag tttgtggaat tggtttaaca taacaaattg gctgtggtat ataaaattat | 1920 |
| | tcataatgat agtaggaggc ttggtaggtt taagaatagt ttttgctgta ctttctatag | 1980 |
| | tgaatagagt taggcaggga tattcaccat tatcgtttca gacccacctc ccaaccccga | 2040 |
| | ggggacccga caggcccgaa ggaatagaag aagaaggtgg agagagagac agagacagat | 2100 |
| | ccattcgatt agtgaacgga tctcgacggt atcggtttta aaagaaaagg ggggattggg | 2160 |
| | gggtacagtg caggggaaag aatagtagac ataatagcaa cagacataca aactaaagaa | 2220 |
| | ttacaaaaac aaattacaaa aattcaaaat tttatcgatt ttatttagtc tccagaaaaa | 2280 |
| | ggggggaatg aaagacccca cctgtaggtt tggcaagcta gcttaagtaa cgccattttg | 2340 |
| | caaggcatgg aaaatacata actgagaata gagaagttca gatcaaggtt aggaacagag | 2400 |
| | agacagcaga atatgggcca aacaggatat ctgtggtaag cagttcctgc cccggctcag | 2460 |
| | ggccaagaac agatggtccc cagatgcggt cccgccctca gcagtttcta gagaaccatc | 2520 |
| | agatgtttcc agggtgcccc aaggacctga aatgaccctg tgccttattt gaactaacca | 2580 |
| | atcagttcgc ttctcgcttc tgttcgcgcg cttctgctcc ccgagctcaa taaaagagcc | 2640 |
| | cacaacccct cactcggcgc gccagtcctc cgatagactg cgtcgcccgg gtaccgatat | 2700 |
| | cacaagtttg tacaaaaaag ctgaacgaga aacgtaaaat gatataaata tcaatatatt | 2760 |
| | aaattagatt ttgcataaaa aacagactac ataatactgt aaaacacaac atatccagtc | 2820 |
| | actatggcgg ccgcattagg caccccaggc tttacacttt atgcttccgg ctcgtataat | 2880 |
| | gtgtggattt tgagttagga tccgtcgaga ttttcaggag ctaaggaagc taaaatggag | 2940 |
| | aaaaaaatca ctggatatac caccgttgat atatcccaat ggcatcgtaa agaacatttt | 3000 |
| | gaggcatttc agtcagttgc tcaatgtacc tataaccaga ccgttcagct ggatattacg | 3060 |
| | gcctttttaa agaccgtaaa gaaaaataag cacaagtttt atccggcctt tattcacatt | 3120 |
| | cttgcccgcc tgatgaatgc tcatccggaa ttccgtatgg caatgaaaga cggtgagctg | 3180 |
| | gtgatatggg atagtgttca cccttgttac accgttttcc atgagcaaac tgaaacgttt | 3240 |
| | tcatcgctct ggagtgaata ccacgacgat ttccggcagt ttctacacat atattcgcaa | 3300 |
| | gatgtggcgt gttacggtga aaacctggcc tatttcccta aagggtttat tgagaatatg | 3360 |
| | tttttcgtct cagccaatcc ctgggtgagt ttcaccagtt ttgatttaaa cgtggccaat | 3420 |
| | atggacaact tcttcgcccc cgttttcacc atgggcaaat attatacgca aggcgacaag | 3480 |
| | gtgctgatgc cgctggcgat tcaggttcat catgccgttt gtgatggctt ccatgtcggc | 3540 |
| | agaatgctta atgaattaca acagtactgc gatgagtggc agggcggggc gtaaacgcgt | 3600 |
| | ggatccggct tactaaaagc cagataacag tatgcgtatt tgcgcgctga tttttgcggt | 3660 |
| | ataagaatat atactgatat gtatacccga agtatgtcaa aaagaggtat gctatgaagc | 3720 |
| | agcgtattac agtgacagtt gacagcgaca gctatcagtt gctcaaggca tatatgatgt | 3780 |
| | caatatctcc ggtctggtaa gcacaaccat gcagaatgaa gcccgtcgtc tgcgtgccga | 3840 |
| | acgctggaaa gcggaaaatc aggaagggat ggctgaggtc gcccggttta ttgaaatgaa | 3900 |
| | cggctctttt gctgacgaga acaggggctg gtgaaatgca gtttaaggtt tacacctata | 3960 |
| | aaagagagag ccgttatcgt ctgtttgtgg atgtacagag tgatattatt gacacgcccg | 4020 |
| | ggcgacggat ggtgatcccc ctggccagtg cacgtctgct gtcagataaa gtctcccgtg | 4080 |
| | aactttaccc ggtggtgcat atcggggatg aaagctggcg catgatgacc accgatatgg | 4140 |
| | ccagtgtgcc ggtctccgtt atcggggaag aagtggctga tctcagccac cgcgaaaatg | 4200 |
| | acatcaaaaa cgccattaac ctgatgttct ggggaatata aatgtcaggc tcccttatac | 4260 |
| | acagccagtc tgcaggtcga ccatagtgac tggatatgtt gtgttttaca gtattatgta | 4320 |
| | gtctgttttt tatgcaaaat ctaatttaat atattgatat ttatatcatt ttacgtttct | 4380 |
| | cgttcagctt tcttgtacaa agtggtgatt cgagttaatt aagctagcct agtgccattt | 4440 |
| | gttcagtggt tcgtagggct ttcccccact gtttggcttt cagttatatg gatgatgtgg | 4500 |
| | tattgggggc caagtctgta cagcatcttg agtccctttt taccgctgtt accaattttc | 4560 |
| | ttttgtcttt gggtatacat ttaaacccta acaaaacaaa gagatggggt tactctctaa | 4620 |
| | attttatggg ttatgtcatt ggatgttatg ggtccttgcc acaagaacac atcatacaaa | 4680 |
| | aaatcaaaga atgttttaga aaatcaaaga ttaacaggcc tattgattgg aaagtatgtc | 4740 |
| | aacgaattgt gggtcttttg ggttttgctg ccccttttac acaatgtggt tatcctgcgt | 4800 |
| | tgatgccttt gtatgcatgt attcaatcta agcaggcttt cactttctcg ccaacttaca | 4860 |
| | aggcctttct gtgtaaacaa tacctgaacc tttaccccgt tgcccggcaa cggccaggtc | 4920 |
| | tgtgccaagt gtttgctgac gcaaccccca ctggctgggg cttggtcatg ggccatcagc | 4980 |
| | gcatgcgtgg aaccttttcg gctcctctgc cgatccatac tgcggaactc ctagccgctt | 5040 |
| | gttttgctcg cagcaggtct ggagcaaaca ttatcgggac tgataactct gttgtcctat | 5100 |
| | cccgcaaata tacatcgttt ccatggctgc taggctgtgc tgccaactgg atcctgcgcg | 5160 |
| | ggacgtcctt tgtttacgtc ccgtcggcgc tgaatcctgc ggacgaccct tctcggggtc | 5220 |
| | gcttgggact ctctcgtccc cttctccgtc tgccgttccg accgaccacg gggcgcacct | 5280 |
| | ctctttacgc ggactccccg tctgtgcctt ctcatctgcc ggaccgtgtg cacttcgctt | 5340 |
| | cacctctgca cgtcgcatgg agaccaccgt gaacgcccac caaatattgc ccaaggtctt | 5400 |
| | acataagagg actcttggac tctcagcaat gtcaacgacc gaccttgagg catacttcaa | 5460 |
| | agactgtttg tttaaagact gggaggagtt gggggaggag attaggttaa aggtctttgt | 5520 |
| | actaggaggc tgtaggcata aattggtctg cgcaccagca ccatggcgca atcactagag | 5580 |
| | cggggtacct ttaagaccaa tgacttacaa ggcagctgta gatcttagcc actttttaaa | 5640 |
| | agaaaagggg ggactggaag ggctaattca ctcccaacga agacaagatc tgctttttgc | 5700 |
| | ttgtactggg tctctctggt tagaccagat ctgagcctgg gagctctctg gctaactagg | 5760 |
| | gaacccactg cttaagcctc aataaagctt gccttgagtg cttcaagtag tgtgtgcccg | 5820 |
| | tctgttgtgt gactctggta actagagatc cctcagaccc ttttagtcag tgtggaaaat | 5880 |
| | ctctagcagt agtagttcat gtcatcttat tattcagtat ttataacttg caaagaaatg | 5940 |
| | aatatcagag agtgagagga acttgtttat tgcagcttat aatggttaca aataaagcaa | 6000 |
| | tagcatcaca aatttcacaa ataaagcatt tttttcactg cattctagtt gtggtttgtc | 6060 |
| | caaactcatc aatgtatctt atcatgtctg gctctagcta tcccgcccct aactccgccc | 6120 |
| | atcccgcccc taactccgcc cagttccgcc cattctccgc cccatggctg actaattttt | 6180 |
| | tttatttatg cagaggccga ggccggatcc cttgagtggc tttcatcctg gagcagactt | 6240 |
| | tgcagtctgt ggactgcaac acaacattgc ctttatgtgt aactcttggc tgaagctctt | 6300 |
| | acaccaatgc tgggggacat gtacctccca ggggcccagg aagactacgg gaggctacac | 6360 |
| | caacgtcaat cagaggggcc tgtgtagcta ccgataagcg gaccctcaag agggcattag | 6420 |
| | caatagtgtt tataaggccc ccttgttaat tcttgaagac gaaagggcct cgtgatacgc | 6480 |
| | ctatttttat aggttaatgt catgataata atggtttctt agacgtcagg tggcactttt | 6540 |
| | cggggaaatg tgcgcggaac ccctatttgt ttatttttct aaatacattc aaatatgtat | 6600 |
| | ccgctcatga gacaataacc ctgataaatg cttcaataat attgaaaaag gaagagtatg | 6660 |
| | agtattcaac atttccgtgt cgcccttatt cccttttttg cggcattttg ccttcctgtt | 6720 |
| | tttgctcacc cagaaacgct ggtgaaagta aaagatgctg aagatcagtt gggtgcacga | 6780 |
| | gtgggttaca tcgaactgga tctcaacagc ggtaagatcc ttgagagttt tcgccccgaa | 6840 |
| | gaacgttttc caatgatgag cacttttaaa gttctgctat gtggcgcggt attatcccgt | 6900 |
| | gttgacgccg ggcaagagca actcggtcgc cgcatacact attctcagaa tgacttggtt | 6960 |
| | gagtactcac cagtcacaga aaagcatctt acggatggca tgacagtaag agaattatgc | 7020 |
| | agtgctgcca taaccatgag tgataacact gcggccaact tacttctgac aacgatcgga | 7080 |
| | ggaccgaagg agctaaccgc ttttttgcac aacatggggg atcatgtaac tcgccttgat | 7140 |
| | cgttgggaac cggagctgaa tgaagccata ccaaacgacg agcgtgacac cacgatgcct | 7200 |
| | gcagcaatgg caacaacgtt gcgcaaacta ttaactggcg aactacttac tctagcttcc | 7260 |
| | cggcaacaat taatagactg gatggaggcg gataaagttg caggaccact tctgcgctcg | 7320 |
| | gcccttccgg ctggctggtt tattgctgat aaatctggag ccggtgagcg tgggtctcgc | 7380 |
| | ggtatcattg cagcactggg gccagatggt aagccctccc gtatcgtagt tatctacacg | 7440 |
| | acggggagtc aggcaactat ggatgaacga aatagacaga tcgctgagat aggtgcctca | 7500 |
| | ctgattaagc attggtaact gtcagaccaa gtttactcat atatacttta gattgattta | 7560 |
| | aaacttcatt tttaatttaa aaggatctag gtgaagatcc tttttgataa tctcatgacc | 7620 |
| | aaaatccctt aacgtgagtt ttcgttccac tgagcgtcag accccgtaga aaagatcaaa | 7680 |
| | ggatcttctt gagatccttt ttttctgcgc gtaatctgct gcttgcaaac aaaaaaacca | 7740 |
| | ccgctaccag cggtggtttg tttgccggat caagagctac caactctttt tccgaaggta | 7800 |
| | actggcttca gcagagcgca gataccaaat actgtccttc tagtgtagcc gtagttaggc | 7860 |
| | caccacttca agaactctgt agcaccgcct acatacctcg ctctgctaat cctgttacca | 7920 |
| | gtggctgctg ccagtggcga taagtcgtgt cttaccgggt tggactcaag acgatagtta | 7980 |
| | ccggataagg cgcagcggtc gggctgaacg gggggttcgt gcacacagcc cagcttggag | 8040 |
| | cgaacgacct acaccgaact gagataccta cagcgtgagc attgagaaag cgccacgctt | 8100 |
| | cccgaaggga gaaaggcgga caggtatccg gtaagcggca gggtcggaac aggagagcgc | 8160 |
| | acgagggagc ttccaggggg aaacgcctgg tatctttata gtcctgtcgg gtttcgccac | 8220 |
| | ctctgacttg agcgtcgatt tttgtgatgc tcgtcagggg ggcggagcct atggaaaaac | 8280 |
| | gccagcaacg cggccttttt acggttcctg gccttttgct ggcctttttg aagctgtccc | 8340 |
| | tgatggtcgt catctacctg cctggacagc atggcctgca acgcgggcat cccgatgccg | 8400 |
| | ccggaagcga gaagaatcat aatggggaag gccatccagc ctcgcgtcg | 8449 |
| SEQ ID NO: 24 | ctttcctgcg ttatcccctg attctgtgga taaccgtatt accgcctttg agtgagctga | 60 |
| (pDONR221 | taccgctcgc cgcagccgaa cgaccgagcg cagcgagtca gtgagcgagg aagcggaaga | 120 |
| vector) | gcgcccaata cgcaaaccgc ctctccccgc gcgttggccg attcattaat gcagctggca | 180 |
| | cgacaggttt cccgactgga aagcgggcag tgagcgcaac gcaattaata cgcgtaccgc | 240 |
| | tagccaggaa gagtttgtag aaacgcaaaa aggccatccg tcaggatggc cttctgctta | 300 |
| | gtttgatgcc tggcagttta tggcgggcgt cctgcccgcc accctccggg ccgttgcttc | 360 |
| | acaacgttca aatccgctcc cggcggattt gtcctactca ggagagcgtt caccgacaaa | 420 |
| | caacagataa aacgaaaggc ccagtcttcc gactgagcct ttcgttttat ttgatgcctg | 480 |
| | gcagttccct actctcgcgt taacgctagc atggatgttt tcccagtcac gacgttgtaa | 540 |
| | aacgacggcc agtcttaagc tcgggcccca aataatgatt ttattttgac tgatagtgac | 600 |
| | ctgttcgttg caacacattg atgagcaatg cttttttata atgccaactt tgtacaaaaa | 660 |
| | agctgaacga gaaacgtaaa atgatataaa tatcaatata ttaaattaga ttttgcataa | 720 |
| | aaaacagact acataatact gtaaaacaca acatatccag tcactatgaa tcaactactt | 780 |
| | agatggtatt agtgacctgt agtcgaccga cagccttcca aatgttcttc gggtgatgct | 840 |
| | gccaacttag tcgaccgaca gccttccaaa tgttcttctc aaacggaatc gtcgtatcca | 900 |
| | gcctactcgc tattgtcctc aatgccgtat taaatcataa aaagaaataa gaaaaagagg | 960 |
| | tgcgagcctc ttttttgtgt gacaaaataa aaacatctac ctattcatat acgctagtgt | 1020 |
| | catagtcctg aaaatcatct gcatcaagaa caatttcaca actcttatac ttttctctta | 1080 |
| | caagtcgttc ggcttcatct ggattttcag cctctatact tactaaacgt gataaagttt | 1140 |
| | ctgtaatttc tactgtatcg acctgcagac tggctgtgta taagggagcc tgacatttat | 1200 |
| | attccccaga acatcaggtt aatggcgttt ttgatgtcat tttcgcggtg gctgagatca | 1260 |
| | gccacttctt ccccgataac ggagaccggc acactggcca tatcggtggt catcatgcgc | 1320 |
| | cagctttcat ccccgatatg caccaccggg taaagttcac gggagacttt atctgacagc | 1380 |
| | agacgtgcac tggccagggg gatcaccatc cgtcgcccgg gcgtgtcaat aatatcactc | 1440 |
| | tgtacatcca caaacagacg ataacggctc tctcttttat aggtgtaaac cttaaactgc | 1500 |
| | atttcaccag cccctgttct cgtcagcaaa agagccgttc atttcaataa accgggcgac | 1560 |
| | ctcagccatc ccttcctgat tttccgcttt ccagcgttcg gcacgcagac gacgggcttc | 1620 |
| | attctgcatg gttgtgctta ccagaccgga gatattgaca tcatatatgc cttgagcaac | 1680 |
| | tgatagctgt cgctgtcaac tgtcactgta atacgctgct tcatagcata cctctttttg | 1740 |
| | acatacttcg ggtatacata tcagtatata ttcttatacc gcaaaaatca gcgcgcaaat | 1800 |
| | acgcatactg ttatctggct tttagtaagc cggatccacg cggcgtttac gccccgccct | 1860 |
| | gccactcatc gcagtactgt tgtaattcat taagcattct gccgacatgg aagccatcac | 1920 |
| | agacggcatg atgaacctga atcgccagcg gcatcagcac cttgtcgcct tgcgtataat | 1980 |
| | atttgcccat ggtgaaaacg ggggcgaaga agttgtccat attggccacg tttaaatcaa | 2040 |
| | aactggtgaa actcacccag ggattggctg agacgaaaaa catattctca ataaaccctt | 2100 |
| | tagggaaata ggccaggttt tcaccgtaac acgccacatc ttgcgaatat atgtgtagaa | 2160 |
| | actgccggaa atcgtcgtgg tattcactcc agagcgatga aaacgtttca gtttgctcat | 2220 |
| | ggaaaacggt gtaacaaggg tgaacactat cccatatcac cagctcaccg tctttcattg | 2280 |
| | ccatacggaa ttccggatga gcattcatca ggcgggcaag aatgtgaata aaggccggat | 2340 |
| | aaaacttgtg cttatttttc tttacggtct ttaaaaaggc cgtaatatcc agctgaacgg | 2400 |
| | tctggttata ggtacattga gcaactgact gaaatgcctc aaaatgttct ttacgatgcc | 2460 |
| | attgggatat atcaacggtg gtatatccag tgattttttt ctccatttta gcttccttag | 2520 |
| | ctcctgaaaa tctcgataac tcaaaaaata cgcccggtag tgatcttatt tcattatggt | 2580 |
| | gaaagttgga acctcttacg tgccgatcaa cgtctcattt tcgccaaaag ttggcccagg | 2640 |
| | gcttcccggt atcaacaggg acaccaggat ttatttattc tgcgaagtga tcttccgtca | 2700 |
| | caggtattta ttcggcgcaa agtgcgtcgg gtgatgctgc caacttagtc gactacaggt | 2760 |
| | cactaatacc atctaagtag ttgattcata gtgactggat atgttgtgtt ttacagtatt | 2820 |
| | atgtagtctg ttttttatgc aaaatctaat ttaatatatt gatatttata tcattttacg | 2880 |
| | tttctcgttc agctttcttg tacaaagttg gcattataag aaagcattgc ttatcaattt | 2940 |
| | gttgcaacga acaggtcact atcagtcaaa ataaaatcat tatttgccat ccagctgata | 3000 |
| | tcccctatag tgagtcgtat tacatggtca tagctgtttc ctggcagctc tggcccgtgt | 3060 |
| | ctcaaaatct ctgatgttac attgcacaag ataaaataat atcatcatga acaataaaac | 3120 |
| | tgtctgctta cataaacagt aatacaaggg gtgttatgag ccatattcaa cgggaaacgt | 3180 |
| | cgaggccgcg attaaattcc aacatggatg ctgatttata tgggtataaa tgggctcgcg | 3240 |
| | ataatgtcgg gcaatcaggt gcgacaatct atcgcttgta tgggaagccc gatgcgccag | 3300 |
| | agttgtttct gaaacatggc aaaggtagcg ttgccaatga tgttacagat gagatggtca | 3360 |
| | gactaaactg gctgacggaa tttatgcctc ttccgaccat caagcatttt atccgtactc | 3420 |
| | ctgatgatgc atggttactc accactgcga tccccggaaa aacagcattc caggtattag | 3480 |
| | aagaatatcc tgattcaggt gaaaatattg ttgatgcgct ggcagtgttc ctgcgccggt | 3540 |
| | tgcattcgat tcctgtttgt aattgtcctt ttaacagcga tcgcgtattt cgtctcgctc | 3600 |
| | aggcgcaatc acgaatgaat aacggtttgg ttgatgcgag tgattttgat gacgagcgta | 3660 |
| | atggctggcc tgttgaacaa gtctggaaag aaatgcataa acttttgcca ttctcaccgg | 3720 |
| | attcagtcgt cactcatggt gatttctcac ttgataacct tatttttgac gaggggaaat | 3780 |
| | taataggttg tattgatgtt ggacgagtcg gaatcgcaga ccgataccag gatcttgcca | 3840 |
| | tcctatggaa ctgcctcggt gagttttctc cttcattaca gaaacggctt tttcaaaaat | 3900 |
| | atggtattga taatcctgat atgaataaat tgcagtttca tttgatgctc gatgagtttt | 3960 |
| | tctaatcaga attggttaat tggttgtaac actggcagag cattacgctg acttgacggg | 4020 |
| | acggcgcaag ctcatgacca aaatccctta acgtgagtta cgcgtcgttc cactgagcgt | 4080 |
| | cagaccccgt agaaaagatc aaaggatctt cttgagatcc tttttttctg cgcgtaatct | 4140 |
| | gctgcttgca aacaaaaaaa ccaccgctac cagcggtggt ttgtttgccg gatcaagagc | 4200 |
| | taccaactct ttttccgaag gtaactggct tcagcagagc gcagatacca aatactgttc | 4260 |
| | ttctagtgta gccgtagtta ggccaccact tcaagaactc tgtagcaccg cctacatacc | 4320 |
| | tcgctctgct aatcctgtta ccagtggctg ctgccagtgg cgataagtcg tgtcttaccg | 4380 |
| | ggttggactc aagacgatag ttaccggata aggcgcagcg gtcgggctga acggggggtt | 4440 |
| | cgtgcacaca gcccagcttg gagcgaacga cctacaccga actgagatac ctacagcgtg | 4500 |
| | agctatgaga aagcgccacg cttcccgaag ggagaaaggc ggacaggtat ccggtaagcg | 4560 |
| | gcagggtcgg aacaggagag cgcacgaggg agcttccagg gggaaacgcc tggtatcttt | 4620 |
| | atagtcctgt cgggtttcgc cacctctgac ttgagcgtcg atttttgtga tgctcgtcag | 4680 |
| | gggggcggag cctatggaaa aacgccagca acgcggcctt tttacggttc ctggcctttt | 4740 |
| | gctggccttt tgctcacatg t | 4761 |
| SEQ ID NO: 25 | aaaaggatct tcacctagat ccttttaaat taaaaatgaa gttttaaatc aatctaaagt | 60 |
| (psPAX2 | atatatgagt aaacttggtc tgacagttac caatgcttaa tcagtgaggc acctatctca | 120 |
| plasmid) | gcgatctgtc tatttcgttc atccatagtt gcctgactcc ccgtcgtgta gataactacg | 180 |
| | atacgggagg gcttaccatc tggccccagt gctgcaatga taccgcgaga cccacgctca | 240 |
| | ccggctccag atttatcagc aataaaccag ccagccggaa gggccgagcg cagaagtggt | 300 |
| | cctgcaactt tatccgcctc catccagtct attaattgtt gccgggaagc tagagtaagt | 360 |
| | agttcgccag ttaatagttt gcgcaacgtt gttgccattg ctacaggcat cgtggtgtca | 420 |
| | cgctcgtcgt ttggtatggc ttcattcagc tccggttccc aacgatcaag gcgagttaca | 480 |
| | tgatccccca tgttgtgcaa aaaagcggtt agctccttcg gtcctccgat cgttgtcaga | 540 |
| | agtaagttgg ccgcagtgtt atcactcatg gttatggcag cactgcataa ttctcttact | 600 |
| | gtcatgccat ccgtaagatg cttttctgtg actggtgagt actcaaccaa gtcattctga | 660 |
| | gaatagtgta tgcggcgacc gagttgctct tgcccggcgt caatacggga taataccgcg | 720 |
| | ccacatagca gaactttaaa agtgctcatc attggaaaac gttcttcggg gcgaaaactc | 780 |
| | tcaaggatct taccgctgtt gagatccagt tcgatgtaac ccactcgtgc acccaactga | 840 |
| | tcttcagcat cttttacttt caccagcgtt tctgggtgag caaaaacagg aaggcaaaat | 900 |
| | gccgcaaaaa agggaataag ggcgacacgg aaatgttgaa tactcatact cttccttttt | 960 |
| | caatattatt gaagcattta tcagggttat tgtctcatga gcggatacat atttgaatgt | 1020 |
| | atttagaaaa ataaacaaat aggggttccg cgcacatttc cccgaaaagt gccacctggt | 1080 |
| | cgacattgat tattgactag ttattaatag taatcaatta cggggtcatt agttcatagc | 1140 |
| | ccatatatgg agttccgcgt tacataactt acggtaaatg gcccgcctgg ctgaccgccc | 1200 |
| | aacgaccccc gcccattgac gtcaataatg acgtatgttc ccatagtaac gccaataggg | 1260 |
| | actttccatt gacgtcaatg ggtggactat ttacggtaaa ctgcccactt ggcagtacat | 1320 |
| | caagtgtatc atatgccaag tacgccccct attgacgtca atgacggtaa atggcccgcc | 1380 |
| | tggcattatg cccagtacat gaccttatgg gactttccta cttggcagta catctacgta | 1440 |
| | ttagtcatcg ctattaccat gggtcgaggt gagccccacg ttctgcttca ctctccccat | 1500 |
| | ctcccccccc tccccacccc caattttgta tttatttatt ttttaattat tttgtgcagc | 1560 |
| | gatgggggcg gggggggggg gggcgcgcgc caggcggggc ggggcggggc gaggggcggg | 1620 |
| | gcggggcgag gcggagaggt gcggcggcag ccaatcagag cggcgcgctc cgaaagtttc | 1680 |
| | cttttatggc gaggcggcgg cggcggcggc cctataaaaa gcgaagcgcg cggcgggcgg | 1740 |
| | gagtcgctgc gttgccttcg ccccgtgccc cgctccgcgc cgcctcgcgc cgcccgcccc | 1800 |
| | ggctctgact gaccgcgtta ctcccacagg tgagcgggcg ggacggccct tctcctccgg | 1860 |
| | gctgtaatta gcgcttggtt taatgacggc tcgtttcttt tctgtggctg cgtgaaagcc | 1920 |
| | ttaaagggct ccgggagggc cctttgtgcg ggggggagcg gctcgggggg tgcgtgcgtg | 1980 |
| | tgtgtgtgcg tggggagcgc cgcgtgcggc ccgcgctgcc cggcggctgt gagcgctgcg | 2040 |
| | ggcgcggcgc ggggctttgt gcgctccgcg tgtgcgcgag gggagcgcgg ccgggggcgg | 2100 |
| | tgccccgcgg tgcggggggg ctgcgagggg aacaaaggct gcgtgcgggg tgtgtgcgtg | 2160 |
| | ggggggtgag cagggggtgt gggcgcggcg gtcgggctgt aacccccccc tgcacccccc | 2220 |
| | tccccgagtt gctgagcacg gcccggcttc gggtgcgggg ctccgtgcgg ggcgtggcgc | 2280 |
| | ggggctcgcc gtgccgggcg gggggtggcg gcaggtgggg gtgccgggcg gggcggggcc | 2340 |
| | gcctcgggcc ggggagggct cgggggaggg gcgcggcggc cccggagcgc cggcggctgt | 2400 |
| | cgaggcgcgg cgagccgcag ccattgcctt ttatggtaat cgtgcgagag ggcgcaggga | 2460 |
| | cttcctttgt cccaaatctg gcggagccga aatctgggag gcgccgccgc accccctcta | 2520 |
| | gcgggcgcgg gcgaagcggt gcggcgccgg caggaaggaa atgggcgggg agggccttcg | 2580 |
| | tgcgtcgccg cgccgccgtc cccttctcca tctccagcct cggggctgcc gcagggggac | 2640 |
| | ggctgccttc gggggggacg gggcagggcg gggttcggct tctggcgtgt gaccggcggc | 2700 |
| | tctagagcct ctgctaacca tgttcatgcc ttcttctttt tcctacagct cctgggcaac | 2760 |
| | gtgctggtta ttgtgctgtc tcatcatttt ggcaaagaat tcgggccggc cgcgttgacg | 2820 |
| | cgcacggcaa gaggcgaggg gcggcgactg gtgagagatg ggtgcgagag cgtcagtatt | 2880 |
| | aagcggggga gaattagatc gatgggaaaa aattcggtta aggccagggg gaaagaaaaa | 2940 |
| | atataaatta aaacatatag tatgggcaag cagggagcta gaacgattcg cagttaatcc | 3000 |
| | tggcctgtta gaaacatcag aaggctgtag acaaatactg ggacagctac aaccatccct | 3060 |
| | tcagacagga tcagaagaac ttagatcatt atataataca gtagcaaccc tctattgtgt | 3120 |
| | gcatcaaagg atagagataa aagacaccaa ggaagcttta gacaagatag aggaagagca | 3180 |
| | aaacaaaagt aagaaaaaag cacagcaagc agcagctgac acaggacaca gcaatcaggt | 3240 |
| | cagccaaaat taccctatag tgcagaacat ccaggggcaa atggtacatc aggccatatc | 3300 |
| | acctagaact ttaaatgcat gggtaaaagt agtagaagag aaggctttca gcccagaagt | 3360 |
| | gatacccatg ttttcagcat tatcagaagg agccacccca caagatttaa acaccatgct | 3420 |
| | aaacacagtg gggggacatc aagcagccat gcaaatgtta aaagagacca tcaatgagga | 3480 |
| | agctgcagaa tgggatagag tgcatccagt gcatgcaggg cctattgcac caggccagat | 3540 |
| | gagagaacca aggggaagtg acatagcagg aactactagt acccttcagg aacaaatagg | 3600 |
| | atggatgaca cataatccac ctatcccagt aggagaaatc tataaaagat ggataatcct | 3660 |
| | gggattaaat aaaatagtaa gaatgtatag ccctaccagc attctggaca taagacaagg | 3720 |
| | accaaaggaa ccctttagag actatgtaga ccgattctat aaaactctaa gagccgagca | 3780 |
| | agcttcacaa gaggtaaaaa attggatgac agaaaccttg ttggtccaaa atgcgaaccc | 3840 |
| | agattgtaag actattttaa aagcattggg accaggagcg acactagaag aaatgatgac | 3900 |
| | agcatgtcag ggagtggggg gacccggcca taaagcaaga gttttggctg aagcaatgag | 3960 |
| | ccaagtaaca aatccagcta ccataatgat acagaaaggc aattttagga accaaagaaa | 4020 |
| | gactgttaag tgtttcaatt gtggcaaaga agggcacata gccaaaaatt gcagggcccc | 4080 |
| | taggaaaaag ggctgttgga aatgtggaaa ggaaggacac caaatgaaag attgtactga | 4140 |
| | gagacaggct aattttttag ggaagatctg gccttcccac aagggaaggc cagggaattt | 4200 |
| | tcttcagagc agaccagagc caacagcccc accagaagag agcttcaggt ttggggaaga | 4260 |
| | gacaacaact ccctctcaga agcaggagcc gatagacaag gaactgtatc ctttagcttc | 4320 |
| | cctcagatca ctctttggca gcgacccctc gtcacaataa agataggggg gcaattaaag | 4380 |
| | gaagctctat tagatacagg agcagatgat acagtattag aagaaatgaa tttgccagga | 4440 |
| | agatggaaac caaaaatgat agggggaatt ggaggtttta tcaaagtagg acagtatgat | 4500 |
| | cagatactca tagaaatctg cggacataaa gctataggta cagtattagt aggacctaca | 4560 |
| | cctgtcaaca taattggaag aaatctgttg actcagattg gctgcacttt aaattttccc | 4620 |
| | attagtccta ttgagactgt accagtaaaa ttaaagccag gaatggatgg cccaaaagtt | 4680 |
| | aaacaatggc cattgacaga agaaaaaata aaagcattag tagaaatttg tacagaaatg | 4740 |
| | gaaaaggaag gaaaaatttc aaaaattggg cctgaaaatc catacaatac tccagtattt | 4800 |
| | gccataaaga aaaaagacag tactaaatgg agaaaattag tagatttcag agaacttaat | 4860 |
| | aagagaactc aagatttctg ggaagttcaa ttaggaatac cacatcctgc agggttaaaa | 4920 |
| | cagaaaaaat cagtaacagt actggatgtg ggcgatgcat atttttcagt tcccttagat | 4980 |
| | aaagacttca ggaagtatac tgcatttacc atacctagta taaacaatga gacaccaggg | 5040 |
| | attagatatc agtacaatgt gcttccacag ggatggaaag gatcaccagc aatattccag | 5100 |
| | tgtagcatga caaaaatctt agagcctttt agaaaacaaa atccagacat agtcatctat | 5160 |
| | caatacatgg atgatttgta tgtaggatct gacttagaaa tagggcagca tagaacaaaa | 5220 |
| | atagaggaac tgagacaaca tctgttgagg tggggattta ccacaccaga caaaaaacat | 5280 |
| | cagaaagaac ctccattcct ttggatgggt tatgaactcc atcctgataa atggacagta | 5340 |
| | cagcctatag tgctgccaga aaaggacagc tggactgtca atgacataca gaaattagtg | 5400 |
| | ggaaaattga attgggcaag tcagatttat gcagggatta aagtaaggca attatgtaaa | 5460 |
| | cttcttaggg gaaccaaagc actaacagaa gtagtaccac taacagaaga agcagagcta | 5520 |
| | gaactggcag aaaacaggga gattctaaaa gaaccggtac atggagtgta ttatgaccca | 5580 |
| | tcaaaagact taatagcaga aatacagaag caggggcaag gccaatggac atatcaaatt | 5640 |
| | tatcaagagc catttaaaaa tctgaaaaca ggaaaatatg caagaatgaa gggtgcccac | 5700 |
| | actaatgatg tgaaacaatt aacagaggca gtacaaaaaa tagccacaga aagcatagta | 5760 |
| | atatggggaa agactcctaa atttaaatta cccatacaaa aggaaacatg ggaagcatgg | 5820 |
| | tggacagagt attggcaagc cacctggatt cctgagtggg agtttgtcaa tacccctccc | 5880 |
| | ttagtgaagt tatggtacca gttagagaaa gaacccataa taggagcaga aactttctat | 5940 |
| | gtagatgggg cagccaatag ggaaactaaa ttaggaaaag caggatatgt aactgacaga | 6000 |
| | ggaagacaaa aagttgtccc cctaacggac acaacaaatc agaagactga gttacaagca | 6060 |
| | attcatctag ctttgcagga ttcgggatta gaagtaaaca tagtgacaga ctcacaatat | 6120 |
| | gcattgggaa tcattcaagc acaaccagat aagagtgaat cagagttagt cagtcaaata | 6180 |
| | atagagcagt taataaaaaa ggaaaaagtc tacctggcat gggtaccagc acacaaagga | 6240 |
| | attggaggaa atgaacaagt agatgggttg gtcagtgctg gaatcaggaa agtactattt | 6300 |
| | ttagatggaa tagataaggc ccaagaagaa catgagaaat atcacagtaa ttggagagca | 6360 |
| | atggctagtg attttaacct accacctgta gtagcaaaag aaatagtagc cagctgtgat | 6420 |
| | aaatgtcagc taaaagggga agccatgcat ggacaagtag actgtagccc aggaatatgg | 6480 |
| | cagctagatt gtacacattt agaaggaaaa gttatcttgg tagcagttca tgtagccagt | 6540 |
| | ggatatatag aagcagaagt aattccagca gagacagggc aagaaacagc atacttcctc | 6600 |
| | ttaaaattag caggaagatg gccagtaaaa acagtacata cagacaatgg cagcaatttc | 6660 |
| | accagtacta cagttaaggc cgcctgttgg tgggcgggga tcaagcagga atttggcatt | 6720 |
| | ccctacaatc cccaaagtca aggagtaata gaatctatga ataaagaatt aaagaaaatt | 6780 |
| | ataggacagg taagagatca ggctgaacat cttaagacag cagtacaaat ggcagtattc | 6840 |
| | atccacaatt ttaaaagaaa aggggggatt ggggggtaca gtgcagggga aagaatagta | 6900 |
| | gacataatag caacagacat acaaactaaa gaattacaaa aacaaattac aaaaattcaa | 6960 |
| | aattttcggg tttattacag ggacagcaga gatccagttt ggaaaggacc agcaaagctc | 7020 |
| | ctctggaaag gtgaaggggc agtagtaata caagataata gtgacataaa agtagtgcca | 7080 |
| | agaagaaaag caaagatcat cagggattat ggaaaacaga tggcaggtga tgattgtgtg | 7140 |
| | gcaagtagac aggatgagga ttaacacatg gaattctgca acaactgctg tttatccatt | 7200 |
| | tcagaattgg gtgtcgacat agcagaatag gcgttactcg acagaggaga gcaagaaatg | 7260 |
| | gagccagtag atcctagact agagccctgg aagcatccag gaagtcagcc taaaactgct | 7320 |
| | tgtaccaatt gctattgtaa aaagtgttgc tttcattgcc aagtttgttt catgacaaaa | 7380 |
| | gccttaggca tctcctatgg caggaagaag cggagacagc gacgaagagc tcatcagaac | 7440 |
| | agtcagactc atcaagcttc tctatcaaag cagtaagtag tacatgtaat gcaacctata | 7500 |
| | atagtagcaa tagtagcatt agtagtagca ataataatag caatagttgt gtggtccata | 7560 |
| | gtaatcatag aatataggaa aatggccgct gatcttcaga cctggaggag gagatatgag | 7620 |
| | ggacaattgg agaagtgaat tatataaata taaagtagta aaaattgaac cattaggagt | 7680 |
| | agcacccacc aaggcaaaga gaagagtggt gcagagagaa aaaagagcag tgggaatagg | 7740 |
| | agctttgttc cttgggttct tgggagcagc aggaagcact atgggcgcag cctcaatgac | 7800 |
| | gctgacggta caggccagac aattattgtc tggtatagtg cagcagcaga acaatttgct | 7860 |
| | gagggctatt gaggcgcaac agcatctgtt gcaactcaca gtctggggca tcaagcagct | 7920 |
| | ccaagcaaga atcctagctg tggaaagata cctaaaggat caacagctcc tagggatttg | 7980 |
| | gggttgctct ggaaaactca tttgcaccac tgctgtgcct tggaatgcta gttggagtaa | 8040 |
| | taaatctctg gaacagatct ggaatcacac gacctggatg gagtgggaca gagaaattaa | 8100 |
| | caattacaca agcttaatac actccttaat tgaagaatcg caaaaccagc aagaaaagaa | 8160 |
| | tgaacaagaa ttattggaat tagataaatg ggcaagtttg tggaattggt ttaacataac | 8220 |
| | aaattggctg tggtatataa aattattcat aatgatagta ggaggcttgg taggtttaag | 8280 |
| | aatagttttt gctgtacttt ctatagtgaa tagagttagg cagggatatt caccattatc | 8340 |
| | gtttcagacc cacctcccaa tcccgagggg acccgacagg cccgaaggaa tagaagaaga | 8400 |
| | aggtggagag agagacagag acagatccat tcgattagtg aacggatcct tggcacttat | 8460 |
| | ctgggacgat ctgcggagcc tgtgcctctt cagctaccac cgcttgagag acttactctt | 8520 |
| | gattgtaacg aggattgtgg aacttctggg acgcaggggg tgggaagccc tcaaatattg | 8580 |
| | gtggaatctc ctacaatatt ggagtcagga gctaaagaat agtgctgtta gcttgctcaa | 8640 |
| | tgccacagcc atagcagtag ctgaggggac agatagggtt atagaagtag tacaaggagc | 8700 |
| | ttgtagagct attcgccaca tacctagaag aataagacag ggcttggaaa ggattttgct | 8760 |
| | ataagctcga aacaaccggt acctctagaa ctatagctag cagatctttt tccctctgcc | 8820 |
| | aaaaattatg gggacatcat gaagcccctt gagcatctga cttctggcta ataaaggaaa | 8880 |
| | tttattttca ttgcaatagt gtgttggaat tttttgtgtc tctcactcgg aaggacatat | 8940 |
| | gggagggcaa atcatttaaa acatcagaat gagtatttgg tttagagttt ggcaacatat | 9000 |
| | gccatatgct ggctgccatg aacaaaggtg gctataaaga ggtcatcagt atatgaaaca | 9060 |
| | gccccctgct gtccattcct tattccatag aaaagccttg acttgaggtt agattttttt | 9120 |
| | tatattttgt tttgtgttat ttttttcttt aacatcccta aaattttcct tacatgtttt | 9180 |
| | actagccaga tttttcctcc tctcctgact actcccagtc atagctgtcc ctcttctctt | 9240 |
| | atgaagatcc ctcgacctgc agcccaagct tggcgtaatc atggtcatag ctgtttcctg | 9300 |
| | tgtgaaattg ttatccgctc acaattccac acaacatacg agccggaagc ataaagtgta | 9360 |
| | aagcctgggg tgcctaatga gtgagctaac tcacattaat tgcgttgcgc tcactgcccg | 9420 |
| | ctttccagtc gggaaacctg tcgtgccagc ggatccgcat ctcaattagt cagcaaccat | 9480 |
| | agtcccgccc ctaactccgc ccatcccgcc cctaactccg cccagttccg cccattctcc | 9540 |
| | gccccatggc tgactaattt tttttattta tgcagaggcc gaggccgcct cggcctctga | 9600 |
| | gctattccag aagtagtgag gaggcttttt tggaggccta ggcttttgca aaaagctaac | 9660 |
| | ttgtttattg cagcttataa tggttacaaa taaagcaata gcatcacaaa tttcacaaat | 9720 |
| | aaagcatttt tttcactgca ttctagttgt ggtttgtcca aactcatcaa tgtatcttat | 9780 |
| | catgtctgga tccgctgcat taatgaatcg gccaacgcgc ggggagaggc ggtttgcgta | 9840 |
| | ttgggcgctc ttccgcttcc tcgctcactg actcgctgcg ctcggtcgtt cggctgcggc | 9900 |
| | gagcggtatc agctcactca aaggcggtaa tacggttatc cacagaatca ggggataacg | 9960 |
| | caggaaagaa catgtgagca aaaggccagc aaaaggccag gaaccgtaaa aaggccgcgt | 10020 |
| | tgctggcgtt tttccatagg ctccgccccc ctgacgagca tcacaaaaat cgacgctcaa | 10080 |
| | gtcagaggtg gcgaaacccg acaggactat aaagatacca ggcgtttccc cctggaagct | 10140 |
| | ccctcgtgcg ctctcctgtt ccgaccctgc cgcttaccgg atacctgtcc gcctttctcc | 10200 |
| | cttcgggaag cgtggcgctt tctcaatgct cacgctgtag gtatctcagt tcggtgtagg | 10260 |
| | tcgttcgctc caagctgggc tgtgtgcacg aaccccccgt tcagcccgac cgctgcgcct | 10320 |
| | tatccggtaa ctatcgtctt gagtccaacc cggtaagaca cgacttatcg ccactggcag | 10380 |
| | cagccactgg taacaggatt agcagagcga ggtatgtagg cggtgctaca gagttcttga | 10440 |
| | agtggtggcc taactacggc tacactagaa ggacagtatt tggtatctgc gctctgctga | 10500 |
| | agccagttac cttcggaaaa agagttggta gctcttgatc cggcaaacaa accaccgctg | 10560 |
| | gtagcggtgg tttttttgtt tgcaagcagc agattacgcg cagaaaaaaa ggatctcaag | 10620 |
| | aagatccttt gatcttttct acggggtctg acgctcagtg gaacgaaaac tcacgttaag | 10680 |
| | ggattttggt catgagatta tca | 10703 |
| SEQ ID NO: 26 | gtcgacggat cgggagatca attccggcac ctgtcctacg agttgcatga taaagaagac | 60 |
| (pCIGO-VSV.G | agtcataagt gcggcgacga tagtcatgcc ccgcgcccac cggaaggagc tgactgggtt | 120 |
| plasmid) | gaaggctctc aagggcatcg gtcgatgcag gaaaaggaca agcagcgaaa attcacgccc | 180 |
| | ccttgggagg tggcggcata tgcaaaggat agcactccca ctctactact gggtatcata | 240 |
| | tgctgactgt atatgcatga ggatagcata tgctacccgg atacagatta ggatagcata | 300 |
| | tactacccag atatagatta ggatagcata tgctacccag atatagatta ggatagccta | 360 |
| | tgctacccag atataaatta ggatagcata tactacccag atatagatta ggatagcata | 420 |
| | tgctacccag atatagatta ggatagccta tgctacccag atatagatta ggatagcata | 480 |
| | tgctacccag atatagatta ggatagcata tgctatccag atatttgggt agtatatgct | 540 |
| | acccagatat aaattaggat agcatatact accctaatct ctattaggat agcatatgct | 600 |
| | acccggatac agattaggat agcatatact acccagatat agattaggat agcatatgct | 660 |
| | acccagatat agattaggat agcctatgct acccagatat aaattaggat agcatatact | 720 |
| | acccagatat agattaggat agcatatgct acccagatat agattaggat agcctatgct | 780 |
| | acccagatat agattaggat agcatatgct atccagatat ttgggtagta tatgctaccc | 840 |
| | atggcaacat tagcccaccg tgctctcagc gacctcgtga atatgaggac caacaaccct | 900 |
| | gtgcttggcg ctcaggcgca agtgtgtgta atttgtcctc cagatcgcag caatcgcgcc | 960 |
| | cctatcttgg cccgcccacc tacttatgca ggtattcccc ggggtgccat tagtggtttt | 1020 |
| | gtgggcaagt ggtttgaccg cagtggttag cggggttaca atcagccaag ttattacacc | 1080 |
| | cttattttac agtccaaaac cgcagggcgg cgtgtggggg ctgacgcgtg cccccactcc | 1140 |
| | acaatttcaa aaaaaagagt ggccacttgt ctttgtttat gggccccatt ggcgtggagc | 1200 |
| | cccgtttaat tttcgggggt gttagagaca accagtggag tccgctgctg tcggcgtcca | 1260 |
| | ctctctttcc ccttgttaca aatagagtgt aacaacatgg ttcacctgtc ttggtccctg | 1320 |
| | cctgggacac atcttaataa ccccagtatc atattgcact aggattatgt gttgcccata | 1380 |
| | gccataaatt cgtgtgagat ggacatccag tctttacggc ttgtccccac cccatggatt | 1440 |
| | tctattgtta aagatattca gaatgtttca ttcctacact agtatttatt gcccaagggg | 1500 |
| | tttgtgaggg ttatattggt gtcatagcac aatgccacca ctgaaccccc cgtccaaatt | 1560 |
| | ttattctggg ggcgtcacct gaaaccttgt tttcgagcac ctcacataca ccttactgtt | 1620 |
| | cacaactcag cagttattct attagctaaa cgaaggagaa tgaagaagca ggcgaagatt | 1680 |
| | caggagagtt cactgcccgc tccttgatct tcagccactg cccttgtgac taaaatggtt | 1740 |
| | cactaccctc gtggaatcct gaccccatgt aaataaaacc gtgacagctc atggggtggg | 1800 |
| | agatatcgct gttccttagg acccttttac taaccctaat tcgatagcat atgcttcccg | 1860 |
| | ttgggtaaca tatgctattg aattagggtt agtctggata gtatatacta ctacccggga | 1920 |
| | agcatatgct acccgtttag ggttaacaag ggggccttat aaacactatt gctaatgccc | 1980 |
| | tcttgagggt ccgcttatcg gtagctacac aggcccctct gattgacgtt ggtgtagcct | 2040 |
| | cccgtagtct tcctgggccc ctgggaggta catgtccccc agcattggtg taagagcttc | 2100 |
| | agccaagagt tacacataaa ggcaatgttg tgttgcagtc cacagactgc aaagtctgct | 2160 |
| | ccaggatgaa agccactcaa gggatcttca atattggcca ttagccatat tattcattgg | 2220 |
| | ttatatagca taaatcaata ttggctattg gccattgcat acgttgtatc tatatcataa | 2280 |
| | tatgtacatt tatattggct catgtccaat atgaccgcca tgttggcatt gattattgac | 2340 |
| | tagttattaa tagtaatcaa ttacggggtc attagttcat agcccatata tggagttccg | 2400 |
| | cgttacataa cttacggtaa atggcccgcc tggctgaccg cccaacgacc cccgcccatt | 2460 |
| | gacgtcaata atgacgtatg ttcccatagt aacgccaata gggactttcc attgacgtca | 2520 |
| | atgggtggag tatttacggt aaactgccca cttggcagta catcaagtgt atcatatgcc | 2580 |
| | aagtccgccc cctattgacg tcaatgacgg taaatggccc gcctggcatt atgcccagta | 2640 |
| | catgacctta cgggactttc ctacttggca gtacatctac gtattagtca tcgctattac | 2700 |
| | catggtgatg cggttttggc agtacaccaa tgggcgtgga tagcggtttg actcacgggg | 2760 |
| | atttccaagt ctccacccca ttgacgtcaa tgggagtttg ttttggcacc aaaatcaacg | 2820 |
| | ggactttcca aaatgtcgta ataaccccgc cccgttgacg caaatgggcg gtaggcgtgt | 2880 |
| | acggtgggag gtctatataa gcagagctcg tttagtgaac cgtcagatca ctagaagctt | 2940 |
| | tattgcggta gtttatcaca gttaaattgc taacgcagtc agtgcttctg acacaacagt | 3000 |
| | ctcgaactta agctgcagaa gttggtcgtg aggcactggg caggtaagta tcaaggttac | 3060 |
| | aagacaggtt taaggagacc aatagaaact gggcttgtcg agacagagaa gactcttgcg | 3120 |
| | tttctgatag gcacctattg gtcttactga catccacttt gcctttctct ccacaggtgt | 3180 |
| | ccactcccag ttcaattaca gctcttaagg ctagagtact taatacgact cactataggc | 3240 |
| | tagcggtacc gagctcggat ccactagtaa cggccgccag tgtgctggaa ttcaacagag | 3300 |
| | atcgatctgt ttccttgaca ctatgaagtg ccttttgtac ttagcctttt tattcattgg | 3360 |
| | ggtgaattgc aagttcacca tagtttttcc acacaaccaa aaaggaaact ggaaaaatgt | 3420 |
| | tccttctaat taccattatt gcccgtcaag ctcagattta aattggcata atgacttaat | 3480 |
| | aggcacagcc atacaagtca aaatgcccaa gagtcacaag gctattcaag cagacggttg | 3540 |
| | gatgtgtcat gcttccaaat gggtcactac ttgtgatttc cgctggtatg gaccgaagta | 3600 |
| | tataacacag tccatccgat ccttcactcc atctgtagaa caatgcaagg aaagcattga | 3660 |
| | acaaacgaaa caaggaactt ggctgaatcc aggcttccct cctcaaagtt gtggatatgc | 3720 |
| | aactgtgacg gatgccgaag cagtgattgt ccaggtgact cctcaccatg tgctggttga | 3780 |
| | tgaatacaca ggagaatggg ttgattcaca gttcatcaac ggaaaatgca gcaattacat | 3840 |
| | atgccccact gtccataact ctacaacctg gcattctgac tataaggtca aagggctatg | 3900 |
| | tgattctaac ctcatttcca tggacatcac cttcttctca gaggacggag agctatcatc | 3960 |
| | cctgggaaag gagggcacag ggttcagaag taactacttt gcttatgaaa ctggaggcaa | 4020 |
| | ggcctgcaaa atgcaatact gcaagcattg gggagtcaga ctcccatcag gtgtctggtt | 4080 |
| | cgagatggct gataaggatc tctttgctgc agccagattc cctgaatgcc cagaagggtc | 4140 |
| | aagtatctct gctccatctc agacctcagt ggatgtaagt ctaattcagg acgttgagag | 4200 |
| | gatcttggat tattccctct gccaagaaac ctggagcaaa atcagagcgg gtcttccaat | 4260 |
| | ctctccagtg gatctcagct atcttgctcc taaaaaccca ggaaccggtc ctgctttcac | 4320 |
| | cataatcaat ggtaccctaa aatactttga gaccagatac atcagagtcg atattgctgc | 4380 |
| | tccaatcctc tcaagaatgg tcggaatgat cagtggaact accacagaaa gggaactgtg | 4440 |
| | ggatgactgg gcaccatatg aagacgtgga aattggaccc aatggagttc tgaggaccag | 4500 |
| | ttcaggatat aagtttcctt tatacatgat tggacatggt atgttggact ccgatcttca | 4560 |
| | tcttagctca aaggctcagg tgttcgaaca tcctcacatt caagacgctg cttcgcaact | 4620 |
| | tcctgatgat gagagtttat tttttggtga tactgggcta tccaaaaatc caatcgagct | 4680 |
| | tgtagaaggt tggttcagta gttggaaaag ctctattgcc tcttttttct ttatcatagg | 4740 |
| | gttaatcatt ggactattct tggttctccg agttggtatc catctttgca ttaaattaaa | 4800 |
| | gcacaccaag aaaagacaga tttatacaga catagagatg aaccgacttg gaaagtaact | 4860 |
| | caaatcctgc acaacagatt cttcatgttt ggaccaaatc aacttgtgat accatgctca | 4920 |
| | aagaggcctc aattatattt gagtttttaa tttttatgga attctgcaga tatccatcac | 4980 |
| | actggcggcc gctcgagcat gcatctagag ggccctattc tatagtgtca cctaaatgct | 5040 |
| | agagctcgct gatcagcctc gactgtgcct tctagttgcc agccatctgt tgtttgcccc | 5100 |
| | tcccccgtgc cttccttgac cctggaaggt gccactccca ctgtcctttc ctaataaaat | 5160 |
| | gaggaaattg catcgcattg tctgagtagg tgtcattcta ttctgggggg tggggtgggg | 5220 |
| | caggacagca agggggagga ttgggaagac aatagcaggc atgctgggga tgcggtgggc | 5280 |
| | tctatggctt ctgaggcgga aagaaccagc tgcattaatg aatcggccaa cgcgcgggga | 5340 |
| | gaggcggttt gcgtattggg cgctcttccg cttcctcgct cactgactcg ctgcgctcgg | 5400 |
| | tcgttcggct gcggcgagcg gtatcagctc actcaaaggc ggtaatacgg ttatccacag | 5460 |
| | aatcagggga taacgcagga aagaacatgt gagcaaaagg ccagcaaaag gccaggaacc | 5520 |
| | gtaaaaaggc cgcgttgctg gcgtttttcc ataggctccg cccccctgac gagcatcaca | 5580 |
| | aaaatcgacg ctcaagtcag aggtggcgaa acccgacagg actataaaga taccaggcgt | 5640 |
| | ttccccctgg aagctccctc gtgcgctctc ctgttccgac cctgccgctt accggatacc | 5700 |
| | tgtccgcctt tctcccttcg ggaagcgtgg cgctttctca atgctcacgc tgtaggtatc | 5760 |
| | tcagttcggt gtaggtcgtt cgctccaagc tgggctgtgt gcacgaaccc cccgttcagc | 5820 |
| | ccgaccgctg cgccttatcc ggtaactatc gtcttgagtc caacccggta agacacgact | 5880 |
| | tatcgccact ggcagcagcc actggtaaca ggattagcag agcgaggtat gtaggcggtg | 5940 |
| | ctacagagtt cttgaagtgg tggcctaact acggctacac tagaaggaca gtatttggta | 6000 |
| | tctgcgctct gctgaagcca gttaccttcg gaaaaagagt tggtagctct tgatccggca | 6060 |
| | aacaaaccac cgctggtagc ggtggttttt ttgtttgcaa gcagcagatt acgcgcagaa | 6120 |
| | aaaaaggatc tcaagaagat cctttgatct tttctacggg gtctgacgct cagtggaacg | 6180 |
| | aaaactcacg ttaagggatt ttggtcatga gattatcaaa aaggatcttc acctagatcc | 6240 |
| | ttttaaatta aaaatgaagt tttaaatcaa tctaaagtat atatgagtaa acttggtctg | 6300 |
| | acagttacca atgcttaatc agtgaggcac ctatctcagc gatctgtcta tttcgttcat | 6360 |
| | ccatagttgc ctgactcccc gtcgtgtaga taactacgat acgggagggc ttaccatctg | 6420 |
| | gccccagtgc tgcaatgata ccgcgagacc cacgctcacc ggctccagat ttatcagcaa | 6480 |
| | taaaccagcc agccggaagg gccgagcgca gaagtggtcc tgcaacttta tccgcctcca | 6540 |
| | tccagtctat taattgttgc cgggaagcta gagtaagtag ttcgccagtt aatagtttgc | 6600 |
| | gcaacgttgt tgccattgct acaggcatcg tggtgtcacg ctcgtcgttt ggtatggctt | 6660 |
| | cattcagctc cggttcccaa cgatcaaggc gagttacatg atcccccatg ttgtgcaaaa | 6720 |
| | aagcggttag ctccttcggt cctccgatcg ttgtcagaag taagttggcc gcagtgttat | 6780 |
| | cactcatggt tatggcagca ctgcataatt ctcttactgt catgccatcc gtaagatgct | 6840 |
| | tttctgtgac tggtgagtac tcaaccaagt cattctgaga atagtgtatg cggcgaccga | 6900 |
| | gttgctcttg cccggcgtca atacgggata ataccgcgcc acatagcaga actttaaaag | 6960 |
| | tgctcatcat tggaaaacgt tcttcggggc gaaaactctc aaggatctta ccgctgttga | 7020 |
| | gatccagttc gatgtaaccc actcgtgcac ccaactgatc ttcagcatct tttactttca | 7080 |
| | ccagcgtttc tgggtgagca aaaacaggaa ggcaaaatgc cgcaaaaaag ggaataaggg | 7140 |
| | cgacacggaa atgttgaata ctcatactct tcctttttca atattattga agcatttatc | 7200 |
| | agggttattg tctcatgagc ggatacatat ttgaatgtat ttagaaaaat aaacaaatag | 7260 |
| | gggttccgcg cacatttccc cgaaaagtgc cacctgac | 7298 |

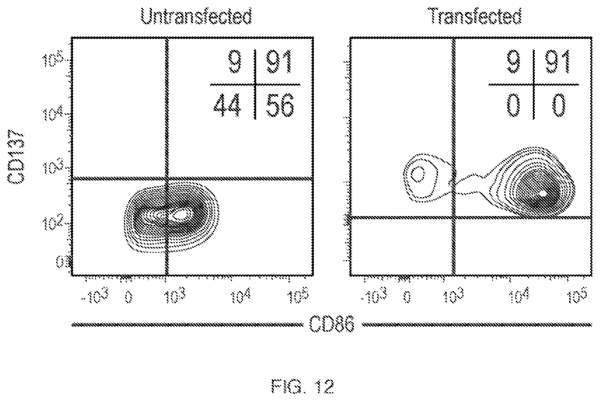

Expression of CD86 and 4-1BBL on engineered MOLM-14 aAPCs (also referred to herein as aMOLM14 aAPCs) was confirmed using flow cytometry (Canto II flow cytometer, Becton, Dickinson, and Co., Franklin Lakes, NJ, USA), with results shown in FIG. 12. aMOLM-14 aAPCs were γ-irradiated at 100 Gy and frozen.

### Example 4—Expansion of Tumor Infiltrating Lymphocytes Using MOLM-14 Artificial Antigen Presenting Cells

Engineered MOLM-14 cells were gamma-irradiated at 100 Gy before co-culturing with TILs. REPs were initiated by culturing TILs with irradiated, engineered MOLM-14 cells at 1:100 ratios in CM2 media containing OKT-3 (30 ng/mL) and IL-2 (3000 IU/mL) for 14 days. At REP harvest, the TIL expansion rates, phenotype for activation and differentiation stage markers, metabolism rate, cytotoxicity and re-rapid expansion protocol (re-REP) assay were measured.

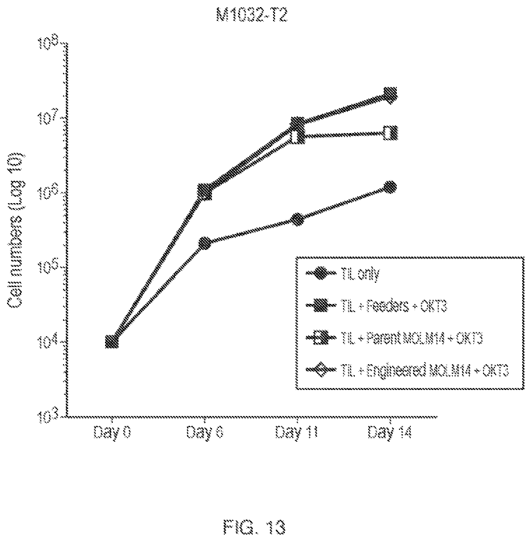

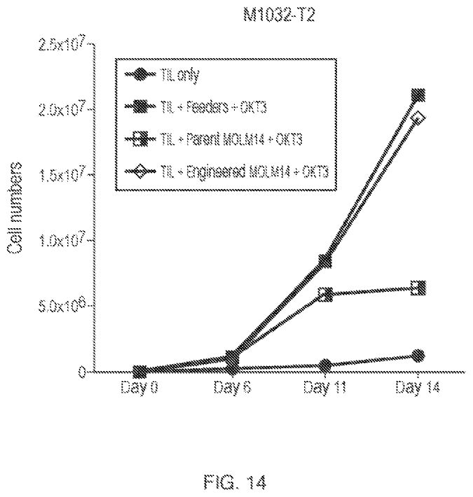

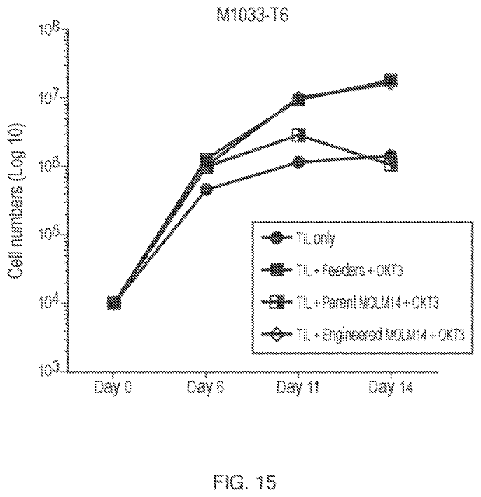

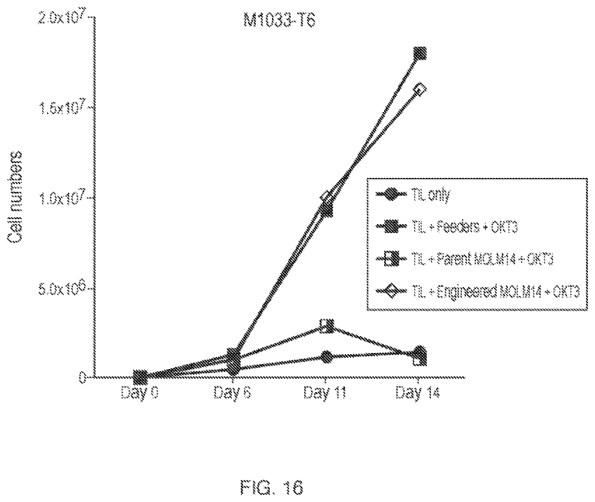

The results are shown in FIG. 13, FIG. 14, FIG. 15, and FIG. 16, where two expansions for two sets of patient TILs are compared. The results with the CD86/4-1BBL modified MOLM-14 cells (labeled “TIL+Engineered MOLM14+OKT3”) are comparable to the PBMC feeders (labeled “TIL+Feeders+OKT3”).

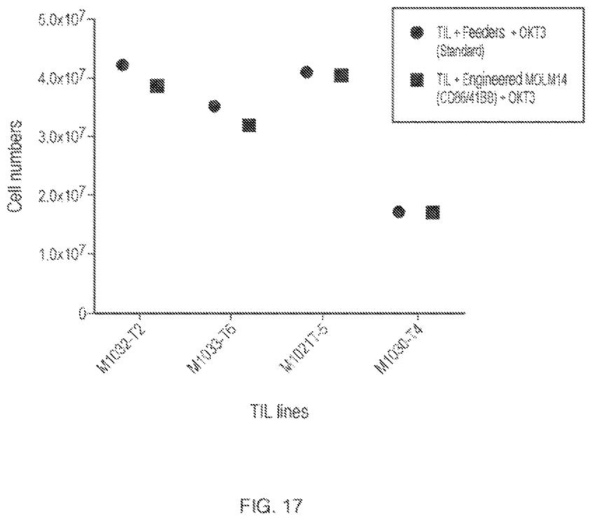

The results at day 14 are compared in FIG. 17, where results from two additional patient TILs are shown. The results indicate that MOLM-14 cells that were engineered with CD86 and 4-1BBL showed similar TIL expansion in the rapid expansion protocol when compared with allogeneic feeder cells. However, TILs cultured with parental MOLM-14 did not expand.

In addition, TILs expanded against MOLM-14 maintained a TIL phenotype and showed potency to kill P815 cells as measured using BRLA, which is described in detail in Example 9. Briefly, luciferin-transduced P815 target cells and TILs of interest were co-cultured with and without anti-CD3 to determine whether tumor reactivity of TILs is through TCR activation (specific killing) or non-specific killing. Following 4 hours of incubation, luciferin was added to the wells and incubated for 5 minutes. After the incubation, bioluminescence intensity was read using a luminometer. The percentage cytotoxicity and percentage survival were calculated using the following formula: % Survival=(experimental survival−minimum)/(maximum signal−minimum signal)×100 or % Cytotoxicity=100−(% Survival).

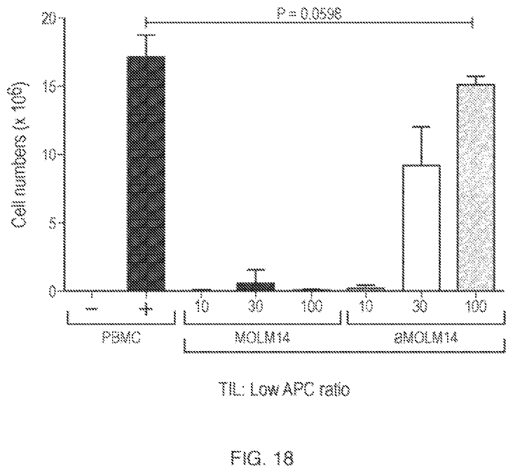

In FIG. 18, the results of expansions performed with low ratios of TILs to MOLM-14 aAPCs are shown in comparison to the results of expansions with PBMC feeders. TILs (2×10⁴) were cultured at different TIL to aAPC or PBMC ratios (1:10, 1:30, and 1:100, denoted “10”, “30”, and “100”, respectively) with parental MOLM-14 (“MOLM14”) cells, MOLM-14 cells transduced to express CD86 and 4-1BBL (“aMOLM14”), or PBMC feeders (“PBMC+”), each with OKT-3 (30 ng/mL) and IL-2 (3000 IU/mL) in a 24-well G-Rex plate. A control was performed using only OKT-3 (30 ng/ml) and IL-2 (3000 IU/mL) (“PBMC-”). Each condition was cultured in triplicate. Cultures were fed with fresh media and IL-2 on Day 4 and 7. Viable cells were counted on Day 7. FIG. 18 shows the mean plus standard deviation (SD) of viable cell numbers counted on Day 11, with a p-value calculated by the student t-test. Additional control experiments were performed using TILs alone, PBMCs alone, and aMOLM-14 cells alone, all of which resulted in undetectable cell numbers (data not shown). The results show that a ratio of 1:100 (TIL:aMOLM14) with OKT-3 and IL-2 yields a similar expansion when compared to PBMC feeders with OKT-3 and IL-2 (p=0.0598).

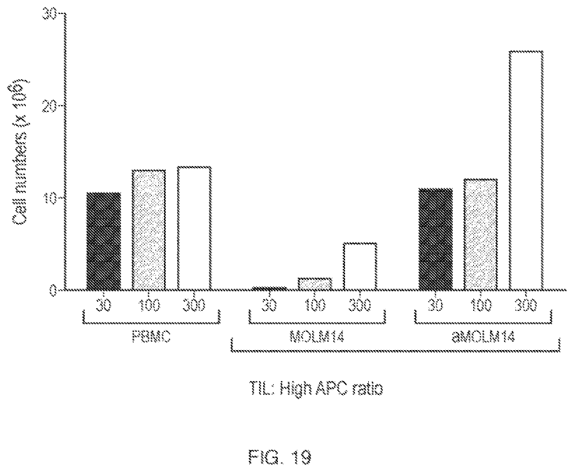

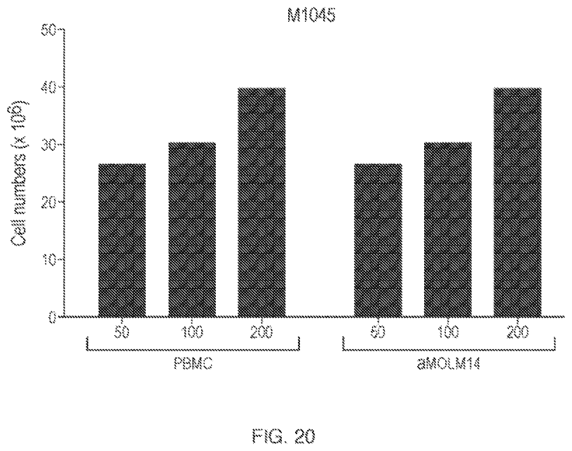

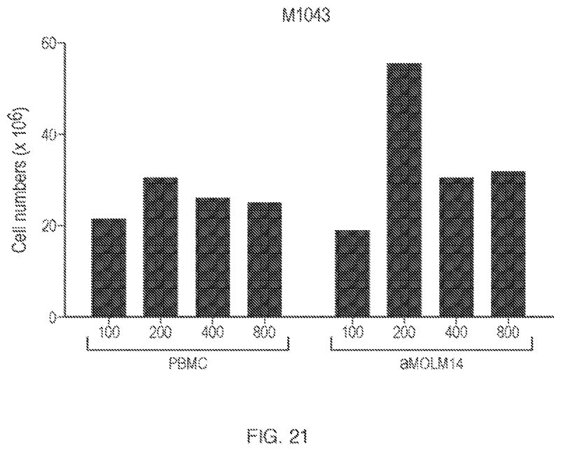

In FIG. 19, the results of expansions performed with higher ratios of TILs to MOLM-14 aAPCs, and otherwise performed as described above for FIG. 18, are shown in comparison to the results of expansions with PBMC feeders. At a ratio of 1:300, the CD86/4-1BBL modified MOLM-14 aAPCs with OKT-3 and IL-2 significantly outperform PBMC feeders with OKT-3 and IL-2. These results were verified using different TIL batches in repeat experiments shown in FIG. 20 and FIG. 21. In particular, as seen in FIG. 21, TIL to aMOLM14 ratios of 1:200 show enhanced TIL expansion compared to PBMC feeders under the same conditions. These results confirm that aMOLM14 aAPCs are unexpectedly superior in terms of expanding the TIL numbers than PBMCs particularly when using TIL:aMOLM14 ratios of 1:200 to 1:300.

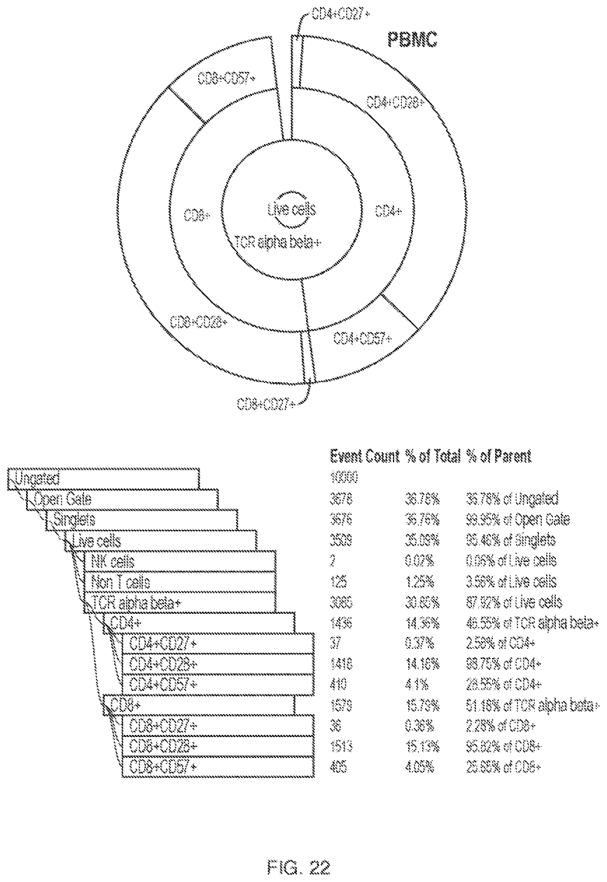

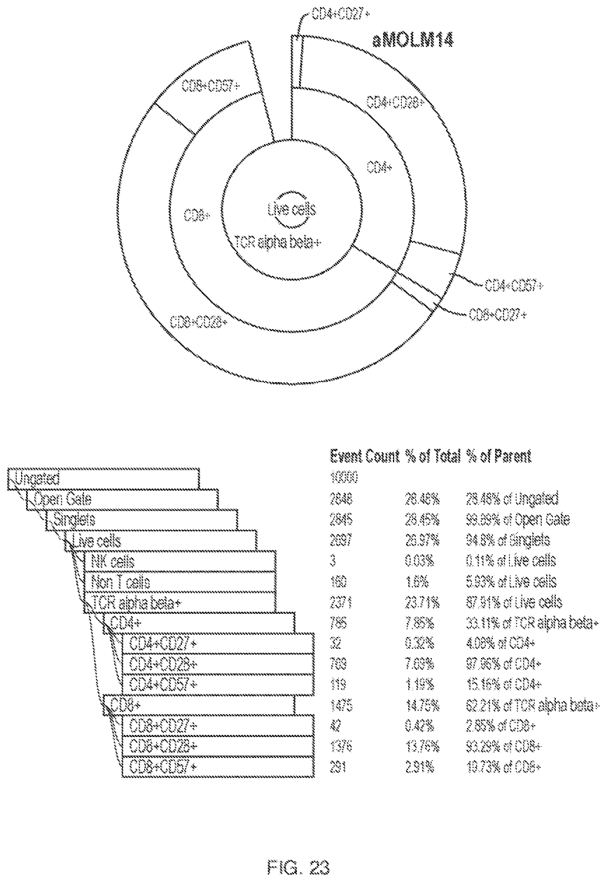

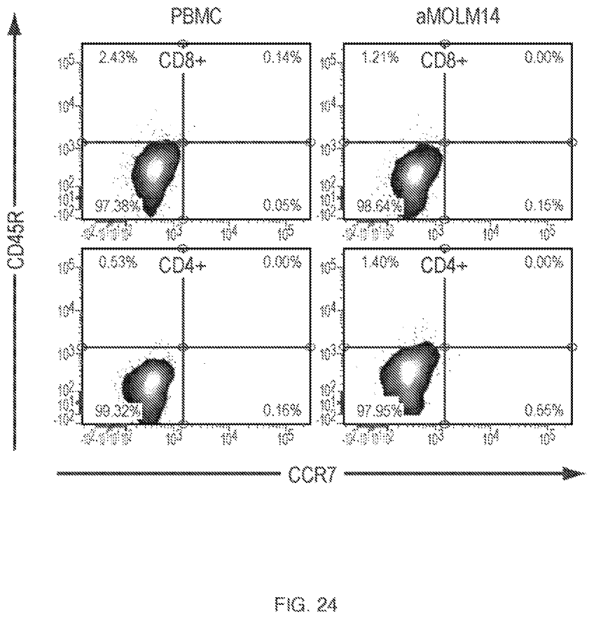

In FIG. 22 and FIG. 23, TILs expanded with aMOLM14 or PBMC were compared by flow cytometry analysis to confirm that the TILs exhibited a similar phenotype and would be expected to perform similarly upon reinfusion into a patient. Briefly, TILs were first stained with L/D Aqua to determine viability. Next, cells were surface stained with TCR □/□ PE-Cy7, CD4 FITC, CD8 PB, CD56 APC, CD28PE, CD27 APC-C7, and CD57-PerCP-Cy5.5. Phenotype analysis was done by gating 10,000 to 100,000 cells according to forward light scattering (FSC)/side light scattering (SSC) using a Canto II flow cytometer (Becton, Dickinson, and Co., Franklin Lakes, NJ, USA). Data was analyzed by Cytobank software to create sunburst diagrams and SPADE (Spanning Tree Progression of Density Normalized Event) analyses. Gates were set based on fluorescence minus one (FMO) controls. TILs expanded against aMOLM14 increases CD8⁺ TILs when compared to PBMC feeders. Without being bound by theory, this enhanced CD8⁺ TIL percentage may be due to the presence of 4-1BBL engineered to MOLM14. There is no difference in the expression of CD28, CD57, and CD27 differentiation markers. Additional flow cytometry data is shown in FIG. 24, and depicts a flow cytometry contour plot showing a memory subset (CD45RA+/−, CCR7+/−) gated on Live, TCR α/β+, CD4⁺ or CD8⁺ TILs, indicating that the memory subset obtained with PBMC feeders is replicated by the aMOLM14 aAPCs.

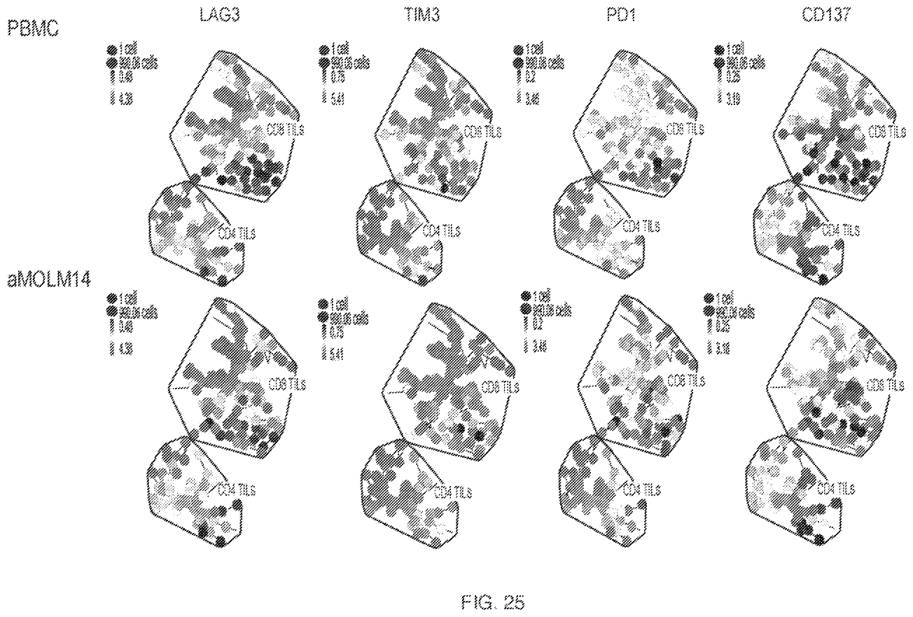

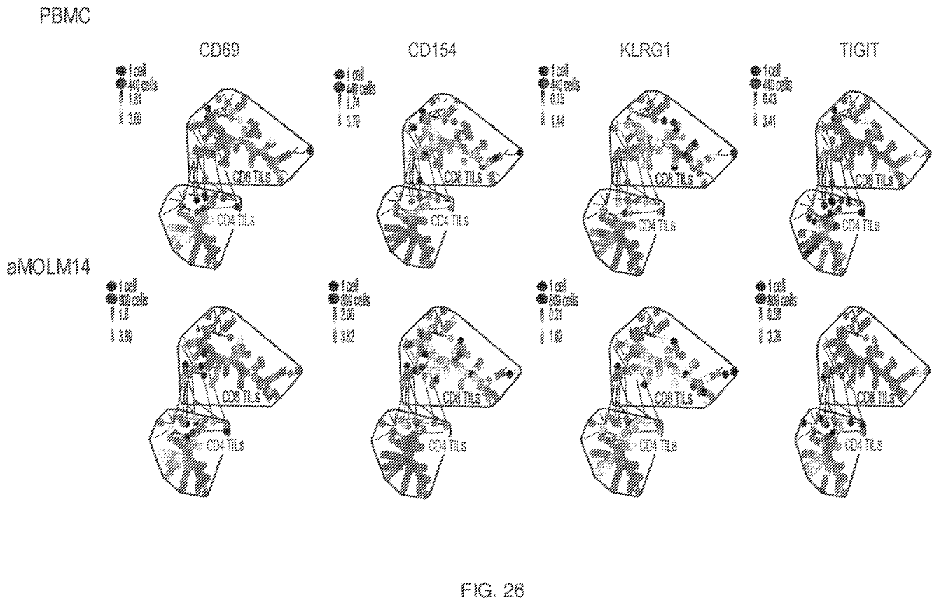

The CD4 and CD8 SPADE tree of TILs expanded with aMOLM14 aAPCs or PBMC feeders using CD3+ cells is shown in FIG. 25 and FIG. 26. The color gradient is proportional to the mean fluorescence intensity (MFI) of LAG3, TIL3, PD1 and CD137 or CD69, CD154, KLRG1 and TIGIT. Without being bound by theory, the results show that two batches of TILs expanded against aMOLM14 had undergone activation, but there was no difference in MFI between the aMOLM14 aAPCs and PBMC feeders, indicating that the aMOLM14 aAPCs effectively replicate the TIL phenotypic results obtained with PBMC feeders.

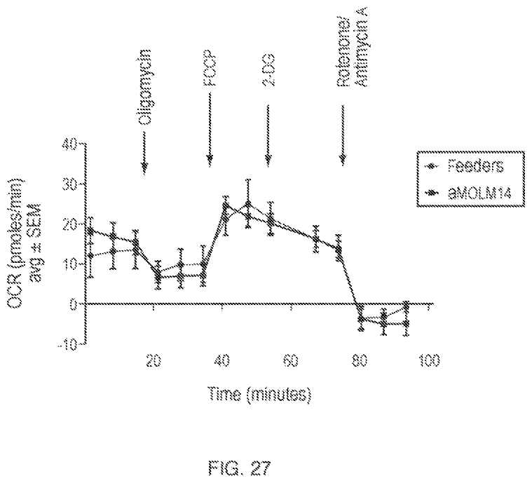

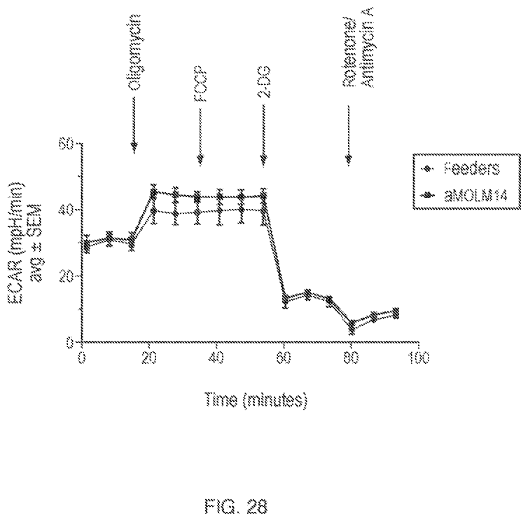

TILs expanded against aMOLM14 or PBMC were also analyzed for metabolic profiles. Oxygen consumption rate (OCR) and extracellular acidification rate (ECAR) of TILs after expansion with irradiated PBMC feeders or aMOLM14 aAPCs were measured using a dual mitochondrial-glycolytic stress test. Briefly, cells were washed in assay medium (XF Assay Medium, Agilent Technologies, Santa Clara, CA, USA), supplemented with 10 mM glucose, 1 mM sodium pyruvate, and 2 mM L-glutamine, at pH 7.4, and then 1×10⁵ viable cells were plated onto an adhesive-coated (Cell-Tak™, Corning) XFp cell culture microplate. Plates were spun to adhere the cells to the plate, then equilibrated at 37° C. in a humidified, non-CO₂ incubator prior to analysis of cellular metabolism. Mitochondrial and glycolytic stress test experiments were performed using a Seahorse XFp Analyzer (Agilent Technologies, Santa Clara, CA, USA), sequentially injecting the following compounds at specified intervals for simultaneous analysis of mitochondrial and glycolytic respiration of the cells: 1 μM oligomycin; 0.5 μM FCCP; 50 mM 2-deoxyglucose; and 0.5 μM each of rotenone and antimycin A. Results were analyzed using WAVE v2.3.0 software (Agilent Technologies, Santa Clara, CA, USA) and GraphPad Prism v6.07 graphing software and are shown in FIG. 27 and FIG. 28, where points represent mean±SEM measured in triplicate. Both TILs grown with aMOLM14 aAPCs and PBMC feeders show similar oxphos and glycolysis behavior. This data suggests that aMOLM14 does not alter the metabolic programming of TILs when compared with PBMC feeders.

### Example 5—Preparation of EM-3 Artificial Antigen Presenting Cells (aEM3 aAPCs)

EM-3 cells were obtained from Creative Bioarray, Inc. (Shirley, NY, USA). To develop an EM-3 based artificial APC, EM-3 cell lines were engineered with CD86, 4-1BBL, and antibody against IgG Fc region (Clone 7C12 or Clone 8B3). Human CD86 and human 4-1BBL/CD137 genes were cloned into commercially-available PLV430G and co-transfected with PDONR221 vectors (Invitrogen) using a lentiviral transduction method. The gateway cloning method was used as described in Katzen, Expert Opin. Drug Disc. 2007, 4, 571-589, to clone hCD86 and hCD137L genes onto the PLV430G and PDONR221 vectors. The 293T cell line was used for lentiviral production, and transduced to EM-3 cell lines. The transfected cells were sorted (S3e Cell Sorter, BioRad, Hercules, CA, USA) using APC-conjugated CD86 and PE-conjugated CD137L to isolate and enrich the cells. The enriched cells were checked for purity by flow cytometry. Single-chain Fv (scFv) antibody clones designated 7C12 and 8B3 were generated against Fc of mouse IgG1, IgG2a and IgG2b (Viva Biotech Ltd., Chicago, IL, USA). The amino acid sequences of these scFv clones are given in Table 7 (SEQ ID NO:27 and SEQ ID NO: 28). The generated scFv clones were screened for Fc binding efficiency against OKT-3, engineered towards pLV4301G containing eGFP as co-reporter to produce lentivirus. The 293T cell line was used for packaging and lentiviral production. Engineered EM-3 (CD86/CD137L) cells were transduced using the lentiviral system and sorted using eGFP. EM37C12CD86CD137L and EM38B3CD86CD137L were regularly assessed for the consistent expression of each transduced molecule by flow cytometry.

TABLE 7
Amino acid sequences of scFv clones 7C12 and 8B3.

| Identifier | | |
|---|---|---|
| (Description) | Sequence (One-Letter Amino Acid Symbols) | |
| SEQ ID NO: 27 | QVQLVQSGGG LVKPGGSLRL SCAASGFNFN DQYMSWIRQA PGKGLEWVSF ISGSGGTTYY | 60 |
| (mFC-7C12 | TDSVKGRFTI SRDNTKDSLY LQMNSLTVED TAVYYCARGG NYYTSVGRGT LVTVSAGGGG | 120 |
| scFv) | SGAPDIQMTQ SPGTLSLSPG ERAILSCRAS QSVSGYLAWY QQKPGQAPRL LIYGASSRAT | 180 |
| | GIPDRFSGSG SGTDFTLTIS SLRPEDIGTY YCKQYINAPF TEGGGTKVEI K | 231 |
| SEQ ID NO: 28 | QVQLQQSGAE VKKPGSSVKV SCKASGGTFS SYAISWVRQA PGQGLEWMGW ISPYNGNTDY | 60 |
| (mFC-8B3 scFv) | AQKVQGRVTL TTDTSTSTAY MELRSLRSDD TAVYYCATGG GTWYSDLWGR GTLVTVSAGG | 120 |
| | GGSGGGGSGG GGSGAPEIVL TQSPSTLSAS VGDRVSITCR ASQSIGGSLA WYQQKPGKAP | 180 |
| | KLLISEASTL ERGVPSRFSG SGSGTDFTLT ISSLQPEDVA TYYCQKYNSV PLTFGPGTKV | 240 |
| | EIK | 243 |

A non-limiting protocol for preparation of aEM3 aAPCs, which may also be adapted for use with aMOLM14 aAPCs, is described in the following paragraphs.

Molecular cloning of plasmids of interest may be performed as follows. To generate DONR vector the following cocktail may be used: B site flanked PCR product or destination vector (e.g., Gateway-adapted lentivector) 50-100 μg; DONR vector (e.g., pDONR222) 50-100 μg; BR Clonase II (Life Technologies) 1 μL; and TE buffer ((1 mM Tris, 0.1 mM EDTA, pH 8.0, q.s. to bring volume to 5 μL). Incubate at room temperature for at least 1 hour. After incubation perform bacterial transformation either by heat shock method or electroporation. To generate destination vector, the following cocktail may be used: recombined pDONR vector (e.g., pDON222-geneX) 50-100 μg, destination vector (e.g., Gateway adapted lentivector) 50-100 μg, LR Clonase II (Life Technologies) 1 μL, and TE buffer ((1 mM Tris, 0.1 mM EDTA, pH 8.0, q.s. to bring volume to 5 μL). Incubate at room temperature for at least 1 hour. After incubation, perform bacterial transformation either by chemical competent transformation/heat shock method.

Transformation and selection of the cloned plasmid may be performed as follows. The chemical competent transformation method may be performed as follows. Prepare nutrient agar plates (LB-Lennox or YT) with antibiotic for selection. Ensure that Recovery Medium (supplied by Lucigen, Middleton, WI, USA) is readily available at room temperature. Optionally, sterile culture tubes may be chilled on ice (e.g., 17 mm×100 mm tubes (14 mL tube)), one tube for each transformation reaction). Remove E. cloni cells (Lucigen) from an −80° C. freezer and thaw completely on wet ice (5-15 minutes). Optionally add 40 μL of E. cloni cells to the chilled culture tube. Add 1-4 μL of DNA sample to the 40 μL of cells. Flick with finger (do not pipet up and down to mix, which can introduce air bubbles and warm the cells). Incubate the cell/DNA mixture on ice for 30 minutes. Heat shock cells by placing the culture tubes in a 42° C. water bath for 45 seconds. Return the 1.7 mL tube or culture tubes to ice for 2 minutes. Add 350 μL room temperature Recovery Medium to the cells or 960 μL of room temperature Recovery Medium to the cells in the culture tube. Place the tubes in a shaking incubator at 250 rpm for 1 hour at 37° C. Plate up to 100% of the transformation mixture on LB-Lennox or YT agar plates containing the appropriate antibiotic. The plating volume may need to be optimized depending on DNA. Incubate the plates overnight at 37° C. Transformed clones can be further grown in any rich culture medium (e.g., LB or TB).

Colonies for Miniprep (Qiagen, Inc., Valencia, CA, USA) may be grown as follows. After colonies have formed from plating recovered transformation reaction of DNA manipulation (e.g. LR reaction), add 1 mL desired TB/antibiotics into desired number of 2 mL Eppendorf microtubes with punctured caps. Pick desired number of colonies using ART LTS 20 μL soft pipette tip (VWR 89031-352) or 10 μL Denville tip. Place tip in 2 mL Eppendorf microtube with punctured cap. Cut the tip so that it fits in tube, close cap, and place tubes on shaker (purple 15 mL tube holder with VWR brand 15 mL tubes). Shake overnight (for no more than 16 hours) at 225 rpm/37° C. After overnight incubation, place each tip in a 1 mL tube in a ClavePak 96 plate from Denville with sterile water in it (to save the tip for making bacterial stock production after the plasmids are screened and selected). Perform Miniprep according to the Qiagen Mini prep kit protocol (Qiagen, Inc., Valencia, CA, USA). Once the plasmids are eluted, restriction digestion is performed to select the right clones. After selecting the plasmids, use the tips saved from the same plasmids clone to grow the E. coli with the plasmid to make bacterial stock.

Lentiviral production may be performed as follows. The following media composition is prepared: 500 mL DMEM/F12 (Sigma); 25 mL FBS Heat Inactivated (HI) (Hyclone); 10 mM HEPES (Life Technologies); 1× Primocin (Invivogen); 1× Plasmocin (Invivogen); and 1×2-mercaptoethanol (Life Technologies). Harvest T75 flasks (Thermo Fisher Scientific) containing 90% confluent 293T cells. Aspirate media. Add 10 ml PBS, rinse gently and aspirate off. Add 2 mL TrypLE Express (Life Technologies) and evenly distribute it over the cell layer, let sit for 3-5 minutes at 37° C. (cell culture incubator). Add 10 mL media and disperse cells by pipetting up and down. Combine if there are multiple flasks. Count cells. If using a hemacytometer to determine concentration, cells/mL=(#counted cells×dilution factor×10⁴). To split back into T75 flasks, determine the time at which the cells will need to be fully confluent and dilute accordingly. (Cells double every 16-18 hours, so 3 days=1/27 dilution). Generally, a multiplication factor of 2.5 per day may be used where confluence is 2×10⁵ cells/cm². Bring volume up to 25 mL of media. To plate for titration of stocks, each well of the assay requires 5×10⁴ cells in 0.4 mL of media. Adjust 293T cells to 2×10⁴/mL in media. Plate 1 mL per well in a 24 well plate. For example, cells plated Monday may be infected on Tuesday and run on the flow cytometer on Friday, and cells plated Thursday are infected Friday and run on the flow cytometer on Monday. To plate for packaging transfections, seed T75 flasks with 6.8×10⁶ cells one day before transfection or 1.7×10⁶ cells on the morning of transfection. (Seeding on the day of transfection may reduce the variation in transfection efficiency). Bring volume in flask up to 25 mL with media. For example, flasks set up Monday are transfected Tuesday, and virus is collected on Thursday and Friday. In some cases (e.g., high titering constructs), the second collection can be omitted. To package lentiviral vectors, each T75 flask transfection requires 2 μg Baculo p35 plasmid (optional; only necessary if packaging a death gene), 2 μg VSV.G env plasmid (e.g., pMD2.G or PCIGO VSV-G); 4.7 μg Gag/polymerase plasmid (e.g., psPAX2 or pCMV-deltaR8.91), and 2.3 μg of the lentiviral vector described above. Determine the amount of VSV and R8.2/9.1 (+/−Baculo) plasmids needed for all samples (make a mixture of these DNAs if preparing many samples). Each T75 transfection requires 90 μL LipofectAmine 2000 (Thermo Fisher Scientific) in 2 mL Opti-MEM medium (Thermo Fisher Scientific). Make a mix containing enough Opti-Mem and LipofectAmine 2000 for all samples. Mix gently and let sit for 5 minutes at room temp, and label as tube A. For each transfection, add packaging DNA and specific lentiviral vector DNA to 500 μL room temperature Opti-MEM medium to a microtube and mix, and label as tube B. Add the 500 μL of DNA from tube B to the 2 mL of the LipofectAmine 2000 mix in tube A and mix gently, and incubate for 20-30 minutes at room temperature. Aspirate media from packaging flasks. Add the 2.5 mL of DNA/Lipofectamine complexes to 5 mL Opti-MEM medium and add to cells (do not pipet directly on cells since 293T cells are only semi adherent). Process plates in small groups to avoid drying. Incubate overnight and change media the next day in the morning. Collect the supernatant after 24 hours of media change. Supernatants can be harvested in a single collection, 48 hours after transfection or as 2 collections, 48 and 72 hours after transfection (in which case, harvests are pooled). If double collection is desired, collect supernatants by pipet on the first day, and replace with 20 mL of fresh media. To avoid flasks drying, work with only 5 flasks at a time. Keep collected supernatants at 4° C. until pooling the next day. Cool supernatants again on the following day and pool as appropriate. Spin the supernatants at 2000 rpm for 5 minutes to sediment any contaminating 293T cells. Filter harvested supernatants through a 0.45 μm or 0.8 μm filter unit containing a pre-filter disc. Use a large enough filtration unit so that the filtration speed is relatively fast. Store at 4° C. until ready to concentrate.

Virus may be concentrated using the PEG-it method (System Biosciences, Inc., Palo Alto, CA 94303) for longer-term storage at −80° C. Collect the supernatant from the transfection plates. Spin down the cell debris in the supernatant. The supernatant may also be filtered to completely remove any packaging cells. Add an amount of PEG-it solution equal to a quarter of the volume of supernatant to the supernatant. Incubate the suspension at 4° C. for overnight. Centrifuge at 3500 rpm (1500 g) at 4° C. for 30 minutes. Remove supernatant and centrifuge at 3500 rpm at 4° C. for 5 minutes. Remove remaining supernatant. Resuspend virus in desired amount of phosphate-buffered saline (PBS) and freeze aliquots at −80° C.

Transduction of cell line using lentivirus may be performed as follows. Adjust cells to be transduced to either: 1×10⁶ suspension cells per well in 24 well plate (1 well per transduction) or 50% confluence for adherent cells (1 well per transduction) in 24 well plate. For suspended cells, adjust concentration of cells to 1×10⁷/mL and plate 100 μL per well in 24 well plate (1 well per transduction). For adherent cells, plate to achieve 50% confluence on day of transduction based on cells/cm² (e.g., for 293T cells, confluence=2×10⁵/cm²). Total volume of transduction per well should be approximately 500 μL with 3-10 μg/mL Polybrene (Hexadimethrine bromide, Sigma-Aldrich Co., St. Louis, MO, USA). The amount of concentrated virus added will depend on the MOI (multiplicity of infection) desired. A typical MOI is 10:1 but this may vary depending on cell type. The transfection well should contain 100 μL of standard media containing either 1×10⁶ suspension cells or 50% confluent cells. For a MOI of 10:1 (e.g., virus activity is 1×10⁸ IU/mL and the target is to infect 1×10⁶ cells, then 1×10⁷ virions or 100 μL of virus is needed). Add standard media to 500 μL. Add Polybrene to 3 μg/mL (primary cells) to 10 μg/mL (tumor cell lines). Spin plate(s) at 1800 rpm for 1.5 to 2 hours at 30° C. Incubate plate(s) at 37° C./5% CO₂ using a Tissue Culture incubator for 5 hours to overnight. Change media. After 72 hours of transduction, if enough cells are available, perform flow cytometric analysis to test the transduction efficiency.

Sorting of aAPCs may be performed as follows. Culture the cells in the media described above until the cell count reaches a minimum of 10-20 million. Take 1×10⁶ cells for each condition and stain with the antibodies for the proteins transduced. Wash the cells and analyze by flow cytometry to test the stability of transduction. Once the expression of protein of interest has been analyzed and confirmed, prepare the rest of the cells for sorting. Sort the cells in an S3 sorter by gating on markers of interest. Culture the sorted cells using the media mentioned above. Before freezing the vial, test the stability of the protein expression of interest. Use Recovery cell culture Freezing media (Invitrogen), to make the cell bank of the same cells. Cells may be banked after each transduction and sorting procedure.

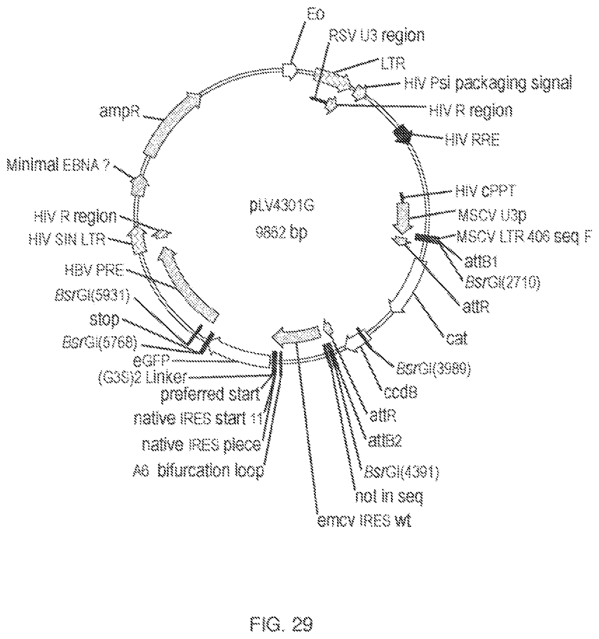

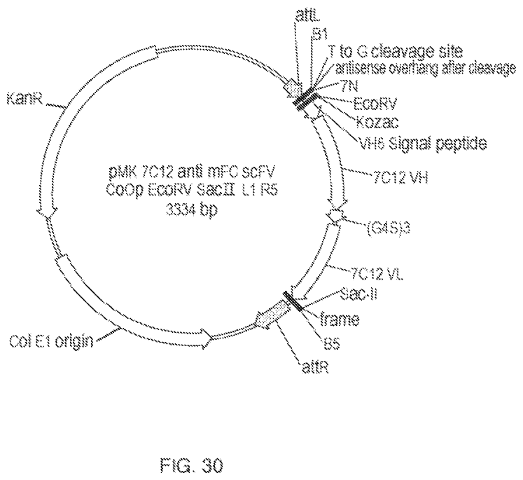

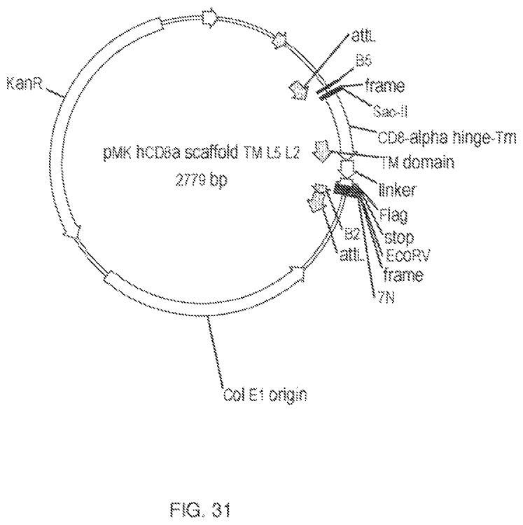

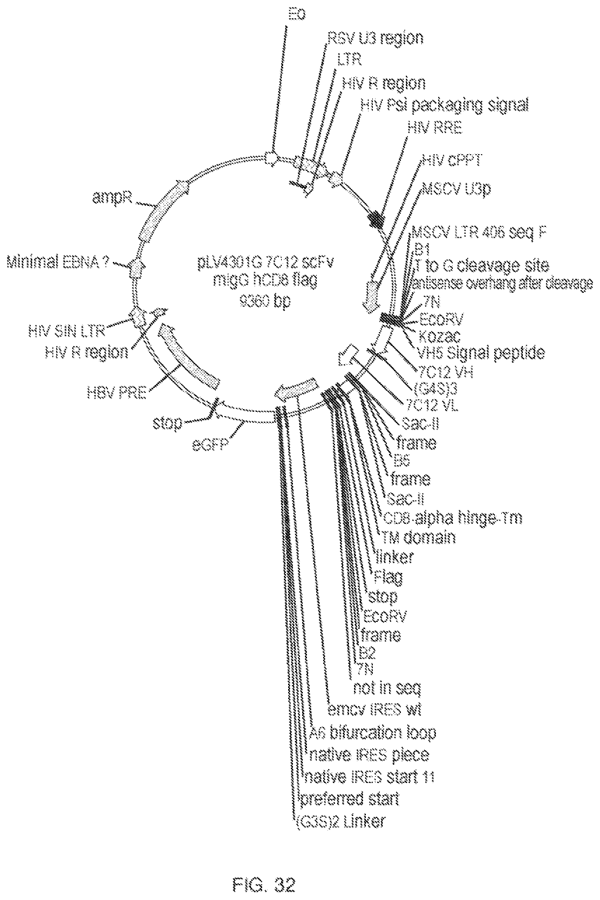

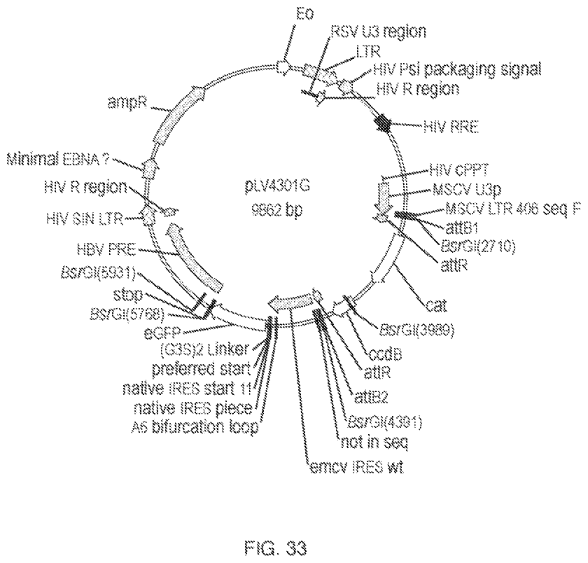

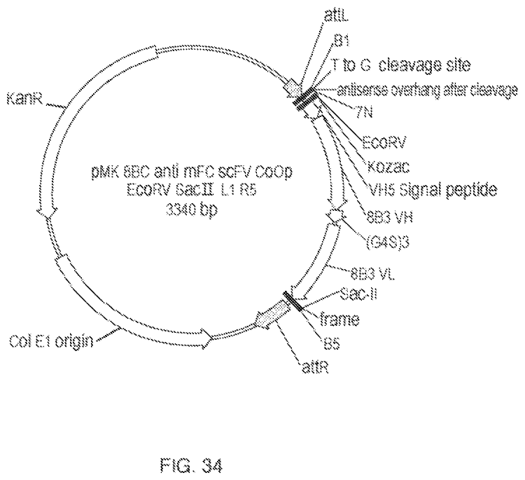

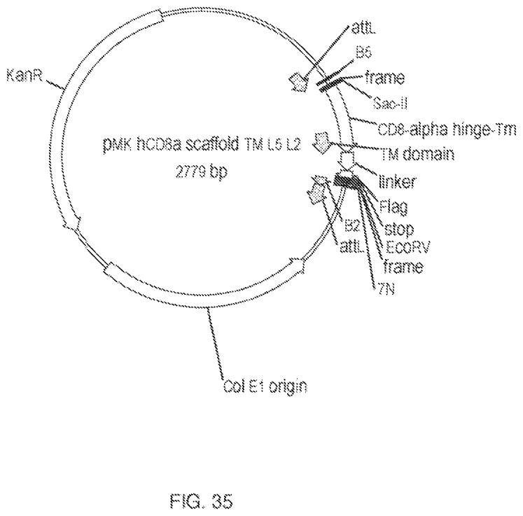

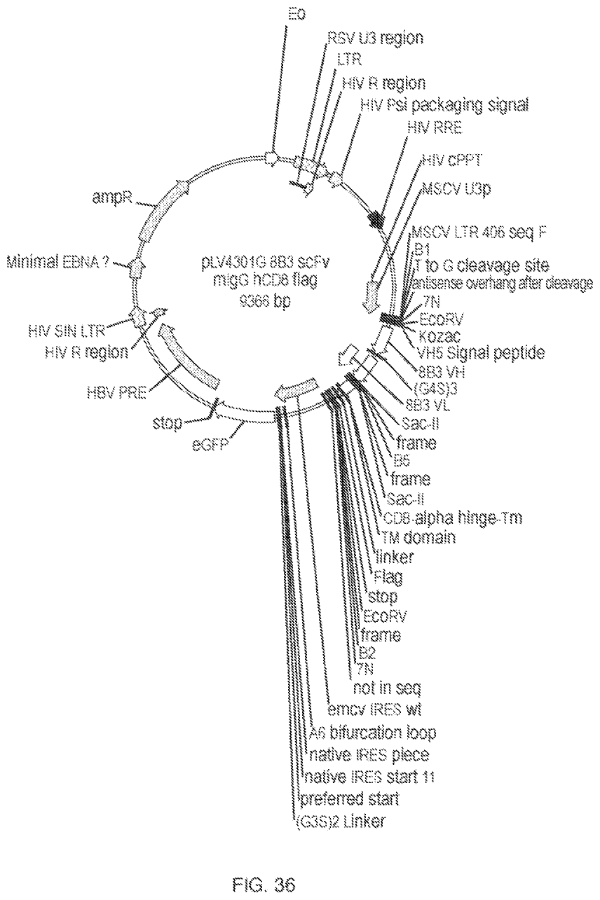

Nucleotide sequence information for the 7C12 and 8B3 scFv clones (SEQ ID NO:29 and SEQ ID NO:30) and their lentiviral vectors are given in Table 8. Sequences used for generation of the pLV4301G 7C12 scFv mIgG hCD8 flag vector are provided as SED IQ NO: 31 to SEQ ID NO:34 and are depicted in FIG. 29 to FIG. 32. Sequences used for generation of the pLV4301G 8B3 scFv mIgG hCD8 flag vector are provided as SEQ ID NO:35 to SEQ ID NO:38 and are depicted in FIG. 33 to FIG. 36.

TABLE 8
Nucleotide sequences for preparation of lentivirus for transduction of aAPCs.

| Identifier | | |
|---|---|---|
| (Description) | Sequence | |
| SEQ ID NO: 29 | caggtgcagc tggtgcagtc tgggggaggc ttggtcaagc ctggagggtc cctgagactc | 60 |
| (mFC-7C12 | tcctgtgcag cctctggatt caatttcaat gaccagtaca tgagttggat ccgccaggct | 120 |
| scFv) | ccagggaagg ggctggagtg ggtttcattc attagtggta gtggtggtac cacatactac | 180 |
| | acagactctg tgaagggccg gttcaccatc tccagggaca acaccaagga ctcattgtat | 240 |
| | ttgcaaatga acagcctgac agtcgaggac acggccgtgt actactgtgc gagaggaggg | 300 |
| | aattattata cttcggtggg ccggggcacc ctggtcaccg tctcggccgg tggcggcgga | 360 |
| | tctggcgcgc cagacatcca gatgacccag tctccaggca ccctgtcttt gtctccaggg | 420 |
| | gaaagagcca tcctctcctg cagggccagt cagagtgtta gcggctacct agcctggtat | 480 |
| | caacagaaac ctggccaggc tcccaggctc ctcatctatg gtgcatccag cagggccact | 540 |
| | ggcatcccag acaggttcag tggcagtggg tctgggacag acttcactct caccatcagc | 600 |
| | agcctgcggc ctgaagatat tggaacatat tactgtaaac agtacattaa tgccccattc | 660 |
| | actttcggcg gcgggaccaa ggtggagatc aaa | 693 |
| SEQ ID NO: 30 | caggtacagc tgcagcagtc aggggctgag gtgaagaagc ctgggtcctc ggtgaaggtc | 60 |
| (mFC-8B3 scFv) | tcctgcaagg cttctggagg caccttcagc agctatgcta tcagctgggt gcgacaggcc | 120 |
| | cctggacaag ggcttgagtg gatgggatgg atcagccctt acaatggtaa cacagattat | 180 |
| | gcacagaagg tccagggcag agtcaccttg accacagaca catccacgag cacagcctac | 240 |
| | atggagctga ggagcctgag atccgacgac acggccgtgt attactgtgc gacaggtggc | 300 |
| | gggacctggt actccgatct ctggggccgt ggcaccctgg tcaccgtctc ggccggtggc | 360 |
| | ggtggcagcg gcggtggtgg gtccggtggc ggcggatctg gcgcgccaga aattgtgctg | 420 |
| | actcagtctc cctccaccct gtctgcatct gtaggagaca gagtcagcat cacttgccgg | 480 |
| | gccagtcaga gtattggtgg gtcgttggcc tggtatcaac aaaagccagg gaaagcccct | 540 |
| | aagctcctga tctctgaggc gtctacttta gagaggggcg tcccatcaag attcagcggc | 600 |
| | agtggatctg ggacagattt cactctcacc atcagcagcc tgcagcctga agatgttgca | 660 |
| | acttattact gtcaaaaata taacagtgtc ccgctcactt tcggccctgg gaccaaggtg | 720 |
| | gagatcaaa | 729 |
| SEQ ID NO: 31 | cgataaccct aattcgatag catatgcttc ccgttgggta acatatgcta ttgaattagg | 60 |
| (destination | gttagtctgg atagtatata ctactacccg ggaagcatat gctacccgtt tagggttcac | 120 |
| vector | cggtgatgcc ggccacgatg cgtccggcgt agaggatcta atgtgagtta gctcactcat | 180 |
| pLV430lG) | taggcacccc aggctttaca ctttatgctt ccggctcgta tgttgtgtgg aattgtgagc | 240 |
| | ggataacaat ttcacacagg aaacagctat gaccatgatt acgccaagcg cgcaattaac | 300 |
| | cctcactaaa gggaacaaaa gctggagctg caagcttaat gtagtcttat gcaatactct | 360 |
| | tgtagtcttg caacatggta acgatgagtt agcaacatgc cttacaagga gagaaaaagc | 420 |
| | accgtgcatg ccgattggtg gaagtaaggt ggtacgatcg tgccttatta ggaaggcaac | 480 |
| | agacgggtct gacatggatt ggacgaacca ctgaattgcc gcattgcaga gatattgtat | 540 |
| | ttaagtgcct agctcgatac ataaacgggt ctctctggtt agaccagatc tgagcctggg | 600 |
| | agctctctgg ctaactaggg aacccactgc ttaagcctca ataaagcttg ccttgagtgc | 660 |
| | ttcaagtagt gtgtgcccgt ctgttgtgtg actctggtaa ctagagatcc ctcagaccct | 720 |
| | tttagtcagt gtggaaaatc tctagcagtg gcgcccgaac agggacttga aagcgaaagg | 780 |
| | gaaaccagag gagctctctc gacgcaggac tcggcttgct gaagcgcgca cggcaagagg | 840 |
| | cgaggggcgg cgactggtga gtacgccaaa aattttgact agcggaggct agaaggagag | 900 |
| | agatgggtgc gagagcgtca gtattaagcg ggggagaatt agatcgcgat gggaaaaaat | 960 |
| | tcggttaagg ccagggggaa agaaaaaata taaattaaaa catatagtat gggcaagcag | 1020 |
| | ggagctagaa cgattcgcag ttaatcctgg cctgttagaa acatcagaag gctgtagaca | 1080 |
| | aatactggga cagctacaac catcccttca gacaggatca gaagaactta gatcattata | 1140 |
| | taatacagta gcaaccctct attgtgtgca tcaaaggata gagataaaag acaccaagga | 1200 |
| | agctttagac aagatagagg aagagcaaaa caaaagtaag accaccgcac agcaagcggc | 1260 |
| | cgctgatctt cagacctgga ggaggagata tgagggacaa ttggagaagt gaattatata | 1320 |
| | aatataaagt agtaaaaatt gaaccattag gagtagcacc caccaaggca aagagaagag | 1380 |
| | tggtgcagag agaaaaaaga gcagtgggaa taggagcttt gttccttggg ttcttgggag | 1440 |
| | cagcaggaag cactatgggc gcagcgtcaa tgacgctgac ggtacaggcc agacaattat | 1500 |
| | tgtctggtat agtgcagcag cagaacaatt tgctgagggc tattgaggcg caacagcatc | 1560 |
| | tgttgcaact cacagtctgg ggcatcaagc agctccaggc aagaatcctg gctgtggaaa | 1620 |
| | gatacctaaa ggatcaacag ctcctgggga tttggggttg ctctggaaaa ctcatttgca | 1680 |
| | ccactgctgt gccttggaat gctagttgga gtaataaatc tctggaacag atttggaatc | 1740 |
| | acacgacctg gatggagtgg gacagagaaa ttaacaatta cacaagctta atacactcct | 1800 |
| | taattgaaga atcgcaaaac cagcaagaaa agaatgaaca agaattattg gaattagata | 1860 |
| | aatgggcaag tttgtggaat tggtttaaca taacaaattg gctgtggtat ataaaattat | 1920 |
| | tcataatgat agtaggaggc ttggtaggtt taagaatagt ttttgctgta ctttctatag | 1980 |
| | tgaatagagt taggcaggga tattcaccat tatcgtttca gacccacctc ccaaccccga | 2040 |
| | ggggacccga caggcccgaa ggaatagaag aagaaggtgg agagagagac agagacagat | 2100 |
| | ccattcgatt agtgaacgga tctcgacggt atcggtttta aaagaaaagg ggggattggg | 2160 |
| | gggtacagtg caggggaaag aatagtagac ataatagcaa cagacataca aactaaagaa | 2220 |
| | ttacaaaaac aaattacaaa aattcaaaat tttatcgatt ttatttagtc tccagaaaaa | 2280 |
| | ggggggaatg aaagacccca cctgtaggtt tggcaagcta gcttaagtaa cgccattttg | 2340 |
| | caaggcatgg aaaatacata actgagaata gagaagttca gatcaaggtt aggaacagag | 2400 |
| | agacagcaga atatgggcca aacaggatat ctgtggtaag cagttcctgc cccggctcag | 2460 |
| | ggccaagaac agatggtccc cagatgcggt cccgccctca gcagtttcta gagaaccatc | 2520 |
| | agatgtttcc agggtgcccc aaggacctga aatgaccctg tgccttattt gaactaacca | 2580 |
| | atcagttcgc ttctcgcttc tgttcgcgcg cttctgctcc ccgagctcaa taaaagagcc | 2640 |
| | cacaacccct cactcggcgc gccagtcctc cgatagactg cgtcgcccgg gtaccgatat | 2700 |
| | cacaagtttg tacaaaaaag ctgaacgaga aacgtaaaat gatataaata tcaatatatt | 2760 |
| | aaattagatt ttgcataaaa aacagactac ataatactgt aaaacacaac atatccagtc | 2820 |
| | actatggcgg ccgcattagg caccccaggc tttacacttt atgcttccgg ctcgtataat | 2880 |
| | gtgtggattt tgagttagga tccgtcgaga ttttcaggag ctaaggaagc taaaatggag | 2940 |
| | aaaaaaatca ctggatatac caccgttgat atatcccaat ggcatcgtaa agaacatttt | 3000 |
| | gaggcatttc agtcagttgc tcaatgtacc tataaccaga ccgttcagct ggatattacg | 3060 |
| | gcctttttaa agaccgtaaa gaaaaataag cacaagtttt atccggcctt tattcacatt | 3120 |
| | cttgcccgcc tgatgaatgc tcatccggaa ttccgtatgg caatgaaaga cggtgagctg | 3180 |
| | gtgatatggg atagtgttca cccttgttac accgttttcc atgagcaaac tgaaacgttt | 3240 |
| | tcatcgctct ggagtgaata ccacgacgat ttccggcagt ttctacacat atattcgcaa | 3300 |
| | gatgtggcgt gttacggtga aaacctggcc tatttcccta aagggtttat tgagaatatg | 3360 |
| | tttttcgtct cagccaatcc ctgggtgagt ttcaccagtt ttgatttaaa cgtggccaat | 3420 |
| | atggacaact tcttcgcccc cgttttcacc atgggcaaat attatacgca aggcgacaag | 3480 |
| | gtgctgatgc cgctggcgat tcaggttcat catgccgttt gtgatggctt ccatgtcggc | 3540 |
| | agaatgctta atgaattaca acagtactgc gatgagtggc agggcggggc gtaaatggat | 3600 |
| | ccggcttact aaaagccaga taacagtatg cgtatttgcg cgctgatttt tgcggtataa | 3660 |
| | gaatatatac tgatatgtat acccgaagta tgtcaaaaag aggtatgcta tgaagcagcg | 3720 |
| | tattacagtg acagttgaca gcgacagcta tcagttgctc aaggcatata tgatgtcaat | 3780 |
| | atctccggtc tggtaagcac aaccatgcag aatgaagccc gtcgtctgcg tgccgaacgc | 3840 |
| | tggaaagcgg aaaatcagga agggatggct gaggtcgccc ggtttattga aatgaacggc | 3900 |
| | tcttttgctg acgagaacag gggctggtga aatgcagttt aaggtttaca cctataaaag | 3960 |
| | agagagccgt tatcgtctgt ttgtggatgt acagagtgat attattgaca cgcccgggcg | 4020 |
| | acggatggtg atccccctgg ccagtgcacg tctgctgtca gataaagtct cccgtgaact | 4080 |
| | ttacccggtg gtgcatatcg gggatgaaag ctggcgcatg atgaccaccg atatggccag | 4140 |
| | tgtgccggtc tccgttatcg gggaagaagt ggctgatctc agccaccgcg aaaatgacat | 4200 |
| | caaaaacgcc attaacctga tgttctgggg aatataaatg tcaggctccc ttatacacag | 4260 |
| | ccagtctgca ggtcgaccat agtgactgga tatgttgtgt tttacagtat tatgtagtct | 4320 |
| | gttttttatg caaaatctaa tttaatatat tgatatttat atcattttac gtttctcgtt | 4380 |
| | cagctttctt gtacaaagtg gtgattcgag ttaattaagt taacgaattc cccccctctc | 4440 |
| | cctccccccc ccctaacgtt actggccgaa gccgcttgga ataaggccgg tgtgcgtttg | 4500 |
| | tctatatgtt attttccacc atattgccgt cttttggcaa tgtgagggcc cggaaacctg | 4560 |
| | gccctgtctt cttgacgagc attcctaggg gtctttcccc tctcgccaaa ggaatgcaag | 4620 |
| | gtctgttgaa tgtcgtgaag gaagcagttc ctctggaagc ttcttgaaga caaacaacgt | 4680 |
| | ctgtagcgac cctttgcagg cagcggaacc ccccacctgg cgacaggtgc ctctgcggcc | 4740 |
| | aaaagccacg tgtataagat acacctgcaa aggcggcaca accccagtgc cacgttgtga | 4800 |
| | gttggatagt tgtggaaaga gtcaaatggc tctcctcaag cgtattcaac aaggggctga | 4860 |
| | aggatgccca gaaggtaccc cattgtatgg gatctgatct ggggcctcgg tgcacatgct | 4920 |
| | ttacatgtgt ttagtcgagg ttaaaaaacg tctaggcccc ccgaaccacg gggacgtggt | 4980 |
| | tttcctttga aaaacacgat gataatatgg ccacaaccat gggaggcgga agcggcggag | 5040 |
| | gctcccctcg aggcaccatg gtgagcaagg gcgaggagct gttcaccggg gtggtgccca | 5100 |
| | tcctggtcga gctggacggc gacgtaaacg gccacaagtt cagcgtgtcc ggcgagggcg | 5160 |
| | agggcgatgc cacctacggc aagctgaccc tgaagttcat ctgcaccacc ggcaagctgc | 5220 |
| | ccgtgccctg gcccaccctc gtgaccaccc tgacctacgg cgtgcagtgc ttcagccgct | 5280 |
| | accccgacca catgaagcag cacgacttct tcaagtccgc catgcccgaa ggctacgtcc | 5340 |
| | aggagcgcac catcttcttc aaggacgacg gcaactacaa gacccgcgcc gaggtgaagt | 5400 |
| | tcgagggcga caccctggtg aaccgcatcg agctgaaggg catcgacttc aaggaggacg | 5460 |
| | gcaacatcct ggggcacaag ctggagtaca actacaacag ccacaacgtc tatatcatgg | 5520 |
| | ccgacaagca gaagaacggc atcaaggtga acttcaagat ccgccacaac atcgaggacg | 5580 |
| | gcagcgtgca gctcgccgac cactaccagc agaacacccc catcggcgac ggccccgtgc | 5640 |
| | tgctgcccga caaccactac ctgagcaccc agtccgccct gagcaaagac cccaacgaga | 5700 |
| | agcgcgatca catggtcctg ctggagttcg tgaccgccgc cgggatcact ctcggcatgg | 5760 |
| | acgagctgta caagtaacgc gtcccgggtc tagagctagc ggtaccatgc attacgtagt | 5820 |
| | cgacgactta attaagctag cctagtgcca tttgttcagt ggttcgtagg gctttccccc | 5880 |
| | actgtttggc tttcagttat atggatgatg tggtattggg ggccaagtct gtacagcatc | 5940 |
| | ttgagtccct ttttaccgct gttaccaatt ttcttttgtc tttgggtata catttaaacc | 6000 |
| | ctaacaaaac aaagagatgg ggttactctc taaattttat gggttatgtc attggatgtt | 6060 |
| | atgggtcctt gccacaagaa cacatcatac aaaaaatcaa agaatgtttt agaaaacttc | 6120 |
| | ctattaacag gcctattgat tggaaagtat gtcaacgaat tgtgggtctt ttgggttttg | 6180 |
| | ctgccccttt tacacaatgt ggttatcctg cgttgatgcc tttgtatgca tgtattcaat | 6240 |
| | ctaagcaggc tttcactttc tcgccaactt acaaggcctt tctgtgtaaa caatacctga | 6300 |
| | acctttaccc cgttgcccgg caacggccag gtctgtgcca agtgtttgct gacgcaaccc | 6360 |
| | ccactggctg gggcttggtc atgggccatc agcgcatgcg tggaaccttt tcggctcctc | 6420 |
| | tgccgatcca tactgcggaa ctcctagccg cttgttttgc tcgcagcagg tctggagcaa | 6480 |
| | acattatcgg gactgataac tctgttgtcc tatcccgcaa atatacatcg tttccatggc | 6540 |
| | tgctaggctg tgctgccaac tggatcctgc gcgggacgtc ctttgtttac gtcccgtcgg | 6600 |
| | cgctgaatcc tgcggacgac ccttctcggg gtcgcttggg actctctcgt ccccttctcc | 6660 |
| | gtctgccgtt ccgaccgacc acggggcgca cctctcttta cgcggactcc ccgtctgtgc | 6720 |
| | cttctcatct gccggaccgt gtgcacttcg cttcacctct gcacgtcgca tggagaccac | 6780 |
| | cgtgaacgcc caccaaatat tgcccaaggt cttacataag aggactcttg gactctcagc | 6840 |
| | aatgtcaacg accgaccttg aggcatactt caaagactgt ttgtttaaag actgggagga | 6900 |
| | gttgggggag gagattaggt taaaggtctt tgtactagga ggctgtaggc ataaattggt | 6960 |
| | ctgcgcacca gcaccatggc gcaatcacta gagcggggta cctttaagac caatgactta | 7020 |
| | caaggcagct gtagatctta gccacttttt aaaagaaaag gggggactgg aagggctaat | 7080 |
| | tcactcccaa cgaagacaag atctgctttt tgcttgtact gggtctctct ggttagacca | 7140 |
| | gatctgagcc tgggagctct ctggctaact agggaaccca ctgcttaagc ctcaataaag | 7200 |
| | cttgccttga gtgcttcaag tagtgtgtgc ccgtctgttg tgtgactctg gtaactagag | 7260 |
| | atccctcaga cccttttagt cagtgtggaa aatctctagc agtagtagtt catgtcatct | 7320 |
| | tattattcag tatttataac ttgcaaagaa atgaatatca gagagtgaga ggaacttgtt | 7380 |
| | tattgcagct tataatggtt acaaataaag caatagcatc acaaatttca caaataaagc | 7440 |
| | atttttttca ctgcattcta gttgtggttt gtccaaactc atcaatgtat cttatcatgt | 7500 |
| | ctggctctag ctatcccgcc cctaactccg cccatcccgc ccctaactcc gcccagttcc | 7560 |
| | gcccattctc cgccccatgg ctgactaatt ttttttattt atgcagaggc cgaggccgga | 7620 |
| | tcccttgagt ggctttcatc ctggagcaga ctttgcagtc tgtggactgc aacacaacat | 7680 |
| | tgcctttatg tgtaactctt ggctgaagct cttacaccaa tgctggggga catgtacctc | 7740 |
| | ccaggggccc aggaagacta cgggaggcta caccaacgtc aatcagaggg gcctgtgtag | 7800 |
| | ctaccgataa gcggaccctc aagagggcat tagcaatagt gtttataagg cccccttgtt | 7860 |
| | aattcttgaa gacgaaaggg cctcgtgata cgcctatttt tataggttaa tgtcatgata | 7920 |
| | ataatggttt cttagacgtc aggtggcact tttcggggaa atgtgcgcgg aacccctatt | 7980 |
| | tgtttatttt tctaaataca ttcaaatatg tatccgctca tgagacaata accctgataa | 8040 |
| | atgcttcaat aatattgaaa aaggaagagt atgagtattc aacatttccg tgtcgccctt | 8100 |
| | attccctttt ttgcggcatt ttgccttcct gtttttgctc acccagaaac gctggtgaaa | 8160 |
| | gtaaaagatg ctgaagatca gttgggtgca cgagtgggtt acatcgaact ggatctcaac | 8220 |
| | agcggtaaga tccttgagag ttttcgcccc gaagaacgtt ttccaatgat gagcactttt | 8280 |
| | aaagttctgc tatgtggcgc ggtattatcc cgtgttgacg ccgggcaaga gcaactcggt | 8340 |
| | cgccgcatac actattctca gaatgacttg gttgagtact caccagtcac agaaaagcat | 8400 |
| | cttacggatg gcatgacagt aagagaatta tgcagtgctg ccataaccat gagtgataac | 8460 |
| | actgcggcca acttacttct gacaacgatc ggaggaccga aggagctaac cgcttttttg | 8520 |
| | cacaacatgg gggatcatgt aactcgcctt gatcgttggg aaccggagct gaatgaagcc | 8580 |
| | ataccaaacg acgagcgtga caccacgatg cctgcagcaa tggcaacaac gttgcgcaaa | 8640 |
| | ctattaactg gcgaactact tactctagct tcccggcaac aattaataga ctggatggag | 8700 |
| | gcggataaag ttgcaggacc acttctgcgc tcggcccttc cggctggctg gtttattgct | 8760 |
| | gataaatctg gagccggtga gcgtgggtct cgcggtatca ttgcagcact ggggccagat | 8820 |
| | ggtaagccct cccgtatcgt agttatctac acgacgggga gtcaggcaac tatggatgaa | 8880 |
| | cgaaatagac agatcgctga gataggtgcc tcactgatta agcattggta actgtcagac | 8940 |
| | caagtttact catatatact ttagattgat ttaaaacttc atttttaatt taaaaggatc | 9000 |
| | taggtgaaga tcctttttga taatctcatg accaaaatcc cttaacgtga gttttcgttc | 9060 |
| | cactgagcgt cagaccccgt agaaaagatc aaaggatctt cttgagatcc tttttttctg | 9120 |
| | cgcgtaatct gctgcttgca aacaaaaaaa ccaccgctac cagcggtggt ttgtttgccg | 9180 |
| | gatcaagagc taccaactct ttttccgaag gtaactggct tcagcagagc gcagatacca | 9240 |
| | aatactgtcc ttctagtgta gccgtagtta ggccaccact tcaagaactc tgtagcaccg | 9300 |
| | cctacatacc tcgctctgct aatcctgtta ccagtggctg ctgccagtgg cgataagtcg | 9360 |
| | tgtcttaccg ggttggactc aagacgatag ttaccggata aggcgcagcg gtcgggctga | 9420 |
| | acggggggtt cgtgcacaca gcccagcttg gagcgaacga cctacaccga actgagatac | 9480 |
| | ctacagcgtg agcattgaga aagcgccacg cttcccgaag ggagaaaggc ggacaggtat | 9540 |
| | ccggtaagcg gcagggtcgg aacaggagag cgcacgaggg agcttccagg gggaaacgcc | 9600 |
| | tggtatcttt atagtcctgt cgggtttcgc cacctctgac ttgagcgtcg atttttgtga | 9660 |
| | tgctcgtcag gggggcggag cctatggaaa aacgccagca acgcggcctt tttacggttc | 9720 |
| | ctggcctttt gctggccttt ttgaagctgt ccctgatggt cgtcatctac ctgcctggac | 9780 |
| | agcatggcct gcaacgcggg catcccgatg ccgccggaag cgagaagaat cataatgggg | 9840 |
| | aaggccatcc agcctcgcgt cg | 9862 |
| SEQ ID NO: 32 | ctaaattgta agcgttaata ttttgttaaa attcgcgtta aatttttgtt aaatcagctc | 60 |
| (donor vector | attttttaac caataggccg aaatcggcaa aatcccttat aaatcaaaag aatagaccga | 120 |
| 1, pMK 7cl2 | gatagggttg agtggccgct acagggcgct cccattcgcc attcaggctg cgcaactgtt | 180 |
| anti mFC scFV | gggaagggcg tttcggtgcg ggcctcttcg ctattacgcc agctggcgaa agggggatgt | 240 |
| CoOp ECORV | gctgcaaggc gattaagttg ggtaacgcca gggttttccc agtcacgacg ttgtaaaacg | 300 |
| SacII L1R5) | acggccagtg agcgcgacgt aatacgactc actatagggc gaattgaagg aaggccgtca | 360 |
| | aggccgcata aataatgatt ttattttgac tgatagtgac ctgttcgttg caacaaattg | 420 |
| | atgagcaatg cttttttata atgccaactt tgtacaaaaa agctgaacga tatcgccacc | 480 |
| | atgggcagca cagccattct ggccctgctg ctggcagtgc tgcagggcgt gtcagctcag | 540 |
| | gtgcagctgg tgcagtctgg cggcggactc gtgaaacctg gcggcagcct gagactgagc | 600 |
| | tgtgccgcca gcggcttcaa cttcaacgac cagtacatga gctggatccg gcaggcccct | 660 |
| | ggcaagggac tggaatgggt gtccttcatc agcggcagcg gcggcaccac ctactacacc | 720 |
| | gatagcgtga agggccggtt caccatcagc cgggacaaca ccaaggacag cctgtacctg | 780 |
| | cagatgaaca gcctgaccgt ggaagatacc gccgtgtact actgcgccag aggcggcaat | 840 |
| | tactacacca gcgtgggcag aggcaccctc gtgacagtgt ctgctggcgg aggcggatca | 900 |
| | ggcggcggag gatcaggggg aggcggaagc ggagcacccg atatccagat gacacagagc | 960 |
| | cccggcaccc tgtctctgag ccctggcgaa agagccatcc tgagctgcag agccagccag | 1020 |
| | agcgtgtccg gatacctggc ttggtatcag cagaagcccg gccaggcccc cagactgctg | 1080 |
| | atctatggcg ccagcagcag agccacaggc atccccgata gattcagcgg ctctggcagc | 1140 |
| | ggcaccgact tcaccctgac aatcagctcc ctgcggcccg aggacatcgg cacctactat | 1200 |
| | tgcaagcagt acatcaacgc ccccttcacc ttcggcggag gcaccaaggt ggaaatcaag | 1260 |
| | ccgcgggcca actttgtata caaaagtgga acgagaaacg taaaatgata taaatatcaa | 1320 |
| | tatattaaat tagattttgc ataaaaaaca gactacataa tactgtaaaa cacaacatat | 1380 |
| | ccagtcacta tgaatcaact acttagatgg tattagtgac ctgtactggg cctcatgggc | 1440 |
| | cttcctttca ctgcccgctt tccagtcggg aaacctgtcg tgccagctgc attaacatgg | 1500 |
| | tcatagctgt ttccttgcgt attgggcgct ctccgcttcc tcgctcactg actcgctgcg | 1560 |
| | ctcggtcgtt cgggtaaagc ctggggtgcc taatgagcaa aaggccagca aaaggccagg | 1620 |
| | aaccgtaaaa aggccgcgtt gctggcgttt ttccataggc tccgcccccc tgacgagcat | 1680 |
| | cacaaaaatc gacgctcaag tcagaggtgg cgaaacccga caggactata aagataccag | 1740 |
| | gcgtttcccc ctggaagctc cctcgtgcgc tctcctgttc cgaccctgcc gcttaccgga | 1800 |
| | tacctgtccg cctttctccc ttcgggaagc gtggcgcttt ctcatagctc acgctgtagg | 1860 |
| | tatctcagtt cggtgtaggt cgttcgctcc aagctgggct gtgtgcacga accccccgtt | 1920 |
| | cagcccgacc gctgcgcctt atccggtaac tatcgtcttg agtccaaccc ggtaagacac | 1980 |
| | gacttatcgc cactggcagc agccactggt aacaggatta gcagagcgag gtatgtaggc | 2040 |
| | ggtgctacag agttcttgaa gtggtggcct aactacggct acactagaag aacagtattt | 2100 |
| | ggtatctgcg ctctgctgaa gccagttacc ttcggaaaaa gagttggtag ctcttgatcc | 2160 |
| | ggcaaacaaa ccaccgctgg tagcggtggt ttttttgttt gcaagcagca gattacgcgc | 2220 |
| | agaaaaaaag gatctcaaga agatcctttg atcttttcta cggggtctga cgctcagtgg | 2280 |
| | aacgaaaact cacgttaagg gattttggtc atgagattat caaaaaggat cttcacctag | 2340 |
| | atccttttaa attaaaaatg aagttttaaa tcaatctaaa gtatatatga gtaaacttgg | 2400 |
| | tctgacagtt attagaaaaa ttcatccagc agacgataaa acgcaatacg ctggctatcc | 2460 |
| | ggtgccgcaa tgccatacag caccagaaaa cgatccgccc attcgccgcc cagttcttcc | 2520 |
| | gcaatatcac gggtggccag cgcaatatcc tgataacgat ccgccacgcc cagacggccg | 2580 |
| | caatcaataa agccgctaaa acggccattt tccaccataa tgttcggcag gcacgcatca | 2640 |
| | ccatgggtca ccaccagatc ttcgccatcc ggcatgctcg ctttcagacg cgcaaacagc | 2700 |
| | tctgccggtg ccaggccctg atgttcttca tccagatcat cctgatccac caggcccgct | 2760 |
| | tccatacggg tacgcgcacg ttcaatacga tgtttcgcct gatgatcaaa cggacaggtc | 2820 |
| | gccgggtcca gggtatgcag acgacgcatg gcatccgcca taatgctcac tttttctgcc | 2880 |
| | ggcgccagat ggctagacag cagatcctga cccggcactt cgcccagcag cagccaatca | 2940 |
| | cggcccgctt cggtcaccac atccagcacc gccgcacacg gaacaccggt ggtggccagc | 3000 |
| | cagctcagac gcgccgcttc atcctgcagc tcgttcagcg caccgctcag atcggttttc | 3060 |
| | acaaacagca ccggacgacc ctgcgcgctc agacgaaaca ccgccgcatc agagcagcca | 3120 |
| | atggtctgct gcgcccaatc atagccaaac agacgttcca cccacgctgc cgggctaccc | 3180 |
| | gcatgcaggc catcctgttc aatcatactc ttcctttttc aatattattg aagcatttat | 3240 |
| | cagggttatt gtctcatgag cggatacata tttgaatgta tttagaaaaa taaacaaata | 3300 |
| | ggggttccgc gcacatttcc ccgaaaagtg ccac | 3334 |
| SEQ ID NO: 33 | ctaaattgta agcgttaata ttttgttaaa attcgcgtta aatttttgtt aaatcagctc | 60 |
| (donor vector | attttttaac caataggccg aaatcggcaa aatcccttat aaatcaaaag aatagaccga | 120 |
| 2, pMK hCD8a | gatagggttg agtggccgct acagggcgct cccattcgcc attcaggctg cgcaactgtt | 180 |
| scaffold TN L5 | gggaagggcg tttcggtgcg ggcctcttcg ctattacgcc agctggcgaa agggggatgt | 240 |
| L2 ) | gctgcaaggc gattaagttg ggtaacgcca gggttttccc agtcacgacg ttgtaaaacg | 300 |
| | acggccagtg agcgcgacgt aatacgactc actatagggc gaattgaagg aaggccgtca | 360 |
| | aggccgcata aataatgatt ttattttgac tgatagtgac ctgttcgttg caacaaattg | 420 |
| | atgagcaatg cttttttata atgcccaact ttgtatacaa aagtggcccg cggacaacaa | 480 |
| | cccctgcccc cagacctcct accccagccc ctacaattgc cagccagcct ctgagcctga | 540 |
| | ggcccgaggc ttgtagacct gctgctggcg gagccgtgca caccagagga ctggatttcg | 600 |
| | cctgcgacat ctacatctgg gcccctctgg ccggcacatg tggcgtgctg ctgctgagcc | 660 |
| | tcgtgatcac cctgtactgc ggctccacca gcggctccgg caagcccggc tctggcgagg | 720 |
| | gctccaccag cggcgactac aaggacgacg atgacaagta ataggatatc ggttcagctt | 780 |
| | tcttgtacaa agttggcatt ataagaaagc attgcttatc aatttgttgc aacgaacagg | 840 |
| | tcactatcag tcaaaataaa atcattattt ctgggcctca tgggccttcc tttcactgcc | 900 |
| | cgctttccag tcgggaaacc tgtcgtgcca gctgcattaa catggtcata gctgtttcct | 960 |
| | tgcgtattgg gcgctctccg cttcctcgct cactgactcg ctgcgctcgg tcgttcgggt | 1020 |
| | aaagcctggg gtgcctaatg agcaaaaggc cagcaaaagg ccaggaaccg taaaaaggcc | 1080 |
| | gcgttgctgg cgtttttcca taggctccgc ccccctgacg agcatcacaa aaatcgacgc | 1140 |
| | tcaagtcaga ggtggcgaaa cccgacagga ctataaagat accaggcgtt tccccctgga | 1200 |
| | agctccctcg tgcgctctcc tgttccgacc ctgccgctta ccggatacct gtccgccttt | 1260 |
| | ctcccttcgg gaagcgtggc gctttctcat agctcacgct gtaggtatct cagttcggtg | 1320 |
| | taggtcgttc gctccaagct gggctgtgtg cacgaacccc ccgttcagcc cgaccgctgc | 1380 |
| | gccttatccg gtaactatcg tcttgagtcc aacccggtaa gacacgactt atcgccactg | 1440 |
| | gcagcagcca ctggtaacag gattagcaga gcgaggtatg taggcggtgc tacagagttc | 1500 |
| | ttgaagtggt ggcctaacta cggctacact agaagaacag tatttggtat ctgcgctctg | 1560 |
| | ctgaagccag ttaccttcgg aaaaagagtt ggtagctctt gatccggcaa acaaaccacc | 1620 |
| | gctggtagcg gtggtttttt tgtttgcaag cagcagatta cgcgcagaaa aaaaggatct | 1680 |
| | caagaagatc ctttgatctt ttctacgggg tctgacgctc agtggaacga aaactcacgt | 1740 |
| | taagggattt tggtcatgag attatcaaaa aggatcttca cctagatcct tttaaattaa | 1800 |
| | aaatgaagtt ttaaatcaat ctaaagtata tatgagtaaa cttggtctga cagttattag | 1860 |
| | aaaaattcat ccagcagacg ataaaacgca atacgctggc tatccggtgc cgcaatgcca | 1920 |
| | tacagcacca gaaaacgatc cgcccattcg ccgcccagtt cttccgcaat atcacgggtg | 1980 |
| | gccagcgcaa tatcctgata acgatccgcc acgcccagac ggccgcaatc aataaagccg | 2040 |
| | ctaaaacggc cattttccac cataatgttc ggcaggcacg catcaccatg ggtcaccacc | 2100 |
| | agatcttcgc catccggcat gctcgctttc agacgcgcaa acagctctgc cggtgccagg | 2160 |
| | ccctgatgtt cttcatccag atcatcctga tccaccaggc ccgcttccat acgggtacgc | 2220 |
| | gcacgttcaa tacgatgttt cgcctgatga tcaaacggac aggtcgccgg gtccagggta | 2280 |
| | tgcagacgac gcatggcatc cgccataatg ctcacttttt ctgccggcgc cagatggcta | 2340 |
| | gacagcagat cctgacccgg cacttcgccc agcagcagcc aatcacggcc cgcttcggtc | 2400 |
| | accacatcca gcaccgccgc acacggaaca ccggtggtgg ccagccagct cagacgcgcc | 2460 |
| | gcttcatcct gcagctcgtt cagcgcaccg ctcagatcgg ttttcacaaa cagcaccgga | 2520 |
| | cgaccctgcg cgctcagacg aaacaccgcc gcatcagagc agccaatggt ctgctgcgcc | 2580 |
| | caatcatagc caaacagacg ttccacccac gctgccgggc tacccgcatg caggccatcc | 2640 |
| | tgttcaatca tactcttcct ttttcaatat tattgaagca tttatcaggg ttattgtctc | 2700 |
| | atgagcggat acatatttga atgtatttag aaaaataaac aaataggggt tccgcgcaca | 2760 |
| | tttccccgaa aagtgccac | 2779 |
| SEQ ID NO: 34 | cgataaccct aattcgatag catatgcttc ccgttgggta acatatgcta ttgaattagg | 60 |
| (Final vector | gttagtctgg atagtatata ctactacccg ggaagcatat gctacccgtt tagggttcac | 120 |
| used for | cggtgatgcc ggccacgatg cgtccggcgt agaggatcta atgtgagtta gctcactcat | 180 |
| lentiviral | taggcacccc aggctttaca ctttatgctt ccggctcgta tgttgtgtgg aattgtgagc | 240 |
| production, | ggataacaat ttcacacagg aaacagctat gaccatgatt acgccaagcg cgcaattaac | 300 |
| pLV430lG 7C12 | cctcactaaa gggaacaaaa gctggagctg caagcttaat gtagtcttat gcaatactct | 360 |
| scFV mlgG hCD8 | tgtagtcttg caacatggta acgatgagtt agcaacatgc cttacaagga gagaaaaagc | 420 |
| flag) | accgtgcatg ccgattggtg gaagtaaggt ggtacgatcg tgccttatta ggaaggcaac | 480 |
| | agacgggtct gacatggatt ggacgaacca ctgaattgcc gcattgcaga gatattgtat | 540 |
| | ttaagtgcct agctcgatac ataaacgggt ctctctggtt agaccagatc tgagcctggg | 600 |
| | agctctctgg ctaactaggg aacccactgc ttaagcctca ataaagcttg ccttgagtgc | 660 |
| | ttcaagtagt gtgtgcccgt ctgttgtgtg actctggtaa ctagagatcc ctcagaccct | 720 |
| | tttagtcagt gtggaaaatc tctagcagtg gcgcccgaac agggacttga aagcgaaagg | 780 |
| | gaaaccagag gagctctctc gacgcaggac tcggcttgct gaagcgcgca cggcaagagg | 840 |
| | cgaggggcgg cgactggtga gtacgccaaa aattttgact agcggaggct agaaggagag | 900 |
| | agatgggtgc gagagcgtca gtattaagcg ggggagaatt agatcgcgat gggaaaaaat | 960 |
| | tcggttaagg ccagggggaa agaaaaaata taaattaaaa catatagtat gggcaagcag | 1020 |
| | ggagctagaa cgattcgcag ttaatcctgg cctgttagaa acatcagaag gctgtagaca | 1080 |
| | aatactggga cagctacaac catcccttca gacaggatca gaagaactta gatcattata | 1140 |
| | taatacagta gcaaccctct attgtgtgca tcaaaggata gagataaaag acaccaagga | 1200 |
| | agctttagac aagatagagg aagagcaaaa caaaagtaag accaccgcac agcaagcggc | 1260 |
| | cgctgatctt cagacctgga ggaggagata tgagggacaa ttggagaagt gaattatata | 1320 |
| | aatataaagt agtaaaaatt gaaccattag gagtagcacc caccaaggca aagagaagag | 1380 |
| | tggtgcagag agaaaaaaga gcagtgggaa taggagcttt gttccttggg ttcttgggag | 1440 |
| | cagcaggaag cactatgggc gcagcgtcaa tgacgctgac ggtacaggcc agacaattat | 1500 |
| | tgtctggtat agtgcagcag cagaacaatt tgctgagggc tattgaggcg caacagcatc | 1560 |
| | tgttgcaact cacagtctgg ggcatcaagc agctccaggc aagaatcctg gctgtggaaa | 1620 |
| | gatacctaaa ggatcaacag ctcctgggga tttggggttg ctctggaaaa ctcatttgca | 1680 |
| | ccactgctgt gccttggaat gctagttgga gtaataaatc tctggaacag atttggaatc | 1740 |
| | acacgacctg gatggagtgg gacagagaaa ttaacaatta cacaagctta atacactcct | 1800 |
| | taattgaaga atcgcaaaac cagcaagaaa agaatgaaca agaattattg gaattagata | 1860 |
| | aatgggcaag tttgtggaat tggtttaaca taacaaattg gctgtggtat ataaaattat | 1920 |
| | tcataatgat agtaggaggc ttggtaggtt taagaatagt ttttgctgta ctttctatag | 1980 |
| | tgaatagagt taggcaggga tattcaccat tatcgtttca gacccacctc ccaaccccga | 2040 |
| | ggggacccga caggcccgaa ggaatagaag aagaaggtgg agagagagac agagacagat | 2100 |
| | ccattcgatt agtgaacgga tctcgacggt atcggtttta aaagaaaagg ggggattggg | 2160 |
| | gggtacagtg caggggaaag aatagtagac ataatagcaa cagacataca aactaaagaa | 2220 |
| | ttacaaaaac aaattacaaa aattcaaaat tttatcgatt ttatttagtc tccagaaaaa | 2280 |
| | ggggggaatg aaagacccca cctgtaggtt tggcaagcta gcttaagtaa cgccattttg | 2340 |
| | caaggcatgg aaaatacata actgagaata gagaagttca gatcaaggtt aggaacagag | 2400 |
| | agacagcaga atatgggcca aacaggatat ctgtggtaag cagttcctgc cccggctcag | 2460 |
| | ggccaagaac agatggtccc cagatgcggt cccgccctca gcagtttcta gagaaccatc | 2520 |
| | agatgtttcc agggtgcccc aaggacctga aatgaccctg tgccttattt gaactaacca | 2580 |
| | atcagttcgc ttctcgcttc tgttcgcgcg cttctgctcc ccgagctcaa taaaagagcc | 2640 |
| | cacaacccct cactcggcgc gccagtcctc cgatagactg cgtcgcccgg gtaccgatat | 2700 |
| | caccaacttt gtacaaaaaa gctgaacgat atcgccacca tgggcagcac agccattctg | 2760 |
| | gccctgctgc tggcagtgct gcagggcgtg tcagctcagg tgcagctggt gcagtctggc | 2820 |
| | ggcggactcg tgaaacctgg cggcagcctg agactgagct gtgccgccag cggcttcaac | 2880 |
| | ttcaacgacc agtacatgag ctggatccgg caggcccctg gcaagggact ggaatgggtg | 2940 |
| | tccttcatca gcggcagcgg cggcaccacc tactacaccg atagcgtgaa gggccggttc | 3000 |
| | accatcagcc gggacaacac caaggacagc ctgtacctgc agatgaacag cctgaccgtg | 3060 |
| | gaagataccg ccgtgtacta ctgcgccaga ggcggcaatt actacaccag cgtgggcaga | 3120 |
| | ggcaccctcg tgacagtgtc tgctggcgga ggcggatcag gcggcggagg atcaggggga | 3180 |
| | ggcggaagcg gagcacccga tatccagatg acacagagcc ccggcaccct gtctctgagc | 3240 |
| | cctggcgaaa gagccatcct gagctgcaga gccagccaga gcgtgtccgg atacctggct | 3300 |
| | tggtatcagc agaagcccgg ccaggccccc agactgctga tctatggcgc cagcagcaga | 3360 |
| | gccacaggca tccccgatag attcagcggc tctggcagcg gcaccgactt caccctgaca | 3420 |
| | atcagctccc tgcggcccga ggacatcggc acctactatt gcaagcagta catcaacgcc | 3480 |
| | cccttcacct tcggcggagg caccaaggtg gaaatcaagc cgcgggccaa ctttgtatac | 3540 |
| | aaaagtggcc cgcggacaac aacccctgcc cccagacctc ctaccccagc ccctacaatt | 3600 |
| | gccagccagc ctctgagcct gaggcccgag gcttgtagac ctgctgctgg cggagccgtg | 3660 |
| | cacaccagag gactggattt cgcctgcgac atctacatct gggcccctct ggccggcaca | 3720 |
| | tgtggcgtgc tgctgctgag cctcgtgatc accctgtact gcggctccac cagcggctcc | 3780 |
| | ggcaagcccg gctctggcga gggctccacc agcggcgact acaaggacga cgatgacaag | 3840 |
| | taataggata tcggttcagc tttcttgtac aaagttggga ttcgagttaa ttaagttaac | 3900 |
| | gaattccccc cctctccctc ccccccccct aacgttactg gccgaagccg cttggaataa | 3960 |
| | ggccggtgtg cgtttgtcta tatgttattt tccaccatat tgccgtcttt tggcaatgtg | 4020 |
| | agggcccgga aacctggccc tgtcttcttg acgagcattc ctaggggtct ttcccctctc | 4080 |
| | gccaaaggaa tgcaaggtct gttgaatgtc gtgaaggaag cagttcctct ggaagcttct | 4140 |
| | tgaagacaaa caacgtctgt agcgaccctt tgcaggcagc ggaacccccc acctggcgac | 4200 |
| | aggtgcctct gcggccaaaa gccacgtgta taagatacac ctgcaaaggc ggcacaaccc | 4260 |
| | cagtgccacg ttgtgagttg gatagttgtg gaaagagtca aatggctctc ctcaagcgta | 4320 |
| | ttcaacaagg ggctgaagga tgcccagaag gtaccccatt gtatgggatc tgatctgggg | 4380 |
| | cctcggtgca catgctttac atgtgtttag tcgaggttaa aaaacgtcta ggccccccga | 4440 |
| | accacgggga cgtggttttc ctttgaaaaa cacgatgata atatggccac aaccatggga | 4500 |
| | ggcggaagcg gcggaggctc ccctcgaggc accatggtga gcaagggcga ggagctgttc | 4560 |
| | accggggtgg tgcccatcct ggtcgagctg gacggcgacg taaacggcca caagttcagc | 4620 |
| | gtgtccggcg agggcgaggg cgatgccacc tacggcaagc tgaccctgaa gttcatctgc | 4680 |
| | accaccggca agctgcccgt gccctggccc accctcgtga ccaccctgac ctacggcgtg | 4740 |
| | cagtgcttca gccgctaccc cgaccacatg aagcagcacg acttcttcaa gtccgccatg | 4800 |
| | cccgaaggct acgtccagga gcgcaccatc ttcttcaagg acgacggcaa ctacaagacc | 4860 |
| | cgcgccgagg tgaagttcga gggcgacacc ctggtgaacc gcatcgagct gaagggcatc | 4920 |
| | gacttcaagg aggacggcaa catcctgggg cacaagctgg agtacaacta caacagccac | 4980 |
| | aacgtctata tcatggccga caagcagaag aacggcatca aggtgaactt caagatccgc | 5040 |
| | cacaacatcg aggacggcag cgtgcagctc gccgaccact accagcagaa cacccccatc | 5100 |
| | ggcgacggcc ccgtgctgct gcccgacaac cactacctga gcacccagtc cgccctgagc | 5160 |
| | aaagacccca acgagaagcg cgatcacatg gtcctgctgg agttcgtgac cgccgccggg | 5220 |
| | atcactctcg gcatggacga gctgtacaag taacgcgtcc cgggtctaga gctagcggta | 5280 |
| | ccatgcatta cgtagtcgac gacttaatta agctagccta gtgccatttg ttcagtggtt | 5340 |
| | cgtagggctt tcccccactg tttggctttc agttatatgg atgatgtggt attgggggcc | 5400 |
| | aagtctgtac agcatcttga gtcccttttt accgctgtta ccaattttct tttgtctttg | 5460 |
| | ggtatacatt taaaccctaa caaaacaaag agatggggtt actctctaaa ttttatgggt | 5520 |
| | tatgtcattg gatgttatgg gtccttgcca caagaacaca tcatacaaaa aatcaaagaa | 5580 |
| | tgttttagaa aacttcctat taacaggcct attgattgga aagtatgtca acgaattgtg | 5640 |
| | ggtcttttgg gttttgctgc cccttttaca caatgtggtt atcctgcgtt gatgcctttg | 5700 |
| | tatgcatgta ttcaatctaa gcaggctttc actttctcgc caacttacaa ggcctttctg | 5760 |
| | tgtaaacaat acctgaacct ttaccccgtt gcccggcaac ggccaggtct gtgccaagtg | 5820 |
| | tttgctgacg caacccccac tggctggggc ttggtcatgg gccatcagcg catgcgtgga | 5880 |
| | accttttcgg ctcctctgcc gatccatact gcggaactcc tagccgcttg ttttgctcgc | 5940 |
| | agcaggtctg gagcaaacat tatcgggact gataactctg ttgtcctatc ccgcaaatat | 6000 |
| | acatcgtttc catggctgct aggctgtgct gccaactgga tcctgcgcgg gacgtccttt | 6060 |
| | gtttacgtcc cgtcggcgct gaatcctgcg gacgaccctt ctcggggtcg cttgggactc | 6120 |
| | tctcgtcccc ttctccgtct gccgttccga ccgaccacgg ggcgcacctc tctttacgcg | 6180 |
| | gactccccgt ctgtgccttc tcatctgccg gaccgtgtgc acttcgcttc acctctgcac | 6240 |
| | gtcgcatgga gaccaccgtg aacgcccacc aaatattgcc caaggtctta cataagagga | 6300 |
| | ctcttggact ctcagcaatg tcaacgaccg accttgaggc atacttcaaa gactgtttgt | 6360 |
| | ttaaagactg ggaggagttg ggggaggaga ttaggttaaa ggtctttgta ctaggaggct | 6420 |
| | gtaggcataa attggtctgc gcaccagcac catggcgcaa tcactagagc ggggtacctt | 6480 |
| | taagaccaat gacttacaag gcagctgtag atcttagcca ctttttaaaa gaaaaggggg | 6540 |
| | gactggaagg gctaattcac tcccaacgaa gacaagatct gctttttgct tgtactgggt | 6600 |
| | ctctctggtt agaccagatc tgagcctggg agctctctgg ctaactaggg aacccactgc | 6660 |
| | ttaagcctca ataaagcttg ccttgagtgc ttcaagtagt gtgtgcccgt ctgttgtgtg | 6720 |
| | actctggtaa ctagagatcc ctcagaccct tttagtcagt gtggaaaatc tctagcagta | 6780 |
| | gtagttcatg tcatcttatt attcagtatt tataacttgc aaagaaatga atatcagaga | 6840 |
| | gtgagaggaa cttgtttatt gcagcttata atggttacaa ataaagcaat agcatcacaa | 6900 |
| | atttcacaaa taaagcattt ttttcactgc attctagttg tggtttgtcc aaactcatca | 6960 |
| | atgtatctta tcatgtctgg ctctagctat cccgccccta actccgccca tcccgcccct | 7020 |
| | aactccgccc agttccgccc attctccgcc ccatggctga ctaatttttt ttatttatgc | 7080 |
| | agaggccgag gccggatccc ttgagtggct ttcatcctgg agcagacttt gcagtctgtg | 7140 |
| | gactgcaaca caacattgcc tttatgtgta actcttggct gaagctctta caccaatgct | 7200 |
| | gggggacatg tacctcccag gggcccagga agactacggg aggctacacc aacgtcaatc | 7260 |
| | agaggggcct gtgtagctac cgataagcgg accctcaaga gggcattagc aatagtgttt | 7320 |
| | ataaggcccc cttgttaatt cttgaagacg aaagggcctc gtgatacgcc tatttttata | 7380 |
| | ggttaatgtc atgataataa tggtttctta gacgtcaggt ggcacttttc ggggaaatgt | 7440 |
| | gcgcggaacc cctatttgtt tatttttcta aatacattca aatatgtatc cgctcatgag | 7500 |
| | acaataaccc tgataaatgc ttcaataata ttgaaaaagg aagagtatga gtattcaaca | 7560 |
| | tttccgtgtc gcccttattc ccttttttgc ggcattttgc cttcctgttt ttgctcaccc | 7620 |
| | agaaacgctg gtgaaagtaa aagatgctga agatcagttg ggtgcacgag tgggttacat | 7680 |
| | cgaactggat ctcaacagcg gtaagatcct tgagagtttt cgccccgaag aacgttttcc | 7740 |
| | aatgatgagc acttttaaag ttctgctatg tggcgcggta ttatcccgtg ttgacgccgg | 7800 |
| | gcaagagcaa ctcggtcgcc gcatacacta ttctcagaat gacttggttg agtactcacc | 7860 |
| | agtcacagaa aagcatctta cggatggcat gacagtaaga gaattatgca gtgctgccat | 7920 |
| | aaccatgagt gataacactg cggccaactt acttctgaca acgatcggag gaccgaagga | 7980 |
| | gctaaccgct tttttgcaca acatggggga tcatgtaact cgccttgatc gttgggaacc | 8040 |
| | ggagctgaat gaagccatac caaacgacga gcgtgacacc acgatgcctg cagcaatggc | 8100 |
| | aacaacgttg cgcaaactat taactggcga actacttact ctagcttccc ggcaacaatt | 8160 |
| | aatagactgg atggaggcgg ataaagttgc aggaccactt ctgcgctcgg cccttccggc | 8220 |
| | tggctggttt attgctgata aatctggagc cggtgagcgt gggtctcgcg gtatcattgc | 8280 |
| | agcactgggg ccagatggta agccctcccg tatcgtagtt atctacacga cggggagtca | 8340 |
| | ggcaactatg gatgaacgaa atagacagat cgctgagata ggtgcctcac tgattaagca | 8400 |
| | ttggtaactg tcagaccaag tttactcata tatactttag attgatttaa aacttcattt | 8460 |
| | ttaatttaaa aggatctagg tgaagatcct ttttgataat ctcatgacca aaatccctta | 8520 |
| | acgtgagttt tcgttccact gagcgtcaga ccccgtagaa aagatcaaag gatcttcttg | 8580 |
| | agatcctttt tttctgcgcg taatctgctg cttgcaaaca aaaaaaccac cgctaccagc | 8640 |
| | ggtggtttgt ttgccggatc aagagctacc aactcttttt ccgaaggtaa ctggcttcag | 8700 |
| | cagagcgcag ataccaaata ctgtccttct agtgtagccg tagttaggcc accacttcaa | 8760 |
| | gaactctgta gcaccgccta catacctcgc tctgctaatc ctgttaccag tggctgctgc | 8820 |
| | cagtggcgat aagtcgtgtc ttaccgggtt ggactcaaga cgatagttac cggataaggc | 8880 |
| | gcagcggtcg ggctgaacgg ggggttcgtg cacacagccc agcttggagc gaacgaccta | 8940 |
| | caccgaactg agatacctac agcgtgagca ttgagaaagc gccacgcttc ccgaagggag | 9000 |
| | aaaggcggac aggtatccgg taagcggcag ggtcggaaca ggagagcgca cgagggagct | 9060 |
| | tccaggggga aacgcctggt atctttatag tcctgtcggg tttcgccacc tctgacttga | 9120 |
| | gcgtcgattt ttgtgatgct cgtcaggggg gcggagccta tggaaaaacg ccagcaacgc | 9180 |
| | ggccttttta cggttcctgg ccttttgctg gcctttttga agctgtccct gatggtcgtc | 9240 |
| | atctacctgc ctggacagca tggcctgcaa cgcgggcatc ccgatgccgc cggaagcgag | 9300 |
| | aagaatcata atggggaagg ccatccagcc tcgcgtcg | 9338 |
| SEQ ID NO: 35 | cgataaccct aattcgatag catatgcttc ccgttgggta acatatgcta ttgaattagg | 60 |
| (destination | gttagtctgg atagtatata ctactacccg ggaagcatat gctacccgtt tagggttcac | 120 |
| vector, | cggtgatgcc ggccacgatg cgtccggcgt agaggatcta atgtgagtta gctcactcat | 180 |
| pLV430lG) | taggcacccc aggctttaca ctttatgctt ccggctcgta tgttgtgtgg aattgtgagc | 240 |
| | ggataacaat ttcacacagg aaacagctat gaccatgatt acgccaagcg cgcaattaac | 300 |
| | cctcactaaa gggaacaaaa gctggagctg caagcttaat gtagtcttat gcaatactct | 360 |
| | tgtagtcttg caacatggta acgatgagtt agcaacatgc cttacaagga gagaaaaagc | 420 |
| | accgtgcatg ccgattggtg gaagtaaggt ggtacgatcg tgccttatta ggaaggcaac | 480 |
| | agacgggtct gacatggatt ggacgaacca ctgaattgcc gcattgcaga gatattgtat | 540 |
| | ttaagtgcct agctcgatac ataaacgggt ctctctggtt agaccagatc tgagcctggg | 600 |
| | agctctctgg ctaactaggg aacccactgc ttaagcctca ataaagcttg ccttgagtgc | 660 |
| | ttcaagtagt gtgtgcccgt ctgttgtgtg actctggtaa ctagagatcc ctcagaccct | 720 |
| | tttagtcagt gtggaaaatc tctagcagtg gcgcccgaac agggacttga aagcgaaagg | 780 |
| | gaaaccagag gagctctctc gacgcaggac tcggcttgct gaagcgcgca cggcaagagg | 840 |
| | cgaggggcgg cgactggtga gtacgccaaa aattttgact agcggaggct agaaggagag | 900 |
| | agatgggtgc gagagcgtca gtattaagcg ggggagaatt agatcgcgat gggaaaaaat | 960 |
| | tcggttaagg ccagggggaa agaaaaaata taaattaaaa catatagtat gggcaagcag | 1020 |
| | ggagctagaa cgattcgcag ttaatcctgg cctgttagaa acatcagaag gctgtagaca | 1080 |
| | aatactggga cagctacaac catcccttca gacaggatca gaagaactta gatcattata | 1140 |
| | taatacagta gcaaccctct attgtgtgca tcaaaggata gagataaaag acaccaagga | 1200 |
| | agctttagac aagatagagg aagagcaaaa caaaagtaag accaccgcac agcaagcggc | 1260 |
| | cgctgatctt cagacctgga ggaggagata tgagggacaa ttggagaagt gaattatata | 1320 |
| | aatataaagt agtaaaaatt gaaccattag gagtagcacc caccaaggca aagagaagag | 1380 |
| | tggtgcagag agaaaaaaga gcagtgggaa taggagcttt gttccttggg ttcttgggag | 1440 |
| | cagcaggaag cactatgggc gcagcgtcaa tgacgctgac ggtacaggcc agacaattat | 1500 |
| | tgtctggtat agtgcagcag cagaacaatt tgctgagggc tattgaggcg caacagcatc | 1560 |
| | tgttgcaact cacagtctgg ggcatcaagc agctccaggc aagaatcctg gctgtggaaa | 1620 |
| | gatacctaaa ggatcaacag ctcctgggga tttggggttg ctctggaaaa ctcatttgca | 1680 |
| | ccactgctgt gccttggaat gctagttgga gtaataaatc tctggaacag atttggaatc | 1740 |
| | acacgacctg gatggagtgg gacagagaaa ttaacaatta cacaagctta atacactcct | 1800 |
| | taattgaaga atcgcaaaac cagcaagaaa agaatgaaca agaattattg gaattagata | 1860 |
| | aatgggcaag tttgtggaat tggtttaaca taacaaattg gctgtggtat ataaaattat | 1920 |
| | tcataatgat agtaggaggc ttggtaggtt taagaatagt ttttgctgta ctttctatag | 1980 |
| | tgaatagagt taggcaggga tattcaccat tatcgtttca gacccacctc ccaaccccga | 2040 |
| | ggggacccga caggcccgaa ggaatagaag aagaaggtgg agagagagac agagacagat | 2100 |
| | ccattcgatt agtgaacgga tctcgacggt atcggtttta aaagaaaagg ggggattggg | 2160 |
| | gggtacagtg caggggaaag aatagtagac ataatagcaa cagacataca aactaaagaa | 2220 |
| | ttacaaaaac aaattacaaa aattcaaaat tttatcgatt ttatttagtc tccagaaaaa | 2280 |
| | ggggggaatg aaagacccca cctgtaggtt tggcaagcta gcttaagtaa cgccattttg | 2340 |
| | caaggcatgg aaaatacata actgagaata gagaagttca gatcaaggtt aggaacagag | 2400 |
| | agacagcaga atatgggcca aacaggatat ctgtggtaag cagttcctgc cccggctcag | 2460 |
| | ggccaagaac agatggtccc cagatgcggt cccgccctca gcagtttcta gagaaccatc | 2520 |
| | agatgtttcc agggtgcccc aaggacctga aatgaccctg tgccttattt gaactaacca | 2580 |
| | atcagttcgc ttctcgcttc tgttcgcgcg cttctgctcc ccgagctcaa taaaagagcc | 2640 |
| | cacaacccct cactcggcgc gccagtcctc cgatagactg cgtcgcccgg gtaccgatat | 2700 |
| | cacaagtttg tacaaaaaag ctgaacgaga aacgtaaaat gatataaata tcaatatatt | 2760 |
| | aaattagatt ttgcataaaa aacagactac ataatactgt aaaacacaac atatccagtc | 2820 |
| | actatggcgg ccgcattagg caccccaggc tttacacttt atgcttccgg ctcgtataat | 2880 |
| | gtgtggattt tgagttagga tccgtcgaga ttttcaggag ctaaggaagc taaaatggag | 2940 |
| | aaaaaaatca ctggatatac caccgttgat atatcccaat ggcatcgtaa agaacatttt | 3000 |
| | gaggcatttc agtcagttgc tcaatgtacc tataaccaga ccgttcagct ggatattacg | 3060 |
| | gcctttttaa agaccgtaaa gaaaaataag cacaagtttt atccggcctt tattcacatt | 3120 |
| | cttgcccgcc tgatgaatgc tcatccggaa ttccgtatgg caatgaaaga cggtgagctg | 3180 |
| | gtgatatggg atagtgttca cccttgttac accgttttcc atgagcaaac tgaaacgttt | 3240 |
| | tcatcgctct ggagtgaata ccacgacgat ttccggcagt ttctacacat atattcgcaa | 3300 |
| | gatgtggcgt gttacggtga aaacctggcc tatttcccta aagggtttat tgagaatatg | 3360 |
| | tttttcgtct cagccaatcc ctgggtgagt ttcaccagtt ttgatttaaa cgtggccaat | 3420 |
| | atggacaact tcttcgcccc cgttttcacc atgggcaaat attatacgca aggcgacaag | 3480 |
| | gtgctgatgc cgctggcgat tcaggttcat catgccgttt gtgatggctt ccatgtcggc | 3540 |
| | agaatgctta atgaattaca acagtactgc gatgagtggc agggcggggc gtaaatggat | 3600 |
| | ccggcttact aaaagccaga taacagtatg cgtatttgcg cgctgatttt tgcggtataa | 3660 |
| | gaatatatac tgatatgtat acccgaagta tgtcaaaaag aggtatgcta tgaagcagcg | 3720 |
| | tattacagtg acagttgaca gcgacagcta tcagttgctc aaggcatata tgatgtcaat | 3780 |
| | atctccggtc tggtaagcac aaccatgcag aatgaagccc gtcgtctgcg tgccgaacgc | 3840 |
| | tggaaagcgg aaaatcagga agggatggct gaggtcgccc ggtttattga aatgaacggc | 3900 |
| | tcttttgctg acgagaacag gggctggtga aatgcagttt aaggtttaca cctataaaag | 3960 |
| | agagagccgt tatcgtctgt ttgtggatgt acagagtgat attattgaca cgcccgggcg | 4020 |
| | acggatggtg atccccctgg ccagtgcacg tctgctgtca gataaagtct cccgtgaact | 4080 |
| | ttacccggtg gtgcatatcg gggatgaaag ctggcgcatg atgaccaccg atatggccag | 4140 |
| | tgtgccggtc tccgttatcg gggaagaagt ggctgatctc agccaccgcg aaaatgacat | 4200 |
| | caaaaacgcc attaacctga tgttctgggg aatataaatg tcaggctccc ttatacacag | 4260 |
| | ccagtctgca ggtcgaccat agtgactgga tatgttgtgt tttacagtat tatgtagtct | 4320 |
| | gttttttatg caaaatctaa tttaatatat tgatatttat atcattttac gtttctcgtt | 4380 |
| | cagctttctt gtacaaagtg gtgattcgag ttaattaagt taacgaattc cccccctctc | 4440 |
| | cctccccccc ccctaacgtt actggccgaa gccgcttgga ataaggccgg tgtgcgtttg | 4500 |
| | tctatatgtt attttccacc atattgccgt cttttggcaa tgtgagggcc cggaaacctg | 4560 |
| | gccctgtctt cttgacgagc attcctaggg gtctttcccc tctcgccaaa ggaatgcaag | 4620 |
| | gtctgttgaa tgtcgtgaag gaagcagttc ctctggaagc ttcttgaaga caaacaacgt | 4680 |
| | ctgtagcgac cctttgcagg cagcggaacc ccccacctgg cgacaggtgc ctctgcggcc | 4740 |
| | aaaagccacg tgtataagat acacctgcaa aggcggcaca accccagtgc cacgttgtga | 4800 |
| | gttggatagt tgtggaaaga gtcaaatggc tctcctcaag cgtattcaac aaggggctga | 4860 |
| | aggatgccca gaaggtaccc cattgtatgg gatctgatct ggggcctcgg tgcacatgct | 4920 |
| | ttacatgtgt ttagtcgagg ttaaaaaacg tctaggcccc ccgaaccacg gggacgtggt | 4980 |
| | tttcctttga aaaacacgat gataatatgg ccacaaccat gggaggcgga agcggcggag | 5040 |
| | gctcccctcg aggcaccatg gtgagcaagg gcgaggagct gttcaccggg gtggtgccca | 5100 |
| | tcctggtcga gctggacggc gacgtaaacg gccacaagtt cagcgtgtcc ggcgagggcg | 5160 |
| | agggcgatgc cacctacggc aagctgaccc tgaagttcat ctgcaccacc ggcaagctgc | 5220 |
| | ccgtgccctg gcccaccctc gtgaccaccc tgacctacgg cgtgcagtgc ttcagccgct | 5280 |
| | accccgacca catgaagcag cacgacttct tcaagtccgc catgcccgaa ggctacgtcc | 5340 |
| | aggagcgcac catcttcttc aaggacgacg gcaactacaa gacccgcgcc gaggtgaagt | 5400 |
| | tcgagggcga caccctggtg aaccgcatcg agctgaaggg catcgacttc aaggaggacg | 5460 |
| | gcaacatcct ggggcacaag ctggagtaca actacaacag ccacaacgtc tatatcatgg | 5520 |
| | ccgacaagca gaagaacggc atcaaggtga acttcaagat ccgccacaac atcgaggacg | 5580 |
| | gcagcgtgca gctcgccgac cactaccagc agaacacccc catcggcgac ggccccgtgc | 5640 |
| | tgctgcccga caaccactac ctgagcaccc agtccgccct gagcaaagac cccaacgaga | 5700 |
| | agcgcgatca catggtcctg ctggagttcg tgaccgccgc cgggatcact ctcggcatgg | 5760 |
| | acgagctgta caagtaacgc gtcccgggtc tagagctagc ggtaccatgc attacgtagt | 5820 |
| | cgacgactta attaagctag cctagtgcca tttgttcagt ggttcgtagg gctttccccc | 5880 |
| | actgtttggc tttcagttat atggatgatg tggtattggg ggccaagtct gtacagcatc | 5940 |
| | ttgagtccct ttttaccgct gttaccaatt ttcttttgtc tttgggtata catttaaacc | 6000 |
| | ctaacaaaac aaagagatgg ggttactctc taaattttat gggttatgtc attggatgtt | 6060 |
| | atgggtcctt gccacaagaa cacatcatac aaaaaatcaa agaatgtttt agaaaacttc | 6120 |
| | ctattaacag gcctattgat tggaaagtat gtcaacgaat tgtgggtctt ttgggttttg | 6180 |
| | ctgccccttt tacacaatgt ggttatcctg cgttgatgcc tttgtatgca tgtattcaat | 6240 |
| | ctaagcaggc tttcactttc tcgccaactt acaaggcctt tctgtgtaaa caatacctga | 6300 |
| | acctttaccc cgttgcccgg caacggccag gtctgtgcca agtgtttgct gacgcaaccc | 6360 |
| | ccactggctg gggcttggtc atgggccatc agcgcatgcg tggaaccttt tcggctcctc | 6420 |
| | tgccgatcca tactgcggaa ctcctagccg cttgttttgc tcgcagcagg tctggagcaa | 6480 |
| | acattatcgg gactgataac tctgttgtcc tatcccgcaa atatacatcg tttccatggc | 6540 |
| | tgctaggctg tgctgccaac tggatcctgc gcgggacgtc ctttgtttac gtcccgtcgg | 6600 |
| | cgctgaatcc tgcggacgac ccttctcggg gtcgcttggg actctctcgt ccccttctcc | 6660 |
| | gtctgccgtt ccgaccgacc acggggcgca cctctcttta cgcggactcc ccgtctgtgc | 6720 |
| | cttctcatct gccggaccgt gtgcacttcg cttcacctct gcacgtcgca tggagaccac | 6780 |
| | cgtgaacgcc caccaaatat tgcccaaggt cttacataag aggactcttg gactctcagc | 6840 |
| | aatgtcaacg accgaccttg aggcatactt caaagactgt ttgtttaaag actgggagga | 6900 |
| | gttgggggag gagattaggt taaaggtctt tgtactagga ggctgtaggc ataaattggt | 6960 |
| | ctgcgcacca gcaccatggc gcaatcacta gagcggggta cctttaagac caatgactta | 7020 |
| | caaggcagct gtagatctta gccacttttt aaaagaaaag gggggactgg aagggctaat | 7080 |
| | tcactcccaa cgaagacaag atctgctttt tgcttgtact gggtctctct ggttagacca | 7140 |
| | gatctgagcc tgggagctct ctggctaact agggaaccca ctgcttaagc ctcaataaag | 7200 |
| | cttgccttga gtgcttcaag tagtgtgtgc ccgtctgttg tgtgactctg gtaactagag | 7260 |
| | atccctcaga cccttttagt cagtgtggaa aatctctagc agtagtagtt catgtcatct | 7320 |
| | tattattcag tatttataac ttgcaaagaa atgaatatca gagagtgaga ggaacttgtt | 7380 |
| | tattgcagct tataatggtt acaaataaag caatagcatc acaaatttca caaataaagc | 7440 |
| | atttttttca ctgcattcta gttgtggttt gtccaaactc atcaatgtat cttatcatgt | 7500 |
| | ctggctctag ctatcccgcc cctaactccg cccatcccgc ccctaactcc gcccagttcc | 7560 |
| | gcccattctc cgccccatgg ctgactaatt ttttttattt atgcagaggc cgaggccgga | 7620 |
| | tcccttgagt ggctttcatc ctggagcaga ctttgcagtc tgtggactgc aacacaacat | 7680 |
| | tgcctttatg tgtaactctt ggctgaagct cttacaccaa tgctggggga catgtacctc | 7740 |
| | ccaggggccc aggaagacta cgggaggcta caccaacgtc aatcagaggg gcctgtgtag | 7800 |
| | ctaccgataa gcggaccctc aagagggcat tagcaatagt gtttataagg cccccttgtt | 7860 |
| | aattcttgaa gacgaaaggg cctcgtgata cgcctatttt tataggttaa tgtcatgata | 7920 |
| | ataatggttt cttagacgtc aggtggcact tttcggggaa atgtgcgcgg aacccctatt | 7980 |
| | tgtttatttt tctaaataca ttcaaatatg tatccgctca tgagacaata accctgataa | 8040 |
| | atgcttcaat aatattgaaa aaggaagagt atgagtattc aacatttccg tgtcgccctt | 8100 |
| | attccctttt ttgcggcatt ttgccttcct gtttttgctc acccagaaac gctggtgaaa | 8160 |
| | gtaaaagatg ctgaagatca gttgggtgca cgagtgggtt acatcgaact ggatctcaac | 8220 |
| | agcggtaaga tccttgagag ttttcgcccc gaagaacgtt ttccaatgat gagcactttt | 8280 |
| | aaagttctgc tatgtggcgc ggtattatcc cgtgttgacg ccgggcaaga gcaactcggt | 8340 |
| | cgccgcatac actattctca gaatgacttg gttgagtact caccagtcac agaaaagcat | 8400 |
| | cttacggatg gcatgacagt aagagaatta tgcagtgctg ccataaccat gagtgataac | 8460 |
| | actgcggcca acttacttct gacaacgatc ggaggaccga aggagctaac cgcttttttg | 8520 |
| | cacaacatgg gggatcatgt aactcgcctt gatcgttggg aaccggagct gaatgaagcc | 8580 |
| | ataccaaacg acgagcgtga caccacgatg cctgcagcaa tggcaacaac gttgcgcaaa | 8640 |
| | ctattaactg gcgaactact tactctagct tcccggcaac aattaataga ctggatggag | 8700 |
| | gcggataaag ttgcaggacc acttctgcgc tcggcccttc cggctggctg gtttattgct | 8760 |
| | gataaatctg gagccggtga gcgtgggtct cgcggtatca ttgcagcact ggggccagat | 8820 |
| | ggtaagccct cccgtatcgt agttatctac acgacgggga gtcaggcaac tatggatgaa | 8880 |
| | cgaaatagac agatcgctga gataggtgcc tcactgatta agcattggta actgtcagac | 8940 |
| | caagtttact catatatact ttagattgat ttaaaacttc atttttaatt taaaaggatc | 9000 |
| | taggtgaaga tcctttttga taatctcatg accaaaatcc cttaacgtga gttttcgttc | 9060 |
| | cactgagcgt cagaccccgt agaaaagatc aaaggatctt cttgagatcc tttttttctg | 9120 |
| | cgcgtaatct gctgcttgca aacaaaaaaa ccaccgctac cagcggtggt ttgtttgccg | 9180 |
| | gatcaagagc taccaactct ttttccgaag gtaactggct tcagcagagc gcagatacca | 9240 |
| | aatactgtcc ttctagtgta gccgtagtta ggccaccact tcaagaactc tgtagcaccg | 9300 |
| | cctacatacc tcgctctgct aatcctgtta ccagtggctg ctgccagtgg cgataagtcg | 9360 |
| | tgtcttaccg ggttggactc aagacgatag ttaccggata aggcgcagcg gtcgggctga | 9420 |
| | acggggggtt cgtgcacaca gcccagcttg gagcgaacga cctacaccga actgagatac | 9480 |
| | ctacagcgtg agcattgaga aagcgccacg cttcccgaag ggagaaaggc ggacaggtat | 9540 |
| | ccggtaagcg gcagggtcgg aacaggagag cgcacgaggg agcttccagg gggaaacgcc | 9600 |
| | tggtatcttt atagtcctgt cgggtttcgc cacctctgac ttgagcgtcg atttttgtga | 9660 |
| | tgctcgtcag gggggcggag cctatggaaa aacgccagca acgcggcctt tttacggttc | 9720 |
| | ctggcctttt gctggccttt ttgaagctgt ccctgatggt cgtcatctac ctgcctggac | 9780 |
| | agcatggcct gcaacgcggg catcccgatg ccgccggaag cgagaagaat cataatgggg | 9840 |
| | aaggccatcc agcctcgcgt cg | 9862 |
| SEQ ID NO: 36 | ctaaattgta agcgttaata ttttgttaaa attcgcgtta aatttttgtt aaatcagctc | 60 |
| (donor vector | attttttaac caataggccg aaatcggcaa aatcccttat aaatcaaaag aatagaccga | 120 |
| 1, pMK 8B3 | gatagggttg agtggccgct acagggcgct cccattcgcc attcaggctg cgcaactgtt | 180 |
| anti mFC scFV | gggaagggcg tttcggtgcg ggcctcttcg ctattacgcc agctggcgaa agggggatgt | 240 |
| CoOp ECORV | gctgcaaggc gattaagttg ggtaacgcca gggttttccc agtcacgacg ttgtaaaacg | 300 |
| SacII L1R5) | acggccagtg agcgcgacgt aatacgactc actatagggc gaattgaagg aaggccgtca | 360 |
| | aggccgcata aataatgatt ttattttgac tgatagtgac ctgttcgttg caacaaattg | 420 |
| | atgagcaatg cttttttata atgccaactt tgtacaaaaa agctgaacga tatcgccacc | 480 |
| | atgggcagca cagccattct ggccctgctg ctggcagtgc tgcagggcgt gtcagctcag | 540 |
| | gtgcagctgc agcagtctgg cgccgaagtg aagaaacccg gcagcagcgt gaaggtgtcc | 600 |
| | tgcaaggcta gcggcggcac cttcagcagc tacgccattt cttgggtgcg ccaggcccct | 660 |
| | ggacagggcc tggaatggat gggctggatc agcccctaca acggcaacac cgactacgcc | 720 |
| | cagaaagtgc agggcagagt gaccctgacc accgacacca gcacctccac cgcctacatg | 780 |
| | gaactgcgga gcctgagaag cgacgacacc gccgtgtact actgtgccac aggcggcgga | 840 |
| | acctggtaca gcgatctgtg gggcagaggc accctcgtga cagtgtctgc tggcggcgga | 900 |
| | ggatctggcg gaggcggaag tggcggggga ggaagcggag cacctgagat cgtgctgacc | 960 |
| | cagagcccta gcacactgag cgccagcgtg ggcgacagag tgtccatcac ctgtagagcc | 1020 |
| | agccagagca tcggaggcag cctggcctgg tatcagcaga agcctggcaa ggcccccaag | 1080 |
| | ctgctgatct ctgaggccag caccctggaa agaggcgtgc ccagcagatt ttccggcagc | 1140 |
| | ggctctggca ccgacttcac cctgacaatc agcagcctgc agcccgagga cgtggccacc | 1200 |
| | tactactgcc agaagtacaa cagcgtgccc ctgaccttcg gccctggcac caaggtggaa | 1260 |
| | atcaagccgc gggccaactt tgtatacaaa agtggaacga gaaacgtaaa atgatataaa | 1320 |
| | tatcaatata ttaaattaga ttttgcataa aaaacagact acataatact gtaaaacaca | 1380 |
| | acatatccag tcactatgaa tcaactactt agatggtatt agtgacctgt actgggcctc | 1440 |
| | atgggccttc ctttcactgc ccgctttcca gtcgggaaac ctgtcgtgcc agctgcatta | 1500 |
| | acatggtcat agctgtttcc ttgcgtattg ggcgctctcc gcttcctcgc tcactgactc | 1560 |
| | gctgcgctcg gtcgttcggg taaagcctgg ggtgcctaat gagcaaaagg ccagcaaaag | 1620 |
| | gccaggaacc gtaaaaaggc cgcgttgctg gcgtttttcc ataggctccg cccccctgac | 1680 |
| | gagcatcaca aaaatcgacg ctcaagtcag aggtggcgaa acccgacagg actataaaga | 1740 |
| | taccaggcgt ttccccctgg aagctccctc gtgcgctctc ctgttccgac cctgccgctt | 1800 |
| | accggatacc tgtccgcctt tctcccttcg ggaagcgtgg cgctttctca tagctcacgc | 1860 |
| | tgtaggtatc tcagttcggt gtaggtcgtt cgctccaagc tgggctgtgt gcacgaaccc | 1920 |
| | cccgttcagc ccgaccgctg cgccttatcc ggtaactatc gtcttgagtc caacccggta | 1980 |
| | agacacgact tatcgccact ggcagcagcc actggtaaca ggattagcag agcgaggtat | 2040 |
| | gtaggcggtg ctacagagtt cttgaagtgg tggcctaact acggctacac tagaagaaca | 2100 |
| | gtatttggta tctgcgctct gctgaagcca gttaccttcg gaaaaagagt tggtagctct | 2160 |
| | tgatccggca aacaaaccac cgctggtagc ggtggttttt ttgtttgcaa gcagcagatt | 2220 |
| | acgcgcagaa aaaaaggatc tcaagaagat cctttgatct tttctacggg gtctgacgct | 2280 |
| | cagtggaacg aaaactcacg ttaagggatt ttggtcatga gattatcaaa aaggatcttc | 2340 |
| | acctagatcc ttttaaatta aaaatgaagt tttaaatcaa tctaaagtat atatgagtaa | 2400 |
| | acttggtctg acagttatta gaaaaattca tccagcagac gataaaacgc aatacgctgg | 2460 |
| | ctatccggtg ccgcaatgcc atacagcacc agaaaacgat ccgcccattc gccgcccagt | 2520 |
| | tcttccgcaa tatcacgggt ggccagcgca atatcctgat aacgatccgc cacgcccaga | 2580 |
| | cggccgcaat caataaagcc gctaaaacgg ccattttcca ccataatgtt cggcaggcac | 2640 |
| | gcatcaccat gggtcaccac cagatcttcg ccatccggca tgctcgcttt cagacgcgca | 2700 |
| | aacagctctg ccggtgccag gccctgatgt tcttcatcca gatcatcctg atccaccagg | 2760 |
| | cccgcttcca tacgggtacg cgcacgttca atacgatgtt tcgcctgatg atcaaacgga | 2820 |
| | caggtcgccg ggtccagggt atgcagacga cgcatggcat ccgccataat gctcactttt | 2880 |
| | tctgccggcg ccagatggct agacagcaga tcctgacccg gcacttcgcc cagcagcagc | 2940 |
| | caatcacggc ccgcttcggt caccacatcc agcaccgccg cacacggaac accggtggtg | 3000 |
| | gccagccagc tcagacgcgc cgcttcatcc tgcagctcgt tcagcgcacc gctcagatcg | 3060 |
| | gttttcacaa acagcaccgg acgaccctgc gcgctcagac gaaacaccgc cgcatcagag | 3120 |
| | cagccaatgg tctgctgcgc ccaatcatag ccaaacagac gttccaccca cgctgccggg | 3180 |
| | ctacccgcat gcaggccatc ctgttcaatc atactcttcc tttttcaata ttattgaagc | 3240 |
| | atttatcagg gttattgtct catgagcgga tacatatttg aatgtattta gaaaaataaa | 3300 |
| | caaatagggg ttccgcgcac atttccccga aaagtgccac | 3340 |
| SEQ ID NO: 37 | ctaaattgta agcgttaata ttttgttaaa attcgcgtta aatttttgtt aaatcagctc | 60 |
| (donor vector | attttttaac caataggccg aaatcggcaa aatcccttat aaatcaaaag aatagaccga | 120 |
| 2, pMK hCD8a | gatagggttg agtggccgct acagggcgct cccattcgcc attcaggctg cgcaactgtt | 180 |
| scaffold TN L5 | gggaagggcg tttcggtgcg ggcctcttcg ctattacgcc agctggcgaa agggggatgt | 240 |
| L2 ) | gctgcaaggc gattaagttg ggtaacgcca gggttttccc agtcacgacg ttgtaaaacg | 300 |
| | acggccagtg agcgcgacgt aatacgactc actatagggc gaattgaagg aaggccgtca | 360 |
| | aggccgcata aataatgatt ttattttgac tgatagtgac ctgttcgttg caacaaattg | 420 |
| | atgagcaatg cttttttata atgcccaact ttgtatacaa aagtggcccg cggacaacaa | 480 |
| | cccctgcccc cagacctcct accccagccc ctacaattgc cagccagcct ctgagcctga | 540 |
| | ggcccgaggc ttgtagacct gctgctggcg gagccgtgca caccagagga ctggatttcg | 600 |
| | cctgcgacat ctacatctgg gcccctctgg ccggcacatg tggcgtgctg ctgctgagcc | 660 |
| | tcgtgatcac cctgtactgc ggctccacca gcggctccgg caagcccggc tctggcgagg | 720 |
| | gctccaccag cggcgactac aaggacgacg atgacaagta ataggatatc ggttcagctt | 780 |
| | tcttgtacaa agttggcatt ataagaaagc attgcttatc aatttgttgc aacgaacagg | 840 |
| | tcactatcag tcaaaataaa atcattattt ctgggcctca tgggccttcc tttcactgcc | 900 |
| | cgctttccag tcgggaaacc tgtcgtgcca gctgcattaa catggtcata gctgtttcct | 960 |
| | tgcgtattgg gcgctctccg cttcctcgct cactgactcg ctgcgctcgg tcgttcgggt | 1020 |
| | aaagcctggg gtgcctaatg agcaaaaggc cagcaaaagg ccaggaaccg taaaaaggcc | 1080 |
| | gcgttgctgg cgtttttcca taggctccgc ccccctgacg agcatcacaa aaatcgacgc | 1140 |
| | tcaagtcaga ggtggcgaaa cccgacagga ctataaagat accaggcgtt tccccctgga | 1200 |
| | agctccctcg tgcgctctcc tgttccgacc ctgccgctta ccggatacct gtccgccttt | 1260 |
| | ctcccttcgg gaagcgtggc gctttctcat agctcacgct gtaggtatct cagttcggtg | 1320 |
| | taggtcgttc gctccaagct gggctgtgtg cacgaacccc ccgttcagcc cgaccgctgc | 1380 |
| | gccttatccg gtaactatcg tcttgagtcc aacccggtaa gacacgactt atcgccactg | 1440 |
| | gcagcagcca ctggtaacag gattagcaga gcgaggtatg taggcggtgc tacagagttc | 1500 |
| | ttgaagtggt ggcctaacta cggctacact agaagaacag tatttggtat ctgcgctctg | 1560 |
| | ctgaagccag ttaccttcgg aaaaagagtt ggtagctctt gatccggcaa acaaaccacc | 1620 |
| | gctggtagcg gtggtttttt tgtttgcaag cagcagatta cgcgcagaaa aaaaggatct | 1680 |
| | caagaagatc ctttgatctt ttctacgggg tctgacgctc agtggaacga aaactcacgt | 1740 |
| | taagggattt tggtcatgag attatcaaaa aggatcttca cctagatcct tttaaattaa | 1800 |
| | aaatgaagtt ttaaatcaat ctaaagtata tatgagtaaa cttggtctga cagttattag | 1860 |
| | aaaaattcat ccagcagacg ataaaacgca atacgctggc tatccggtgc cgcaatgcca | 1920 |
| | tacagcacca gaaaacgatc cgcccattcg ccgcccagtt cttccgcaat atcacgggtg | 1980 |
| | gccagcgcaa tatcctgata acgatccgcc acgcccagac ggccgcaatc aataaagccg | 2040 |
| | ctaaaacggc cattttccac cataatgttc ggcaggcacg catcaccatg ggtcaccacc | 2100 |
| | agatcttcgc catccggcat gctcgctttc agacgcgcaa acagctctgc cggtgccagg | 2160 |
| | ccctgatgtt cttcatccag atcatcctga tccaccaggc ccgcttccat acgggtacgc | 2220 |
| | gcacgttcaa tacgatgttt cgcctgatga tcaaacggac aggtcgccgg gtccagggta | 2280 |
| | tgcagacgac gcatggcatc cgccataatg ctcacttttt ctgccggcgc cagatggcta | 2340 |
| | gacagcagat cctgacccgg cacttcgccc agcagcagcc aatcacggcc cgcttcggtc | 2400 |
| | accacatcca gcaccgccgc acacggaaca ccggtggtgg ccagccagct cagacgcgcc | 2460 |
| | gcttcatcct gcagctcgtt cagcgcaccg ctcagatcgg ttttcacaaa cagcaccgga | 2520 |
| | cgaccctgcg cgctcagacg aaacaccgcc gcatcagagc agccaatggt ctgctgcgcc | 2580 |
| | caatcatagc caaacagacg ttccacccac gctgccgggc tacccgcatg caggccatcc | 2640 |
| | tgttcaatca tactcttcct ttttcaatat tattgaagca tttatcaggg ttattgtctc | 2700 |
| | atgagcggat acatatttga atgtatttag aaaaataaac aaataggggt tccgcgcaca | 2760 |
| | tttccccgaa aagtgccac | 2779 |
| SEQ ID NO: 38 | cgataaccct aattcgatag catatgcttc ccgttgggta acatatgcta ttgaattagg | 60 |
| (Final vector | gttagtctgg atagtatata ctactacccg ggaagcatat gctacccgtt tagggttcac | 120 |
| used for | cggtgatgcc ggccacgatg cgtccggcgt agaggatcta atgtgagtta gctcactcat | 180 |
| lentiviral | taggcacccc aggctttaca ctttatgctt ccggctcgta tgttgtgtgg aattgtgagc | 240 |
| production, | ggataacaat ttcacacagg aaacagctat gaccatgatt acgccaagcg cgcaattaac | 300 |
| pLV430lG 8B3 | cctcactaaa gggaacaaaa gctggagctg caagcttaat gtagtcttat gcaatactct | 360 |
| scFV mlgG hCD8 | tgtagtcttg caacatggta acgatgagtt agcaacatgc cttacaagga gagaaaaagc | 420 |
| flag) | accgtgcatg ccgattggtg gaagtaaggt ggtacgatcg tgccttatta ggaaggcaac | 480 |
| | agacgggtct gacatggatt ggacgaacca ctgaattgcc gcattgcaga gatattgtat | 540 |
| | ttaagtgcct agctcgatac ataaacgggt ctctctggtt agaccagatc tgagcctggg | 600 |
| | agctctctgg ctaactaggg aacccactgc ttaagcctca ataaagcttg ccttgagtgc | 660 |
| | ttcaagtagt gtgtgcccgt ctgttgtgtg actctggtaa ctagagatcc ctcagaccct | 720 |
| | tttagtcagt gtggaaaatc tctagcagtg gcgcccgaac agggacttga aagcgaaagg | 780 |
| | gaaaccagag gagctctctc gacgcaggac tcggcttgct gaagcgcgca cggcaagagg | 840 |
| | cgaggggcgg cgactggtga gtacgccaaa aattttgact agcggaggct agaaggagag | 900 |
| | agatgggtgc gagagcgtca gtattaagcg ggggagaatt agatcgcgat gggaaaaaat | 960 |
| | tcggttaagg ccagggggaa agaaaaaata taaattaaaa catatagtat gggcaagcag | 1020 |
| | ggagctagaa cgattcgcag ttaatcctgg cctgttagaa acatcagaag gctgtagaca | 1080 |
| | aatactggga cagctacaac catcccttca gacaggatca gaagaactta gatcattata | 1140 |
| | taatacagta gcaaccctct attgtgtgca tcaaaggata gagataaaag acaccaagga | 1200 |
| | agctttagac aagatagagg aagagcaaaa caaaagtaag accaccgcac agcaagcggc | 1260 |
| | cgctgatctt cagacctgga ggaggagata tgagggacaa ttggagaagt gaattatata | 1320 |
| | aatataaagt agtaaaaatt gaaccattag gagtagcacc caccaaggca aagagaagag | 1380 |
| | tggtgcagag agaaaaaaga gcagtgggaa taggagcttt gttccttggg ttcttgggag | 1440 |
| | cagcaggaag cactatgggc gcagcgtcaa tgacgctgac ggtacaggcc agacaattat | 1500 |
| | tgtctggtat agtgcagcag cagaacaatt tgctgagggc tattgaggcg caacagcatc | 1560 |
| | tgttgcaact cacagtctgg ggcatcaagc agctccaggc aagaatcctg gctgtggaaa | 1620 |
| | gatacctaaa ggatcaacag ctcctgggga tttggggttg ctctggaaaa ctcatttgca | 1680 |
| | ccactgctgt gccttggaat gctagttgga gtaataaatc tctggaacag atttggaatc | 1740 |
| | acacgacctg gatggagtgg gacagagaaa ttaacaatta cacaagctta atacactcct | 1800 |
| | taattgaaga atcgcaaaac cagcaagaaa agaatgaaca agaattattg gaattagata | 1860 |
| | aatgggcaag tttgtggaat tggtttaaca taacaaattg gctgtggtat ataaaattat | 1920 |
| | tcataatgat agtaggaggc ttggtaggtt taagaatagt ttttgctgta ctttctatag | 1980 |
| | tgaatagagt taggcaggga tattcaccat tatcgtttca gacccacctc ccaaccccga | 2040 |
| | ggggacccga caggcccgaa ggaatagaag aagaaggtgg agagagagac agagacagat | 2100 |
| | ccattcgatt agtgaacgga tctcgacggt atcggtttta aaagaaaagg ggggattggg | 2160 |
| | gggtacagtg caggggaaag aatagtagac ataatagcaa cagacataca aactaaagaa | 2220 |
| | ttacaaaaac aaattacaaa aattcaaaat tttatcgatt ttatttagtc tccagaaaaa | 2280 |
| | ggggggaatg aaagacccca cctgtaggtt tggcaagcta gcttaagtaa cgccattttg | 2340 |
| | caaggcatgg aaaatacata actgagaata gagaagttca gatcaaggtt aggaacagag | 2400 |
| | agacagcaga atatgggcca aacaggatat ctgtggtaag cagttcctgc cccggctcag | 2460 |
| | ggccaagaac agatggtccc cagatgcggt cccgccctca gcagtttcta gagaaccatc | 2520 |
| | agatgtttcc agggtgcccc aaggacctga aatgaccctg tgccttattt gaactaacca | 2580 |
| | atcagttcgc ttctcgcttc tgttcgcgcg cttctgctcc ccgagctcaa taaaagagcc | 2640 |
| | cacaacccct cactcggcgc gccagtcctc cgatagactg cgtcgcccgg gtaccgatat | 2700 |
| | caccaacttt gtacaaaaaa gctgaacgat atcgccacca tgggcagcac agccattctg | 2760 |
| | gccctgctgc tggcagtgct gcagggcgtg tcagctcagg tgcagctgca gcagtctggc | 2820 |
| | gccgaagtga agaaacccgg cagcagcgtg aaggtgtcct gcaaggctag cggcggcacc | 2880 |
| | ttcagcagct acgccatttc ttgggtgcgc caggcccctg gacagggcct ggaatggatg | 2940 |
| | ggctggatca gcccctacaa cggcaacacc gactacgccc agaaagtgca gggcagagtg | 3000 |
| | accctgacca ccgacaccag cacctccacc gcctacatgg aactgcggag cctgagaagc | 3060 |
| | gacgacaccg ccgtgtacta ctgtgccaca ggcggcggaa cctggtacag cgatctgtgg | 3120 |
| | ggcagaggca ccctcgtgac agtgtctgct ggcggcggag gatctggcgg aggcggaagt | 3180 |
| | ggcgggggag gaagcggagc acctgagatc gtgctgaccc agagccctag cacactgagc | 3240 |
| | gccagcgtgg gcgacagagt gtccatcacc tgtagagcca gccagagcat cggaggcagc | 3300 |
| | ctggcctggt atcagcagaa gcctggcaag gcccccaagc tgctgatctc tgaggccagc | 3360 |
| | accctggaaa gaggcgtgcc cagcagattt tccggcagcg gctctggcac cgacttcacc | 3420 |
| | ctgacaatca gcagcctgca gcccgaggac gtggccacct actactgcca gaagtacaac | 3480 |
| | agcgtgcccc tgaccttcgg ccctggcacc aaggtggaaa tcaagccgcg ggccaacttt | 3540 |
| | gtatacaaaa gtggcccgcg gacaacaacc cctgccccca gacctcctac cccagcccct | 3600 |
| | acaattgcca gccagcctct gagcctgagg cccgaggctt gtagacctgc tgctggcgga | 3660 |
| | gccgtgcaca ccagaggact ggatttcgcc tgcgacatct acatctgggc ccctctggcc | 3720 |
| | ggcacatgtg gcgtgctgct gctgagcctc gtgatcaccc tgtactgcgg ctccaccagc | 3780 |
| | ggctccggca agcccggctc tggcgagggc tccaccagcg gcgactacaa ggacgacgat | 3840 |
| | gacaagtaat aggatatcgg ttcagctttc ttgtacaaag ttgggattcg agttaattaa | 3900 |
| | gttaacgaat tccccccctc tccctccccc ccccctaacg ttactggccg aagccgcttg | 3960 |
| | gaataaggcc ggtgtgcgtt tgtctatatg ttattttcca ccatattgcc gtcttttggc | 4020 |
| | aatgtgaggg cccggaaacc tggccctgtc ttcttgacga gcattcctag gggtctttcc | 4080 |
| | cctctcgcca aaggaatgca aggtctgttg aatgtcgtga aggaagcagt tcctctggaa | 4140 |
| | gcttcttgaa gacaaacaac gtctgtagcg accctttgca ggcagcggaa ccccccacct | 4200 |
| | ggcgacaggt gcctctgcgg ccaaaagcca cgtgtataag atacacctgc aaaggcggca | 4260 |
| | caaccccagt gccacgttgt gagttggata gttgtggaaa gagtcaaatg gctctcctca | 4320 |
| | agcgtattca acaaggggct gaaggatgcc cagaaggtac cccattgtat gggatctgat | 4380 |
| | ctggggcctc ggtgcacatg ctttacatgt gtttagtcga ggttaaaaaa cgtctaggcc | 4440 |
| | ccccgaacca cggggacgtg gttttccttt gaaaaacacg atgataatat ggccacaacc | 4500 |
| | atgggaggcg gaagcggcgg aggctcccct cgaggcacca tggtgagcaa gggcgaggag | 4560 |
| | ctgttcaccg gggtggtgcc catcctggtc gagctggacg gcgacgtaaa cggccacaag | 4620 |
| | ttcagcgtgt ccggcgaggg cgagggcgat gccacctacg gcaagctgac cctgaagttc | 4680 |
| | atctgcacca ccggcaagct gcccgtgccc tggcccaccc tcgtgaccac cctgacctac | 4740 |
| | ggcgtgcagt gcttcagccg ctaccccgac cacatgaagc agcacgactt cttcaagtcc | 4800 |
| | gccatgcccg aaggctacgt ccaggagcgc accatcttct tcaaggacga cggcaactac | 4860 |
| | aagacccgcg ccgaggtgaa gttcgagggc gacaccctgg tgaaccgcat cgagctgaag | 4920 |
| | ggcatcgact tcaaggagga cggcaacatc ctggggcaca agctggagta caactacaac | 4980 |
| | agccacaacg tctatatcat ggccgacaag cagaagaacg gcatcaaggt gaacttcaag | 5040 |
| | atccgccaca acatcgagga cggcagcgtg cagctcgccg accactacca gcagaacacc | 5100 |
| | cccatcggcg acggccccgt gctgctgccc gacaaccact acctgagcac ccagtccgcc | 5160 |
| | ctgagcaaag accccaacga gaagcgcgat cacatggtcc tgctggagtt cgtgaccgcc | 5220 |
| | gccgggatca ctctcggcat ggacgagctg tacaagtaac gcgtcccggg tctagagcta | 5280 |
| | gcggtaccat gcattacgta gtcgacgact taattaagct agcctagtgc catttgttca | 5340 |
| | gtggttcgta gggctttccc ccactgtttg gctttcagtt atatggatga tgtggtattg | 5400 |
| | ggggccaagt ctgtacagca tcttgagtcc ctttttaccg ctgttaccaa ttttcttttg | 5460 |
| | tctttgggta tacatttaaa ccctaacaaa acaaagagat ggggttactc tctaaatttt | 5520 |
| | atgggttatg tcattggatg ttatgggtcc ttgccacaag aacacatcat acaaaaaatc | 5580 |
| | aaagaatgtt ttagaaaact tcctattaac aggcctattg attggaaagt atgtcaacga | 5640 |
| | attgtgggtc ttttgggttt tgctgcccct tttacacaat gtggttatcc tgcgttgatg | 5700 |
| | cctttgtatg catgtattca atctaagcag gctttcactt tctcgccaac ttacaaggcc | 5760 |
| | tttctgtgta aacaatacct gaacctttac cccgttgccc ggcaacggcc aggtctgtgc | 5820 |
| | caagtgtttg ctgacgcaac ccccactggc tggggcttgg tcatgggcca tcagcgcatg | 5880 |
| | cgtggaacct tttcggctcc tctgccgatc catactgcgg aactcctagc cgcttgtttt | 5940 |
| | gctcgcagca ggtctggagc aaacattatc gggactgata actctgttgt cctatcccgc | 6000 |
| | aaatatacat cgtttccatg gctgctaggc tgtgctgcca actggatcct gcgcgggacg | 6060 |
| | tcctttgttt acgtcccgtc ggcgctgaat cctgcggacg acccttctcg gggtcgcttg | 6120 |
| | ggactctctc gtccccttct ccgtctgccg ttccgaccga ccacggggcg cacctctctt | 6180 |
| | tacgcggact ccccgtctgt gccttctcat ctgccggacc gtgtgcactt cgcttcacct | 6240 |
| | ctgcacgtcg catggagacc accgtgaacg cccaccaaat attgcccaag gtcttacata | 6300 |
| | agaggactct tggactctca gcaatgtcaa cgaccgacct tgaggcatac ttcaaagact | 6360 |
| | gtttgtttaa agactgggag gagttggggg aggagattag gttaaaggtc tttgtactag | 6420 |
| | gaggctgtag gcataaattg gtctgcgcac cagcaccatg gcgcaatcac tagagcgggg | 6480 |
| | tacctttaag accaatgact tacaaggcag ctgtagatct tagccacttt ttaaaagaaa | 6540 |
| | aggggggact ggaagggcta attcactccc aacgaagaca agatctgctt tttgcttgta | 6600 |
| | ctgggtctct ctggttagac cagatctgag cctgggagct ctctggctaa ctagggaacc | 6660 |
| | cactgcttaa gcctcaataa agcttgcctt gagtgcttca agtagtgtgt gcccgtctgt | 6720 |
| | tgtgtgactc tggtaactag agatccctca gaccctttta gtcagtgtgg aaaatctcta | 6780 |
| | gcagtagtag ttcatgtcat cttattattc agtatttata acttgcaaag aaatgaatat | 6840 |
| | cagagagtga gaggaacttg tttattgcag cttataatgg ttacaaataa agcaatagca | 6900 |
| | tcacaaattt cacaaataaa gcattttttt cactgcattc tagttgtggt ttgtccaaac | 6960 |
| | tcatcaatgt atcttatcat gtctggctct agctatcccg cccctaactc cgcccatccc | 7020 |
| | gcccctaact ccgcccagtt ccgcccattc tccgccccat ggctgactaa ttttttttat | 7080 |
| | ttatgcagag gccgaggccg gatcccttga gtggctttca tcctggagca gactttgcag | 7140 |
| | tctgtggact gcaacacaac attgccttta tgtgtaactc ttggctgaag ctcttacacc | 7200 |
| | aatgctgggg gacatgtacc tcccaggggc ccaggaagac tacgggaggc tacaccaacg | 7260 |
| | tcaatcagag gggcctgtgt agctaccgat aagcggaccc tcaagagggc attagcaata | 7320 |
| | gtgtttataa ggcccccttg ttaattcttg aagacgaaag ggcctcgtga tacgcctatt | 7380 |
| | tttataggtt aatgtcatga taataatggt ttcttagacg tcaggtggca cttttcgggg | 7440 |
| | aaatgtgcgc ggaaccccta tttgtttatt tttctaaata cattcaaata tgtatccgct | 7500 |
| | catgagacaa taaccctgat aaatgcttca ataatattga aaaaggaaga gtatgagtat | 7560 |
| | tcaacatttc cgtgtcgccc ttattccctt ttttgcggca ttttgccttc ctgtttttgc | 7620 |
| | tcacccagaa acgctggtga aagtaaaaga tgctgaagat cagttgggtg cacgagtggg | 7680 |
| | ttacatcgaa ctggatctca acagcggtaa gatccttgag agttttcgcc ccgaagaacg | 7740 |
| | ttttccaatg atgagcactt ttaaagttct gctatgtggc gcggtattat cccgtgttga | 7800 |
| | cgccgggcaa gagcaactcg gtcgccgcat acactattct cagaatgact tggttgagta | 7860 |
| | ctcaccagtc acagaaaagc atcttacgga tggcatgaca gtaagagaat tatgcagtgc | 7920 |
| | tgccataacc atgagtgata acactgcggc caacttactt ctgacaacga tcggaggacc | 7980 |
| | gaaggagcta accgcttttt tgcacaacat gggggatcat gtaactcgcc ttgatcgttg | 8040 |
| | ggaaccggag ctgaatgaag ccataccaaa cgacgagcgt gacaccacga tgcctgcagc | 8100 |
| | aatggcaaca acgttgcgca aactattaac tggcgaacta cttactctag cttcccggca | 8160 |
| | acaattaata gactggatgg aggcggataa agttgcagga ccacttctgc gctcggccct | 8220 |
| | tccggctggc tggtttattg ctgataaatc tggagccggt gagcgtgggt ctcgcggtat | 8280 |
| | cattgcagca ctggggccag atggtaagcc ctcccgtatc gtagttatct acacgacggg | 8340 |
| | gagtcaggca actatggatg aacgaaatag acagatcgct gagataggtg cctcactgat | 8400 |
| | taagcattgg taactgtcag accaagttta ctcatatata ctttagattg atttaaaact | 8460 |
| | tcatttttaa tttaaaagga tctaggtgaa gatccttttt gataatctca tgaccaaaat | 8520 |
| | cccttaacgt gagttttcgt tccactgagc gtcagacccc gtagaaaaga tcaaaggatc | 8580 |
| | ttcttgagat cctttttttc tgcgcgtaat ctgctgcttg caaacaaaaa aaccaccgct | 8640 |
| | accagcggtg gtttgtttgc cggatcaaga gctaccaact ctttttccga aggtaactgg | 8700 |
| | cttcagcaga gcgcagatac caaatactgt ccttctagtg tagccgtagt taggccacca | 8760 |
| | cttcaagaac tctgtagcac cgcctacata cctcgctctg ctaatcctgt taccagtggc | 8820 |
| | tgctgccagt ggcgataagt cgtgtcttac cgggttggac tcaagacgat agttaccgga | 8880 |
| | taaggcgcag cggtcgggct gaacgggggg ttcgtgcaca cagcccagct tggagcgaac | 8940 |
| | gacctacacc gaactgagat acctacagcg tgagcattga gaaagcgcca cgcttcccga | 9000 |
| | agggagaaag gcggacaggt atccggtaag cggcagggtc ggaacaggag agcgcacgag | 9060 |
| | ggagcttcca gggggaaacg cctggtatct ttatagtcct gtcgggtttc gccacctctg | 9120 |
| | acttgagcgt cgatttttgt gatgctcgtc aggggggcgg agcctatgga aaaacgccag | 9180 |
| | caacgcggcc tttttacggt tcctggcctt ttgctggcct ttttgaagct gtccctgatg | 9240 |
| | gtcgtcatct acctgcctgg acagcatggc ctgcaacgcg ggcatcccga tgccgccgga | 9300 |
| | agcgagaaga atcataatgg ggaaggccat ccagcctcgc gtcg | 9344 |
| SEQ ID NO: 39 | gtcgacggat cgggagatct cccgatcccc tatggtgcac tctcagtaca atctgctctg | 60 |
| (pLenti-C-Myc- | atgccgcata gttaagccag tatctgctcc ctgcttgtgt gttggaggtc gctgagtagt | 120 |
| DDK OX40L) | gcgcgagcaa aatttaagct acaacaaggc aaggcttgac cgacaattgc atgaagaatc | 180 |
| | tgcttagggt taggcgtttt gcgctgcttc gcgatgtacg ggccagatat cgcgttgaca | 240 |
| | ttgattattg actagttatt aatagtaatc aattacgggg tcattagttc atagcccata | 300 |
| | tatggagttc cgcgttacat aacttacggt aaatggcccg cctggctgac cgcccaacga | 360 |
| | cccccgccca ttgacgtcaa taatgacgta tgttcccata gtaacgccaa tagggacttt | 420 |
| | ccattgacgt caatgggtgg agtatttacg gtaaactgcc cacttggcag tacatcaagt | 480 |
| | gtatcatatg ccaagtacgc cccctattga cgtcaatgac ggtaaatggc ccgcctggca | 540 |
| | ttatgcccag tacatgacct tatgggactt tcctacttgg cagtacatct acgtattagt | 600 |
| | catcgctatt accatggtga tgcggttttg gcagtacatc aatgggcgtg gatagcggtt | 660 |
| | tgactcacgg ggatttccaa gtctccaccc cattgacgtc aatgggagtt tgttttggca | 720 |
| | ccaaaatcaa cgggactttc caaaatgtcg taacaactcc gccccattga cgcaaatggg | 780 |
| | cggtaggcgt gtacggtggg aggtctatat aagcagcgcg ttttgcctgt actgggtctc | 840 |
| | tctggttaga ccagatctga gcctgggagc tctctggcta actagggaac ccactgctta | 900 |
| | agcctcaata aagcttgcct tgagtgcttc aagtagtgtg tgcccgtctg ttgtgtgact | 960 |
| | ctggtaacta gagatccctc agaccctttt agtcagtgtg gaaaatctct agcagtggcg | 1020 |
| | cccgaacagg gacttgaaag cgaaagggaa accagaggag ctctctcgac gcaggactcg | 1080 |
| | gcttgctgaa gcgcgcacgg caagaggcga ggggcggcga ctggtgagta cgccaaaaat | 1140 |
| | tttgactagc ggaggctaga aggagagaga tgggtgcgag agcgtcagta ttaagcgggg | 1200 |
| | gagaattaga tcgcgatggg aaaaaattcg gttaaggcca gggggaaaga aaaaatataa | 1260 |
| | attaaaacat atagtatggg caagcaggga gctagaacga ttcgcagtta atcctggcct | 1320 |
| | gttagaaaca tcagaaggct gtagacaaat actgggacag ctacaaccat cccttcagac | 1380 |
| | aggatcagaa gaacttagat cattatataa tacagtagca accctctatt gtgtgcatca | 1440 |
| | aaggatagag ataaaagaca ccaaggaagc tttagacaag atagaggaag agcaaaacaa | 1500 |
| | aagtaagacc accgcacagc aagcggccgg ccgctgatct tcagacctgg aggaggagat | 1560 |
| | atgagggaca attggagaag tgaattatat aaatataaag tagtaaaaat tgaaccatta | 1620 |
| | ggagtagcac ccaccaaggc aaagagaaga gtggtgcaga gagaaaaaag agcagtggga | 1680 |
| | ataggagctt tgttccttgg gttcttggga gcagcaggaa gcactatggg cgcagcgtca | 1740 |
| | atgacgctga cggtacaggc cagacaatta ttgtctggta tagtgcagca gcagaacaat | 1800 |
| | ttgctgaggg ctattgaggc gcaacagcat ctgttgcaac tcacagtctg gggcatcaag | 1860 |
| | cagctccagg caagaatcct ggctgtggaa agatacctaa aggatcaaca gctcctgggg | 1920 |
| | atttggggtt gctctggaaa actcatttgc accactgctg tgccttggaa tgctagttgg | 1980 |
| | agtaataaat ctctggaaca gatttggaat cacacgacct ggatggagtg ggacagagaa | 2040 |
| | attaacaatt acacaagctt aatacactcc ttaattgaag aatcgcaaaa ccagcaagaa | 2100 |
| | aagaatgaac aagaattatt ggaattagat aaatgggcaa gtttgtggaa ttggtttaac | 2160 |
| | ataacaaatt ggctgtggta tataaaatta ttcataatga tagtaggagg cttggtaggt | 2220 |
| | ttaagaatag tttttgctgt actttctata gtgaatagag ttaggcaggg atattcacca | 2280 |
| | ttatcgtttc agacccacct cccaaccccg aggggacccg acaggcccga aggaatagaa | 2340 |
| | gaagaaggtg gagagagaga cagagacaga tccattcgat tagtgaacgg atcggcactg | 2400 |
| | cgtgcgccaa ttctgcagac aaatggcagt attcatccac aattttaaaa gaaaaggggg | 2460 |
| | gattgggggg tacagtgcag gggaaagaat agtagacata atagcaacag acatacaaac | 2520 |
| | taaagaatta caaaaacaaa ttacaaaaat tcaaaatttt cgggtttatt acagggacag | 2580 |
| | cagagatcca gtttggttag taccgggccc gctctagaca tgtccaatat gaccgccatg | 2640 |
| | ttgacattga ttattgacta gttattaata gtaatcaatt acggggtcat tagttcatag | 2700 |
| | cccatatatg gagttccgcg ttacataact tacggtaaat ggcccgcctg gctgaccgcc | 2760 |
| | caacgacccc cgcccattga cgtcaataat gacgtatgtt cccatagtaa cgccaatagg | 2820 |
| | gactttccat tgacgtcaat gggtggagta tttacggtaa actgcccact tggcagtaca | 2880 |
| | tcaagtgtat catatgccaa gtccgccccc tattgacgtc aatgacggta aatggcccgc | 2940 |
| | ctggcattat gcccagtaca tgaccttacg ggactttcct acttggcagt acatctacgt | 3000 |
| | attagtcatc gctattacca tggtgatgcg gttttggcag tacaccaatg ggcgtggata | 3060 |
| | gcggtttgac tcacggggat ttccaagtct ccaccccatt gacgtcaatg ggagtttgtt | 3120 |
| | ttggcaccaa aatcaacggg actttccaaa atgtcgtaat aaccccgccc cgttgacgca | 3180 |
| | aatgggcggt aggcgtgtac ggtgggaggt ctatataagc agagctcgtt tagtgaaccg | 3240 |
| | tcagaatttt gtaatacgac tcactatagg gcggccggga attcgtcgac tggatccggt | 3300 |
| | accgaggaga tctgccgccg cgatcgccat ggaaagggtc caacccctgg aagagaatgt | 3360 |
| | gggaaatgca gccaggccaa gattcgagag gaacaagcta ttgctggtgg cctctgtaat | 3420 |
| | tcagggactg gggctgctcc tgtgcttcac ctacatctgc ctgcacttct ctgctcttca | 3480 |
| | ggtatcacat cggtatcctc gaattcaaag tatcaaagta caatttaccg aatataagaa | 3540 |
| | ggagaaaggt ttcatcctca cttcccaaaa ggaggatgaa atcatgaagg tgcagaacaa | 3600 |
| | ctcagtcatc atcaactgtg atgggtttta tctcatctcc ctgaagggct acttctccca | 3660 |
| | ggaagtcaac attagccttc attaccagaa ggatgaggag cccctcttcc aactgaagaa | 3720 |
| | ggtcaggtct gtcaactcct tgatggtggc ctctctgact tacaaagaca aagtctactt | 3780 |
| | gaatgtgacc actgacaata cctccctgga tgacttccat gtgaatggcg gagaactgat | 3840 |
| | tcttatccat caaaatcctg gtgaattctg tgtccttacg cgtacgcggc cgctcgagca | 3900 |
| | gaaactcatc tcagaagagg atctggcagc aaatgatatc ctggattaca aggatgacga | 3960 |
| | cgataaggtt taaacggccg gccgcggtct gtacaagtag gattcgtcga gggacctaat | 4020 |
| | aacttcgtat agcatacatt atacgaagtt atacatgttt aagggttccg gttccactag | 4080 |
| | gtacaattcg atatcaagct tatcgataat caacctctgg attacaaaat ttgtgaaaga | 4140 |
| | ttgactggta ttcttaacta tgttgctcct tttacgctat gtggatacgc tgctttaatg | 4200 |
| | cctttgtatc atgctattgc ttcccgtatg gctttcattt tctcctcctt gtataaatcc | 4260 |
| | tggttgctgt ctctttatga ggagttgtgg cccgttgtca ggcaacgtgg cgtggtgtgc | 4320 |
| | actgtgtttg ctgacgcaac ccccactggt tggggcattg ccaccacctg tcagctcctt | 4380 |
| | tccgggactt tcgctttccc cctccctatt gccacggcgg aactcatcgc cgcctgcctt | 4440 |
| | gcccgctgct ggacaggggc tcggctgttg ggcactgaca attccgtggt gttgtcgggg | 4500 |
| | aaatcatcgt cctttccttg gctgctcgcc tgtgttgcca cctggattct gcgcgggacg | 4560 |
| | tccttctgct acgtcccttc ggccctcaat ccagcggacc ttccttcccg cggcctgctg | 4620 |
| | ccggctctgc ggcctcttcc gcgtcttcgc cttcgccctc agacgagtcg gatctccctt | 4680 |
| | tgggccgcct ccccgcatcg ataccgtcga cctcgatcga gacctagaaa aacatggagc | 4740 |
| | aatcacaagt agcaatacag cagctaccaa tgctgattgt gcctggctag aagcacaaga | 4800 |
| | ggaggaggag gtgggttttc cagtcacacc tcaggtacct ttaagaccaa tgacttacaa | 4860 |
| | ggcagctgta gatcttagcc actttttaaa agaaaagggg ggactggaag ggctaattca | 4920 |
| | ctcccaacga agacaagata tccttgatct gtggatctac cacacacaag gctacttccc | 4980 |
| | tgattggcag aactacacac cagggccagg gatcagatat ccactgacct ttggatggtg | 5040 |
| | ctacaagcta gtaccagttg agcaagagaa ggtagaagaa gccaatgaag gagagaacac | 5100 |
| | ccgcttgtta caccctgtga gcctgcatgg gatggatgac ccggagagag aagtattaga | 5160 |
| | gtggaggttt gacagccgcc tagcatttca tcacatggcc cgagagctgc atccggactg | 5220 |
| | tactgggtct ctctggttag accagatctg agcctgggag ctctctggct aactagggaa | 5280 |
| | cccactgctt aagcctcaat aaagcttgcc ttgagtgctt caagtagtgt gtgcccgtct | 5340 |
| | gttgtgtgac tctggtaact agagatccct cagacccttt tagtcagtgt ggaaaatctc | 5400 |
| | tagcagcatg tgagcaaaag gccagcaaaa ggccaggaac cgtaaaaagg ccgcgttgct | 5460 |
| | ggcgtttttc cataggctcc gcccccctga cgagcatcac aaaaatcgac gctcaagtca | 5520 |
| | gaggtggcga aacccgacag gactataaag ataccaggcg tttccccctg gaagctccct | 5580 |
| | cgtgcgctct cctgttccga ccctgccgct taccggatac ctgtccgcct ttctcccttc | 5640 |
| | gggaagcgtg gcgctttctc atagctcacg ctgtaggtat ctcagttcgg tgtaggtcgt | 5700 |
| | tcgctccaag ctgggctgtg tgcacgaacc ccccgttcag cccgaccgct gcgccttatc | 5760 |
| | cggtaactat cgtcttgagt ccaacccggt aagacacgac ttatcgccac tggcagcagc | 5820 |
| | cactggtaac aggattagca gagcgaggta tgtaggcggt gctacagagt tcttgaagtg | 5880 |
| | gtggcctaac tacggctaca ctagaagaac agtatttggt atctgcgctc tgctgaagcc | 5940 |
| | agttaccttc ggaaaaagag ttggtagctc ttgatccggc aaacaaacca ccgctggtag | 6000 |
| | cggtggtttt tttgtttgca agcagcagat tacgcgcaga aaaaaaggat ctcaagaaga | 6060 |
| | tcctttgatc ttttctacgg ggtctgacgc tcagtggaac gaaaactcac gttaagggat | 6120 |
| | tttggtcatg attacgcccc gccctgccac tcatcgcagt actgttgtaa ttcattaagc | 6180 |
| | attctgccga catggaagcc atcacaaacg gcatgatgaa cctgaatcgc cagcggcatc | 6240 |
| | agcaccttgt cgccttgcgt ataatatttg cccatggtga aaacgggggc gaagaagttg | 6300 |
| | tccatattgg ccacgtttaa atcaaaactg gtgaaactca cccagggatt ggctgagacg | 6360 |
| | aaaaacatat tctcaataaa ccctttaggg aaataggcca ggttttcacc gtaacacgcc | 6420 |
| | acatcttgcg aatatatgtg tagaaactgc cggaaatcgt cgtggtattc actccagagc | 6480 |
| | gatgaaaacg tttcagtttg ctcatggaaa acggtgtaac aagggtgaac actatcccat | 6540 |
| | atcaccagct caccgtcttt cattgccata cggaactccg gatgagcatt catcaggcgg | 6600 |
| | gcaagaatgt gaataaaggc cggataaaac ttgtgcttat ttttctttac ggtctttaaa | 6660 |
| | aaggccgtaa tatccagctg aacggtctgg ttataggtac attgagcaac tgactgaaat | 6720 |
| | gcctcaaaat gttctttacg atgccattgg gatatatcaa cggtggtata tccagtgatt | 6780 |
| | tttttctcca tactcttcct ttttcaatat tattgaagca tttatcaggg ttattgtctc | 6840 |
| | atgagcggat acatatttga atgtatttag aaaaataaac aaataggggt cccgcgcaca | 6900 |
| | tttccccgaa aagtgccacc tgac | 6924 |

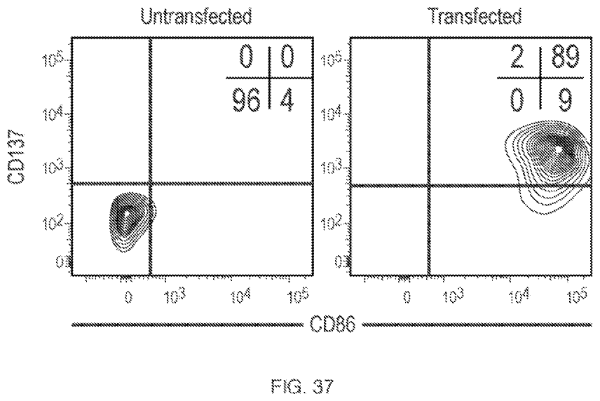

In the preparations of engineered EM-3 aAPCs (also referred to herein as aEM3 aAPCs) used for the experiments described herein, expression of CD86 and 4-1BBL was confirmed using flow cytometry (Canto II flow cytometer, Becton, Dickinson, and Co., Franklin Lakes, NJ, USA), with results shown in FIG. 37. aEM3 aAPCs were γ-irradiated at 100 Gy and frozen.

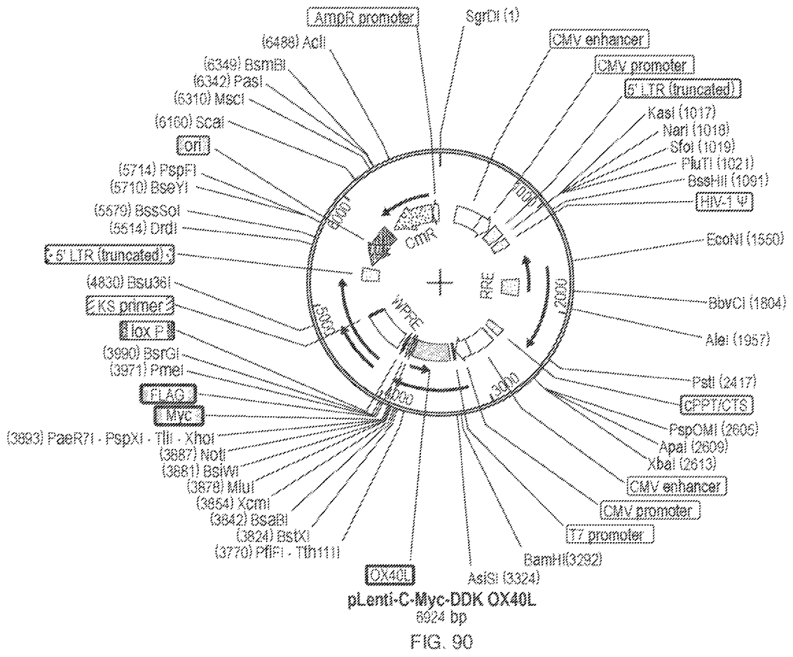

aEM-3 cells previously transduced to express CD86, antibody against IgG Fc region, and 4-1BBL (or optionally without 4-1BBL), as described above, were genetically engineered with a co-stimulatory human OX-40L using a similar lentiviral transduction approach. To generate lentivirus containing human OX-40L, pLenti-C-Myc-DDK OX40L (PS100064, Origene, SEQ ID NO:39, FIG. 90) vector together with the VSV-G envelope plasmid (pCIGO-VSV.G) were co-transfected into a Phoenix-GP (ATCC CRL-3215) cell line using PolyJet (Signagen Laboratories, Rockville, MD, USA). The supernatants were harvested 60 hours later and concentrated using Amicon Ultra-15 Centrifugal Filter Unit with Ultracel-100 membrane. aEM-3 cells were then infected with concentrated lentivirus and further expanded for five days. The cells were stained with PE-conjugated anti-human OX40L, Brilliant Violet 421-conjugated anti-human CD137L (if 4-1BBL is included in the prior aEM-3 cells), and PE/Cy7 conjugated anti-human CD86 and sorted based on the expression of GFP, OX40L, CD137L (when included), and CD86 using a S3e Cell Sorter (Bio-Rad, Inc., Hercules, CA, USA). The purity of sorted cells was further validated using flow cytometry. The enriched cells were checked for purity by flow cytometry.

### Example 6—Expansion of Tumor Infiltrating Lymphocytes Using EM-3 Artificial Antigen Presenting Cells

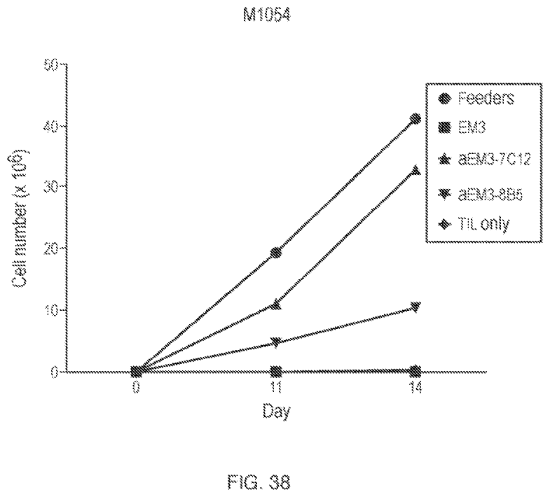

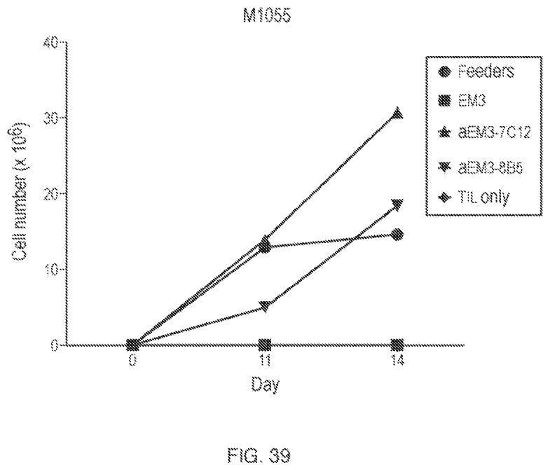

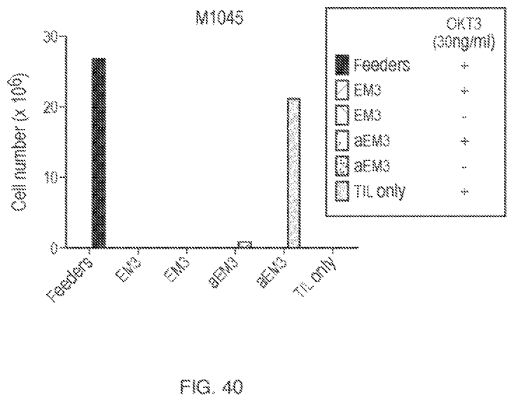

Experiments were performed to test the ability of EM-3 aAPCs (aEM3) to expand TILs. TIL were co-cultured with aEM3 (7C12 or 8B3) at a ratio of 1:100 ratio plus OKT-3 (30 mg/mL) and IL-2 (3000 IU/mL). Cells were counted on Day 11 and 14. The results are plotted for two batches of TILs in FIG. 38 and FIG. 39. In addition, TILs were co-cultured with aEM3 or PBMC feeders at a 1:100 ratio with IL-2 (3000 IU/mL) with or without OKT-3 (30 mg/mL). The results are plotted in FIG. 40, where the bar graph shows cell numbers determined on Day 11.

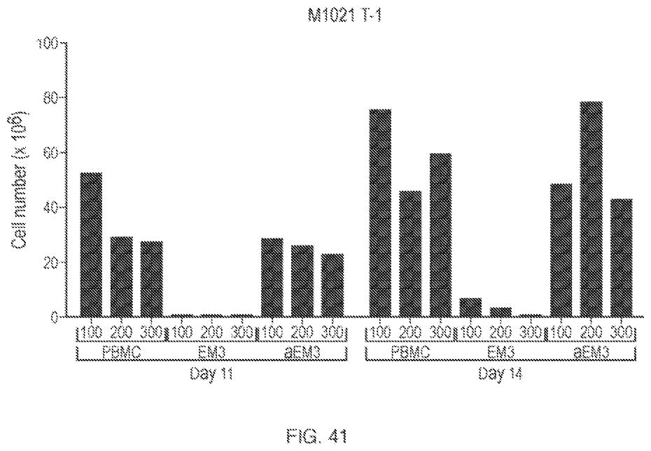

FIG. 41 illustrates the results of TIL expansions with EM-3 aAPCs (aEM3) at different TIL:aAPC ratios. The results show that aEM3 aAPCs perform comparably to and in some cases better than PBMCs, particularly at ratios of 1:200 at longer culture times (14 days).

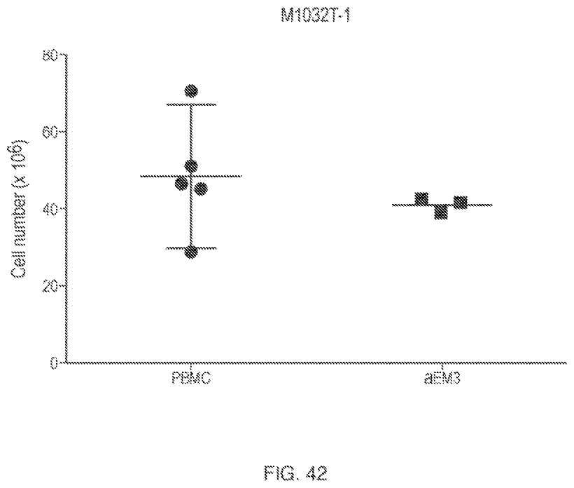

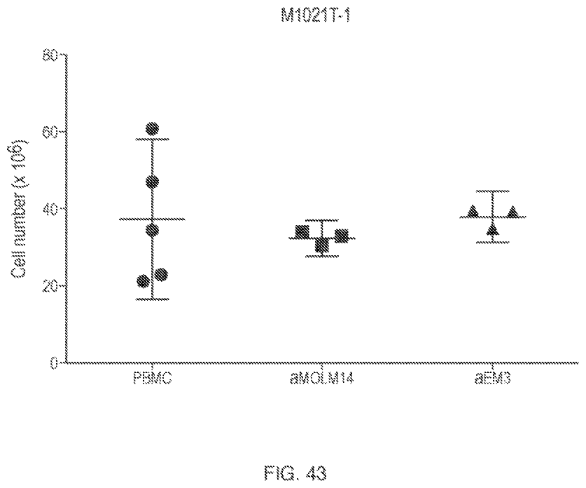

FIG. 42 illustrates the low variability in cell counts from TIL expansions with EM-3 aAPCs (aEM3) in comparison to PBMC feeders. TILs (2×10⁴) were co-cultured with five different PBMC feeder lots or aEM3 (in triplicate) at 1:100 ratio with IL-2 (3000 IU/mL) in a G-Rex 24 well plate. The graph shows viable cell numbers (mean) with 95% confidence interval counted on Day 14. FIG. 43 compares the results of TIL expansions with EM-3 aAPCs and MOLM-14 aAPCs, to illustrate variability in cell counts for both aEM3 and aMOLM14 in comparison to TILs (2×10⁴) were co-cultured with five different PBMC feeder lots or aMOLM14 (in triplicate) or aEM3 (also in triplicate) at 1:100 ratio with IL-2 (3000 IU/mL) in a G-Rex 24 well plate. Viable cells were counted on day 14, and the graph shows viable cell numbers (mean) with 95% confidence interval. The aEM3 and aMOLM14 results indicate that much greater consistency can be obtained with both aAPCs compared to the PBMC feeder approach preferred in the prior art.

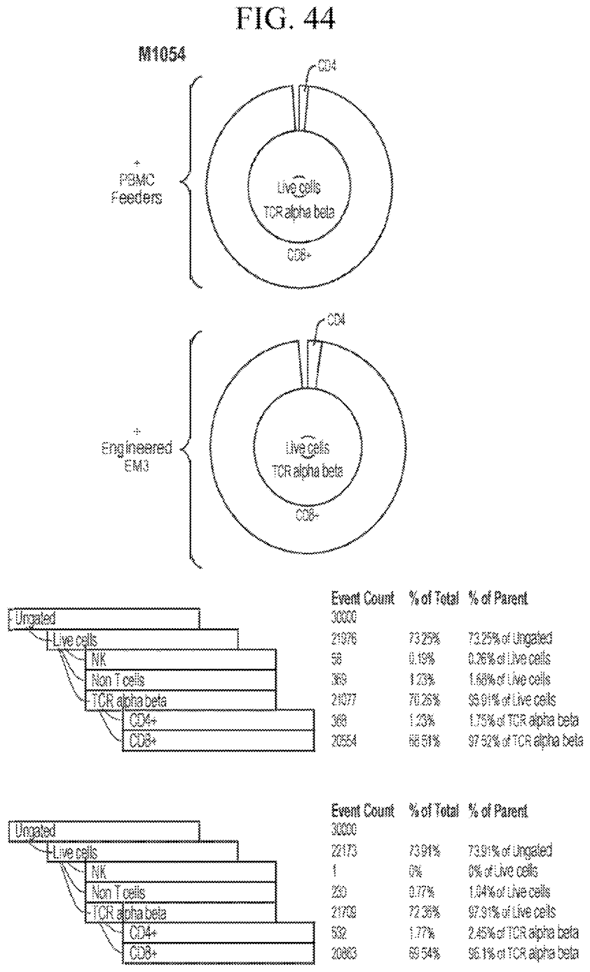

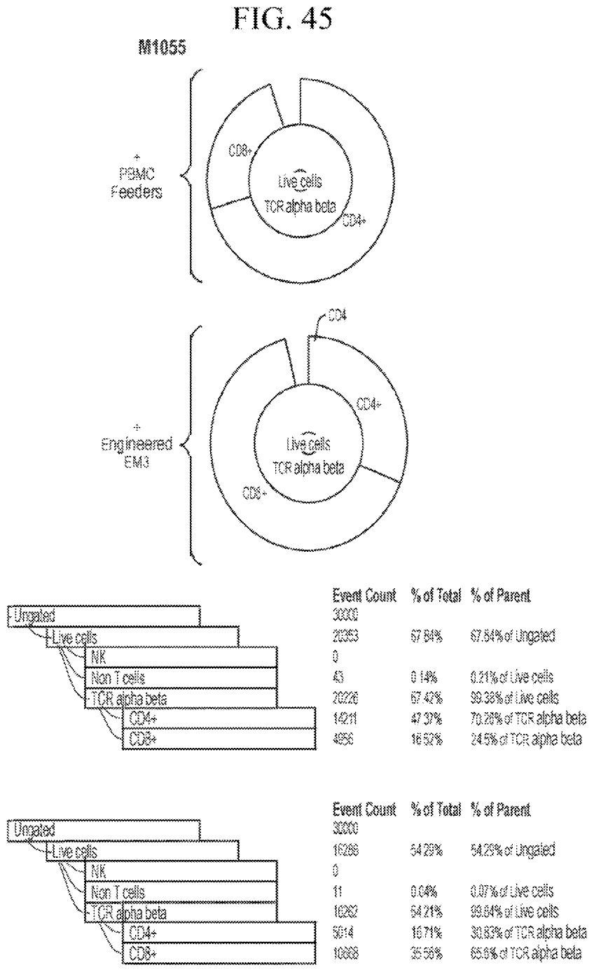

TILs expanded against aEM3 or PBMC feeders were used for flow cytometry analysis using 4 different panels (differentiation panels 1 and 2, T cell activation panels 1 and 2). Briefly, TILs were first stained with L/D Aqua to determine viability. Next, cells were surface stained with TCR 1/PE-Cy7, CD4 FITC, CD8 PB, CD56 APC, CD28 PE, CD27 APC-Cy7, and CD57-PerCP-Cy5.5 for differentiation panel 1; CD45RA PE-Cy7, CD8a PerCP/Cy5, CCR7 PE, CD4 FITC, CD3 APC-Cy7, CD38 APC, and HLA-DR PB, for differentiation panel 2; CD137 PE-Cy7, CD8a PerCP-Cy5.5, Lag3 PE, CD4 FITC, CD3 APC-Cy7, PD1 APC, and Tim-3 BV421 for T cell activation panel 1; or CD69 PE-Cy7, CD8a PerCP/Cy5.5, TIGIT PE, CD4 FITC, CD3 APC-Cy7, KLRG1 ALEXA 647, and CD154 BV421 for T cell activation panel 2. Phenotype analysis was done by gating 10,000 to 100,000 cells according to FSC/SSC using the Canto II flow cytometer. Data was analyzed using Cytobank software (Cytobank, Inc., Santa Clara, CA, USA) to create sunburst diagrams and SPADE (Spanning-tree Progression Analysis of Density-normalized Events) plots. Gates were set based on fluorescence minus one (FMO) controls. SPADE plots were generated with the group of cells, characterized in a form of related nodes based on the expression level of surface markers. CD4⁺ and CD8⁺ TIL subsets were determined based on CD3⁺ gating, and trees were generated. Sunburst visualizations are shown in FIG. 44 and FIG. 45. FIG. 44 shows that TILs expanded against aEM3 aAPCs maintained the CD8⁺ phenotype when compared to the same TILs expanded against PBMC feeders. FIG. 45 shows the results of a second batch of TILs from a different patient expanded against aEM3 aAPCs, where a clear increase of CD8⁺ cells (65.6%) is seen in comparison to the results from expansion using PBMC feeders (25%).

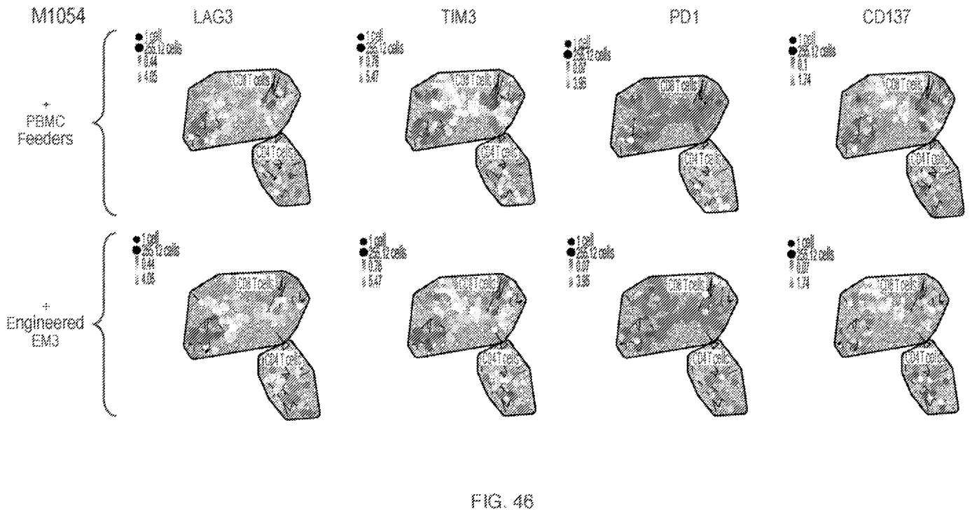

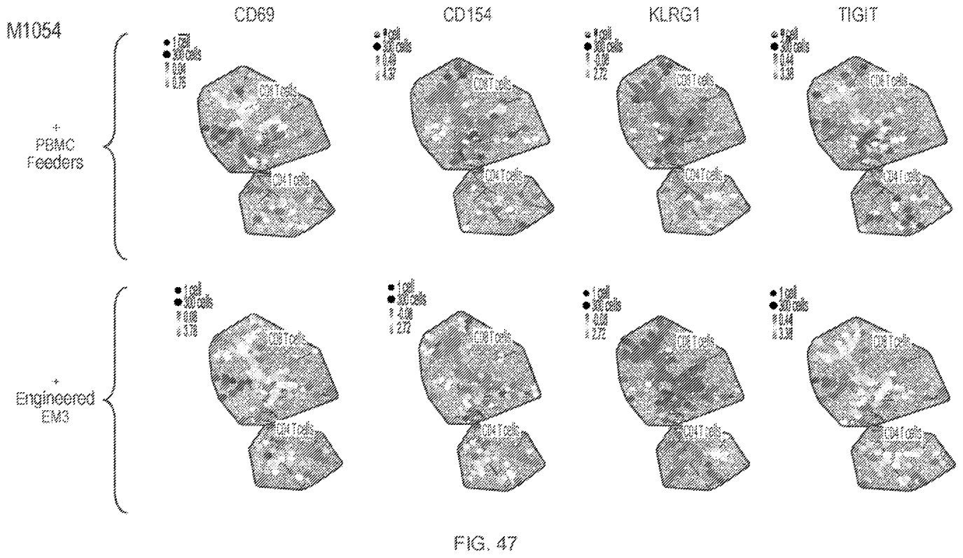

The CD4 and CD8 SPADE tree of TILs expanded with aEM3 aAPCs or PBMC feeders using CD3+ cells is shown in FIG. 46 and FIG. 47. The color gradient is proportional to the mean fluorescence intensity (MFI) of LAG3, TIL3, PD1 and CD137 or CD69, CD154, KLRG1 and TIGIT. Without being bound by theory, the results show that TILs expanded with aEM3 aAPCs had undergone activation, but there was no difference in MFI between the aEM3 aAPCs and PBMC feeders, indicating that the aEM3 aAPCs effectively replicate the phenotypic results obtained with PBMC feeders.

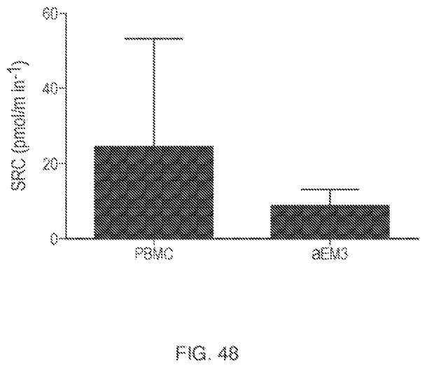

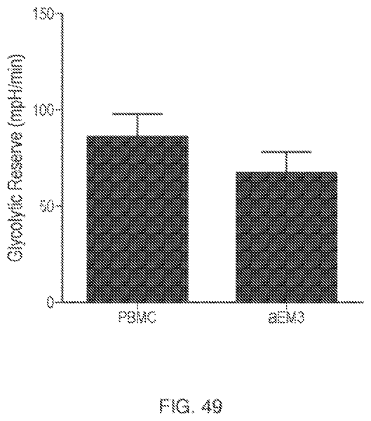

Spare respiratory capacity (SRC) and glycolytic reserve were also evaluated for TILs expanded with aEM3 aAPCs in comparison to PBMC feeders, with results shown in FIG. 48 and FIG. 49. The Seahorse XF Cell Mito Stress Test measures mitochondrial function by directly measuring the oxygen consumption rate (OCR) of cells, using modulators of respiration that target components of the electron transport chain in the mitochondria. The test compounds (oligomycin, FCCP, and a mix of rotenone and antimycin A, described below) are serially injected to measure ATP production, maximal respiration, and non-mitochondrial respiration, respectively. Proton leak and spare respiratory capacity are then calculated using these parameters and basal respiration. Each modulator targets a specific component of the electron transport chain. Oligomycin inhibits ATP synthase (complex V) and the decrease in OCR following injection of oligomycin correlates to the mitochondrial respiration associated with cellular ATP production. Carbonyl cyanide-4 (trifluoromethoxy)phenylhydrazone (FCCP) is an uncoupling agent that collapses the proton gradient and disrupts the mitochondrial membrane potential. As a result, electron flow through the electron transport chain is uninhibited and oxygen is maximally consumed by complex IV. The FCCP-stimulated OCR can then be used to calculate spare respiratory capacity, defined as the difference between maximal respiration and basal respiration. Spare respiratory capacity (SRC) is a measure of the ability of the cell to respond to increased energy demand. The third injection is a mix of rotenone, a complex I inhibitor, and antimycin A, a complex III inhibitor. This combination shuts down mitochondrial respiration and enables the calculation of nonmitochondrial respiration driven by processes outside the mitochondria.

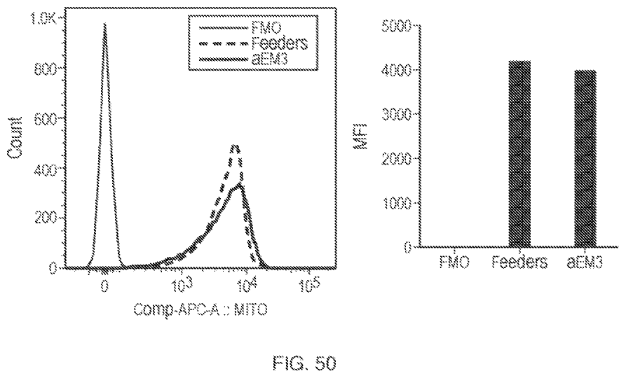

FIG. 50 illustrates a mitochondrial stain of Live TILs expanded against PBMC feeders or aEM3 aAPCs. MitoTracker dye stains mitochondria in live cells and its accumulation is dependent upon membrane potential. TILs expanded against PBMC feeders or aEM3 were stained L/D Aqua followed by MitoTracker red dye. The data show MitoTracker positive (MFI) cells gated on live population,

### Example 7—Comparison of Engineered MOLM-14 (aMOLM14) and EM-3 (aEM3) aAPCs

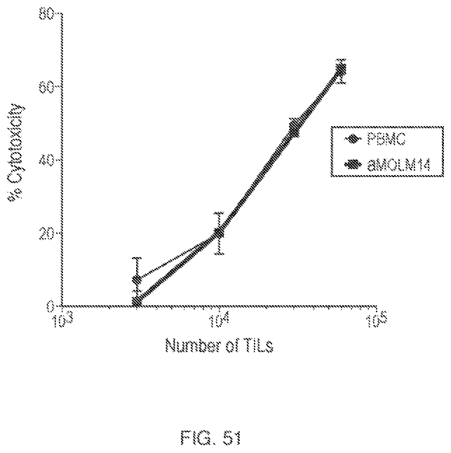

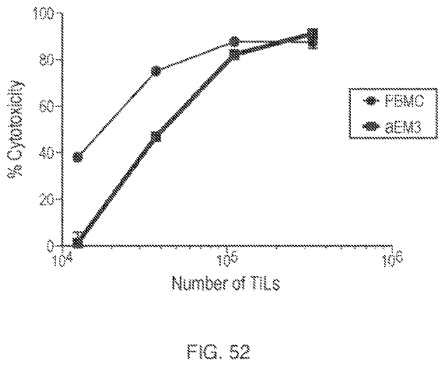

TILs expanded with PBMC feeders and aMOLM14 and aEM3 aAPCs, as described in the previous examples, were assessed for functional activity using the BRLA for cytotoxic potency. The P815 BRLA is described in detail in Example 9. The results are shown in FIG. 51 and FIG. 52, and show that TILs expanded with aAPCs have similar functional properties (and expected clinical efficacy) to those expanded with PBMC feeders.

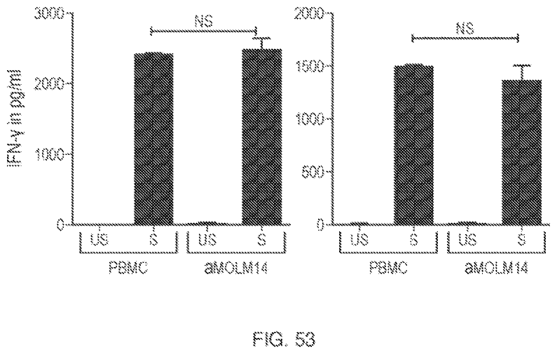

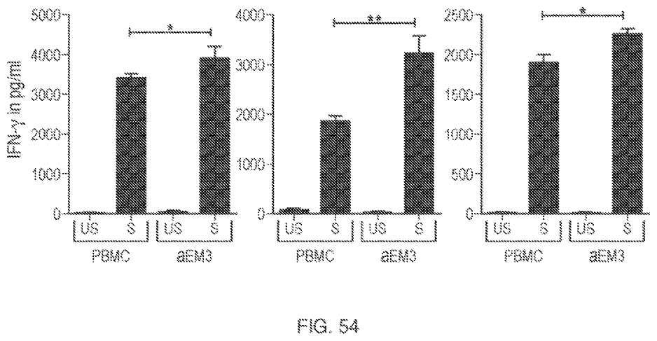

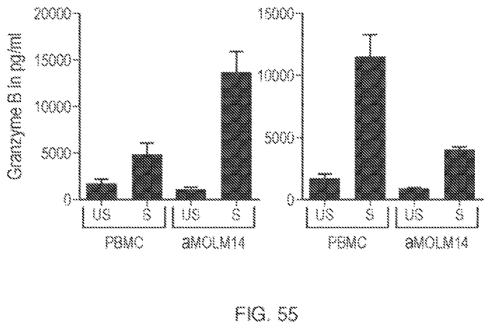

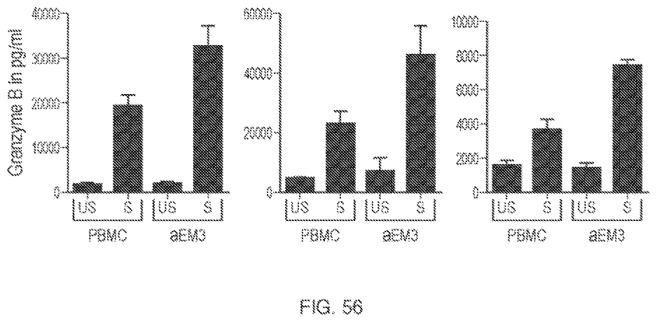

IFN-γ release and Granzyme B release from TILs expanded with PBMC feeders and aMOLM14 and aEM3 aAPCs as described above was also assessed following overnight stimulation with microbeads coated with anti-CD3/CD28/4-1BB. The IFN-γ release results are shown in FIG. 53 and FIG. 54, and the Granzyme B release results are shown in FIG. 55 and FIG. 56. Significant and surprising increases in IFN-γ release and Granzyme B release were observed for TILs expanded with aEM3 aAPCs relative to those expanded with PBMC feeders, but not for TILs expanded by aMOLM14 aAPCs. Without being bound by theory, this suggests that TILs cultured with aEM3 aAPCs may be more active in vivo as a cancer therapy. Most other differences observed were not statistically significant.

The results of TIL expansions with aEM3 and aMOLM14 aAPCs are summarized in Table 9.

TABLE 9
Summary of TIL expansion results with aAPCs.

| | | Fold Expansion | | Relative | CD8 (%) | | CD4 (%) | | Relative | Relative |
|---|---|---|---|---|---|---|---|---|---|---|
| aAPC | TIL# | PBMC | aAPC | expansion | PBMC | aAPC | PBMC | aAPC | CD8 | CD4 |
| aMOLM14 | M1032-T2 | 2112 | 1936 | 0.92 | 53 | 65 | 44 | 27 | 1.226 | 0.614 |
| | M1033-T6 | 1761 | 1598 | 0.91 | 50 | 57 | 36 | 40 | 1.140 | 1.111 |
| | M1021T-5 | 2053 | 2024 | 0.99 | 91 | 82 | 8 | 17 | 0.901 | 2.125 |
| | M1030T-4 | 860 | 853 | 0.99 | 46 | 78 | 51 | 12 | 1.696 | 0.235 |
| | M1045 | 858* | 758* | 0.88 | — | — | — | — | — | — |
| | M1021T-1 | 1866 | 1620 | 0.87 | — | — | — | — | — | — |
| | M1032T-1 | 2423 | 2049 | 0.85 | — | — | — | — | — | — |
| | M1042 | 1278 | 1704 | 1.33 | 8 | 8 | 88 | 89 | 0.919 | 1.015 |
| | M1043 | 1601 | 1587 | 0.99 | 90 | 87 | 5 | 5 | 0.968 | 0.947 |
| aEM3 | M1054 | 2058 | 1647 | 0.80 | 98 | 96 | 2 | 2 | 0.981 | 1.400 |
| | M1055 | 729 | 1533 | 2.10 | 25 | 66 | 70 | 31 | 2.694 | 0.441 |
| | M1021T-1 | 2985 | 2805 | 0.94 | 87 | 75 | 10 | 20 | 0.862 | 2.000 |
| | M1045 | 1336 | 1047 | 0.78 | — | — | — | — | — | — |

### Example 8—Preparation of Master Cell Banks for aEM3 and aMOLM14 aAPCs

aEM3 and aMOLM14 aAPCs may be grown in the following media compositions to produce master cell banks, which may be further grown in this media for supply of aAPCs: 500 mL of Dulbecco's Modified Eagle Medium DMEM/F12 (Sigma-Aldrich, St. Louis, MO, USA), 50 mL fetal bovine serum (FBS) Heat Inactivated (HI) (Hyclone); 10 mM 4-(2-hydroxyethyl)-1-piperazineethanesulfonic acid (HEPES buffer) (Life Technologies); 1× Primocin (Invivogen); 1× Plasmocin (Invivogen), and 1×2-mercaptoethanol (Life Technologies).

The aAPCs described herein, including aEM3 and aMOLM14 aAPCs, may also be grown from a master cell bank using any suitable method known in the art for the growth of cells. In an embodiment, aAPCs are thawed and are then expanded in a medium of 80-90% RPMI 1640+10-20% h.i. FBS (fetal bovine serum) by splitting saturated culture 1:2 to 1:3 every 2-3 days, seeding out at about 0.5-1×10⁶ cells/mL in 24-well plates, and maintaining at about 0.5-1.5×10⁶ cells/mL, with incubation at 37° C. and 5% CO₂.

Further steps that may be employed to use the aAPCs of certain embodiments of the present invention in the production of human therapies are known in the art and include cell line characterization (HLA high resolution typing); cytokine release testing; testing of human serum to replace FBS to grow aAPC; testing freezing media to freeze aAPCs; master cell banking (including raw material testing and stability testing); standardization of irradiation (including irradiation dose (1000, 3000, 5000, 10000, 15000 rad), fresh versus frozen aAPCs, and with/without TILs); stability of aAPC; development of a panel to evaluate the contamination of aAPCs; development of molecular biology assays (qPCR, DNA sequencing); testing of TIL expansions from different tumor types, including melanoma, cervical, and head and neck cancer (using a G-Rex 5M); potency, purity, and identity testing; Mycoplasma and sterility assays; microbiological testing (USP/EP sterility, bioburden and endotoxin assays); and adventitious viral agent testing.

### Example 9—Methods of Expanding TILs and Treating Cancer with Expanded TILs

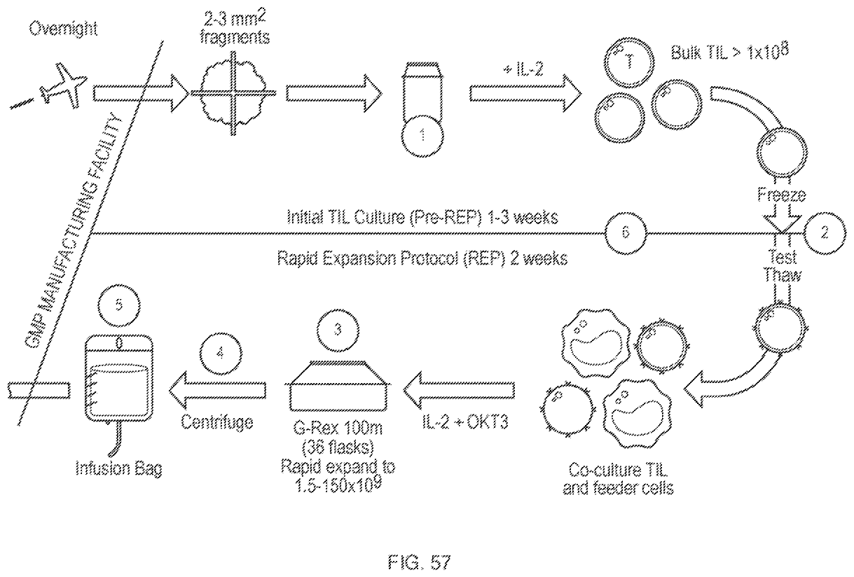

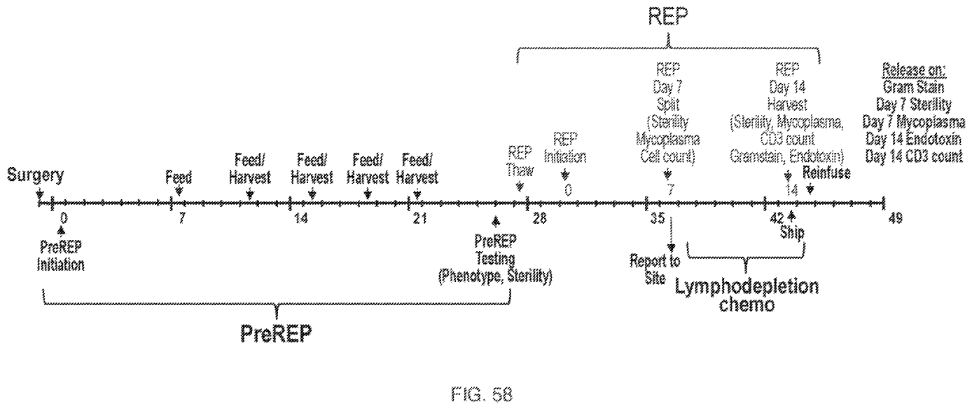

TILs may be expanded using the aAPCs of certain embodiments of the present invention, such as aEM3 and aMOLM14 aAPCs, using any of the expansion methods described herein. For example, a method for expanding TILs is depicted in FIG. 57. The expansion of TILs using aAPCs may be further combined with any method of treating cancer in a patient described herein. A method for expanding TILs and treating a patient with expanded TILs, wherein the expansion makes use of aAPCs (including aEM3 and aMOLM14 aAPCs), is shown in FIG. 58.

### Example 10—P815 Bioluminescent Redirected Lysis Assay

In this example, the development of a surrogate target cell line to evaluate the lytic potential of TILs in a Bioluminescent Redirected Lysis Assay (BRLA) is described. The BRLA enables assessment of T cell mediated killing in the absence of autologous tumor cells. Cytolytic activity can be assessed with and without engaging the T cell receptor in one to four hours, assessing T cell killing engaging the T cell receptor and without so-called lymphokine activated killer activities (LAK).

Mouse mastocytoma P815 cells expressing the endogenous CD16 Fc receptor can bind anti-CD38 (OKT-3), providing a potent TCR activation signal as a target cell line. The P815 Clone G6 was transduced with a lentiviral vector based on eGFP and firefly luciferase, sorted and cloned using the BD FACSAria II. Clone G6 was selected based on eGFP intensity analyzed using an Intellicyt iQue Screener. Target cells and TILs of interest were co-cultured+/−OKT-3 to assess TCR activation (specific killing) or non-specific (lymphokine activated killing, LAK) respectively. Following 4 hours of incubation, firefly luciferin ((4S)-2-(6-hydroxy-1,3-benzothiazol-2-yl)-4,5-dihydrothiazole-4-carboxylic acid, commercially available from multiple sources) was added to the wells and incubated for 5 minutes. Bioluminescence intensity was read using a luminometer. Percent cytotoxicity and survival were calculated using the following formula: % Survival=(experimental survival−minimum)/(maximum signal−minimum signal)×100; % Cytotoxicity=100−(% Survival). Interferon gamma release in the media supernatant of co-cultured TILs was analyzed by ELISA, and LAMP1 (CD107a, clone eBioH4A3) expression on TILs was analyzed on a flow cytometer to evaluate the cytotoxic potency of TILs.

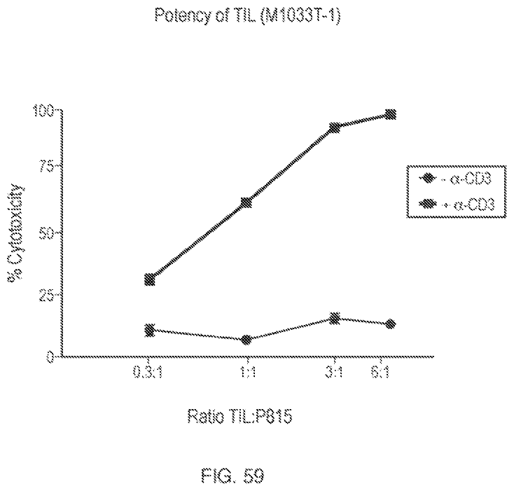

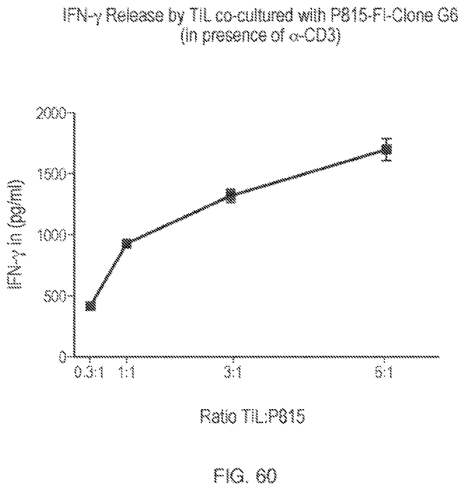

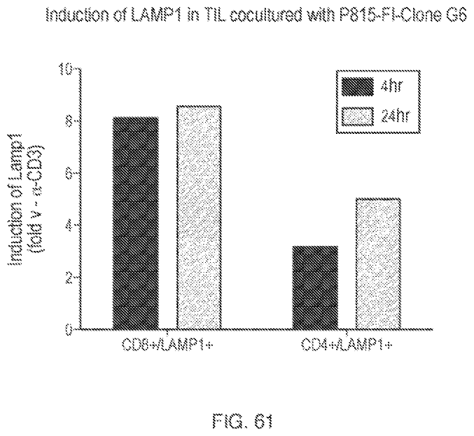

Results are shown in FIG. 59 to FIG. 75. FIG. 59 illustrates percent toxicity of TIL batch M1033T-1 co-cultured with P815 Clone G6 (with and without anti-CD3) at individual effector:target ratios by BRLA. FIG. 60 illustrates enzyme-linked immunosorbent assay (ELISA) data showing the amount of IFN-γ released against different ratios of effector to target cells. FIG. 61 illustrates LAMP1(%) expressed by TIL batch M1033T-1 when co-cultured with P815 Clone G6 in the presence of anti-CD3 at a ratio of 1:1 effector to target cells for 4 hours and 24 hours co-culture.

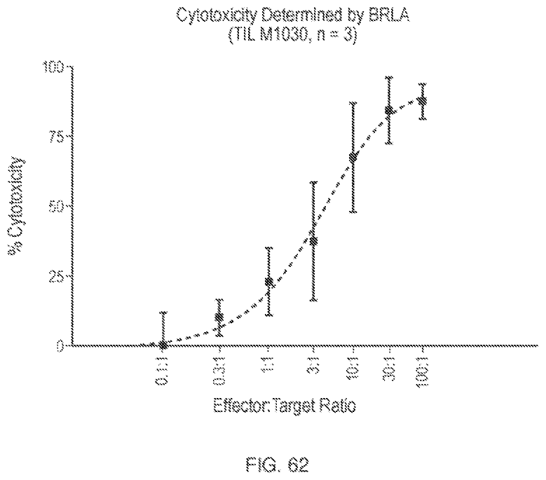

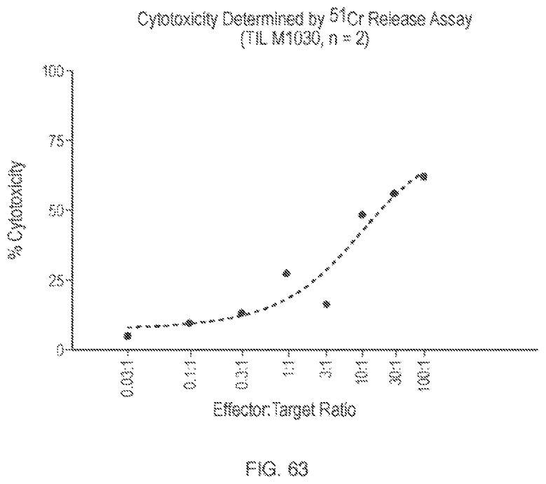

The results were confirmed using a second TIL batch as shown in FIG. 62, which illustrates BRLA for TIL batch M1030. The cytotoxicity (measured as LU₅₀/1×10⁶ TIL) by BRLA is 26±16. FIG. 63 illustrates the results of a standard chromium release assay for TIL batch M1030. The cytotoxicity (measured as LU₅₀/1×10⁶ TIL) by chromium release assay is 22.

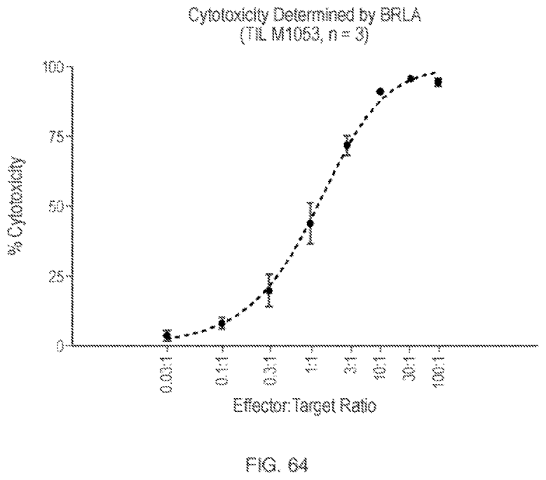

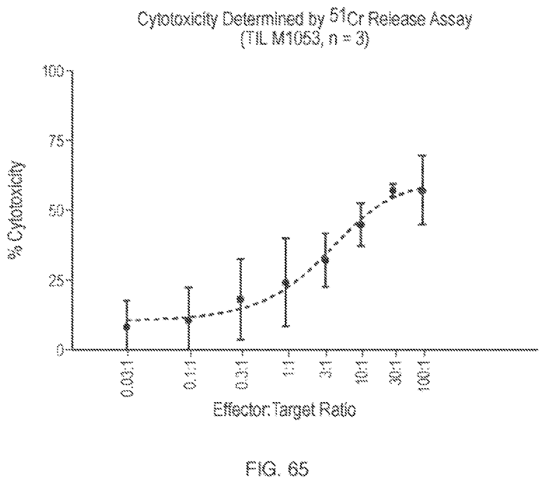

Results were further confirmed using a third TIL batch. FIG. 64 illustrates BRLA results for TIL batch M1053, showing lytic units of the TILs by BRLA as 70±17. FIG. 65 illustrates the results of a standard chromium release assay for TIL batch M1053, showing lytic unit of the TILs by chromium assay as 14±5. Comparison of two assay results shows the comparable performance of the BRLA result to the chromium release assay result.

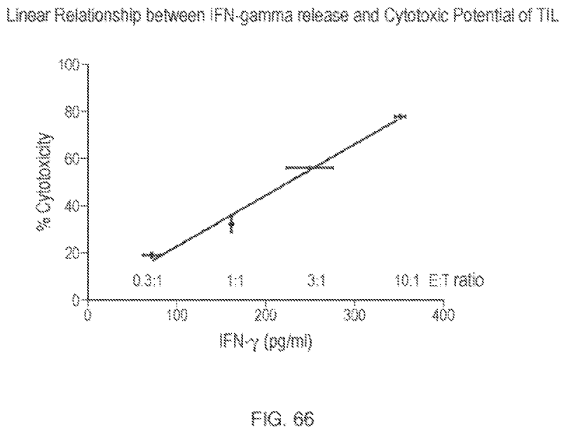

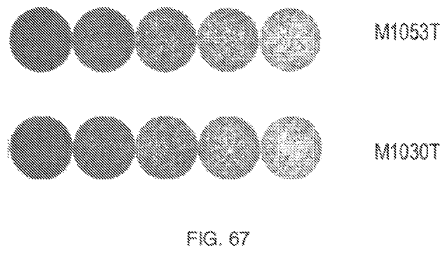

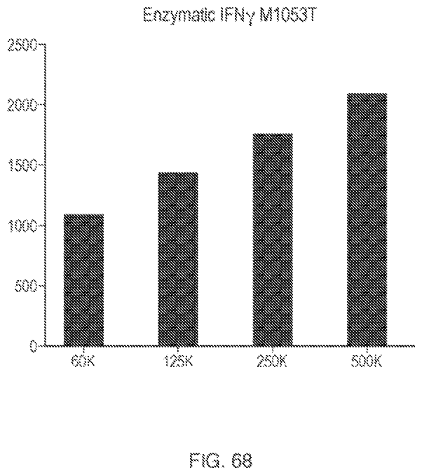

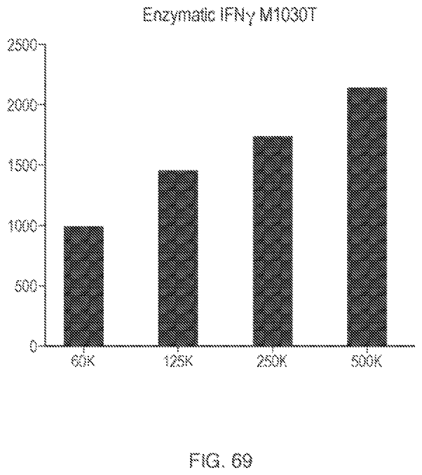

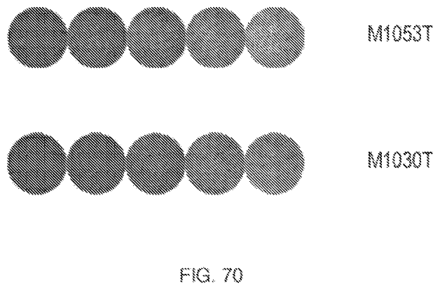

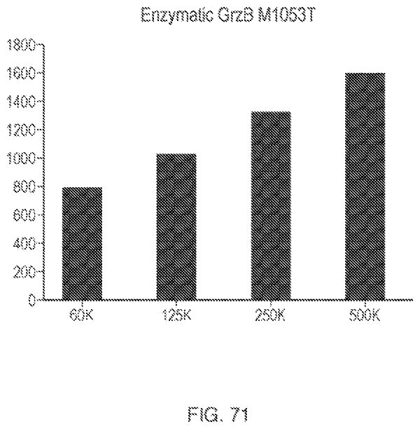

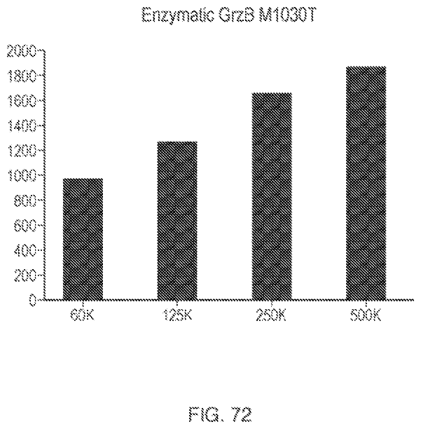

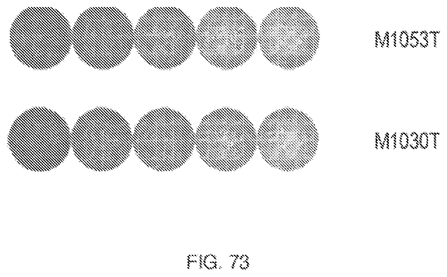

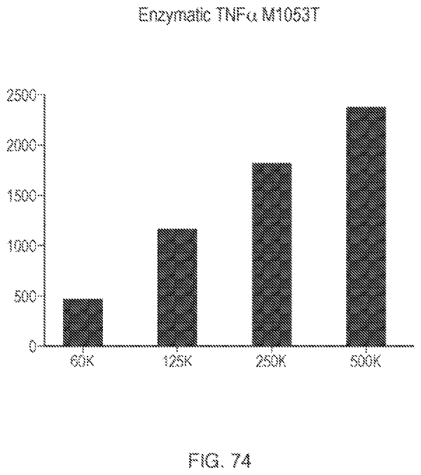

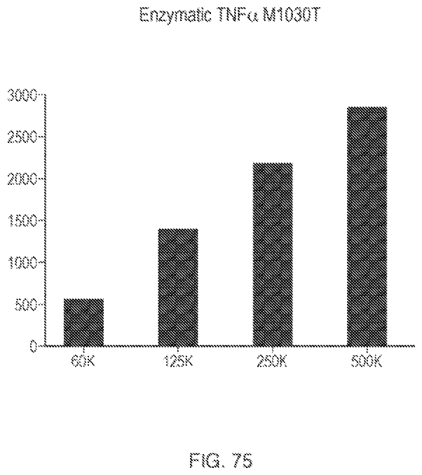

FIG. 66 illustrates the linear relationship between IFN-γ release and cytotoxic potential of TILs. FIG. 67 illustrates ELISpot results for IFN-γ. FIG. 68 illustrates enzymatic IFN-γ release for TIL batch M1053. FIG. 69 illustrates enzymatic IFN-γ release for TIL batch M1030. FIG. 70 illustrates ELISpot data showing Granzyme B release by M1053T and M1030T. FIG. 71 illustrates enzymatic Granzyme B release for TIL batch M1053. FIG. 72 illustrates enzymatic Granzyme B release for TIL batch M1030. FIG. 73 illustrates ELISpot data showing TNF-α release by M1053T and M1030T. FIG. 74 illustrates enzymatic TNF-α release for TIL batch M1053. FIG. 75 illustrates enzymatic TNF-α release for TIL batch M1030. The data in FIG. 66 to FIG. 76 confirms the potency of these batches of TILs as also shown by the BRLA.

In conclusion, the BRLA requires no radionuclides and is as efficient and sensitive as traditional cytotoxicity assays. Flow cytometric assessment of Lamp1 expression on TILs at individual time points demonstrates degranulation of cytotoxic T cells relative to the potency shown by BRLA. The BRLA demonstrates similar to better potency than standard chromium release assay. BRLA also enables evaluation of the potency of TIL lytic activity. Comparison of BRLA with chromium release assay shows the efficiency and reliability of BRLA. BRLA has a linear relationship with IFNγ release by TILs. Release assay of IFN-γ, TNFα and Granzyme B by ELISpot is consistent with the cytotoxic efficiency of the TILs evaluated by BRLA.

### Example 11—Process for Weaning EM3 Cells from FBS to hAB Serum

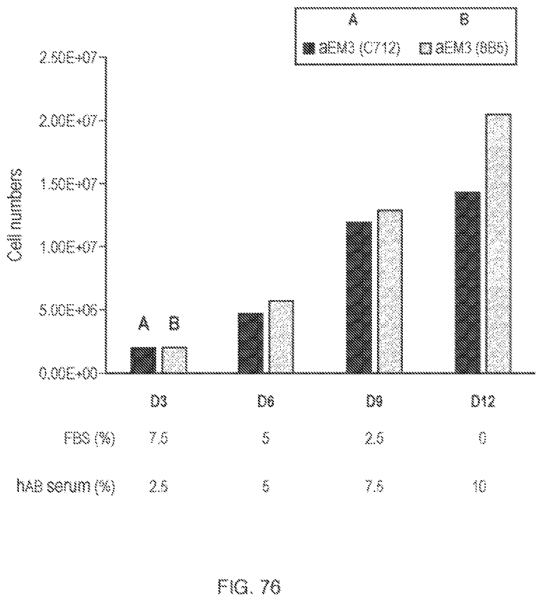

In order to avoid reactivity, some cell lines may need to be weaned from one medium to another. Here, EM3 cells are weaned from FBS to hAB serum to avoid reactivity. As shown in FIG. 76, aEM3 cells were successfully weaned off of FBS to hAB serum.

### Example 12—Freezing Media Formulation Optimization

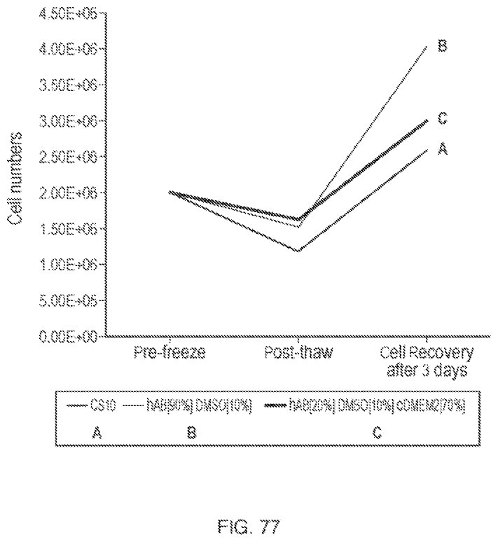

To cryobank EM3 cells cultured as described herein, methods were freezing media formulation were optimized. As shown in FIG. 77, three freezing media were used and their effect on cell numbers were counted. The cell media utilized included CryStor 10 (Biolife Solutions (CS10)) (A), hAB [90%] and DMSO [10%] (B), and hAB [20%] with DMSO [10%] and cDMEM2 [70%] (C). FIG. 77 demonstrates that the formulation of human AB serum (90%) and DMSO (10%) provided for unexpectedly increased EM3 cell numbers after 3 days of recovery.

### Example 13—Growth of aEM3 Cells in GREX Flasks

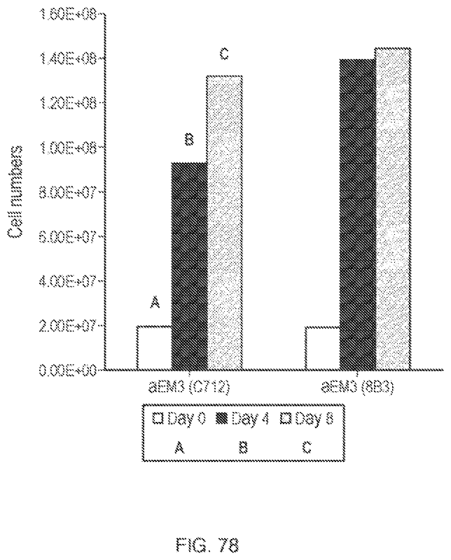

aEM3 cells were cultured in gas permeable cell culture flasks (i.e., GREX flasks (Wilson Wolf Manufacturing)) and the effect on cell doubling time was observed over an 8 day time course. As shown in FIG. 78, the GREX flasks provided for rapid growth of aEM3 cells.

### Example 14—Flow Panel Analysis to Determine aEM3 Cell Purity

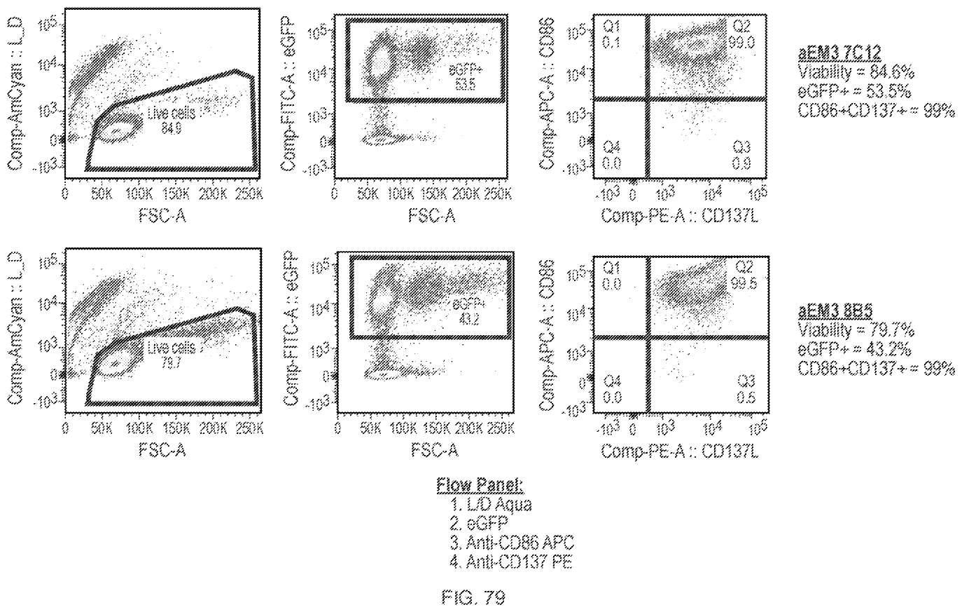

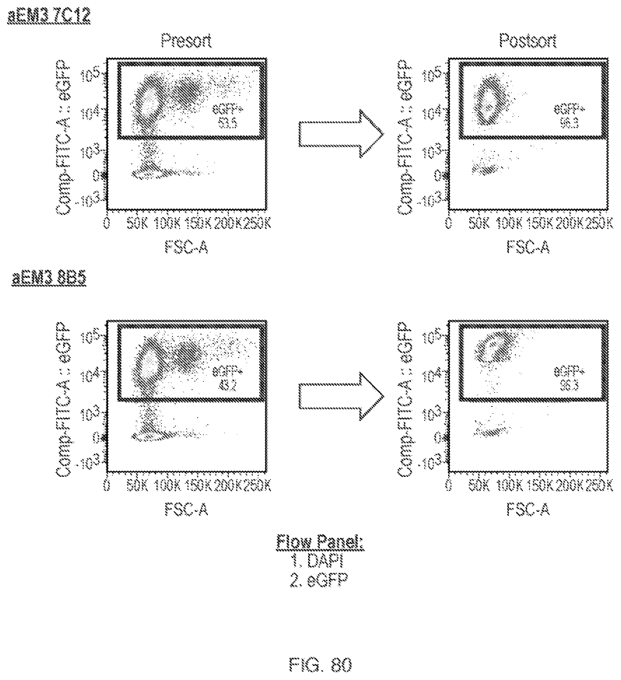

To determine the purity of cells cultured according to the processes described herein, a flow panel analysis was used to determine the purity of aEM3 aAPCs. The results of such analysis are described in FIGS. 79 and 80. As shown in FIG. 80, before sorting aEM3 cell populations were 53.5% and 43.2% eGFP+ for aEM3 7C12 and aEM3 8B5 cells, respectively. Postsorting, cell populations was improved to 96.8% and 96.3% eGFP+ for aEM3 7C12 and aEM3 8B5 cells, respectively (FIG. 80).

### Example 15—aEM3 Feeder Cells as an Alternative to PBMC Feeders

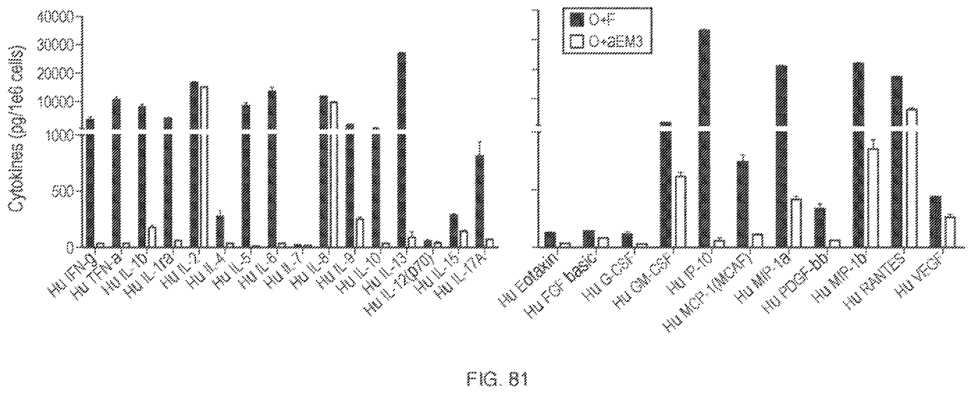

As described herein, aEM3 cells may be used as an alternative for PBMC feeders, resulting in unexpectedly different properties for both TIL expansion process and the resulting TILs. To compare differences in cytokine expression, PBMCs and aEM3 cells were stimulated by treatment with OKT-3. As shown in FIG. 81, aEM3 cells displayed a comparatively different cytokine expression profile as compared to PBMCs. Surprisingly, the aEM3 cells of the present invention provide efficacious TILs (as shown herein) without reproducing the same cytokine secretion properties of TILs expanded with conventional PBMCs.

### Example 16—Comparison Between Complete Media and Serum Free Media TIL Expansion

In order to optimize the TIL expansion protocols, several TIL expansion experiments were performed as described herein, but with serum free media rather than complete media (CM1).

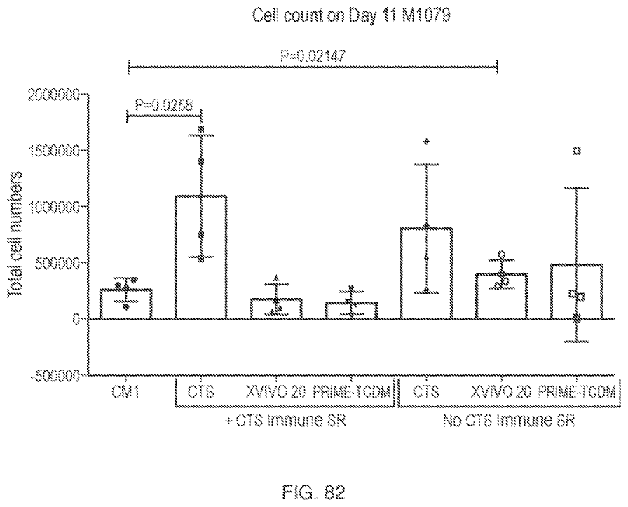

In one experiment, tissue fragments were cultured in a single well with CM1 or various serum free media with 300 IU/mL of IL-2. Cells were then counted on Day 11 before initiating REP. The various serum free media used included Prime CDM (Irvine), CTS Optimizer (ThermoFisher), and Xvivo-20 (Lonza). As shown in FIG. 82, TIL expansion (PreREP) with CTS provided increased cell numbers as compared to CM1.

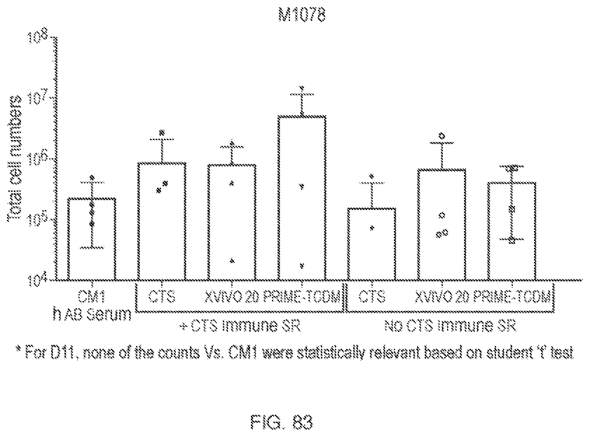

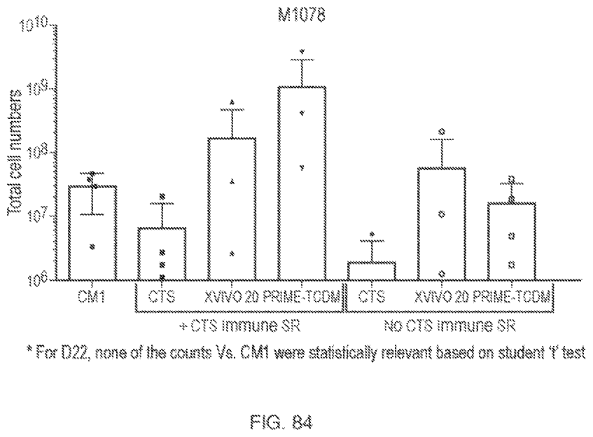

Additionally, tissue fragments were cultured with CM1 or various serum free media with 6000 IU/mL of IL-2 until Day 11. REP was then initiated on Day 11 using PBMC feeders, OKT-3, and IL-2, and culture was split on Day 16. Cultures were then terminated at the end of Day 22. The various serum free media used included Prime CDM (Irvine), CTS Optimizer (ThermoFisher), and Xvivo-20 (Lonza). As shown in FIG. 83 and FIG. 84, when counting cells at Days 11 and Day 22, respectively, TIL expansion (PreREP) with Prime CDM provided increased cell numbers as compared to CM1.

### Example 17—Growth of aAPCs in Serum Free Media as Compared to Serum-Based Media

In order to optimize aAPC growth and maintenance protocols in the absence of serum, aEM3 cells were cultured using various serum free media.

aEM3 cells were cultured in 24 well plates at 1×10⁶ cells per well for 3 days using general cell culture protocols as described herein, with the exception that that one group of cells were provided with serum-based media (cDMEM (10% hSerum) and the other groups of cells were provided with serum free media. The serum free media utilized for the study included CTS OpTmizer (ThermoFisher), Xvivo 20 (Lonza), Prime-TCDM (Irvine), and XFSM (MesenCult) media. Cells were then counted on Day 3.

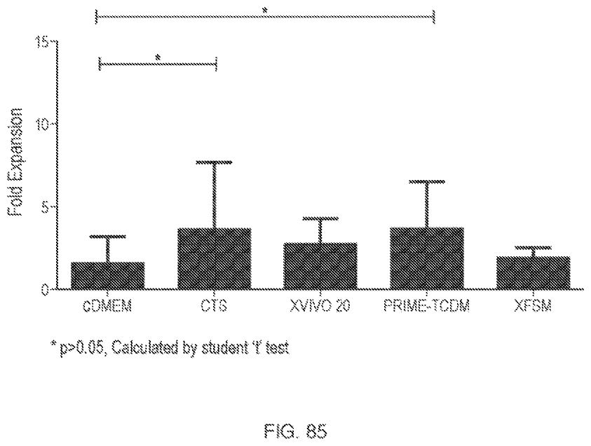

As shown in FIG. 85, CTS OpTmizer and Prime-TCDM serum free media provided cell growth that was comparable to serum-based media (i.e., cDMEM (10% hSerum). Therefore, serum free media is an effective alternative for growing and maintaining aAPCs as compared to serum-based media.

### Example 18—Propagation, Maintenance, and Cryopreservation of aAPCs

In this example, procedures are provided for the preparation and preservation of aAPCs. Specifically, aEM3 cells from a cell line designated TIL-Rs3 were propagated and cryopreserved.

Thawing and recovery of aEM3 cells may be accomplished using the following non-limiting procedure. Cyropreserved aEM3 cells are warmed slowly in pre-warmed media (37° C.) that is prepared from CTS OpTmizer Basal Media (Thermo Fisher), CTS OpTmizer Cell Supplement (Thermo Fisher), Gentamicin (Lonza), and Glutamax (Life Technologies). The suspended cells are then centrifuged at 1500 rpm for 5 minutes at 4° C. The resulting supernatant is discarded and the remaining aEM3 cells are resuspended in the foregoing media and plated (5×10⁶ cells/10 mL per well of a 6 well plate).

Propagation of aEM3 cells may be accomplished using the following non-limiting procedure. Aliquots of the foregoing media are prepared in gas permeable cell culture flasks (i.e., GREX 10 flasks (Wilson Wolf Manufacturing)). The plated aEM3 cells are washed by centrifugation (i.e., 1500 rpm for 5 minutes at 4° C.), resuspended in media, and added to the GREX flasks at cell density of 1-2×10⁶ cells/mL. The aEM3 cell suspension was diluted with 30 mL of media and the GREX flasks were then incubated for 3-4 days at 37° C. under CO₂. After 3-4 days, the GREX flasks were removed from the incubator and placed in a biological safety cabinet (BSC). The cultured aEM3 cells are carefully extracted from the GREX flasks by pipette and the resulting extraction is centrifuged to provide the increased number of aEM3 cells, which may be resuspended at a cell density of 10-20×10⁶ cells per GREX 10 flask.

An alternative cryopreservation of aEM3 cells may be accomplished using the following non-limiting procedure. The foregoing GREX 10 flasks containing the aEM3 cells are removed from the incubator and placed in a BSC. The cultured aEM3 cells are carefully extracted from the GREX flasks by pipette and the resulting extraction is centrifuged to provide the increased number of aEM3 cells, which is resuspended in a volume of CryStor 10 (Biolife Solutions) to provide a concentration of 10-100×10⁶ cells/vial in cryovials. The aEM3 cell suspensions may be placed in a freezing container and transferred to a −80° C. freezer.

### Example 19—Demonstration of Rapid Recovery of aEM3 Cells Following Cryopreservation

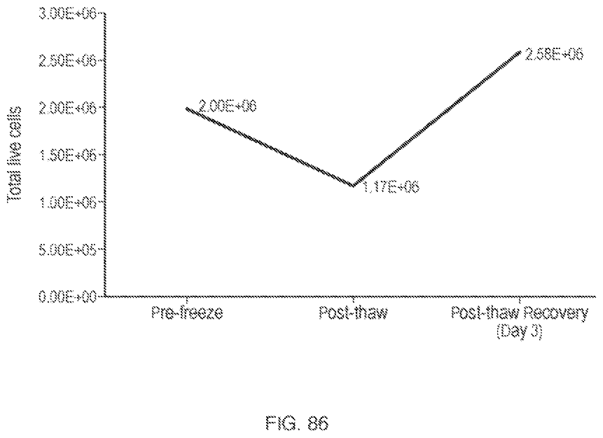

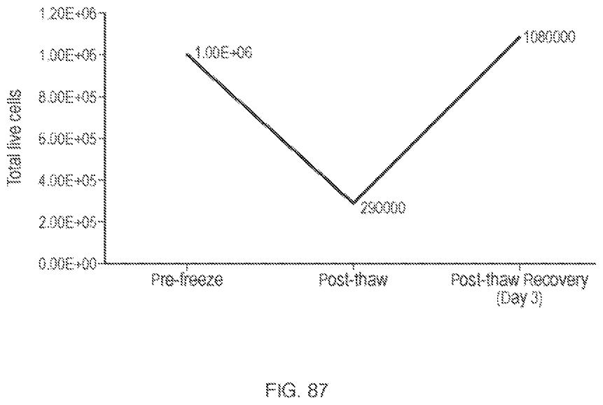

aEM3 cells from the TIL-R3 cell line (1-2×10⁶ cells) were cryopreserved according to the procedure set forth in Example 18 using CS-10 cryopreservation media. Vials of such cells were then thawed and the cells were counted. Cell counts were taken pre-freeze, post-thaw, and 3 days after thaw (i.e., Post-Thaw Recovery). As shown in FIG. 86 and FIG. 87, the total live cell counts recovered rapidly post thaw in two separate experiments.

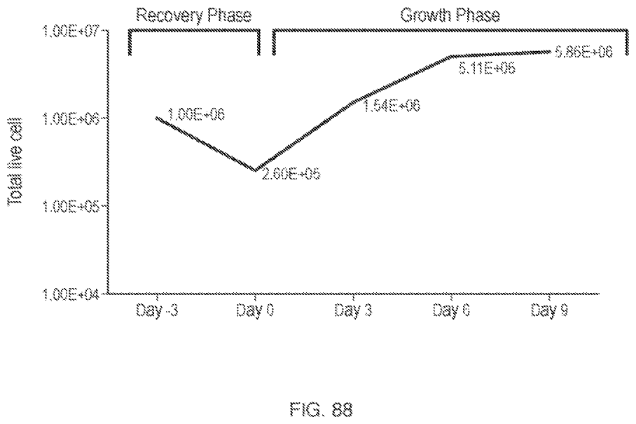

TIL-R3 cells (1×10⁶ cells) were thawed (Day 3 post-thaw) and plated at a density of 0.5×10⁶/cm² in each well of a 24 well plate. On day 0 and 3, viable cells were counted and recorded. On the first passage (Day 6), cells were split at the density of 2×10⁶ cells/cm² or 0.5×10⁶ cells/cm². At the end of the first passage, a cell count was performed. The resulting cell counts are shown in FIG. 88, which demonstrate both a recovery phase post-thaw and a growth phase.

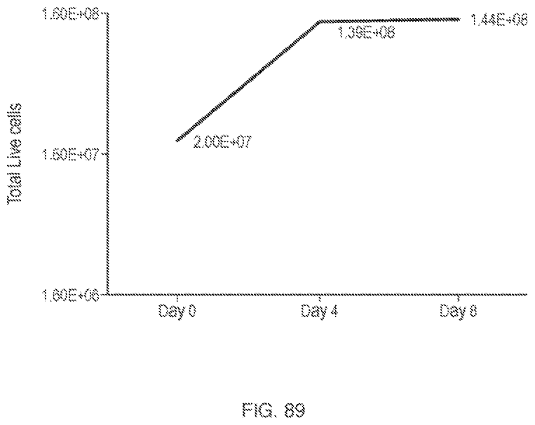

Furthermore, TIL-R3 cells (20×10⁶ cells) were cultured at a density of 2×10⁶/cm² in GREX 10 flasks according to the procedure described in Example 18. On days 4 and 8, live cells were counted and recorded. The resulting cell counts are shown in FIG. 89, which demonstrates a growth phase for the cells following cryopreservation that reaches a plateau between days 4 and 8 when the cells reached a density of 13.9×10⁶ cells/cm².

### Example 20—CD8 Skewness, Expansion Performance, and CD3 Contamination of TILs Cultured with aEM3 aAPCs

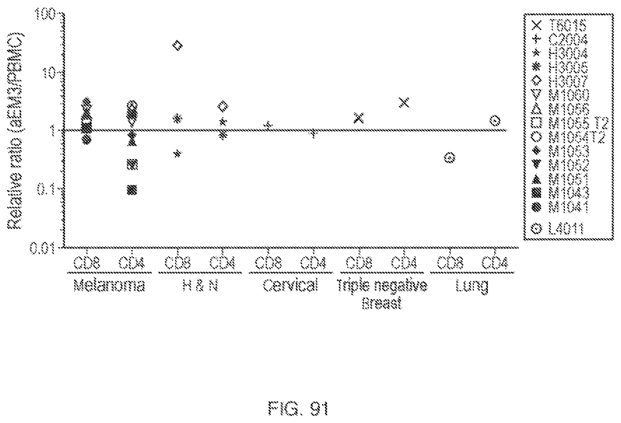

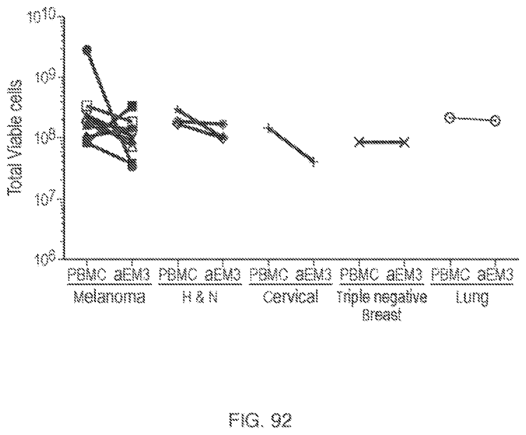

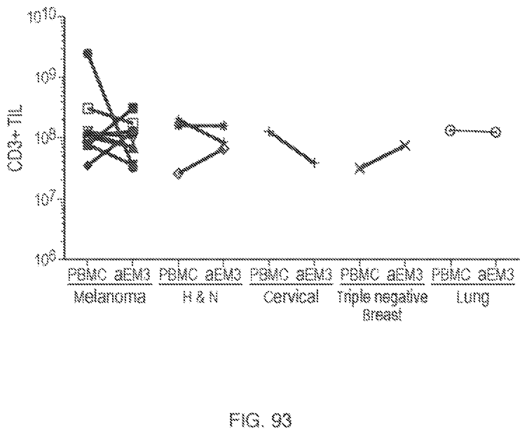

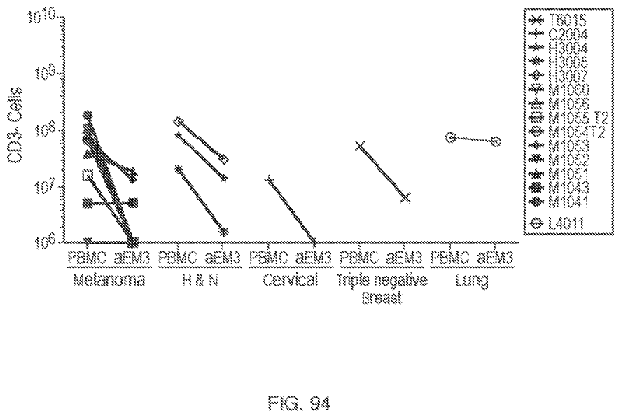

Fifteen different PreREP TIL lines (0.4×10⁵ cells) were co-cultured with either aEM3 aAPCs (as described herein) or PBMC feeders (10×10⁶), OKT3 (30 ng/mL) and IL-2 (3000 IU/mL) and cultures were split on Day 5 using 6 well Grex plates. Cultures were sampled at day 11 and analyzed by flow cytometry. The relative ratio of CD8⁺ cells was calculated by the formula (CD8% aEM3)/(CD8% PBMC). The results shown in FIG. 91 indicate that TILs cultured with aEM3 cells surprisingly promote CD8⁺ skewing and an improved TIL product. Additional results of these experiments are shown in FIG. 92, FIG. 93, and FIG. 94, where the results shown that TILs cultured with aEM3 aAPCs displayed comparable expansion and less non-CD3+ cell contamination in comparison to TILs cultured with PBMC feeders.

### Example 21—Telomere Length Measurement

Genomic DNA was isolated from pre-REP or post-REP (magnetic bead sorted for CD3⁺) TILs for a qPCR (quantitative polymerase chain reaction) assay to measure telomere length. The real time qPCR method is described in Cawthon, Nucleic Acids Res. 2002, 30 (10), e47; and Yang, et al., Leukemia, 2013, 27, 897-906. Briefly, the telomere repeat copy number to single gene copy number (T/S) ratio was determined using an PCR thermal cycler (Bio-Rad Laboratories, Inc.) in a 96-well format. Ten ng of genomic DNA was used for either the telomere or hemoglobin (hgb) PCR reaction and the primers used were as follows:

Tel-1b primer
(SEQ ID NO: 40)
(CGG TTT GTT TGG GTT TGG GTT TGG GTT TGG GTT TGG
GTT);
Tel-2b primer
(SEQ ID NO: 41)
(GGC TTG CCT TAC CCT TAC CCT TAC CCT TAC CCT TAC
CCT);
hgb1 primer
(SEQ ID NO: 42)
(GCT TCT GAC ACA ACT GTG TTC ACT AGC);
and
hgb2 primer
(SEQ ID NO: 43)
(CAC CAA CTT CAT CCA CGT TCA CC).

All samples were analyzed by both the telomere and hemoglobin reactions, and the analysis was performed in triplicate on the same plate. In addition to the test samples, each 96-well plate contained a five-point standard curve from 0.08 ng to 250 ng using genomic DNA isolated from the 1301 human T-cell leukemia cell line (available from Sigma and ATCC). The T/S ratio (−dCt) for each sample was calculated by subtracting the median hemoglobin threshold cycle (Ct) value from the median telomere Ct value. The relative T/S ratio (−ddCt) was determined by subtracting the T/S ratio of the 10.0 ng standard curve point from the T/S ratio of each unknown sample.

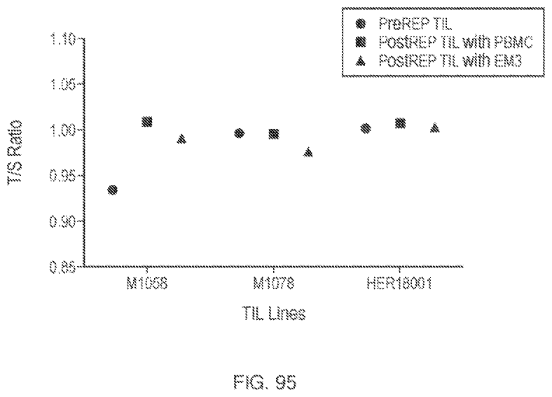

Results are shown in FIG. 95. Each data point shown is the median measurement of relative T/S ratio. The results indicate that TILs cultured with aEM3 maintain their telomere length.

1. (canceled)

2. An isolated artificial antigen presenting cell (aAPC) comprising a K562 myeloid cell, an MOLM14 cell, or an EM-3 cell, wherein the aAPC is stably transduced with one or more viral vectors comprising a nucleic acid encoding a 4-1BBL polypeptide, wherein the aAPC expresses the 4-1BBL polypeptide on its surface, wherein the aAPC is capable of expanding a tumor infiltrating lymphocyte (TIL), when co-cultured with the aAPC, into a population of TILs, wherein the aAPC preferentially increases CD8+ TILs, and wherein
i) the K562 myeloid cell endogenously expresses CD58;
ii) the MOLM14 cell endogenously expresses CD64; and/or
iii) the EM-3 cell endogenously expresses ICOS-L.

3. The isolated aAPC of claim 2, comprising a K562 myeloid cell.

4. The isolated aAPC of claim 2, wherein the one or more viral vectors comprise a lentiviral or retroviral vector.

5. The isolated aAPC of claim 2, wherein the one or more viral vectors further comprise a nucleic acid encoding an interleukin.

6. The isolated aAPC of claim 5, wherein the interleukin is IL-21.

7. The isolated aAPC of claim 2, wherein the 4-1BBL polypeptide comprises an amino acid sequence having at least 90% identity to the amino acid sequence depicted in SEQ ID NO: 9.

8. The isolated aAPC of claim 2, wherein the 4-1BBL polypeptide comprises the amino acid sequence depicted in SEQ ID NO: 9.

9. The isolated aAPC of claim 2, wherein the nucleic acid comprises the nucleic acid sequence depicted in SEQ ID NO: 16.

10. The isolated aAPC of claim 2, capable of increasing IFN-γ release and/or Granzyme B release from the expanded population of TILs.

11. The isolated aAPC of claim 10, wherein the isolated aAPC is more effective than a PBMC feeder cell for increasing IFN-γ release and/or Granzyme B release from the expanded population of TILs.

12. The isolated aAPC of claim 2, wherein the co-culturing is performed with a cell culture medium comprising IL-2.

13. The isolated aAPC of claim 2, wherein the co-culturing is performed with a cell culture medium comprising OKT-3.

14. The isolated aAPC of claim 2, wherein the EM-3 cell further endogenously expresses CD58 and one or more of HLA (human leukocyte antigen)-A, HLA-B, or HLA-C.

15. The isolated aAPC of claim 2, wherein the one or more viral vectors further comprise a nucleic acid encoding one or more of: CD86, OX40L, and an anti-OKT-3 antibody or antibody fragment.

16. An isolated artificial antigen presenting cell (aAPC) comprising a K562 myeloid cell endogenously expressing CD58, wherein the aAPC expresses, via stable transduction with one or more viral vectors, 4-1BBL and IL-21, wherein the aAPC expresses the 4-1BBL on its surface, wherein the aAPC is capable of expanding a tumor infiltrating lymphocyte (TIL), when co-cultured with the TIL, into a population of TILs, and wherein the aAPC preferentially increases CD8+ TILs.

17. A method of expanding tumor infiltrating lymphocytes (TILs), the method comprising the step of contacting a population of TILs with a population of the isolated aAPC of claim 16, wherein the population of TILs is expanded, and wherein the expanded TILs are preferentially CD8+ TILs.

18. A method of expanding tumor infiltrating lymphocytes (TILs), the method comprising the step of contacting a population of TILs with a population of the isolated aAPC of claim 2, wherein the population of TILs is expanded, and wherein the expanded TILs are preferentially CD8+ TILs.

19. The method of claim 18, wherein the expanded TILs exhibit an increased IFN-γ release and/or Granzyme B release.

20. The method of claim 18, wherein the expansion is performed using a gas permeable container.

21. The method of claim 18, comprising
(a) obtaining a first population of TILs from a tumor sample from a patient having a cancer; and
(b) performing a rapid expansion (REP) of the first population of TILs using the population of the isolated aAPCs.

22. The method of claim 18, comprising
(a) obtaining a first population of TILs from a tumor sample from a patient;
(b) performing an initial expansion (pre-REP) of the first population of TILs into a second population of TILs; and
(c) performing a rapid expansion (REP) of the second population of TILs into a third population of TILs, using the population of the isolated aAPCs.

23. The method of claim 22, wherein the initial expansion in step (b) is performed with a cell culture medium comprising IL-2.

24. The method of claim 22, wherein the initial expansion in step (b) is performed for about 1 to about 3 weeks, and wherein the rapid expansion in step (c) is performed for about or less than 14 days.

25. The method of claim 18, further comprising cryopreserving the expanded TILs.

26. A method of treating a patient having a cancer with a population of tumor infiltrating lymphocytes (TILs), comprising the steps of:
(a) obtaining a first population of TILs from a tumor sample from the patient;
(b) performing a rapid expansion (REP) of the first population of TILs into a second population of TILs, using a population of the isolated aAPCs of claim 2; and
(c) administering a therapeutically effective portion of the second population of TILs to the patient.

27. A method of treating a patient having a cancer with a population of tumor infiltrating lymphocytes (TILs), comprising the steps of:
(a) obtaining a first population of TILs from a tumor sample from the patient;
(b) performing an initial expansion (pre-REP) of the first population of TILs into a second population of TILs; and
(c) performing a rapid expansion (REP) of the second population of TILs into a third population of TILs, using a population of the isolated aAPCs of claim 2; and
(d) administering a therapeutically effective portion of the third population of TILs to the patient.

28. The method of claim 26, wherein the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer, renal cancer, renal cell carcinoma, pancreatic cancer, and glioblastoma.

29. The method of claim 27, wherein the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer, renal cancer, renal cell carcinoma, pancreatic cancer, and glioblastoma.

30. A method of treating a patient having a cancer with a population of tumor infiltrating lymphocytes (TILs), comprising the steps of:
(a) obtaining a first population of TILs from a tumor sample from the patient;
(b) performing a rapid expansion (REP) of the first population of TILs into a second population of TILs, using a population of the isolated aAPCs of claim 16; and
(c) administering a therapeutically effective portion of the second population of TILs to the patient.


SEQUENCE LISTING (43 sequences)

SEQ ID NO: 1 - protein, 450 aa, synthetic construct
  REGION 1..450: note = Muromonab heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, synthetic construct
  REGION 1..213: note = Muromonab light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, synthetic construct
  REGION 1..134: note = recombinant human IL-2
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, synthetic construct
  REGION 1..132: note = aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 153 aa, synthetic construct
  REGION 1..153: note = recombinant human IL-7
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 6 - protein, 115 aa, synthetic construct
  REGION 1..115: note = recombinant human IL-15
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 132 aa, synthetic construct
  REGION 1..132: note = IL-21
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 8 - protein, 323 aa, synthetic construct
  REGION 1..323: note = human CD86
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 9 - protein, 254 aa, synthetic construct
  REGION 1..254: note = human 4-1BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 183 aa, synthetic construct
  REGION 1..183: note = human OX40L
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 11 - protein, 220 aa, synthetic construct
  REGION 1..220: note = human CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 12 - protein, 223 aa, synthetic construct
  REGION 1..223: note = human CTLA-4
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 13 - protein, 255 aa, synthetic construct
  REGION 1..255: note = human 4-1BB
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 14 - protein, 277 aa, synthetic construct
  REGION 1..277: note = human OX40
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 15 - DNA, 7569 bp, synthetic construct
  misc_feature 1..7569: note = pLV430G human 4-1BBL vector
  source 1..7569: mol_type = other DNA
cgataaccctaattcgatagcatatgcttcccgttgggtaacatatgctattgaattagggttagtctggatagtatatactactacccgggaagcatatgctacccgtttagggttcaccggtgatgccggccacgatgcgtccggcgtagaggatctaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttttaaaagaaaaggggggattggggggtacagtgcaggggaaagaatagtagacataatagcaacagacatacaaactaaagaattacaaaaacaaattacaaaaattcaaaattttatcgattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgatatcaacaagtttgtacaaaaaagcaggcttcgccaccatggaatacgcctctgatgccagcctggaccccgaagctccttggcctcctgcccctagagccagagcctgtagagtgctgccttgggctctggtggctggccttctccttctgctgctgctggccgctgcctgcgctgtgtttctggcttgtccttgggccgtgtcaggcgccagagcttctcctggatctgccgccagccccagactgagagagggacctgagctgagccccgatgatcctgccggactgctggatctgagacagggcatgttcgcccagctggtggcccagaacgtgctgctgatcgatggccccctgagctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatccggctctgtgtctctggcactgcatctgcagcccctgagatctgctgcaggcgctgctgcactggccctgacagtggacctgcctccagcctctagcgaggccagaaactccgcattcgggtttcaaggcagactgctgcacctgtctgccggccagagactgggagtgcatctgcacacagaggccagagccagacacgcctggcagctgacacagggcgctacagtgctgggcctgttcagagtgacccccgaaattccagccggcctgcccagccctagaagcgagtaggacccagctttcttgtacaaagtggtgattcgagttaattaagctagcctagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatggcgcaatcactagagcggggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccggatcccttgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcctttttgaagctgtccctgatggtcgtcatctacctgcctggacagcatggcctgcaacgcgggcatcccgatgccgccggaagcgagaagaatcataatggggaaggccatccagcctcgcgtcg

SEQ ID NO: 16 - DNA, 765 bp, synthetic construct
  misc_feature 1..765: note = 4-1BBL CoOP
  source 1..765: mol_type = other DNA
atggaatacgcctctgatgccagcctggaccccgaagctccttggcctcctgcccctagagccagagcctgtagagtgctgccttgggctctggtggctggccttctccttctgctgctgctggccgctgcctgcgctgtgtttctggcttgtccttgggccgtgtcaggcgccagagcttctcctggatctgccgccagccccagactgagagagggacctgagctgagccccgatgatcctgccggactgctggatctgagacagggcatgttcgcccagctggtggcccagaacgtgctgctgatcgatggccccctgagctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatccggctctgtgtctctggcactgcatctgcagcccctgagatctgctgcaggcgctgctgcactggccctgacagtggacctgcctccagcctctagcgaggccagaaactccgcattcgggtttcaaggcagactgctgcacctgtctgccggccagagactgggagtgcatctgcacacagaggccagagccagacacgcctggcagctgacacagggcgctacagtgctgggcctgttcagagtgacccccgaaattccagccggcctgcccagccctagaagcgagtag

SEQ ID NO: 17 - DNA, 832 bp, synthetic construct
  misc_feature 1..832: note = 4-1BBL PRCP
  source 1..832: mol_type = other DNA
ggggacaagtttgtacaaaaaagcaggcttcgccaccatggaatacgcctctgatgccagcctggaccccgaagctccttggcctcctgcccctagagccagagcctgtagagtgctgccttgggctctggtggctggccttctccttctgctgctgctggccgctgcctgcgctgtgtttctggcttgtccttgggccgtgtcaggcgccagagcttctcctggatctgccgccagccccagactgagagagggacctgagctgagccccgatgatcctgccggactgctggatctgagacagggcatgttcgcccagctggtggcccagaacgtgctgctgatcgatggccccctgagctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatccggctctgtgtctctggcactgcatctgcagcccctgagatctgctgcaggcgctgctgcactggccctgacagtggacctgcctccagcctctagcgaggccagaaactccgcattcgggtttcaaggcagactgctgcacctgtctgccggccagagactgggagtgcatctgcacacagaggccagagccagacacgcctggcagctgacacagggcgctacagtgctgggcctgttcagagtgacccccgaaattccagccggcctgcccagccctagaagcgagtaggacccagctttcttgtacaaagtggtcccc

SEQ ID NO: 18 - DNA, 7776 bp, synthetic construct
  misc_feature 1..7776: note = pLV430G human CD86 vector
  source 1..7776: mol_type = other DNA
cgataaccctaattcgatagcatatgcttcccgttgggtaacatatgctattgaattagggttagtctggatagtatatactactacccgggaagcatatgctacccgtttagggttcaccggtgatgccggccacgatgcgtccggcgtagaggatctaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttttaaaagaaaaggggggattggggggtacagtgcaggggaaagaatagtagacataatagcaacagacatacaaactaaagaattacaaaaacaaattacaaaaattcaaaattttatcgattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgatatcaacaagtttgtacaaaaaagcaggcttcgccaccatgggcctgagcaacatcctgttcgtgatggccttcctgctgtccggagccgcccctctgaagatccaggcctacttcaacgagaccgccgacctgccctgccagttcgccaacagccagaaccagagcctgagcgaactggtggtgttctggcaggaccaggaaaacctggtcctgaacgaggtgtacctgggcaaagaaaagttcgacagcgtgcacagcaagtacatgggccggaccagcttcgacagcgacagctggaccctgcggctgcacaacctgcagatcaaggacaagggcctgtaccagtgcatcatccaccacaagaaacccaccggcatgatcagaatccaccagatgaacagcgagctgtccgtgctggccaacttcagccagcccgagatcgtgcccatcagcaacatcaccgagaacgtgtacatcaacctgacctgcagcagcatccacggctaccccgagcccaagaaaatgagcgtgctgctgcggaccaagaacagcaccatcgagtacgacggcgtgatgcagaaaagccaggacaacgtgaccgagctgtacgacgtgagcatcagcctgagcgtgagcttccccgacgtgaccagcaacatgaccatcttttgcatcctggaaaccgacaagacccggctgctgtccagccccttcagcatcgagctggaagatccccagccccctcccgaccacatcccctggatcaccgccgtgctgcccaccgtgatcatctgcgtgatggtgttctgcctgatcctgtggaagtggaagaagaagaagcggcctaggaacagctacaagtgcggcaccaacaccatggaacgggaggaaagcgagcagaccaagaagcgggagaagatccacatccccgagcggagcgacgaggcccagcgggtgttcaagagcagcaagaccagcagctgcgacaagagcgacacctgcttctaggacccagctttcttgtacaaagtggtgattcgagttaattaagctagcctagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatggcgcaatcactagagcggggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccggatcccttgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcctttttgaagctgtccctgatggtcgtcatctacctgcctggacagcatggcctgcaacgcgggcatcccgatgccgccggaagcgagaagaatcataatggggaaggccatccagcctcgcgtcg

SEQ ID NO: 19 - DNA, 969 bp, synthetic construct
  misc_feature 1..969: note = CD86 CoOP
  source 1..969: mol_type = other DNA
atgggcctgagcaacatcctgttcgtgatggccttcctgctgtccggagccgcccctctgaagatccaggcctacttcaacgagaccgccgacctgccctgccagttcgccaacagccagaaccagagcctgagcgaactggtggtgttctggcaggaccaggaaaacctggtcctgaacgaggtgtacctgggcaaagaaaagttcgacagcgtgcacagcaagtacatgggccggaccagcttcgacagcgacagctggaccctgcggctgcacaacctgcagatcaaggacaagggcctgtaccagtgcatcatccaccacaagaaacccaccggcatgatcagaatccaccagatgaacagcgagctgtccgtgctggccaacttcagccagcccgagatcgtgcccatcagcaacatcaccgagaacgtgtacatcaacctgacctgcagcagcatccacggctaccccgagcccaagaaaatgagcgtgctgctgcggaccaagaacagcaccatcgagtacgacggcgtgatgcagaaaagccaggacaacgtgaccgagctgtacgacgtgagcatcagcctgagcgtgagcttccccgacgtgaccagcaacatgaccatcttttgcatcctggaaaccgacaagacccggctgctgtccagccccttcagcatcgagctggaagatccccagccccctcccgaccacatcccctggatcaccgccgtgctgcccaccgtgatcatctgcgtgatggtgttctgcctgatcctgtggaagtggaagaagaagaagcggcctaggaacagctacaagtgcggcaccaacaccatggaacgggaggaaagcgagcagaccaagaagcgggagaagatccacatccccgagcggagcgacgaggcccagcgggtgttcaagagcagcaagaccagcagctgcgacaagagcgacacctgcttc

SEQ ID NO: 20 - DNA, 1039 bp, synthetic construct
  misc_feature 1..1039: note = CD86 PCRP
  source 1..1039: mol_type = other DNA
ggggacaagtttgtacaaaaaagcaggcttcgccaccatgggcctgagcaacatcctgttcgtgatggccttcctgctgtccggagccgcccctctgaagatccaggcctacttcaacgagaccgccgacctgccctgccagttcgccaacagccagaaccagagcctgagcgaactggtggtgttctggcaggaccaggaaaacctggtcctgaacgaggtgtacctgggcaaagaaaagttcgacagcgtgcacagcaagtacatgggccggaccagcttcgacagcgacagctggaccctgcggctgcacaacctgcagatcaaggacaagggcctgtaccagtgcatcatccaccacaagaaacccaccggcatgatcagaatccaccagatgaacagcgagctgtccgtgctggccaacttcagccagcccgagatcgtgcccatcagcaacatcaccgagaacgtgtacatcaacctgacctgcagcagcatccacggctaccccgagcccaagaaaatgagcgtgctgctgcggaccaagaacagcaccatcgagtacgacggcgtgatgcagaaaagccaggacaacgtgaccgagctgtacgacgtgagcatcagcctgagcgtgagcttccccgacgtgaccagcaacatgaccatcttttgcatcctggaaaccgacaagacccggctgctgtccagccccttcagcatcgagctggaagatccccagccccctcccgaccacatcccctggatcaccgccgtgctgcccaccgtgatcatctgcgtgatggtgttctgcctgatcctgtggaagtggaagaagaagaagcggcctaggaacagctacaagtgcggcaccaacaccatggaacgggaggaaagcgagcagaccaagaagcgggagaagatccacatccccgagcggagcgacgaggcccagcgggtgttcaagagcagcaagaccagcagctgcgacaagagcgacacctgcttctaggacccagctttcttgtacaaagtggtcccc

SEQ ID NO: 21 - DNA, 3526 bp, synthetic construct
  misc_feature 1..3526: note = pDONR221 CD86 vector
  source 1..3526: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacacattgatgagcaatgcttttttataatgcacaagtttgtacaaaaaagcaggcttcgccaccatgggcctgagcaacatcctgttcgtgatggccttcctgctgtccggagccgcccctctgaagatccaggcctacttcaacgagaccgccgacctgccctgccagttcgccaacagccagaaccagagcctgagcgaactggtggtgttctggcaggaccaggaaaacctggtcctgaacgaggtgtacctgggcaaagaaaagttcgacagcgtgcacagcaagtacatgggccggaccagcttcgacagcgacagctggaccctgcggctgcacaacctgcagatcaaggacaagggcctgtaccagtgcatcatccaccacaagaaacccaccggcatgatcagaatccaccagatgaacagcgagctgtccgtgctggccaacttcagccagcccgagatcgtgcccatcagcaacatcaccgagaacgtgtacatcaacctgacctgcagcagcatccacggctaccccgagcccaagaaaatgagcgtgctgctgcggaccaagaacagcaccatcgagtacgacggcgtgatgcagaaaagccaggacaacgtgaccgagctgtacgacgtgagcatcagcctgagcgtgagcttccccgacgtgaccagcaacatgaccatcttttgcatcctggaaaccgacaagacccggctgctgtccagccccttcagcatcgagctggaagatccccagccccctcccgaccacatcccctggatcaccgccgtgctgcccaccgtgatcatctgcgtgatggtgttctgcctgatcctgtggaagtggaagaagaagaagcggcctaggaacagctacaagtgcggcaccaacaccatggaacgggaggaaagcgagcagaccaagaagcgggagaagatccacatccccgagcggagcgacgaggcccagcgggtgttcaagagcagcaagaccagcagctgcgacaagagcgacacctgcttctaggacccagctttcttgtacaaagtggtcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 22 - DNA, 3319 bp, synthetic construct
  misc_feature 1..3319: note = pDONR221 4-1BBL vector
  source 1..3319: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacacattgatgagcaatgcttttttataatgcacaagtttgtacaaaaaagcaggcttcgccaccatggaatacgcctctgatgccagcctggaccccgaagctccttggcctcctgcccctagagccagagcctgtagagtgctgccttgggctctggtggctggccttctccttctgctgctgctggccgctgcctgcgctgtgtttctggcttgtccttgggccgtgtcaggcgccagagcttctcctggatctgccgccagccccagactgagagagggacctgagctgagccccgatgatcctgccggactgctggatctgagacagggcatgttcgcccagctggtggcccagaacgtgctgctgatcgatggccccctgagctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatccggctctgtgtctctggcactgcatctgcagcccctgagatctgctgcaggcgctgctgcactggccctgacagtggacctgcctccagcctctagcgaggccagaaactccgcattcgggtttcaaggcagactgctgcacctgtctgccggccagagactgggagtgcatctgcacacagaggccagagccagacacgcctggcagctgacacagggcgctacagtgctgggcctgttcagagtgacccccgaaattccagccggcctgcccagccctagaagcgagtaggacccagctttcttgtacaaagtggtcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 23 - DNA, 8449 bp, synthetic construct
  misc_feature 1..8449: note = pLV430G vector
  source 1..8449: mol_type = other DNA
cgataaccctaattcgatagcatatgcttcccgttgggtaacatatgctattgaattagggttagtctggatagtatatactactacccgggaagcatatgctacccgtttagggttcaccggtgatgccggccacgatgcgtccggcgtagaggatctaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttttaaaagaaaaggggggattggggggtacagtgcaggggaaagaatagtagacataatagcaacagacatacaaactaaagaattacaaaaacaaattacaaaaattcaaaattttatcgattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgatatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgattcgagttaattaagctagcctagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatggcgcaatcactagagcggggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccggatcccttgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcctttttgaagctgtccctgatggtcgtcatctacctgcctggacagcatggcctgcaacgcgggcatcccgatgccgccggaagcgagaagaatcataatggggaaggccatccagcctcgcgtcg

SEQ ID NO: 24 - DNA, 4761 bp, synthetic construct
  misc_feature 1..4761: note = pDONR221 vector
  source 1..4761: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacacattgatgagcaatgcttttttataatgccaactttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccacgcggcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - DNA, 10703 bp, synthetic construct
  misc_feature 1..10703: note = psPAX2 plasmid
  source 1..10703: mol_type = other DNA
aaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgggccggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaagcaagaatcctagctgtggaaagatacctaaaggatcaacagctcctagggatttggggttgctctggaaaactcatttgcaccactgctgtgccttggaatgctagttggagtaataaatctctggaacagatctggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagctcgaaacaaccggtacctctagaactatagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatca

SEQ ID NO: 26 - DNA, 7298 bp, synthetic construct
  misc_feature 1..7298: note = pCIGO-VSV.G plasmid
  source 1..7298: mol_type = other DNA
gtcgacggatcgggagatcaattccggcacctgtcctacgagttgcatgataaagaagacagtcataagtgcggcgacgatagtcatgccccgcgcccaccggaaggagctgactgggttgaaggctctcaagggcatcggtcgatgcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcaagggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggtaccgagctcggatccactagtaacggccgccagtgtgctggaattcaacagagatcgatctgtttccttgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 27 - protein, 231 aa, synthetic construct
  REGION 1..231: note = mFC-7C12 scFv
QVQLVQSGGGLVKPGGSLRLSCAASGFNFNDQYMSWIRQAPGKGLEWVSFISGSGGTTYYTDSVKGRFTISRDNTKDSLYLQMNSLTVEDTAVYYCARGGNYYTSVGRGTLVTVSAGGGGSGAPDIQMTQSPGTLSLSPGERAILSCRASQSVSGYLAWYQQKPGQAPRLLIYGASSRATGIPDRFSGSGSGTDFTLTISSLRPEDIGTYYCKQYINAPFTFGGGTKVEIK

SEQ ID NO: 28 - protein, 243 aa, synthetic construct
  REGION 1..243: note = mFC-8B3 scFv
QVQLQQSGAEVKKPGSSVKVSCKASGGTFSSYAISWVRQAPGQGLEWMGWISPYNGNTDYAQKVQGRVTLTTDTSTSTAYMELRSLRSDDTAVYYCATGGGTWYSDLWGRGTLVTVSAGGGGSGGGGSGGGGSGAPEIVLTQSPSTLSASVGDRVSITCRASQSIGGSLAWYQQKPGKAPKLLISEASTLERGVPSRFSGSGSGTDFTLTISSLQPEDVATYYCQKYNSVPLTFGPGTKVEIK

SEQ ID NO: 29 - DNA, 693 bp, synthetic construct
  misc_feature 1..693: note = mFC-7C12 scFv
  source 1..693: mol_type = other DNA
caggtgcagctggtgcagtctgggggaggcttggtcaagcctggagggtccctgagactctcctgtgcagcctctggattcaatttcaatgaccagtacatgagttggatccgccaggctccagggaaggggctggagtgggtttcattcattagtggtagtggtggtaccacatactacacagactctgtgaagggccggttcaccatctccagggacaacaccaaggactcattgtatttgcaaatgaacagcctgacagtcgaggacacggccgtgtactactgtgcgagaggagggaattattatacttcggtgggccggggcaccctggtcaccgtctcggccggtggcggcggatctggcgcgccagacatccagatgacccagtctccaggcaccctgtctttgtctccaggggaaagagccatcctctcctgcagggccagtcagagtgttagcggctacctagcctggtatcaacagaaacctggccaggctcccaggctcctcatctatggtgcatccagcagggccactggcatcccagacaggttcagtggcagtgggtctgggacagacttcactctcaccatcagcagcctgcggcctgaagatattggaacatattactgtaaacagtacattaatgccccattcactttcggcggcgggaccaaggtggagatcaaa

SEQ ID NO: 30 - DNA, 729 bp, synthetic construct
  misc_feature 1..729: note = mFC-8B3 scFv
  source 1..729: mol_type = other DNA
caggtacagctgcagcagtcaggggctgaggtgaagaagcctgggtcctcggtgaaggtctcctgcaaggcttctggaggcaccttcagcagctatgctatcagctgggtgcgacaggcccctggacaagggcttgagtggatgggatggatcagcccttacaatggtaacacagattatgcacagaaggtccagggcagagtcaccttgaccacagacacatccacgagcacagcctacatggagctgaggagcctgagatccgacgacacggccgtgtattactgtgcgacaggtggcgggacctggtactccgatctctggggccgtggcaccctggtcaccgtctcggccggtggcggtggcagcggcggtggtgggtccggtggcggcggatctggcgcgccagaaattgtgctgactcagtctccctccaccctgtctgcatctgtaggagacagagtcagcatcacttgccgggccagtcagagtattggtgggtcgttggcctggtatcaacaaaagccagggaaagcccctaagctcctgatctctgaggcgtctactttagagaggggcgtcccatcaagattcagcggcagtggatctgggacagatttcactctcaccatcagcagcctgcagcctgaagatgttgcaacttattactgtcaaaaatataacagtgtcccgctcactttcggccctgggaccaaggtggagatcaaa

SEQ ID NO: 31 - DNA, 9862 bp, synthetic construct
  misc_feature 1..9862: note = destination vector pLV4301G
  source 1..9862: mol_type = other DNA
cgataaccctaattcgatagcatatgcttcccgttgggtaacatatgctattgaattagggttagtctggatagtatatactactacccgggaagcatatgctacccgtttagggttcaccggtgatgccggccacgatgcgtccggcgtagaggatctaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttttaaaagaaaaggggggattggggggtacagtgcaggggaaagaatagtagacataatagcaacagacatacaaactaaagaattacaaaaacaaattacaaaaattcaaaattttatcgattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgatatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgattcgagttaattaagttaacgaattccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggaggcggaagcggcggaggctcccctcgaggcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgcgtcccgggtctagagctagcggtaccatgcattacgtagtcgacgacttaattaagctagcctagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatggcgcaatcactagagcggggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccggatcccttgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcctttttgaagctgtccctgatggtcgtcatctacctgcctggacagcatggcctgcaacgcgggcatcccgatgccgccggaagcgagaagaatcataatggggaaggccatccagcctcgcgtcg

SEQ ID NO: 32 - DNA, 3334 bp, synthetic construct
  misc_feature 1..3334: note = donor vector 1, pMK 7c12 anti mFC scFV CoOp ECORV SacII L1R5
  source 1..3334: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcataaataatgattttattttgactgatagtgacctgttcgttgcaacaaattgatgagcaatgcttttttataatgccaactttgtacaaaaaagctgaacgatatcgccaccatgggcagcacagccattctggccctgctgctggcagtgctgcagggcgtgtcagctcaggtgcagctggtgcagtctggcggcggactcgtgaaacctggcggcagcctgagactgagctgtgccgccagcggcttcaacttcaacgaccagtacatgagctggatccggcaggcccctggcaagggactggaatgggtgtccttcatcagcggcagcggcggcaccacctactacaccgatagcgtgaagggccggttcaccatcagccgggacaacaccaaggacagcctgtacctgcagatgaacagcctgaccgtggaagataccgccgtgtactactgcgccagaggcggcaattactacaccagcgtgggcagaggcaccctcgtgacagtgtctgctggcggaggcggatcaggcggcggaggatcagggggaggcggaagcggagcacccgatatccagatgacacagagccccggcaccctgtctctgagccctggcgaaagagccatcctgagctgcagagccagccagagcgtgtccggatacctggcttggtatcagcagaagcccggccaggcccccagactgctgatctatggcgccagcagcagagccacaggcatccccgatagattcagcggctctggcagcggcaccgacttcaccctgacaatcagctccctgcggcccgaggacatcggcacctactattgcaagcagtacatcaacgcccccttcaccttcggcggaggcaccaaggtggaaatcaagccgcgggccaactttgtatacaaaagtggaacgagaaacgtaaaatgatataaatatcaatatattaaattagattttgcataaaaaacagactacataatactgtaaaacacaacatatccagtcactatgaatcaactacttagatggtattagtgacctgtactgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 33 - DNA, 2779 bp, synthetic construct
  misc_feature 1..2779: note = donor vector 2, pMK hCD8a scaffold TN L5 L2
  source 1..2779: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcataaataatgattttattttgactgatagtgacctgttcgttgcaacaaattgatgagcaatgcttttttataatgcccaactttgtatacaaaagtggcccgcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcggctccaccagcggctccggcaagcccggctctggcgagggctccaccagcggcgactacaaggacgacgatgacaagtaataggatatcggttcagctttcttgtacaaagttggcattataagaaagcattgcttatcaatttgttgcaacgaacaggtcactatcagtcaaaataaaatcattatttctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 34 - DNA, 9338 bp, synthetic construct
  misc_feature 1..9338: note = Final vector used for lentiviral production, pLV4301G 7C12 scFV mIgG hCD8 flag
  source 1..9338: mol_type = other DNA
cgataaccctaattcgatagcatatgcttcccgttgggtaacatatgctattgaattagggttagtctggatagtatatactactacccgggaagcatatgctacccgtttagggttcaccggtgatgccggccacgatgcgtccggcgtagaggatctaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttttaaaagaaaaggggggattggggggtacagtgcaggggaaagaatagtagacataatagcaacagacatacaaactaaagaattacaaaaacaaattacaaaaattcaaaattttatcgattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgatatcaccaactttgtacaaaaaagctgaacgatatcgccaccatgggcagcacagccattctggccctgctgctggcagtgctgcagggcgtgtcagctcaggtgcagctggtgcagtctggcggcggactcgtgaaacctggcggcagcctgagactgagctgtgccgccagcggcttcaacttcaacgaccagtacatgagctggatccggcaggcccctggcaagggactggaatgggtgtccttcatcagcggcagcggcggcaccacctactacaccgatagcgtgaagggccggttcaccatcagccgggacaacaccaaggacagcctgtacctgcagatgaacagcctgaccgtggaagataccgccgtgtactactgcgccagaggcggcaattactacaccagcgtgggcagaggcaccctcgtgacagtgtctgctggcggaggcggatcaggcggcggaggatcagggggaggcggaagcggagcacccgatatccagatgacacagagccccggcaccctgtctctgagccctggcgaaagagccatcctgagctgcagagccagccagagcgtgtccggatacctggcttggtatcagcagaagcccggccaggcccccagactgctgatctatggcgccagcagcagagccacaggcatccccgatagattcagcggctctggcagcggcaccgacttcaccctgacaatcagctccctgcggcccgaggacatcggcacctactattgcaagcagtacatcaacgcccccttcaccttcggcggaggcaccaaggtggaaatcaagccgcgggccaactttgtatacaaaagtggcccgcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcggctccaccagcggctccggcaagcccggctctggcgagggctccaccagcggcgactacaaggacgacgatgacaagtaataggatatcggttcagctttcttgtacaaagttgggattcgagttaattaagttaacgaattccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggaggcggaagcggcggaggctcccctcgaggcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgcgtcccgggtctagagctagcggtaccatgcattacgtagtcgacgacttaattaagctagcctagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatggcgcaatcactagagcggggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccggatcccttgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcctttttgaagctgtccctgatggtcgtcatctacctgcctggacagcatggcctgcaacgcgggcatcccgatgccgccggaagcgagaagaatcataatggggaaggccatccagcctcgcgtcg

SEQ ID NO: 35 - DNA, 9862 bp, synthetic construct; identical to SEQ ID NO: 31
  misc_feature 1..9862: note = destination vector, pLV4301G
  source 1..9862: mol_type = other DNA

SEQ ID NO: 36 - DNA, 3340 bp, synthetic construct
  misc_feature 1..3340: note = donor vector 1, pMK 8B3 anti mFC scFV CoOp ECORV SacII L1R5
  source 1..3340: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcataaataatgattttattttgactgatagtgacctgttcgttgcaacaaattgatgagcaatgcttttttataatgccaactttgtacaaaaaagctgaacgatatcgccaccatgggcagcacagccattctggccctgctgctggcagtgctgcagggcgtgtcagctcaggtgcagctgcagcagtctggcgccgaagtgaagaaacccggcagcagcgtgaaggtgtcctgcaaggctagcggcggcaccttcagcagctacgccatttcttgggtgcgccaggcccctggacagggcctggaatggatgggctggatcagcccctacaacggcaacaccgactacgcccagaaagtgcagggcagagtgaccctgaccaccgacaccagcacctccaccgcctacatggaactgcggagcctgagaagcgacgacaccgccgtgtactactgtgccacaggcggcggaacctggtacagcgatctgtggggcagaggcaccctcgtgacagtgtctgctggcggcggaggatctggcggaggcggaagtggcgggggaggaagcggagcacctgagatcgtgctgacccagagccctagcacactgagcgccagcgtgggcgacagagtgtccatcacctgtagagccagccagagcatcggaggcagcctggcctggtatcagcagaagcctggcaaggcccccaagctgctgatctctgaggccagcaccctggaaagaggcgtgcccagcagattttccggcagcggctctggcaccgacttcaccctgacaatcagcagcctgcagcccgaggacgtggccacctactactgccagaagtacaacagcgtgcccctgaccttcggccctggcaccaaggtggaaatcaagccgcgggccaactttgtatacaaaagtggaacgagaaacgtaaaatgatataaatatcaatatattaaattagattttgcataaaaaacagactacataatactgtaaaacacaacatatccagtcactatgaatcaactacttagatggtattagtgacctgtactgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 37 - DNA, 2779 bp, synthetic construct; identical to SEQ ID NO: 33
  misc_feature 1..2779: note = donor vector 2, pMK hCD8a scaffold TN L5 L2
  source 1..2779: mol_type = other DNA

SEQ ID NO: 38 - DNA, 9344 bp, synthetic construct
  misc_feature 1..9344: note = Final vector used for lentiviral production, pLV4301G 8B3 scFV mIgG hCD8 flag
  source 1..9344: mol_type = other DNA
cgataaccctaattcgatagcatatgcttcccgttgggtaacatatgctattgaattagggttagtctggatagtatatactactacccgggaagcatatgctacccgtttagggttcaccggtgatgccggccacgatgcgtccggcgtagaggatctaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttttaaaagaaaaggggggattggggggtacagtgcaggggaaagaatagtagacataatagcaacagacatacaaactaaagaattacaaaaacaaattacaaaaattcaaaattttatcgattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgatatcaccaactttgtacaaaaaagctgaacgatatcgccaccatgggcagcacagccattctggccctgctgctggcagtgctgcagggcgtgtcagctcaggtgcagctgcagcagtctggcgccgaagtgaagaaacccggcagcagcgtgaaggtgtcctgcaaggctagcggcggcaccttcagcagctacgccatttcttgggtgcgccaggcccctggacagggcctggaatggatgggctggatcagcccctacaacggcaacaccgactacgcccagaaagtgcagggcagagtgaccctgaccaccgacaccagcacctccaccgcctacatggaactgcggagcctgagaagcgacgacaccgccgtgtactactgtgccacaggcggcggaacctggtacagcgatctgtggggcagaggcaccctcgtgacagtgtctgctggcggcggaggatctggcggaggcggaagtggcgggggaggaagcggagcacctgagatcgtgctgacccagagccctagcacactgagcgccagcgtgggcgacagagtgtccatcacctgtagagccagccagagcatcggaggcagcctggcctggtatcagcagaagcctggcaaggcccccaagctgctgatctctgaggccagcaccctggaaagaggcgtgcccagcagattttccggcagcggctctggcaccgacttcaccctgacaatcagcagcctgcagcccgaggacgtggccacctactactgccagaagtacaacagcgtgcccctgaccttcggccctggcaccaaggtggaaatcaagccgcgggccaactttgtatacaaaagtggcccgcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcggctccaccagcggctccggcaagcccggctctggcgagggctccaccagcggcgactacaaggacgacgatgacaagtaataggatatcggttcagctttcttgtacaaagttgggattcgagttaattaagttaacgaattccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggaggcggaagcggcggaggctcccctcgaggcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgcgtcccgggtctagagctagcggtaccatgcattacgtagtcgacgacttaattaagctagcctagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatggcgcaatcactagagcggggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccggatcccttgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcctttttgaagctgtccctgatggtcgtcatctacctgcctggacagcatggcctgcaacgcgggcatcccgatgccgccggaagcgagaagaatcataatggggaaggccatccagcctcgcgtcg

SEQ ID NO: 39 - DNA, 6924 bp, synthetic construct
  misc_feature 1..6924: note = pLenti-C-Myc-DDK OX40L
  source 1..6924: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccatggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtccttacgcgtacgcggccgctcgagcagaaactcatctcagaagaggatctggcagcaaatgatatcctggattacaaggatgacgacgataaggtttaaacggccggccgcggtctgtacaagtaggattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatactcttcctttttcaatattattgaagcatttatcagggttattgtctcatgagcggatacatatttgaatgtatttagaaaaataaacaaataggggtcccgcgcacatttccccgaaaagtgccacctgac

SEQ ID NO: 40 - DNA, 39 bp, synthetic construct
  misc_feature 1..39: note = Tel-1b primer
  source 1..39: mol_type = other DNA
cggtttgtttgggtttgggtttgggtttgggtttgggtt

SEQ ID NO: 41 - DNA, 39 bp, synthetic construct
  misc_feature 1..39: note = Tel-2b primer
  source 1..39: mol_type = other DNA
ggcttgccttacccttacccttacccttacccttaccct

SEQ ID NO: 42 - DNA, 27 bp, synthetic construct
  misc_feature 1..27: note = hgb1 primer
  source 1..27: mol_type = other DNA
gcttctgacacaactgtgttcactagc

SEQ ID NO: 43 - DNA, 23 bp, synthetic construct
  misc_feature 1..23: note = hgb2 primer
  source 1..23: mol_type = other DNA
caccaacttcatccacgttcacc